(19) United States
(12) Patent Application Publication
(10) Pub. No.: US 2026/0049279 A1
(43) Pub. Date: Feb. 19, 2026

(54) ANAPLASTIC LYMPHOMA KINASE (ALK) SPECIFIC T CELL RECEPTORS AND METHODS OF USE THEREOF

(71) Applicant: The Children's Medical Center Corporation, Boston, MA (US)

(72) Inventors: Roberto Chiarle, Boston, MA (US); Rafael B. Blasco-Patino, Boston, MA (US); Carmen Mecca, Boston, MA (US)

(73) Assignee: The Children's Medical Center Corporation, Boston, MA (US)

(21) Appl. No.: 19/377,691

(22) Filed: Nov. 3, 2025

Related U.S. Application Data
(63) Continuation of application No. PCT/US2024/027841, filed on May 3, 2024.
(60) Provisional application No. 63/500,457, filed on May 5, 2023.
(60) Provisional application No. 63/568,379, filed on Mar. 21, 2024.

(51) Int. Cl. C12N 5/0783; A61K 35/17; A61K 40/32; A61K 40/42; C07K 14/725; C07K 16/40; C12N 15/86

(52) U.S. Cl. CPC C12N 5/0636; A61K 35/17; A61K 40/32; A61K 40/4251; C07K 14/7051; C07K 16/40; C12N 15/86; A61K 2239/13; C07K 2317/565; C12N 2510/00

(57) ABSTRACT

The invention features polypeptides and/or transgenic effector cells including T cell receptors (TCRs) which specifically bind anaplastic lymphoma kinase (ALK) antigens or peptide sequences, and the use of such polypeptides and/or transgenic effector cells and TCRs specific to anaplastic lymphoma kinase (ALK) antigens or peptide sequences in compositions and methods for treating ALK-positive neoplasias such as Non-Small Cell Lung Cancers (NSCLCs).

### CROSS-REFERENCE TO RELATED APPLICATIONS

The present application is a continuation under 35 U.S.C. § 111 (a) of PCT International Patent Application No. PCT/US2024/027841, filed May 3, 2024, designating the United States and published in English, which claims priority to and the benefit of U.S. Provisional Application Nos. 63/500,457, filed May 5, 2023, and U.S. Provisional Application No. 63/568,379, filed Mar. 21, 2024, each of which is incorporated herein by this reference in their entirety.

### SEQUENCE LISTING

This application contains a Sequence Listing which has been submitted electronically in XML format and is hereby incorporated by reference in its entirety. The Sequence Listing XML file, created on Jul. 30, 2024, is named 167705-033102PCT_SL.xml, and is 29,586,166 bytes in size.

### BACKGROUND OF THE INVENTION

Anaplastic Lymphoma Kinase (ALK) chromosomal translocations represent common oncogenic drivers across multiple tumor types, including anaplastic large cell lymphoma (ALCL) and non-small cell lung cancer (NSCLC), which is the leading cause of cancer-related death worldwide in both women and men. Over the last decade, several ALK tyrosine kinase inhibitors (TKIs), including crizotinib, alectinib, ceritinib, brigatinib, and lorlatinib received regulatory approval for the treatment of ALK-positive lung cancers, having been demonstrated to extend survival compared to standard chemotherapies in this patient population. However, although ALK inhibitors have improved clinical outcomes in these patients, their benefit is limited by the development of acquired resistance, which invariably develops within a few years of treatment. In addition, approximately 20% of these patients exhibit primary resistance to these targeted therapies. Therefore, there is an urgent need for additional therapeutic strategies beyond ALK TKIs for this patient population.

The advent of immune checkpoint inhibitors represented a major opportunity for the treatment of patients with advanced solid tumors, including NSCLC, and several hematologic malignancies. Unfortunately, although PD-(L)1 blockade has significantly improved the survival of cancer patients in general, only a fraction of patients with ALK+ cancers respond to this immunotherapy approach or other immune-checkpoint blockade approaches. In addition, specifically among patients with advanced ALK-positive lung cancers, immune-checkpoint blockade has been associated with a high rate of primary resistance and hyper-progression, which further highlights the need for novel and highly effective therapeutic interventions for patients with ALK-positive cancers.

### SUMMARY OF THE INVENTION

As described below, the present invention features T cell receptors that specifically bind ALK peptides, and methods of using such T cell receptors for treating anaplastic lymphoma kinase (ALK) positive neoplasias.

In one aspect, the disclosure provides a transgenic effector cell containing a heterologous polynucleotide encoding a T cell receptor (TCR) polypeptide that specifically binds an anaplastic lymphoma kinase (ALK) peptide in the context of specific HLA molecules.

In another aspect, the disclosure provides a polypeptide that specifically binds an ALK peptide or an antigen binding fragment thereof, the polypeptide containing:
(i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least about 85%, 90%, 95%, 96%, 97%, 98%, 99%, or 100% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least about 85%, 90%, 95%, 96%, 97%, 98%, 99%, or 100% identity thereto; and/or
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto.

In another aspect, the disclosure provides a polynucleotide encoding the polypeptide of any previous aspect or any other aspect of the invention delineated herein.

In another aspect, the disclosure provides a vector containing the polynucleotide of the prior aspect. In an embodiment, the disclosure provides an expression vector, where the vector is a viral vector (e.g., a retroviral or lentiviral vector). In an embodiment, the viral vector is a murine stem cell virus (MSCV) vector. In an embodiment, the vector is a mammalian expression vector. In an embodiment, the vector further contains a promoter that drives expression in a T cell or NK cell. In an embodiment, the promoter is a MSCV promoter.

In another aspect, the disclosure provides a pharmaceutical composition containing an effector cell containing a heterologous polynucleotide of any previous aspect. In an embodiment the heterologous polynucleotide is encoding a T-cell receptor (TCR) polypeptide that specifically binds an anaplastic lymphoma kinase (ALK) peptide.

In another aspect, the disclosure provides a pharmaceutical composition containing an antibody that specifically binds an ALK peptide, or an antigen binding fragment thereof, the antibody containing:
(i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least about 85%, 90%, 95%, 96%, 97%, 98%, 99%, or 100% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least about 85%, 90%, 95%, 96%, 97%, 98%, 99%, or 100% identity thereto; and/or
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto.

In one embodiment, the antibody is fused or conjugated to a cytotoxic agent.

In another aspect, the disclosure provides a method for treating a subject having an ALK-positive neoplasia, the method involving administering to the subject an effective amount of a transgenic effector cell of any previous aspect. In one embodiment, the transgenic effector cell contains a heterologous polynucleotide encoding a T-cell receptor (TCR) polypeptide that specifically binds an anaplastic lymphoma kinase (ALK) peptide, thereby treating the subject.

In another aspect, the disclosure provides a method for treating a subject having an ALK-positive neoplasia that is resistant to ALK tyrosine kinase inhibitor therapy, the method involving administering to the subject identified as resistant to ALK tyrosine kinase inhibitor therapy, an effective amount of a transgenic effector cell of any previous aspect. In one embodiment (e.g., a transgenic effector cell containing a heterologous polynucleotide encoding a T-cell receptor (TCR) polypeptide that specifically binds an anaplastic lymphoma kinase (ALK) peptide), thereby treating the subject.

In yet another aspect, the disclosure provides a method for reducing metastasis or inhibiting the development of metastasis in a subject having an ALK-positive neoplasia, the method containing administering to the subject a transgenic effector cell of any previous aspect, (e.g., an effector cell containing a heterologous polynucleotide encoding a T-cell receptor (TCR) polypeptide that specifically binds an anaplastic lymphoma kinase (ALK) peptide), thereby reducing or inhibiting the development of metastasis in the subject.

In yet another aspect, the disclosure provides a method of killing an ALK-positive neoplastic cell involving contacting the ALK positive neoplastic cell with a transgenic effector cell a transgenic effector cell of any previous aspect (e.g., an effector cell containing a heterologous polynucleotide encoding a T-cell receptor (TCR) polypeptide that specifically binds an anaplastic lymphoma kinase (ALK) peptide), thereby killing the ALK-positive neoplastic cell.

In yet another aspect, the disclosure provides a method of reducing or inhibiting the growth of an ALK positive neoplasia containing contacting the ALK positive neoplasia with a transgenic effector cell of any previous aspect (e.g., an effector cell containing a heterologous polynucleotide encoding a T-cell receptor (TCR) polypeptide that specifically binds an anaplastic lymphoma kinase (ALK) peptide), thereby reducing or inhibiting the growth of an ALK positive neoplasia.

In yet another aspect, the disclosure provides a method for treating an human leukocyte antigen (HLA) serotype A*02:01 subject having an anaplastic lymphoma kinase (ALK)-rearranged Non-Small Cell Lung Cancer (NSCLC) or anaplastic large cell lymphoma (ALCL), the method involving administering to the subject a a transgenic effector cell containing a heterologous polynucleotide encoding a T-cell receptor (TCR) polypeptide that specifically binds an anaplastic lymphoma kinase (ALK) peptide, thereby treating the subject.

In yet another aspect, the disclosure provides a method for treating an HLA-B*07:02 subject having an anaplastic lymphoma kinase (ALK)-rearranged Non-Small Cell Lung Cancer (NSCLC) or anaplastic large cell lymphoma (ALCL), the method involving administering to the subject a transgenic effector cell containing a heterologous polynucleotide encoding a T-cell receptor (TCR) polypeptide that specifically binds an anaplastic lymphoma kinase (ALK) peptide, thereby treating the subject.

In yet another aspect, the disclosure provides a method for treating an HLA-A*02:01 subject having an anaplastic lymphoma kinase (ALK)-rearranged Non-Small Cell Lung Cancer (NSCLC) or anaplastic large cell lymphoma (ALCL) that is resistant to ALK tyrosine kinase inhibitor therapy, the method involving administering to the subject identified as resistant to ALK tyrosine kinase inhibitor therapy a transgenic effector cell containing a heterologous polynucleotide encoding a T-cell receptor (TCR) polypeptide that specifically binds an anaplastic lymphoma kinase (ALK) peptide, thereby treating the subject.

In yet another aspect, the disclosure provides a method for treating an HLA-B*07:02 subject having an anaplastic lymphoma kinase (ALK)-rearranged Non-Small Cell Lung Cancer (NSCLC) or anaplastic large cell lymphoma (ALCL) that is resistant to ALK tyrosine kinase inhibitor therapy, the method involving administering to the subject identified as resistant to ALK tyrosine kinase inhibitor therapy a transgenic effector cell containing a heterologous polynucleotide encoding a T-cell receptor (TCR) polypeptide that specifically binds an anaplastic lymphoma kinase (ALK) peptide, thereby treating the subject.

In yet another aspect, the disclosure provides a method for reducing metastasis in an HLA-A*02:01 subject having an anaplastic lymphoma kinase (ALK)-rearranged Non-Small Cell Lung Cancer (NSCLC) or anaplastic large cell lymphoma (ALCL), the method involving administering to the subject a transgenic effector cell of any previous aspect (e.g., a cell containing a heterologous polynucleotide encoding a T-cell receptor (TCR) polypeptide that specifically binds an anaplastic lymphoma kinase (ALK) peptide), thereby reducing metastasis in the subject.

In yet another aspect, the disclosure provides a method for reducing metastasis in an HLA-B*07:02 subject having an anaplastic lymphoma kinase (ALK)-rearranged Non-Small Cell Lung Cancer (NSCLC) or anaplastic large cell lymphoma (ALCL), the method involving administering to the subject a transgenic effector cell of any previous aspect (e.g., a cell containing a heterologous polynucleotide encoding a T-cell receptor (TCR) polypeptide that specifically binds an anaplastic lymphoma kinase (ALK) peptide), thereby reducing metastasis in the subject.

In yet another aspect, the disclosure provides a method for reducing metastasis in an HLA-A*02:01 subject having an anaplastic lymphoma kinase (ALK)-rearranged Non-Small Cell Lung Cancer (NSCLC) or anaplastic large cell lymphoma (ALCL) that is resistant to ALK tyrosine kinase inhibitor therapy, the method involving administering to the subject identified as resistant to ALK tyrosine kinase inhibitor therapy a transgenic effector cell of any previous aspect (e.g., a cell containing a heterologous polynucleotide encoding a T-cell receptor (TCR) polypeptide that specifically binds an anaplastic lymphoma kinase (ALK) peptide), thereby reducing metastasis in the subject.

In yet another aspect, the disclosure provides a method for treating metastasis an HLA-B*07:02 subject having an anaplastic lymphoma kinase (ALK)-rearranged Non-Small Cell Lung Cancer (NSCLC) or anaplastic large cell lymphoma (ALCL) that is resistant to ALK tyrosine kinase inhibitor therapy, the method involving administering to the subject identified as resistant to ALK tyrosine kinase inhibitor therapy of any previous aspect (e.g., a cell containing a heterologous polynucleotide encoding a T-cell receptor (TCR) polypeptide that specifically binds an anaplastic lymphoma kinase (ALK) peptide), thereby reducing metastasis in the subject. In various embodiments of the above aspects, the metastasis is a central nervous system, liver, lymph node, or kidney metastasis. In various embodiments of the above aspects, the metastasis is reduced relative to an untreated control subject. In various embodiments of the above aspects, the T cell receptor polypeptide comprises:
(i) a complementarity determining region (CDR) 1, wherein the sequence of CDR 1 is any CDR I sequence disclosed in SEQ ID NOs 2969-4452, 20513-21122, 28105-28240, 29793-29820, or 30321-30416, or any sequence that has at least about 85%, 90%, 95%, 96%, 97%, 98%, 99%, or 100% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least about 85%, 90%, 95%, 96%, 97%, 98%, 99%, or 100% identity thereto; and/or
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto.

In various embodiments of the above aspects, the method further involves administering to the subject lorlatinib and/or an immune checkpoint inhibitor (ICI) selected from the group consisting of an anti-PD1 antibody, an anti-PDL1 antibody, and an anti-CTLA-4 antibody. In various embodiments of the above aspects, the ALK rearrangement is an echinoderm microtubule-associate protein-like 4-ALK rearrangement (EML4-ALK).

In yet another aspect, the disclosure provides a method of killing an ALK-positive neoplastic cell, the method comprising contacting the ALK-positive neoplastic cell with a transgenic effector cell comprising a heterologous polynucleotide encoding a TCR polypeptide that specifically binds an ALK peptide,
wherein the TCR polypeptide comprises each of:
   (i) a complementarity determining region (CDR) 1, wherein the sequence of CDR 1 is any CDR I sequence disclosed in SEQ ID NOs 2969-4452, 20513-21122, 28105-28240, 29793-29820, or 30321-30416, or any sequence that has at least about 85%, 90%, 95%, 96%, 97%, 98%, 99%, or 100% identity thereto;
   (ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least 85% identity thereto; and
   (iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto.

In yet another aspect, the disclosure provides a method of reducing the growth of an ALK-positive neoplasia in a selected subject, the method comprising contacting the ALK-positive neoplasia with a transgenic effector cell comprising a heterologous polynucleotide encoding a TCR polypeptide that specifically binds an ALK peptide,
wherein the TCR polypeptide comprises each of:
   (i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least about 85%, 90%, 95%, 96%, 97%, 98%, 99%, or 100% identity thereto;
   (ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least about 85%, 90%, 95%, 96%, 97%, 98%, 99%, or 100% identity thereto; and
   (iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto,
and wherein the subject is selected for expression of an HLA-B*07:02 serotype.

In yet another aspect, the disclosure provides a method of reducing the growth of an ALK-positive neoplasia in a selected subject, the method comprising contacting the ALK-positive neoplasia with a transgenic effector cell comprising a heterologous polynucleotide encoding a TCR polypeptide that specifically binds an ALK peptide,
wherein the TCR polypeptide comprises each of:
   (i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least about 85%, 90%, 95%, 96%, 97%, 98%, 99%, or 100% identity thereto;
   (ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least about 85%, 90%, 95%, 96%, 97%, 98%, 99%, or 100% identity thereto; and
   (iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto, and wherein the subject is selected for expression of an HLA-A*02:01 serotype.

In yet another aspect, the disclosure provides a method of treating a selected subject having an ALK-positive neoplasia, the method comprising administering to the subject an effective amount of a transgenic effector cell comprising a heterologous polynucleotide encoding a TCR polypeptide that specifically binds an ALK peptide,
wherein the TCR polypeptide comprises each of:
   (i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least about 85%, 90%, 95%, 96%, 97%, 98%, 99%, or 100% identity thereto;
   (ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least about 85%, 90%, 95%, 96%, 97%, 98%, 99%, or 100% identity thereto; and
   (iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto,
and wherein the subject is selected for expression of an HLA-B*07:02 serotype.

In yet another aspect, the disclosure provides a method of treating a selected subject having an ALK-positive neoplasia, the method comprising administering to the subject an effective amount of a transgenic effector cell comprising a heterologous polynucleotide encoding a TCR polypeptide that specifically binds an ALK peptide,
wherein the TCR polypeptide comprises each of:
   (i) a complementarity determining region (CDR) 1, wherein the sequence of CDR 1 is any CDR I sequence disclosed in SEQ ID NOs 2969-4452, 20513-21122, 28105-28240, 29793-29820, or 30321-30416, or any sequence that has at least about 85%, 90%, 95%, 96%, 97%, 98%, 99%, or 100% identity thereto;
   (ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least about 85%, 90%, 95%, 96%, 97%, 98%, 99%, or 100% identity thereto; and
   (iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto,
and wherein the subject is selected for expression of an HLA-A*02:01 serotype.

In yet another aspect, the disclosure provides a kit comprising an agent for administration to a subject with one or more ALK-positive cancers, wherein the agent comprises the pharmaceutical composition of any previous aspect.

In one embodiment of any of the above aspects or any other aspect of the invention delineated herein, the ALK peptide has at least about 85%, 90%, 95%, 96%, 97%, 98%, 99%, or 100% amino acid sequence identity to a peptide selected from the following: RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); AMLDLLHVA (SEQ ID NO: 32961); GGDLKSFLRETRPRPSQPSSLAMLDLLHVA (SEQ ID NO: 32962); FNHQNIVRCIGVSL (SEQ ID NO: 32963); and GGDLKSFLRETRPRPSQPSSLAM (SEQ ID NO: 32964). In one embodiment of any of the above aspects or any other aspect of the invention delineated herein, the T cell receptor polypeptide contains:
(i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least about 85%, 90%, 95%, 96%, 97%, 98%, 99%, or 100% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least about 85%, 90%, 95%, 96%, 97%, 98%, 99%, or 100% identity thereto; and/or
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto.

In another embodiment, the T cell receptor polypeptide comprises each of:
(i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least 85%, 90%, 95%, 96%, 97%, 98%, 99%, or 100% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least 85%, 90%, 95%, 96%, 97%, 98%, 99%, or 100% identity thereto; and
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least about 85%, 90%, 95%, 96%, 97%, 98%, 99%, or 100% identity thereto.

In other embodiments, the effector cell is an NK cell or T cell (e.g., a CD8+ or CD4+ T cell). In other embodiments, the effector cell is an autologous or allogeneic cell. In other embodiments, the cell further contains a mutation which reduces the expression of an endogenous T cell receptor alpha constant (TRAC), T cell receptor beta constant 1 (TRBC1), and/or T cell receptor beta constant 2 (TRBC2) gene. In other embodiments, the TCR polypeptide is humanized or is non-humanized. In other embodiments, the ALK peptide has at least about 90% amino acid sequence identity to a peptide selected from the group consisting of RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); AMLDLLHVA (SEQ ID NO: 32961); GGDLKSFLRETRPRPSQPSSLAMLDLLHVA (SEQ ID NO: 32962); FNHQNIVRCIGVSL (SEQ ID NO: 32963); and GGDLKSFLRETRPRPSQPSSLAM (SEQ ID NO: 32964). In other embodiments, the polypeptide is an antibody or antigen binding fragment thereof. In other embodiments, the polypeptide is a T cell receptor polypeptide or fragment thereof. In one embodiment of any of the above aspects or any other aspect of the invention delineated herein, a composition (e.g., a pharmaceutical composition) or kit of any previous aspect, further contains a tyrosine kinase inhibitor (TKI), an immune checkpoint inhibitor (ICI), an ALK peptide vaccine, or a CAR-T cell having specificity for an ALK peptide sequence. In embodiments, the TKI is crizotinib, alectinib, ceritinib, brigatinib, ensartinib, entrectinib, the ICI is selected from the group consisting of a programmed cell death protein 1 (PD-1) inhibitor, a programmed death-ligand 1 (PD-L1) inhibitor, and a cytotoxic T-lymphocyte-associated antigen-4 (CTLA-4) inhibitor.

In embodiments of any of the above aspects or any other aspect of the invention delineated herein, the metastasis is a central nervous system, liver, lymph node, or kidney metastasis. In embodiments of any of the above aspects the effector cell is an autologous or allogeneic cell. In embodiments of any of the above aspects, the effector cell further contains a mutation which reduces the expression of an endogenous T cell receptor alpha constant (TRAC), T cell receptor beta constant 1 (TRBC1), and/or T cell receptor beta constant 2 (TRBC2) gene. In embodiments of any of the above aspects, the TCR polypeptide is humanized or is non-humanized. In embodiments of any of the above aspects, the method further involves administering to the subject a tyrosine kinase inhibitor (TKI), an immune checkpoint inhibitor (ICI), an ALK peptide vaccine, and/or a CAR-T cell having specificity for an ALK peptide sequence is simultaneously or sequentially administered to the subject. In embodiments of any of the above aspects, the neoplasia is non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, or melanoma. In embodiments of any of the above aspects, the method further involves administering IFN-γ or a STING agonist (e.g., ADU-S100). In embodiments of any of the above aspects, the subject expresses an HLA class I allele. In embodiments of any of the above aspects, the ALK-positive neoplasia comprises an oncogenic ALK gene fusion, rearrangement, duplication, or mutation. In embodiments of any of the above aspects, the ALK rearrangement is a nucleophosmin-ALK rearrangement (NPM-ALK) or an echinoderm microtubule-associate protein-like 4-ALK rearrangement (EML4-ALK). In embodiments of any of the above aspects, survival of the subject is extended relative to a reference subject. In embodiments of any of the above aspects, the transgenic effector cell is administered in an amount effective to induce complete remission (CR) or partial remission of the ALK-positive neoplasia in the subject, or is effective to induce stable disease (SD) of the ALK-positive neoplasia in the subject.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “ADU-S100” is meant a compound having the structure

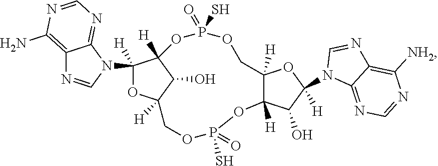 [SMILES: Nc1ncnc2c1ncn2[C@@H]1O[C@@H]2CO[P@](=O)(S)O[C@@H]3[C@H](O)[C@@H](CO[P@@](=O)(S)O[C@H]2[C@H]1O)O[C@H]3n1cnc2c(N)ncnc21]

corresponding to CAS No. 1638241-89-0, and pharmaceutically acceptable salts thereof having activity as a stimulator of interferon genes (STING).

By “alectinib” is meant a compound having the structure

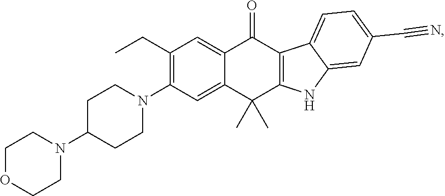 [SMILES: CCc1cc2c(cc1N1CCC(N3CCOCC3)CC1)C(C)(C)c1[nH]c3cc(C#N)ccc3c1C2=O]

corresponding to CAS No. 1256580-46-7, and pharmaceutically acceptable salts thereof having activity as a tyrosine kinase inhibitor (TKI).

By “ALK positive” is meant having detectable ALK polypeptide or polynucleotide expression. Methods for measuring ALK expression are described, for example, in Vernersson, et al. “Characterization of the expression of the ALK receptor tyrosine kinase in mice,” Gene Expr Patterns, 6:448-461 (2005) and in Dirks, et al. “Expression and functional analysis of the anaplastic lymphoma kinase (ALK) gene in tumor cell lines,” Int. J. Cancer, 100:49-56 (2002), the disclosures of which are incorporated herein by reference in their entirities for all purposes. In embodiments, an ALK positive cell contains a change to the structure of the ALK gene. In some cases, an ALK positive cell expresses ALK at higher levels than a reference cell (e.g., a healthy non-neoplastic cell).

By “brigatinib” is meant a compound having the structure

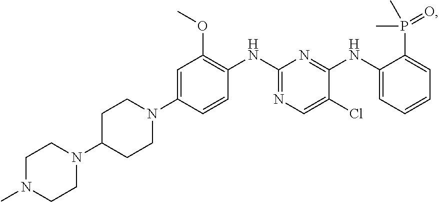 [SMILES: COc1cc(N2CCC(N3CCN(C)CC3)CC2)ccc1Nc1ncc(Cl)c(Nc2ccccc2P(C)(C)=O)n1]

corresponding to CAS No. 1197953-54-0, and pharmaceutically acceptable salts thereof having activity as a tyrosine kinase inhibitor (TKI).

By “ceritinib” is meant a compound having the structure

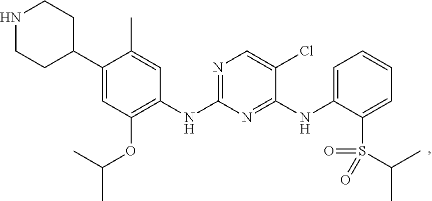 [SMILES: Cc1cc(Nc2ncc(Cl)c(Nc3ccccc3S(=O)(=O)C(C)C)n2)c(OC(C)C)cc1C1CCNCC1]

corresponding to CAS No. 1032900-25-6, and pharmaceutically acceptable salts thereof, having activity as a tyrosine kinase inhibitor (TKI).

By “crizotinib” is meant a compound having the structure

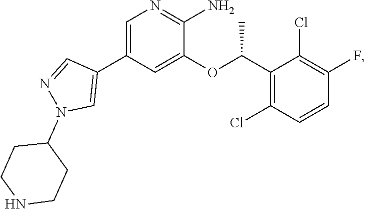 [SMILES: C[C@@H](Oc1cc(-c2cnn(C3CCNCC3)c2)cnc1N)c1c(Cl)ccc(F)c1Cl]

corresponding to CAS No. 877399-52-5, and pharmaceutically acceptable salts thereof having activity as a tyrosine kinase inhibitor (TKI).

By “ensartinib” is meant a compound having the structure

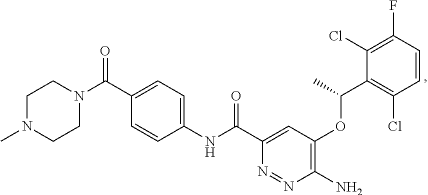 [SMILES: C[C@@H](Oc1cc(C(=O)Nc2ccc(C(=O)N3CCN(C)CC3)cc2)nnc1N)c1c(Cl)ccc(F)c1Cl]

corresponding to CAS No. 1365267-27-1, and pharmaceutically acceptable salts thereof, having activity as a tyrosine kinase inhibitor (TKI).

By “entrectinib” is meant a compound having the structure

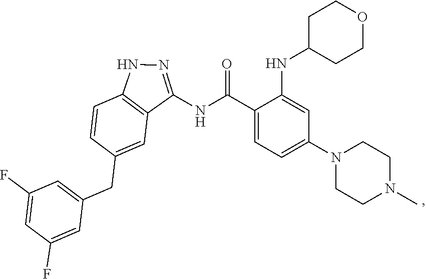 [SMILES: CN1CCN(c2ccc(C(=O)Nc3n[nH]c4ccc(Cc5cc(F)cc(F)c5)cc34)c(NC3CCOCC3)c2)CC1]

corresponding to CAS No. 1108743-60-7, and pharmaceutically acceptable salts thereof, having activity as a tyrosine kinase inhibitor (TKI).

By “lorlatinib,” “LORBRENAR,” or “LORVIQUAR” is meant a compound having the structure

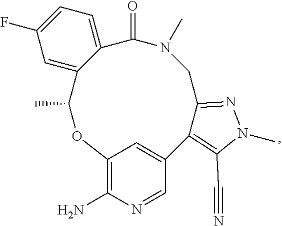 [SMILES: C[C@H]1Oc2cc(cnc2N)-c2c(nn(C)c2C#N)CN(C)C(=O)c2ccc(F)cc21]

corresponding to CAS No. 1454846-35-5, and pharmaceutically acceptable salts thereof having activity as a tyrosine kinase inhibitor (TKI).

By “adjuvant” is meant a substance or vehicle that non-specifically enhances the immune response to an antigen. Adjuvants may include a suspension of minerals (e.g., alum, aluminum hydroxide, or phosphate) on which antigen is adsorbed; or water-in-oil emulsion in which antigen solution is emulsified in mineral oil (e.g., Freund's incomplete adjuvant), sometimes with the inclusion of killed mycobacteria (Freund's complete adjuvant) to further enhance antigenicity. Immunostimulatory oligonucleotides or modifications thereof (such as those including a CpG motif) can also be used as adjuvants (see, e.g., U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include, without limitation, interleukin-1 (IL-2), the protein memory T-cell attractant “Regulated on Activation. Normal T Expressed and Secreted” (RANTES), granulocyte-macrophage-colony stimulating factor (GM-CSF), tumor necrosis factor-alpha (TNF-α), interferon-gamma (IFN-γ), granulocyte-colony stimulation factor (G-CSF), lymphocyte function-associated antigen 3 (LFA-3, also called CD58), cluster of differentiation antigen 72 (CD72), (a negative regulator of B-cell responsiveness), peripheral membrane protein, B7-1 (B7-1, also called CD80), peripheral membrane protein, B7-2 (B7-2, also called CD86), the TNF ligand superfamily member 4 ligand (OX40L) or the type 2 transmembrane glycoprotein receptor belonging to the TNF superfamily (4-1BBL). In some embodiments, the adjuvant may be conjugated to an amphiphile as described in H. Liu et al., Structure-based programming of lymph-node targeting in molecular vaccines. Nature 507, 5199522 (2014). In some embodiments, the amphiphile conjugated to the adjuvant is N-hydroxy succinimidyl ester-end-functionalized poly(ethylene glycol)-lipid (NHS-PEG2KDa-DSPE).

By “administer” is meant giving, supplying, or dispensing a composition, agent, therapeutic and the like to a subject, or applying or bringing the composition and the like into contact with the subject. Administering or administration may be accomplished by any of a number of routes, such as, for example, without limitation, topical, oral, subcutaneous, intramuscular, intraperitoneal, intravenous (IV), injection, intrathecal, intramuscular, dermal, intradermal, intracranial, inhalation, rectal, intravaginal, or intraocular.

By “adoptive cell transfer” or “ACT” is meant a process in which immune effector cells (e.g. T cells) are isolated and engineered to recognize a specific antigen (i.e., “engineered immune cells”), then expanded and reintroduced to a subject. Immune effector cells (e.g., T cells) used for ACT may be “autologous,” derived from the subject to be treated, or “allogeneic” (sometimes called “homologous”), derived from a donor subject with an immunogenic profile similar enough not to be rejected by the subject receiving ACT. In some embodiments, cells to be transferred in ACT are engineered cells expressing T cell receptors, as described herein.

By “agent” is meant any cell, small molecule chemical compound, antibody, nucleic acid molecule, peptide, polypeptide, or fragments thereof. In some embodiments, the polypeptide is a T cell receptor. In some embodiments, the polypeptide is an antibody. In some embodiments, the cell is an immune effector comprising a heterologous T cell receptor.

By “ALK polypeptide” or “ALK peptide” is meant a protein or fragment thereof having at least 85% amino acid identity to an anaplastic lymphoma kinase (ALK) amino acid sequence associated with GenBank Accessions No.: BAD92714.1, ACY79563.1, or ACI47591.1, and that is capable of inducing an ALK-specific immune response in an immunized subject. In some embodiments, the ALK polypeptide is at least 85%, at least 90%, at least 95%, at least 98%, at least 99%, or 100% identical to the ALK protein in Homo sapiens. In embodiments, the ALK peptide contains about, at least about, and/or nor more than about 5, 6, 7, 8, 9, 10, 11, 12, 13, 14, 15, 20, 25, 30, 35, 40, 45, or 50 amino acids. Exemplary ALK full-length amino acid sequences from Homo sapiens are provided below (see GenBank Accessions No. BAD92714.1, ACY79563.1, and ACI47591.1):

>BAD92714.1 anaplastic lymphoma kinase Ki-1
variant, partial [Homo sapiens] (ALK
cytoplasmic portion in bold font)
(SEQ ID NO: 32965)
TASSGGMGAIGLLWLLPLLLSTAAVGSGMGTGQRAGSPAAGPPLQ
PREPLSYSRLQRKSLAVDFVVPSLFRVYARDLLLPPSSSELKAGR
PEARGSLALDCAPLLRLLGPAPGVSWTAGSPAPAEARTLSRVLKG
GSVRKLRRAKQLVLELGEEAILEGCVGPPGEAAVGLLQFNLSELF
SWWIRQGEGRLRIRLMPEKKASEVGREGRLSAAIRASQPRLLFQI
FGTGHSSLESPTNMPSPSPDYFTWNLTWIMKDSFPFLSHRSRYGL
ECSFDFPCELEYSPPLHDLRNQSWSWRRIPSEEASQMDLLDGPGA
ERSKEMPRGSFLLLNTSADSKHTILSPWMRSSSEHCTLAVSVHRH
LQPSGRYIAQLLPHNEAAREILLMPTPGKHGWTVLQGRIGRPDNP
FRVALEYISSGNRSLSAVDFFALKNCSEGTSPGSKMALQSSFTCW
NGTVLQLGQACDFHQDCAQGEDESQMCRKLPVGFYCNFEDGFCGW
TQGTLSPHTPQWQVRTLKDARFQDHQDHALLLSTTDVPASESATV
TSATFPAPIKSSPCELRMSWLIRGVLRGNVSLVLVENKTGKEQGR
MVWHVAAYEGLSLWQWMVLPLLDVSDRFWLQMVAWWGQGSRAIVA
FDNISISLDCYLTISGEDKILQNTAPKSRNLFERNPNKELKPGEN
SPRQTPIFDPTVHWLFTTCGASGPHGPTQAQCNNAYQNSNLSVEV
GSEGPLKGIQIWKVPATDTYSISGYGAAGGKGGKNTMMRSHGVSV
LGIFNLEKDDMLYILVGQQGEDACPSTNQLIQKVCIGENNVIEEE
IRVNRSVHEWAGGGGGGGGATYVFKMKDGVPVPLIIAAGGGGRAY
GAKTDTFHPERLENNSSVLGLNGNSGAAGGGGGWNDNTSLLWAGK
SLQEGATGGHSCPQAMKKWGWETRGGFGGGGGGCSSGGGGGGYIG
GNAASNNDPEMDGEDGVSFISPLGILYTPALKVMEGHGEVNIKHY
LNCSHCEVDECHMDPESHKVICFCDHGTVLAEDGVSCIVSPTPEP
HLPLSLILSVVTSALVAALVLAFSGIMIVYRRKHQELQAMQMELQ
SPEYKLSKLRTSTIMTDYNPNYCFAGKTSSISDLKEVPRKNITLI
RGLGHGAFGEVYEGQVSGMPNDPSPLQVAVKTLPEVCSEQDELDF
LMEALIISKFNHQNIVRCIGVSLQSLPRFILLELMAGGDLKSFLR
ETRPRPSQPSSLAMLDLLHVARDIACGCQYLEENHFIHRDIAARN
CLLTCPGPGRVAKIGDFGMARDIYRASYYRKGGCAMLPVKWMPPE
AFMEGIFTSKTDTWSFGVLLWEIFSLGYMPYPSKSNQEVLEFVTS
GGRMDPPKNCPGPVYRIMTQCWQHQPEDRPNFAIILERIEYCTQD
PDVINTALPIEYGPLVEEEEKVPVRPKDPEGVPPLLVSQQAKREE
ERSPAAPPPLPTTSSGKAAKKPTAAEVSVRVPRGPAVEGGHVNMA
FSQSNPPSELHRVHGSRNKPTSLWNPTYGSWFTEKPTKKNNPIAK
KEPHERGNLGLEGSCTVPPNVATGRLPGASLLLEPSSLTANMKEV
PLFRLRHFPCGNVNYGYQQQGLPLEAATAPGAGHYEDTILKSKNS
MNQPGP.
>ACY79563.1 mutant anaplastic lymphoma receptor
tyrosine kinase [Homo sapiens]
(SEQ ID NO: 32966)
MGAIGLLWLLPLLLSTAAVGSGMGTGQRAGSPAAGPPLQPREPLS
YSRLQRKSLAVDFVVPSLFRVYARDLLLPPSSSELKAGRPEARGS
LALDCAPLLRLLGPAPGVSWTAGSPAPAEARTLSRVLKGGSVRKL
RRAKQLVLELGEEAILEGCVGPPGEAAVGLLQFNLSELFSWWIRQ
GEGRLRIRLMPEKKASEVGREGRLSAAIRASQPRLLFQIFGTGHS
SLESPTNMPSPSPDYFTWNLTWIMKDSFPFLSHRSRYGLECSFDF
PCELEYSPPLHDLRNQSWSWRRIPSEEASQMDLLDGPGAERSKEM
PRGSFLLLNTSADSKHTILSPWMRSSSEHCTLAVSVHRHLQPSGR
YIAQLLPHNEAAREILLMPTPGKHGWTVLQGRIGRPDNPFRVALE
YISSGNRSLSAVDFFALKNCSEGTSPGSKMALQSSFTCWNGTVLQ
LGQACDFHQDCAQGEDESQMCRKLPVGFYCNFEDGFCGWTQGTLS
PHTPQWQVRTLKDARFQDHQDHALLLSTTDVPASESATVTSATFP
APIKSSPCELRMSWLIRGVLRGNVSLVLVENKTGKEQGRMVWHVA
AYEGLSLWQWMVLPLLDVSDRFWLQMVAWWGQGSRAIVAFDNISI
SLDCYLTISGEDKILQNTAPKSRNLFERNPNKELKPGENSPRQTP
IFDPTVHWLFTTCGASGPHGPTQAQCNNAYQNSNLSVEVGSEGPL
KGIQIWKVPATDTYSISGYGAAGGKGGKNTMMRSHGVSVLGIFNL
EKDDMLYILVGQQGEDACPSTNQLIQKVCIGENNVIEEEIRVNRS
VHEWAGGGGGGGGATYVFKMKDGVPVPLIIAAGGGGRAYGAKTDT
FHPERLENNSSVLGLNGNSGAAGGGGGWNDNTSLLWAGKSLQEGA
TGGHSCPQAMKKWGWETRGGFGGGGGGCSSGGGGGGYIGGNAASN
NDPEMDGEDGVSFISPLGILYTPALKVMEGHGEVNIKHYLNCSHC
EVDECHMDPESHKVICFCDHGTVLAEDGVSCIVSPTPEPHLPLSL
ILSVVTSALVAALVLAFSGIMIVYRRKHQELQAMQMELQSPEYKL
SKLRTSTIMTDYNPNYCFAGKTSSISDLKEVPRKNITLIRGLGHG
AFGEVYEGQVSGMPNDPSPLQVAVKTLPEVCSEQDELDFLMEALI
ISKFNHQNIVRCIGVSLQSLPRFILLELMVGGDLKSFLRETRPRP
SQPSSLAMLDLLHVARDIACGCQYLEENHFIHRDIAARNCLLTCP
GPGRVAKIGDFGMARDIYRASYYRKGGCAMLPVKWMPPEAFMEGI
FTSKTDTWSFGVLLWEIFSLGYMPYPSKSNQEVLEFVTSGGRMDP
PKNCPGPVYRIMTQCWQHQPEDRPNFAIILERIEYCTQDPDVINT
ALPIEYGPLVEEEEKVPVRPKDPEGVPPLLVSQQAKREEERSPAA
PPPLPTTSSGKAAKKPTAAEISVRVPRGPAVEGGHVNMAFSQSNP
PSELHKVHGSRNKPTSLWNPTYGSWFTEKPTKKNNPIAKKEPHDR
GNLGLEGSCTVPPNVATGRLPGASLLLEPSSLTANMKEVPLFRLR
HFPCGNVNYGYQQQGLPLEAATAPGAGHYEDTILKSKNSMNQPGP.
>ACI47591.1 anaplastic lymphoma kinase
[Homo sapiens]
(SEQ ID NO: 32967)
MGAIGLLWLLPLLLSTAAVGSGMGTGQRAGSPAAGPPLQPREPLS
YSRLQRKSLAVDFVVPSLFRVYARDLLLPPSSSELKAGRPEARGS
LALDCAPLLRLLGPAPGVSWTAGSPAPAEARTLSRVLKGGSVRKL
RRAKQLVLELGEEAILEGCVGPPGEAAVGLLQFNLSELFSWWIRQ
GEGRLRIRLMPEKKASEVGREGRLSAAIRASQPRLLFQIFGTGHS
SLESPTNMPSPSPDYFTWNLTWIMKDSFPFLSHRSRYGLECSFDF
PCELEYSPPLHDLRNQSWSWRRIPSEEASQMDLLDGPGAERSKEM
PRGSFLLLNTSADSKHTILSPWMRSSSEHCTLAVSVHRHLQPSGR
YIAQLLPHNEAAREILLMPTPGKHGWTVLQGRIGRPDNPFRVALE
YISSGNRSLSAVDFFALKNCSEGTSPGSKMALQSSFTCWNGTVLQ
LGQACDFHQDCAQGEDESQMCRKLPVGFYCNFEDGFCGWTQGTLS
PHTPQWQVRTLKDARFQDHQDHALLLSTTDVPASESATVTSATFP
APIKSSPCELRMSWLIRGVLRGNVSLVLVENKTGKEQGRMVWHVA
AYEGLSLWQWMVLPLLDVSDRFWLQMVAWWGQGSRAIVAFDNISI
SLDCYLTISGEDKILQNTAPKSRNLFERNPNKELKPGENSPRQTP
IFDPTVHWLFTTCGASGPHGPTQAQCNNAYQNSNLSVEVGSEGPL
KGIQIWKVPATDTYSISGYGAAGGKGGKNTMMRSHGVSVLGIFNL
EKDDMLYILVGQQGEDACPSTNQLIQKVCIGENNVIEEEIRVNRS
VHEWAGGGGGGGGATYVFKMKDGVPVPLIIAAGGGGRAYGAKTDT
FHPERLENNSSVLGLNGNSGAAGGGGGWNDNTSLLWAGKSLQEGA
TGGHSCPQAMKKWGWETRGGFGGGGGGCSSGGGGGGYIGGNAASN
NDPEMDGEDGVSFISPLGILYTPALKVMEGHGEVNIKHYLNCSHC
EVDECHMDPESHKVICFCDHGTVLAEDGVSCIVSPTPEPHLPLSL
ILSVVTSALVAALVLAFSGIMIVYRRMHQELQAMQMELQSPEYKL
SKLRTSTIMTDYNPNYCFAGKTSSISDLKEVPRKNITLIRGLGHG
AFGEVYEGQVSGMPNDPSPLQVAVKTLPEVCSEQDELDFLMEALI
ISKFNHQNIVRCIGVSLQSLPRFILLELMAGGDLKSFLRETRPRP
SQPSSLAMLDLLHVARDIACGCQYLEENHFIHRDIAARNCLLTCP
GPGRVAKIGDFGMARDIYRASYYRKGGCAMLPVKWMPPEAFMEGI
FTSKTDTWSFGVLLWEIFSLGYMPYPSKSNQEVLEFVTSGGRMDP
PKNCPGPVYRIMTQCWQHQPEDRPNFAIILERIEYCTQDPDVINT
ALPIEYGPLVEEEEKVPVRPKDPEGVPPLLVSQQAKREEERSPAA
PPPLPTTSSGKAAKKPTAAEISVRVPRGPAVEGGHVNMAFSQSNP
PSELHKVHGSRNKPTSLWNPTYGSWFTEKPTKKNNPIAKKEPHDR
GNLGLEGSCTVPPNVATGRLPGASLLLEPSSLTANMKEVPLFRLR
HFPCGNVNYGYQQQGLPLEAATAPGAGHYEDTILKSKNSMNQPGP

An exemplary Homo sapiens ALK amino acid sequence from GenBank™ accession no. NP_004295 is provided below:

```
(SEQ ID NO: 32968)
      1 MGAIGLLWLL PLLLSTAAVG SGMGTGQRAG SPAAGPPLQP REPLSYSRLQ RKSLAVDFVV
    61 PSLFRVYARD LLLPPSSSEL KAGRPEARGS LALDCAPLLR LLGPAPGVSW TAGSPAPAEA
  121 RTLSRVLKGG SVRKLRRAKQ LVLELGEEAI LEGCVGPPGE AAVGLLQFNL SELFSWWIRQ
  181 GEGRLRIRLM PEKKASEVGR EGRLSAAIRA SQPRLLFQIF GTGHSSLESP TNMPSPSPDY
  241 FTWNLTWIMK DSFPFLSHRS RYGLECSFDF PCELEYSPPL HDLRNQSWSW RRIPSEEASQ
  301 MDLLDGPGAE RSKEMPRGSF LLLNTSADSK HTILSPWMRS SSEHCTLAVS VHRHLQPSGR
  361 YIAQLLPHNE AAREILLMPT PGKHGWTVLQ GRIGRPDNPF RVALEYISSG NRSLSAVDFF
  421 ALKNCSEGTS PGSKMALQSS FTCWNGTVLQ LGQACDFHQD CAQGEDESQM CRKLPVGFYC
  481 NFEDGFCGWT QGTLSPHTPQ WQVRTLKDAR FQDHQDHALL LSTTDVPASE SATVTSATFP
  541 APIKSSPCEL RMSWLIRGVL RGNVSLVLVE NKTGKEQGRM VWHVAAYEGL SLWQWMVLPL
  601 LDVSDRFWLQ MVAWWGQGSR AIVAFDNISI SLDCYLTISG EDKILQNTAP KSRNLFERNP
  661 NKELKPGENS PRQTPIFDPT VHWLFTTCGA SGPHGPTQAQ CNNAYQNSNL SVEVGSEGPL
  721 KGIQIWKVPA TDTYSISGYG AAGGKGGKNT MMRSHGVSVL GIFNLEKDDM LYILVGQQGE
  781 DACPSTNQLI QKVCIGENNV IEEEIRVNRS VHEWAGGGGG GGGATYVFKM KDGVPVPLII
  841 AAGGGGRAYG AKTDTFHPER LENNSSVLGL NGNSGAAGGG GGWNDNTSLL WAGKSLQEGA
  901 TGGHSCPQAM KKWGWETRGG FGGGGGGCSS GGGGGGYIGG NAASNNDPEM DGEDGVSFIS
  961 PLGILYTPAL KVMEGHGEVN IKHYLNCSHC EVDECHMDPE SHKVICFCDH GTVLAEDGVS
1021 CIVSPTPEPH LPLSLILSVV TSALVAALVL AFSGIMIVYR RKHQELQAMQ MELQSPEYKL
1081 SKLRTSTIMT DYNPNYCFAG KTSSISDLKE VPRKNITLIR GLGHGAFGEV YEGQVSGMPN
1141 DPSPLQVAVK TLPEVCSEQD ELDFLMEALI ISKFNHQNIV RCIGVSLQSL PRFILLELMA
1201 GGDLKSFLRE TRPRPSQPSS LAMLDLLHVA RDIACGCQYL EENHFIHRDI AARNCLLTCP
1261 GPGRVAKIGD FGMARDIYRA SYYRKGGCAM LPVKWMPPEA FMEGIFTSKT DTWSFGVLLW
1321 EIFSLGYMPY PSKSNQEVLE FVTSGGRMDP PKNCPGPVYR IMTQCWQHQP EDRPNFAIIL
1381 ERIEYCTQDP DVINTALPIE YGPLVEEEEK VPVRPKDPEG VPPLLVSQQA KREEERSPAA
1441 PPPLPTTSSG KAAKKPTAAE ISVRVPRGPA VEGGHVNMAF SQSNPPSELH KVHGSRNKPT
1501 SLWNPTYGSW FTEKPTKKNN PIAKKEPHDR GNLGLEGSCT VPPNVATGRL PGASLLLEPS
1561 SLTANMKEVP LFRLRHFPCG NVNYGYQQQG LPLEAATAPG AGHYEDTILK SKNSMNQPGP
```

An exemplary Homo sapiens ALK polypeptide sequence from UniProt Accession No. Q9UM73 is provided below (extracellular domain (amino acids 19-1038) provided in bold font):

(SEQ ID NO: 33008)
MGAIGLLWLLPLLLSTAAVGSGMGTGQRAGSPAAGPPLQPREPLSY
SRLQRKSLAVDFVVPSLFRVYARDLLLPPSSSELKAGRPEARGSL
ALDCAPLLRLLGPAPGVSWTAGSPAPAEARTLSRVLKGGSVRKLR
RAKQLVLELGEEAILEGCVGPPGEAAVGLLQFNLSELFSWWIRQG
EGRLRIRLMPEKKASEVGREGRLSAAIRASQPRLLFQIFGTGHSS
LESPTNMPSPSPDYFTWNLTWIMKDSFPFLSHRSRYGLECSFDFP
CELEYSPPLHDLRNQSWSWRRIPSEEASQMDLLDGPGAERSKEMP
RGSFLLLNTSADSKHTILSPWMRSSSEHCTLAVSVHRHLQPSGRY
IAQLLPHNEAAREILLMPTPGKHGWTVLQGRIGRPDNPFRVALEY
ISSGNRSLSAVDFFALKNCSEGTSPGSKMALQSSFTCWNGTVLQL
GQACDFHQDCAQGEDESQMCRKLPVGFYCNFEDGFCGWTQGTLSP
HTPQWQVRTLKDARFQDHQDHALLLSTTDVPASESATVTSATFPA
PIKSSPCELRMSWLIRGVLRGNVSLVLVENKTGKEQGRMVWHVAA
YEGLSLWQWMVLPLLDVSDRFWLQMVAWWGQGSRAIVAFDNISIS
LDCYLTISGEDKILQNTAPKSRNLFERNPNKELKPGENSPRQTPI
FDPTVHWLFTTCGASGPHGPTQAQCNNAYQNSNLSVEVGSEGPLK
GIQIWKVPATDTYSISGYGAAGGKGGKNTMMRSHGVSVLGIFNLE
KDDMLYILVGQQGEDACPSTNQLIQKVCIGENNVIEEEIRVNRSV
HEWAGGGGGGGGATYVFKMKDGVPVPLIIAAGGGGRAYGAKTDTF
HPERLENNSSVLGLNGNSGAAGGGGGWNDNTSLLWAGKSLQEGAT
GGHSCPQAMKKWGWETRGGFGGGGGGCSSGGGGGGYIGGNAASNN
DPEMDGEDGVSFISPLGILYTPALKVMEGHGEVNIKHYLNCSHCE
VDECHMDPESHKVICFCDHGTVLAEDGVSCIVSPTPEPHLPLSLI
LSVVTSALVAALVLAFSGIMIVYRRKHQELQAMQMELQSPEYKLS
KLRTSTIMTDYNPNYCFAGKTSSISDLKEVPRKNITLIRGLGHGA
FGEVYEGQVSGMPNDPSPLQVAVKTLPEVCSEQDELDFLMEALII
SKFNHQNIVRCIGVSLQSLPRFILLELMAGGDLKSFLRETRPRPS
QPSSLAMLDLLHVARDIACGCQYLEENHFIHRDIAARNCLLTCPG
PGRVAKIGDFGMARDIYRASYYRKGGCAMLPVKWMPPEAFMEGIF
TSKTDTWSFGVLLWEIFSLGYMPYPSKSNQEVLEFVTSGGRMDPP
KNCPGPVYRIMTQCWQHQPEDRPNFAIILERIEYCTQDPDVINTA
LPIEYGPLVEEEEKVPVRPKDPEGVPPLLVSQQAKREEERSPAAP
PPLPTTSSGKAAKKPTAAEISVRVPRGPAVEGGHVNMAFSQSNPP
SELHKVHGSRNKPTSLWNPTYGSWFTEKPTKKNNPIAKKEPHDRG
NLGLEGSCTVPPNVATGRLPGASLLLEPSSLTANMKEVPLFRLRH
FPCGNVNYGYQQQGLPLEAATAPGAGHYEDTILKSKNSMNQPGP

An exemplary ALK full-length amino acid sequence from Mus musculus is provided below:

| (SEQ ID NO: 32969) | |
|---|---|
| 1 | MGQRQSILKR CPEGSFLLLN TSADSKHTIL |
| | SPWMRSSSDH CTLAVSVHRH LQPSGRYVAQ |
| 61 | LLPHNEAGRE ILLVPTPGKH GWTVLQGRVG |
| | RPANPFRVAL EYISSGNRSL SAVDFFALKN |
| 121 | CSEGTSPGSK MALQSSFTCW NGTVLQLGQA |
| | CDFHQDCAQG EDEGQLCSKL PAGFYCNFEN |
| 181 | GFCGWTQSPL SPHMPRWQVR TLRDAHSQGH |
| | QGRALLLSTT DILASEGATV TSATFPAPMK |
| 241 | NSPCELRMSW LIRGVLRGNV SLVLVENKTG |
| | KEQSRTVWHV ATDEGLSLWQ HTVLSLLDVT |
| 301 | DRFWLQIVTW WGPGSRATVG FDNISISLDC |
| | YLTISGEEKM SLNSVPKSRN LFEKNPNKES |
| 361 | KSWANISGPT PIFDPTVHWL FTTCGASGPH |
| | GPTQAQCNNA YQNSNLSVVV GSEGPLKGVQ |
| 421 | IWKVPATDTY SISGYGAAGG KGGKNTMMRS |
| | HGVSVLGIFN LEKGDTLYIL VGQQGEDACP |
| 481 | RANQLIQKVC VGENNVIEEE IRVNRSVHEW |
| | AGGGGGGGGA TYVFKMKDGV PVPLIIAAGG |
| 541 | GGRAYGAKTE TFHPERLESN SSVLGLNGNS |
| | GAAGGGGGWN DNTSLLWAGK SLLEGAAGGH |
| 601 | SCPQAMKKWG WETRGGFGGG GGGGAPQVEE |
| | AEDI |

By “anaplastic lymphoma kinase (ALK) peptide” is meant an immunogenic fragment of an ALK polypeptide.

An exemplary ALK peptide amino sequence is as follows: RPRPSQPSSL (SEQ ID NO: 32957) (RPRshort).

An exemplary ALK peptide amino sequence is as follows: IVRCIGVSL (SEQ ID NO: 32958) (IVRshort).

An exemplary ALK peptide amino sequence is as follows: VPRKNITLI (SEQ ID NO: 32959).

An exemplary ALK peptide amino sequence is as follows: TAAEVSVRV (SEQ ID NO: 32960).

An exemplary ALK peptide amino sequence is as follows: AMLDLLHVA (SEQ ID NO: 32961).

An exemplary ALK peptide amino sequence is as follows:

(SEQ ID NO: 32962)
GGDLKSFLRETRPRPSQPSSLAMLDLLHVA.

An exemplary ALK peptide amino sequence is as follows: FNHQNIVRCIGVSL (SEQ ID NO: 32963) (IVRlong).

An exemplary ALK peptide amino sequence is as follows:

(SEQ ID NO: 32964)
GGDLKSFLRETRPRPSQPSSLAM.
(RPRlong)

By “ALK polynucleotide” is meant any nucleic acid molecule encoding an ALK polypeptide or fragment thereof. Exemplary full-length ALK nucleic acid sequences from Homo sapiens are provided below (see GenBank Accessions No.: AB209477.4, GU128155.1, and EU788003.1):

>AB209477.4:472-5352 Homo sapiens mRNA for anaplastic
lymphoma kinase Ki-1 variant protein, partial cds
(SEQ ID NO: 32970)
ACGGCCTCCTCCGGCGGGATGGGAGCCATCGGGCTCCTGTGGCTCCTGCCGCTGCTGCTTTCCA
CGGCAGCTGTGGGCTCCGGGATGGGGACCGGCCAGCGCGCGGGCTCCCCAGCTGCGGGGCCGCC
GCTGCAGCCCCGGGAGCCACTCAGCTACTCGCGCCTGCAGAGGAAGAGTCTGGCAGTTGACTTC
GTGGTGCCCTCGCTCTTCCGTGTCTACGCCCGGGACCTACTGCTGCCACCATCCTCCTCGGAGC
TGAAGGCTGGCAGGCCCGAGGCCCGCGGCTCGCTAGCTCTGGACTGCGCCCCGCTGCTCAGGTT
GCTGGGGCCGGCGCCGGGGGTCTCCTGGACCGCCGGTTCACCAGCCCCGGCAGAGGCCCGGACG
CTGTCCAGGGTGCTGAAGGGCGGCTCCGTGCGCAAGCTCCGGCGTGCCAAGCAGTTGGTGCTGG
AGCTGGGCGAGGAGGCGATCTTGGAGGGTTGCGTCGGGCCCCCCGGGGAGGCGGCTGTGGGGCT
GCTCCAGTTCAATCTCAGCGAGCTGTTCAGTTGGTGGATTCGCCAAGGCGAAGGGCGACTGAGG
ATCCGCCTGATGCCCGAGAAGAAGGCGTCGGAAGTGGGCAGAGAGGGAAGGCTGTCCGCGGCAA
TTCGCGCCTCCCAGCCCCGCCTTCTCTTCCAGATCTTCGGGACTGGTCATAGCTCCTTGGAATC
ACCAACAAACATGCCATCTCCTTCTCCTGATTATTTTACATGGAATCTCACCTGGATAATGAAA
GACTCCTTCCCTTTCCTGTCTCATCGCAGCCGATATGGTCTGGAGTGCAGCTTTGACTTCCCCT
GTGAGCTGGAGTATTCCCCTCCACTGCATGACCTCAGGAACCAGAGCTGGTCCTGGCGCCGCAT
CCCCTCCGAGGAGGCCTCCCAGATGGACTTGCTGGATGGGCCTGGGGCAGAGCGTTCTAAGGAG
ATGCCCAGAGGCTCCTTTCTCCTTCTCAACACCTCAGCTGACTCCAAGCACACCATCCTGAGTC
CGTGGATGAGGAGCAGCAGTGAGCACTGCACACTGGCCGTCTCGGTGCACAGGCACCTGCAGCC
CTCTGGAAGGTACATTGCCCAGCTGCTGCCCCACAACGAGGCTGCAAGAGAGATCCTCCTGATG
CCCACTCCAGGGAAGCATGGTTGGACAGTGCTCCAGGGAAGAATCGGGCGTCCAGACAACCCAT
TTCGAGTGGCCCTGGAATACATCTCCAGTGGAAACCGCAGCTTGTCTGCAGTGGACTTCTTTGC
CCTGAAGAACTGCAGTGAAGGAACATCCCCAGGCTCCAAGATGGCCCTGCAGAGCTCCTTCACT
TGTTGGAATGGGACAGTCCTCCAGCTTGGGCAGGCCTGTGACTTCCACCAGGACTGTGCCCAGG
GAGAAGATGAGAGCCAGATGTGCCGGAAACTGCCTGTGGGTTTTTACTGCAACTTTGAAGATGG
CTTCTGTGGCTGGACCCAAGGCACACTGTCACCCCACACTCCTCAGTGGCAGGTCAGGACCCTA
AAGGATGCCCGGTTCCAGGACCACCAAGACCATGCTCTATTGCTCAGTACCACTGATGTCCCCG
CTTCTGAAAGTGCTACAGTGACCAGTGCTACGTTTCCTGCACCGATCAAGAGCTCTCCATGTGA
GCTCCGAATGTCCTGGCTCATTCGTGGAGTCTTGAGGGGAAACGTGTCCTTGGTGCTAGTGGAG
AACAAAACCGGGAAGGAGCAAGGCAGGATGGTCTGGCATGTCGCCGCCTATGAAGGCTTGAGCC
TGTGGCAGTGGATGGTGTTGCCTCTCCTCGATGTGTCTGACAGGTTCTGGCTGCAGATGGTCGC
ATGGTGGGGACAAGGATCCAGAGCCATCGTGGCTTTTGACAATATCTCCATCAGCCTGGACTGC
TACCTCACCATTAGCGGAGAGGACAAGATCCTGCAGAATACAGCACCCAAATCAAGAAACCTGT
TTGAGAGAAACCCAAACAAGGAGCTGAAACCCGGGGAAAATTCACCAAGACAGACCCCCATCTT
TGACCCTACAGTTCATTGGCTGTTCACCACATGTGGGGCCAGCGGGCCCCATGGCCCCACCCAG
GCACAGTGCAACAACGCCTACCAGAACTCCAACCTGAGCGTGGAGGTGGGGAGCGAGGGCCCCC
TGAAAGGCATCCAGATCTGGAAGGTGCCAGCCACCGACACCTACAGCATCTCGGGCTACGGAGC
TGCTGGCGGGAAAGGCGGGAAGAACACCATGATGCGGTCCCACGGCGTGTCTGTGCTGGGCATC
TTCAACCTGGAGAAGGATGACATGCTGTACATCCTGGTTGGGCAGCAGGGAGAGGACGCCTGCC
CCAGTACAAACCAGTTAATCCAGAAAGTCTGCATTGGAGAGAACAATGTGATAGAAGAAGAAAT
CCGTGTGAACAGAAGCGTGCATGAGTGGGCAGGAGGCGGAGGAGGAGGGGGTGGAGCCACCTAC
GTATTTAAGATGAAGGATGGAGTGCCGGTGCCCCTGATCATTGCAGCCGGAGGTGGTGGCAGGG
CCTACGGGGCCAAGACAGACACGTTCCACCCAGAGAGACTGGAGAATAACTCCTCGGTTCTAGG
GCTAAACGGCAATTCCGGAGCCGCAGGTGGTGGAGGTGGCTGGAATGATAACACTTCCTTGCTC
TGGGCCGGAAAATCTTTGCAGGAGGGTGCCACCGGAGGACATTCCTGCCCCCAGGCCATGAAGA
AGTGGGGGTGGGAGACAAGAGGGGGTTTCGGAGGGGGTGGAGGGGGGTGCTCCTCAGGTGGAGG
AGGCGGAGGATATATAGGCGGCAATGCAGCCTCAAACAATGACCCCGAAATGGATGGGGAAGAT
GGGGTTTCCTTCATCAGTCCACTGGGCATCCTGTACACCCCAGCTTTAAAAGTGATGGAAGGCC
ACGGGGAAGTGAATATTAAGCATTATCTAAACTGCAGTCACTGTGAGGTAGACGAATGTCACAT
GGACCCTGAAAGCCACAAGGTCATCTGCTTCTGTGACCACGGGACGGTGCTGGCTGAGGATGGC
GTCTCCTGCATTGTGTCACCCACCCCGGAGCCACACCTGCCACTCTCGCTGATCCTCTCTGTGG
TGACCTCTGCCCTCGTGGCCGCCCTGGTCCTGGCTTTCTCCGGCATCATGATTGTGTACCGCCG
GAAGCACCAGGAGCTGCAAGCCATGCAGATGGAGCTGCAGAGCCCTGAGTACAAGCTGAGCAAG
CTCCGCACCTCGACCATCATGACCGACTACAACCCCAACTACTGCTTTGCTGGCAAGACCTCCT
CCATCAGTGACCTGAAGGAGGTGCCGCGGAAAAACATCACCCTCATTCGGGGTCTGGGCCATGG
AGCCTTTGGGGAGGTGTATGAAGGCCAGGTGTCCGGAATGCCCAACGACCCAAGCCCCCTGCAA
GTGGCTGTGAAGACGCTGCCTGAAGTGTGCTCTGAACAGGACGAACTGGATTTCCTCATGGAAG
CCCTGATCATCAGCAAATTCAACCACCAGAACATTGTTCGCTGCATTGGGGTGAGCCTGCAATC
CCTGCCCCGGTTCATCCTGCTGGAGCTCATGGCGGGGGGAGACCTCAAGTCCTTCCTCCGAGAG
ACCCGCCCTCGCCCGAGCCAGCCCTCCTCCCTGGCCATGCTGGACCTTCTGCACGTGGCTCGGG
ACATTGCCTGTGGCTGTCAGTATTTGGAGGAAAACCACTTCATCCACCGAGACATTGCTGCCAG
AAACTGCCTCTTGACCTGTCCAGGCCCTGGAAGAGTGGCCAAGATTGGAGACTTCGGGATGGCC
CGAGACATCTACAGGGCGAGCTACTATAGAAAGGGAGGCTGTGCCATGCTGCCAGTTAAGTGGA
TGCCCCCAGAGGCCTTCATGGAAGGAATATTCACTTCTAAAACAGACACATGGTCCTTTGGAGT
GCTGCTATGGGAAATCTTTTCTCTTGGATATATGCCATACCCCAGCAAAAGCAACCAGGAAGTT
CTGGAGTTTGTCACCAGTGGAGGCCGGATGGACCCACCCAAGAACTGCCCTGGGCCTGTATACC
GGATAATGACTCAGTGCTGGCAACATCAGCCTGAAGACAGGCCCAACTTTGCCATCATTTTGGA
GAGGATTGAATACTGCACCCAGGACCCGGATGTAATCAACACCGCTTTGCCGATAGAATATGGT
CCACTTGTGGAAGAGGAAGAGAAAGTGCCTGTGAGGCCCAAGGACCCTGAGGGGGTTCCTCCTC
TCCTGGTCTCTCAACAGGCAAAACGGGAGGAGGAGCGCAGCCCAGCTGCCCCACCACCTCTGCC
TACCACCTCCTCTGGCAAGGCTGCAAAGAAACCCACAGCTGCAGAGGTCTCTGTTCGAGTCCCT
AGAGGGCCGGCCGTGGAAGGGGGACACGTGAATATGGCATTCTCTCAGTCCAACCCTCCTTCGG
AGTTGCACAGGGTCCACGGATCCAGAAACAAGCCCACCAGCTTGTGGAACCCAACGTACGGCTC
CTGGTTTACAGAGAAACCCACCAAAAAGAATAATCCTATAGCAAAGAAGGAGCCACACGAGAGG
GGTAACCTGGGGCTGGAGGGAAGCTGTACTGTCCCACCTAACGTTGCAACTGGGAGACTTCCGG
GGGCCTCACTGCTCCTAGAGCCCTCTTCGCTGACTGCCAATATGAAGGAGGTACCTCTGTTCAG
GCTACGTCACTTCCCTTGTGGGAATGTCAATTACGGCTACCAGCAACAGGGCTTGCCCTTAGAA
GCCGCTACTGCCCCTGGAGCTGGTCATTACGAGGATACCATTCTGAAAAGCAAGAATAGCATGA
ACCAGCCTGGGCCCTGA.
>GU128155.1 Homo sapiens A1200V mutant anaplastic lymphoma
receptor tyrosine kinase (ALK) mRNA, complete cds
(SEQ ID NO: 32971)
ATGGGAGCCATCGGGCTCCTGTGGCTCCTGCCGCTGCTGCTTTCCACGGCAGCTGTGGGCTCCG
GGATGGGGACCGGCCAGCGCGCGGGCTCCCCAGCTGCGGGGCCGCCGCTGCAGCCCCGGGAGCC
ACTCAGCTACTCGCGCCTGCAGAGGAAGAGTCTGGCAGTTGACTTCGTGGTGCCCTCGCTCTTC
CGTGTCTACGCCCGGGACCTACTGCTGCCACCATCCTCCTCGGAGCTGAAGGCTGGCAGGCCCG
AGGCCCGCGGCTCGCTAGCTCTGGACTGCGCCCCGCTGCTCAGGTTGCTGGGGCCGGCGCCGGG
GGTCTCCTGGACCGCCGGTTCACCAGCCCCGGCAGAGGCCCGGACGCTGTCCAGGGTGCTGAAG
GGCGGCTCCGTGCGCAAGCTCCGGCGTGCCAAGCAGTTGGTGCTGGAGCTGGGCGAGGAGGCGA
TCTTGGAGGGTTGCGTCGGGCCCCCCGGGGAGGCGGCTGTGGGGCTGCTCCAGTTCAATCTCAG
CGAGCTGTTCAGTTGGTGGATTCGCCAAGGCGAAGGGCGACTGAGGATCCGCCTGATGCCCGAG
AAGAAGGCGTCGGAAGTGGGCAGAGAGGGAAGGCTGTCCGCGGCAATTCGCGCCTCCCAGCCCC
GCCTTCTCTTCCAGATCTTCGGGACTGGTCATAGCTCCTTGGAATCACCAACAAACATGCCTTC
TCCTTCTCCTGATTATTTTACATGGAATCTCACCTGGATAATGAAAGACTCCTTCCCTTTCCTG
TCTCATCGCAGCCGATATGGTCTGGAGTGCAGCTTTGACTTCCCCTGTGAGCTGGAGTATTCCC
CTCCACTGCATGACCTCAGGAACCAGAGCTGGTCCTGGCGCCGCATCCCCTCCGAGGAGGCCTC
CCAGATGGACTTGCTGGATGGGCCTGGGGCAGAGCGTTCTAAGGAGATGCCCAGAGGCTCCTTT
CTCCTTCTCAACACCTCAGCTGACTCCAAGCACACCATCCTGAGTCCGTGGATGAGGAGCAGCA
GTGAGCACTGCACACTGGCCGTCTCGGTGCACAGGCACCTGCAGCCCTCTGGAAGGTACATTGC
CCAGCTGCTGCCCCACAACGAGGCTGCAAGAGAGATCCTCCTGATGCCCACTCCAGGGAAGCAT
GGTTGGACAGTGCTCCAGGGAAGAATCGGGCGTCCAGACAACCCATTTCGAGTGGCCCTGGAAT
ACATCTCCAGTGGAAACCGCAGCTTGTCTGCAGTGGACTTCTTTGCCCTGAAGAACTGCAGTGA
AGGAACATCCCCAGGCTCCAAGATGGCCCTGCAGAGCTCCTTCACTTGTTGGAATGGGACAGTC
CTCCAGCTTGGGCAGGCCTGTGACTTCCACCAGGACTGTGCCCAGGGAGAAGATGAGAGCCAGA
TGTGCCGGAAACTGCCTGTGGGTTTTTACTGCAACTTTGAAGATGGCTTCTGTGGCTGGACCCA
AGGCACACTGTCACCCCACACTCCTCAATGGCAGGTCAGGACCCTAAAGGATGCCCGGTTCCAG
GACCACCAAGACCATGCTCTATTGCTCAGTACCACTGATGTCCCCGCTTCTGAAAGTGCTACAG
TGACCAGTGCTACGTTTCCTGCACCGATCAAGAGCTCTCCATGTGAGCTCCGAATGTCCTGGCT
CATTCGTGGAGTCTTGAGGGGAAACGTGTCCTTGGTGCTAGTGGAGAACAAAACCGGGAAGGAG
CAAGGCAGGATGGTCTGGCATGTCGCCGCCTATGAAGGCTTGAGCCTGTGGCAGTGGATGGTGT
TGCCTCTCCTCGATGTGTCTGACAGGTTCTGGCTGCAGATGGTCGCATGGTGGGGACAAGGATC
CAGAGCCATCGTGGCTTTTGACAATATCTCCATCAGCCTGGACTGCTACCTCACCATTAGCGGA
GAGGACAAGATCCTGCAGAATACAGCACCCAAATCAAGAAACCTGTTTGAGAGAAACCCAAACA
AGGAGCTGAAACCCGGGGAAAATTCACCAAGACAGACCCCCATCTTTGACCCTACAGTTCATTG
GCTGTTCACCACATGTGGGGCCAGCGGGCCCCATGGCCCCACCCAGGCACAGTGCAACAACGCC
TACCAGAACTCCAACCTGAGCGTGGAGGTGGGGAGCGAGGGCCCCCTGAAAGGCATCCAGATCT
GGAAGGTGCCAGCCACCGACACCTACAGCATCTCGGGCTACGGAGCTGCTGGCGGGAAAGGCGG
GAAGAACACCATGATGCGGTCCCACGGCGTGTCTGTGCTGGGCATCTTCAACCTGGAGAAGGAT
GACATGCTGTACATCCTGGTTGGGCAGCAGGGAGAGGACGCCTGCCCCAGTACAAACCAGTTAA
TCCAGAAAGTCTGCATTGGAGAGAACAATGTGATAGAAGAAGAAATCCGTGTGAACAGAAGCGT
GCATGAGTGGGCAGGAGGCGGAGGAGGAGGGGGTGGAGCCACCTACGTATTTAAGATGAAGGAT
GGAGTGCCGGTGCCCCTGATCATTGCAGCCGGAGGTGGTGGCAGGGCCTACGGGGCCAAGACAG
ACACGTTCCACCCAGAGAGACTGGAGAATAACTCCTCGGTTCTAGGGCTAAACGGCAATTCCGG
AGCCGCAGGTGGTGGAGGTGGCTGGAATGATAACACTTCCTTGCTCTGGGCCGGAAAATCTTTG
CAGGAGGGTGCCACCGGAGGACATTCCTGCCCCCAGGCCATGAAGAAGTGGGGGTGGGAGACAA
GAGGGGGTTTCGGAGGGGGTGGAGGGGGGTGCTCCTCAGGTGGAGGAGGCGGAGGATATATAGG
CGGCAATGCAGCCTCAAACAATGACCCCGAAATGGATGGGGAAGATGGGGTTTCCTTCATCAGT
CCACTGGGCATCCTGTACACCCCAGCTTTAAAAGTGATGGAAGGCCACGGGGAAGTGAATATTA
AGCATTATCTAAACTGCAGTCACTGTGAGGTAGACGAATGTCACATGGACCCTGAAAGCCACAA
GGTCATCTGCTTCTGTGACCACGGGACGGTGCTGGCTGAGGATGGCGTCTCCTGCATTGTGTCA
CCCACCCCGGAGCCACACCTGCCACTCTCGCTGATCCTCTCTGTGGTGACCTCTGCCCTCGTGG
CCGCCCTGGTCCTGGCTTTCTCCGGCATCATGATTGTGTACCGCCGGAAGCACCAGGAGCTGCA
AGCCATGCAGATGGAGCTGCAGAGCCCTGAGTACAAGCTGAGCAAGCTCCGCACCTCGACCATC
ATGACCGACTACAACCCCAACTACTGCTTTGCTGGCAAGACCTCCTCCATCAGTGACCTGAAGG
AGGTGCCGCGGAAAAACATCACCCTCATTCGGGGTCTGGGCCATGGCGCCTTTGGGGAGGTGTA
TGAAGGCCAGGTGTCCGGAATGCCCAACGACCCAAGCCCCCTGCAAGTGGCTGTGAAGACGCTG
CCTGAAGTGTGCTCTGAACAGGACGAACTGGATTTCCTCATGGAAGCCCTGATCATCAGCAAAT
TCAACCACCAGAACATTGTTCGCTGCATTGGGGTGAGCCTGCAATCCCTGCCCCGGTTCATCCT
GCTGGAGCTCATGGTGGGGGGAGACCTCAAGTCCTTCCTCCGAGAGACCCGCCCTCGCCCGAGC
CAGCCCTCCTCCCTGGCCATGCTGGACCTTCTGCACGTGGCTCGGGACATTGCCTGTGGCTGTC
AGTATTTGGAGGAAAACCACTTCATCCACCGAGACATTGCTGCCAGAAACTGCCTCTTGACCTG
TCCAGGCCCTGGAAGAGTGGCCAAGATTGGAGACTTCGGGATGGCCCGAGACATCTACAGGGCG
AGCTACTATAGAAAGGGAGGCTGTGCCATGCTGCCAGTTAAGTGGATGCCCCCAGAGGCCTTCA
TGGAAGGAATATTCACTTCTAAAACAGACACATGGTCCTTTGGAGTGCTGCTATGGGAAATCTT
TTCTCTTGGATATATGCCATACCCCAGCAAAAGCAACCAGGAAGTTCTGGAGTTTGTCACCAGT
GGAGGCCGGATGGACCCACCCAAGAACTGCCCTGGGCCTGTATACCGGATAATGACTCAGTGCT
GGCAACATCAGCCTGAAGACAGGCCCAACTTTGCCATCATTTTGGAGAGGATTGAATACTGCAC
CCAGGACCCGGATGTAATCAACACCGCTTTGCCGATAGAATATGGTCCACTTGTGGAAGAGGAA
GAGAAAGTGCCTGTGAGGCCCAAGGACCCTGAGGGGGTTCCTCCTCTCCTGGTCTCTCAACAGG
CAAAACGGGAGGAGGAGCGCAGCCCAGCTGCCCCACCACCTCTGCCTACCACCTCCTCTGGCAA
GGCTGCAAAGAAACCCACAGCTGCAGAGATCTCTGTTCGAGTCCCTAGAGGGCCGGCCGTGGAA
GGGGGACACGTGAATATGGCATTCTCTCAGTCCAACCCTCCTTCGGAGTTGCACAAGGTCCACG
GATCCAGAAACAAGCCCACCAGCTTGTGGAACCCAACGTACGGCTCCTGGTTTACAGAGAAACC
CACCAAAAAGAATAATCCTATAGCAAAGAAGGAGCCACACGACAGGGGTAACCTGGGGCTGGAG
GGAAGCTGTACTGTCCCACCTAACGTTGCAACTGGGAGACTTCCGGGGGCCTCACTGCTCCTAG
AGCCCTCTTCGCTGACTGCCAATATGAAGGAGGTACCTCTGTTCAGGCTACGTCACTTCCCTTG
TGGGAATGTCAATTACGGCTACCAGCAACAGGGCTTGCCCTTAGAAGCCGCTACTGCCCCTGGA
GCTGGTCATTACGAGGATACCATTCTGAAAAGCAAGAATAGCATGAACCAGCCTGGGCCCTGA.
>EU788003.1:908-5770 Homo sapiens mutant K1062M anaplastic
lymphoma kinase (ALK) mRNA, complete cds
(SEQ ID NO: 32972)
ATGGGAGCCATCGGGCTCCTGTGGCTCCTGCCGCTGCTGCTTTCCACGGCAGCTGTGGGCTCCG
GGATGGGGACCGGCCAGCGCGCGGGCTCCCCAGCTGCGGGGCCGCCGCTGCAGCCCCGGGAGCC
ACTCAGCTACTCGCGCCTGCAGAGGAAGAGTCTGGCAGTTGACTTCGTGGTGCCCTCGCTCTTC
CGTGTCTACGCCCGGGACCTACTGCTGCCACCATCCTCCTCGGAGCTGAAGGCTGGCAGGCCCG
AGGCCCGCGGCTCGCTAGCTCTGGACTGCGCCCCGCTGCTCAGGTTGCTGGGGCCGGCGCCGGG
GGTCTCCTGGACCGCCGGTTCACCAGCCCCGGCAGAGGCCCGGACGCTGTCCAGGGTGCTGAAG
GGCGGCTCCGTGCGCAAGCTCCGGCGTGCCAAGCAGTTGGTGCTGGAGCTGGGCGAGGAGGCGA
TCTTGGAGGGTTGCGTCGGGCCCCCCGGGGAGGCGGCTGTGGGGCTGCTCCAGTTCAATCTCAG
CGAGCTGTTCAGTTGGTGGATTCGCCAAGGCGAAGGGCGACTGAGGATCCGCCTGATGCCCGAG
AAGAAGGCGTCGGAAGTGGGCAGAGAGGGAAGGCTGTCCGCGGCAATTCGCGCCTCCCAGCCCC
GCCTTCTCTTCCAGATCTTCGGGACTGGTCATAGCTCCTTGGAATCACCAACAAACATGCCTTC
TCCTTCTCCTGATTATTTTACATGGAATCTCACCTGGATAATGAAAGACTCCTTCCCTTTCCTG
TCTCATCGCAGCCGATATGGTCTGGAGTGCAGCTTTGACTTCCCCTGTGAGCTGGAGTATTCCC
CTCCACTGCATGACCTCAGGAACCAGAGCTGGTCCTGGCGCCGCATCCCCTCCGAGGAGGCCTC
CCAGATGGACTTGCTGGATGGGCCTGGGGCAGAGCGTTCTAAGGAGATGCCCAGAGGCTCCTTT
CTCCTTCTCAACACCTCAGCTGACTCCAAGCACACCATCCTGAGTCCGTGGATGAGGAGCAGCA
GTGAGCACTGCACACTGGCCGTCTCGGTGCACAGGCACCTGCAGCCCTCTGGAAGGTACATTGC
CCAGCTGCTGCCCCACAACGAGGCTGCAAGAGAGATCCTCCTGATGCCCACTCCAGGGAAGCAT
GGTTGGACAGTGCTCCAGGGAAGAATCGGGCGTCCAGACAACCCATTTCGAGTGGCCCTGGAAT
ACATCTCCAGTGGAAACCGCAGCTTGTCTGCAGTGGACTTCTTTGCCCTGAAGAACTGCAGTGA
AGGAACATCCCCAGGCTCCAAGATGGCCCTGCAGAGCTCCTTCACTTGTTGGAATGGGACAGTC
CTCCAGCTTGGGCAGGCCTGTGACTTCCACCAGGACTGTGCCCAGGGAGAAGATGAGAGCCAGA
TGTGCCGGAAACTGCCTGTGGGTTTTTACTGCAACTTTGAAGATGGCTTCTGTGGCTGGACCCA
AGGCACACTGTCACCCCACACTCCTCAATGGCAGGTCAGGACCCTAAAGGATGCCCGGTTCCAG
GACCACCAAGACCATGCTCTATTGCTCAGTACCACTGATGTCCCCGCTTCTGAAAGTGCTACAG
TGACCAGTGCTACGTTTCCTGCACCGATCAAGAGCTCTCCATGTGAGCTCCGAATGTCCTGGCT
CATTCGTGGAGTCTTGAGGGGAAACGTGTCCTTGGTGCTAGTGGAGAACAAAACCGGGAAGGAG
CAAGGCAGGATGGTCTGGCATGTCGCCGCCTATGAAGGCTTGAGCCTGTGGCAGTGGATGGTGT
TGCCTCTCCTCGATGTGTCTGACAGGTTCTGGCTGCAGATGGTCGCATGGTGGGGACAAGGATC
CAGAGCCATCGTGGCTTTTGACAATATCTCCATCAGCCTGGACTGCTACCTCACCATTAGCGGA
GAGGACAAGATCCTGCAGAATACAGCACCCAAATCAAGAAACCTGTTTGAGAGAAACCCAAACA
AGGAGCTGAAACCCGGGGAAAATTCACCAAGACAGACCCCCATCTTTGACCCTACAGTTCATTG
GCTGTTCACCACATGTGGGGCCAGCGGGCCCCATGGCCCCACCCAGGCACAGTGCAACAACGCC
TACCAGAACTCCAACCTGAGCGTGGAGGTGGGGAGCGAGGGCCCCCTGAAAGGCATCCAGATCT
GGAAGGTGCCAGCCACCGACACCTACAGCATCTCGGGCTACGGAGCTGCTGGCGGGAAAGGCGG
GAAGAACACCATGATGCGGTCCCACGGCGTGTCTGTGCTGGGCATCTTCAACCTGGAGAAGGAT
GACATGCTGTACATCCTGGTTGGGCAGCAGGGAGAGGACGCCTGCCCCAGTACAAACCAGTTAA
TCCAGAAAGTCTGCATTGGAGAGAACAATGTGATAGAAGAAGAAATCCGTGTGAACAGAAGCGT
GCATGAGTGGGCAGGAGGCGGAGGAGGAGGGGGTGGAGCCACCTACGTATTTAAGATGAAGGAT
GGAGTGCCGGTGCCCCTGATCATTGCAGCCGGAGGTGGTGGCAGGGCCTACGGGGCCAAGACAG
ACACGTTCCACCCAGAGAGACTGGAGAATAACTCCTCGGTTCTAGGGCTAAACGGCAATTCCGG
AGCCGCAGGTGGTGGAGGTGGCTGGAATGATAACACTTCCTTGCTCTGGGCCGGAAAATCTTTG
CAGGAGGGTGCCACCGGAGGACATTCCTGCCCCCAGGCCATGAAGAAGTGGGGGTGGGAGACAA
GAGGGGGTTTCGGAGGGGGTGGAGGGGGGTGCTCCTCAGGTGGAGGAGGCGGAGGATATATAGG
CGGCAATGCAGCCTCAAACAATGACCCCGAAATGGATGGGGAAGATGGGGTTTCCTTCATCAGT
CCACTGGGCATCCTGTACACCCCAGCTTTAAAAGTGATGGAAGGCCACGGGGAAGTGAATATTA
AGCATTATCTAAACTGCAGTCACTGTGAGGTAGACGAATGTCACATGGACCCTGAAAGCCACAA
GGTCATCTGCTTCTGTGACCACGGGACGGTGCTGGCTGAGGATGGCGTCTCCTGCATTGTGTCA
CCCACCCCGGAGCCACACCTGCCACTCTCGCTGATCCTCTCTGTGGTGACCTCTGCCCTCGTGG
CCGCCCTGGTCCTGGCTTTCTCCGGCATCATGATTGTGTACCGCCGGATGCACCAGGAGCTGCA
AGCCATGCAGATGGAGCTGCAGAGCCCTGAGTACAAGCTGAGCAAGCTCCGCACCTCGACCATC
ATGACCGACTACAACCCCAACTACTGCTTTGCTGGCAAGACCTCCTCCATCAGTGACCTGAAGG
AGGTGCCGCGGAAAAACATCACCCTCATTCGGGGTCTGGGCCATGGCGCCTTTGGGGAGGTGTA
TGAAGGCCAGGTGTCCGGAATGCCCAACGACCCAAGCCCCCTGCAAGTGGCTGTGAAGACGCTG
CCTGAAGTGTGCTCTGAACAGGACGAACTGGATTTCCTCATGGAAGCCCTGATCATCAGCAAAT
TCAACCACCAGAACATTGTTCGCTGCATTGGGGTGAGCCTGCAATCCCTGCCCCGGTTCATCCT
GCTGGAGCTCATGGCGGGGGGAGACCTCAAGTCCTTCCTCCGAGAGACCCGCCCTCGCCCGAGC
CAGCCCTCCTCCCTGGCCATGCTGGACCTTCTGCACGTGGCTCGGGACATTGCCTGTGGCTGTC
AGTATTTGGAGGAAAACCACTTCATCCACCGAGACATTGCTGCCAGAAACTGCCTCTTGACCTG
TCCAGGCCCTGGAAGAGTGGCCAAGATTGGAGACTTCGGGATGGCCCGAGACATCTACAGGGCG
AGCTACTATAGAAAGGGAGGCTGTGCCATGCTGCCAGTTAAGTGGATGCCCCCAGAGGCCTTCA
TGGAAGGAATATTCACTTCTAAAACAGACACATGGTCCTTTGGAGTGCTGCTATGGGAAATCTT
TTCTCTTGGATATATGCCATACCCCAGCAAAAGCAACCAGGAAGTTCTGGAGTTTGTCACCAGT
GGAGGCCGGATGGACCCACCCAAGAACTGCCCTGGGCCTGTATACCGGATAATGACTCAGTGCT
GGCAACATCAGCCTGAAGACAGGCCCAACTTTGCCATCATTTTGGAGAGGATTGAATACTGCAC
CCAGGACCCGGATGTAATCAACACCGCTTTGCCGATAGAATATGGTCCACTTGTGGAAGAGGAA
GAGAAAGTGCCTGTGAGGCCCAAGGACCCTGAGGGGGTTCCTCCTCTCCTGGTCTCTCAACAGG
CAAAACGGGAGGAGGAGCGCAGCCCAGCTGCCCCACCACCTCTGCCTACCACCTCCTCTGGCAA
GGCTGCAAAGAAACCCACAGCTGCAGAGATCTCTGTTCGAGTCCCTAGAGGGCCGGCCGTGGAA
GGGGGACACGTGAATATGGCATTCTCTCAGTCCAACCCTCCTTCGGAGTTGCACAAGGTCCACG
GATCCAGAAACAAGCCCACCAGCTTGTGGAACCCAACGTACGGCTCCTGGTTTACAGAGAAACC
CACCAAAAAGAATAATCCTATAGCAAAGAAGGAGCCACACGACAGGGGTAACCTGGGGCTGGAG
GGAAGCTGTACTGTCCCACCTAACGTTGCAACTGGGAGACTTCCGGGGGCCTCACTGCTCCTAG
AGCCCTCTTCGCTGACTGCCAATATGAAGGAGGTACCTCTGTTCAGGCTACGTCACTTCCCTTG
TGGGAATGTCAATTACGGCTACCAGCAACAGGGCTTGCCCTTAGAAGCCGCTACTGCCCCTGGA
GCTGGTCATTACGAGGATACCATTCTGAAAAGCAAGAATAGCATGAACCAGCCTGGGCCCTGA

Another exemplary Homo sapiens ALK polynucleotide sequence from GenBank™ accession no. NM_004304 is provided below:

| (SEQ ID NO: 32973) | |
|---|---|
| 1 | agatgcgatc cagcggctct gggggcggca gcggtggtag cagctggtac ctcccgccgc |
| 61 | ctctgttcgg agggtcgcgg ggcaccgagg tgctttccgg ccgccctctg gtcggccacc |
| 121 | caaagccgcg ggcgctgatg atgggtgagg agggggcggc aagatttcgg gcgcccctgc |
| 181 | cctgaacgcc ctcagctgct gccgccgggg ccgctccagt gcctgcgaac tctgaggagc |
| 241 | cgaggcgccg gtgagagcaa ggacgctgca aacttgcgca gcgcgggggc tgggattcac |
| 301 | gcccagaagt tcagcaggca gacagtccga agccttcccg cagcggagag atagcttgag |
| 361 | ggtgcgcaag acggcagcct ccgccctcgg ttcccgccca gaccgggcag aagagcttgg |
| 421 | aggagccaaa aggaacgcaa aaggcggcca ggacagcgtg cagcagctgg gagccgccgt |
| 481 | tctcagcctt aaaagttgca gagattggag gctgccccga gaggggacag accccagctc |
| 541 | cgactgcggg gggcaggaga ggacggtacc caactgccac ctcccttcaa ccatagtagt |
| 601 | tcctctgtac cgagcgcagc gagctacaga cgggggcgcg gcactcggcg cggagagcgg |
| 661 | gaggctcaag gtcccagcca gtgagcccag tgtgcttgag tgtctctgga ctcgcccctg |
| 721 | agcttccagg tctgtttcat ttagactcct gctcgcctcc gtgcagttgg gggaaagcaa |
| 781 | gagacttgcg cgcacgcaca gtcctctgga gatcaggtgg aaggagccgc tgggtaccaa |
| 841 | ggactgttca gagcctcttc ccatctcggg gagagcgaag ggtgaggctg ggcccggaga |
| 901 | gcagtgtaaa cggcctcctc cggcgggatg ggagccatcg ggctcctgtg gctcctgccg |
| 961 | ctgctgcttt ccacggcagc tgtgggctcc gggatgggga ccggccagcg cgcgggctcc |
| 1021 | ccagctgcgg ggccgccgct gcagccccgg gagccactca gctactcgcg cctgcagagg |
| 1081 | aagagtctgg cagttgactt cgtggtgccc tcgctcttcc gtgtctacgc ccgggaccta |
| 1141 | ctgctgccac catcctcctc ggagctgaag gctggcaggc ccgaggcccg cggctcgcta |
| 1201 | gctctggact gcgccccgct gctcaggttg ctggggccgg cgccgggggt ctcctggacc |
| 1261 | gccggttcac cagccccggc agaggcccgg acgctgtcca gggtgctgaa gggcggctcc |
| 1321 | gtgcgcaagc tccggcgtgc caagcagttg gtgctggagc tgggcgagga ggcgatcttg |
| 1381 | gagggttgcg tcgggccccc cggggaggcg gctgtggggc tgctccagtt caatctcagc |
| 1441 | gagctgttca gttggtggat tcgccaaggc gaagggcgac tgaggatccg cctgatgccc |
| 1501 | gagaagaagg cgtcggaagt gggcagagag ggaaggctgt ccgcggcaat tcgcgcctcc |
| 1561 | cagccccgcc ttctcttcca gatcttcggg actggtcata gctccttgga atcaccaaca |
| 1621 | aacatgcctt ctccttctcc tgattatttt acatggaatc tcacctggat aatgaaagac |
| 1681 | tccttccctt tcctgtctca tcgcagccga tatggtctgg agtgcagctt tgacttcccc |
| 1741 | tgtgagctgg agtattcccc tccactgcat gacctcagga accagagctg gtcctggcgc |
| 1801 | cgcatcccct ccgaggaggc ctcccagatg gacttgctgg atgggcctgg ggcagagcgt |
| 1861 | tctaaggaga tgcccagagg ctcctttctc cttctcaaca cctcagctga ctccaagcac |
| 1921 | accatcctga gtccgtggat gaggagcagc agtgagcact gcacactggc cgtctcggtg |
| 1981 | cacaggcacc tgcagccctc tggaaggtac attgcccagc tgctgcccca caacgaggct |
| 2041 | gcaagagaga tcctcctgat gcccactcca gggaagcatg gttggacagt gctccaggga |
| 2101 | agaatcgggc gtccagacaa cccatttcga gtggccctgg aatacatctc cagtggaaac |
| 2161 | cgcagcttgt ctgcagtgga cttctttgcc ctgaagaact gcagtgaagg aacatcccca |
| 2221 | ggctccaaga tggccctgca gagctccttc acttgttgga atgggacagt cctccagctt |
| 2281 | gggcaggcct gtgacttcca ccaggactgt gcccagggag aagatgagag ccagatgtgc |
| 2341 | cggaaactgc ctgtgggttt ttactgcaac tttgaagatg gcttctgtgg ctggacccaa |
| 2401 | ggcacactgt caccccacac tcctcaatgg caggtcagga ccctaaagga tgcccggttc |
| 2461 | caggaccacc aagaccatgc tctattgctc agtaccactg atgtccccgc ttctgaaagt |
| 2521 | gctacagtga ccagtgctac gtttcctgca ccgatcaaga gctctccatg tgagctccga |
| 2581 | atgtcctggc tcattcgtgg agtcttgagg ggaaacgtgt ccttggtgct agtggagaac |
| 2641 | aaaaccggga aggagcaagg caggatggtc tggcatgtcg ccgcctatga aggcttgagc |
| 2701 | ctgtggcagt ggatggtgtt gcctctcctc gatgtgtctg acaggttctg gctgcagatg |
| 2761 | gtcgcatggt ggggacaagg atccagagcc atcgtggctt ttgacaatat ctccatcagc |
| 2821 | ctggactgct acctcaccat tagcggagag gacaagatcc tgcagaatac agcacccaaa |
| 2881 | tcaagaaacc tgtttgagag aaacccaaac aaggagctga aacccgggga aaattcacca |
| 2941 | agacagaccc ccatctttga ccctacagtt cattggctgt tcaccacatg tggggccagc |
| 3001 | gggccccatg gccccaccca ggcacagtgc aacaacgcct accagaactc caacctgagc |
| 3061 | gtggaggtgg ggagcgaggg ccccctgaaa ggcatccaga tctggaaggt gccagccacc |
| 3121 | gacacctaca gcatctcggg ctacggagct gctggcggga aaggcgggaa gaacaccatg |
| 3181 | atgcggtccc acggcgtgtc tgtgctgggc atcttcaacc tggagaagga tgacatgctg |
| 3241 | tacatcctgg ttgggcagca gggagaggac gcctgcccca gtacaaacca gttaatccag |
| 3301 | aaagtctgca ttggagagaa caatgtgata gaagaagaaa tccgtgtgaa cagaagcgtg |
| 3361 | catgagtggg caggaggcgg aggaggaggg ggtggagcca cctacgtatt taagatgaag |
| 3421 | gatggagtgc cggtgcccct gatcattgca gccggaggtg gtggcagggc ctacggggcc |
| 3481 | aagacagaca cgttccaccc agagagactg gagaataact cctcggttct agggctaaac |
| 3541 | ggcaattccg gagccgcagg tggtggaggt ggctggaatg ataacacttc cttgctctgg |
| 3601 | gccggaaaat ctttgcagga gggtgccacc ggaggacatt cctgccccca ggccatgaag |
| 3661 | aagtgggggt gggagacaag agggggtttc ggagggggtg gaggggggtg ctcctcaggt |
| 3721 | ggaggaggcg gaggatatat aggcggcaat gcagcctcaa acaatgaccc cgaaatggat |
| 3781 | ggggaagatg gggtttcctt catcagtcca ctgggcatcc tgtacacccc agctttaaaa |
| 3841 | gtgatggaag gccacgggga agtgaatatt aagcattatc taaactgcag tcactgtgag |
| 3901 | gtagacgaat gtcacatgga ccctgaaagc cacaaggtca tctgcttctg tgaccacggg |
| 3961 | acggtgctgg ctgaggatgg cgtctcctgc attgtgtcac ccaccccgga gccacacctg |
| 4021 | ccactctcgc tgatcctctc tgtggtgacc tctgccctcg tggccgccct ggtcctggct |
| 4081 | ttctccggca tcatgattgt gtaccgccgg aagcaccagg agctgcaagc catgcagatg |
| 4141 | gagctgcaga gccctgagta caagctgagc aagctccgca cctcgaccat catgaccgac |
| 4201 | tacaacccca actactgctt tgctggcaag acctcctcca tcagtgacct gaaggaggtg |
| 4261 | ccgcggaaaa acatcaccct cattcggggt ctgggccatg gcgcctttgg ggaggtgtat |
| 4321 | gaaggccagg tgtccggaat gcccaacgac ccaagccccc tgcaagtggc tgtgaagacg |
| 4381 | ctgcctgaag tgtgctctga acaggacgaa ctggatttcc tcatggaagc cctgatcatc |
| 4441 | agcaaattca accaccagaa cattgttcgc tgcattgggg tgagcctgca atccctgccc |
| 4501 | cggttcatcc tgctggagct catggcgggg ggagacctca agtccttcct ccgagagacc |
| 4561 | cgccctcgcc cgagccagcc ctcctccctg gccatgctgg accttctgca cgtggctcgg |
| 4621 | gacattgcct gtggctgtca gtatttggag gaaaaccact tcatccaccg agacattgct |
| 4681 | gccagaaact gcctcttgac ctgtccaggc cctggaagag tggccaagat tggagacttc |
| 4741 | gggatggccc gagacatcta cagggcgagc tactatagaa agggaggctg tgccatgctg |
| 4801 | ccagttaagt ggatgccccc agaggccttc atggaaggaa tattcacttc taaaacagac |
| 4861 | acatggtcct ttggagtgct gctatgggaa atcttttctc ttggatatat gccatacccc |
| 4921 | agcaaaagca accaggaagt tctggagttt gtcaccagtg gaggccggat ggacccaccc |
| 4981 | aagaactgcc ctgggcctgt ataccggata atgactcagt gctggcaaca tcagcctgaa |
| 5041 | gacaggccca actttgccat cattttggag aggattgaat actgcaccca ggacccggat |
| 5101 | gtaatcaaca ccgctttgcc gatagaatat ggtccacttg tggaagagga agagaaagtg |
| 5161 | cctgtgaggc ccaaggaccc tgagggggtt cctcctctcc tggtctctca acaggcaaaa |
| 5221 | cgggaggagg agcgcagccc agctgcccca ccacctctgc ctaccacctc ctctggcaag |
| 5281 | gctgcaaaga aacccacagc tgcagagatc tctgttcgag tccctagagg gccggccgtg |
| 5341 | gaagggggac acgtgaatat ggcattctct cagtccaacc ctccttcgga gttgcacaag |
| 5401 | gtccacggat ccagaaacaa gcccaccagc ttgtggaacc caacgtacgg ctcctggttt |
| 5461 | acagagaaac ccaccaaaaa gaataatcct atagcaaaga aggagccaca cgacaggggt |
| 5521 | aacctggggc tggagggaag ctgtactgtc ccacctaacg ttgcaactgg gagacttccg |
| 5581 | ggggcctcac tgctcctaga gccctcttcg ctgactgcca atatgaagga ggtacctctg |
| 5641 | ttcaggctac gtcacttccc ttgtgggaat gtcaattacg gctaccagca acagggcttg |
| 5701 | cccttagaag ccgctactgc ccctggagct ggtcattacg aggataccat tctgaaaagc |
| 5761 | aagaatagca tgaaccagcc tgggccctga gctcggtcgc acactcactt ctcttccttg |
| 5821 | ggatccctaa gaccgtggag gagagagagg caatggctcc ttcacaaacc agagaccaaa |
| 5881 | tgtcacgttt tgttttgtgc caacctattt tgaagtacca ccaaaaaagc tgtattttga |
| 5941 | aaatgcttta gaaaggtttt gagcatgggt tcatcctatt ctttcgaaag aagaaaatat |
| 6001 | cataaaaatg agtgataaat acaaggccca gatgtggttg cataaggttt ttatgcatgt |
| 6061 | ttgttgtata cttccttatg cttctttcaa attgtgtgtg ctctgcttca atgtagtcag |
| 6121 | aattagctgc ttctatgttt catagttggg gtcatagatg tttccttgcc ttgttgatgt |
| 6181 | ggacatgagc catttgaggg gagagggaac ggaaataaag gagttatttg taatgactaa |

An exemplary Mus musculus ALK nucleic acid sequence from GenBank™ accession no. NM_007439.2 is provided below:

| (SEQ ID NO: 32974) | |
|---|---|
| 1 | gtgttcacgc ccagaagttc agcgggcagg gtgatcgatc cgaagacttc ctgcagcgga |
| 61 | ggtcacttga gggggcgcta gaaagcagcc ccctccggtg gtccttgcct agacctggga |
| 121 | aggagcgcag aggaggtgac aggagcggag gacgtgggca agacagtgac cgactcggag |
| 181 | ccacggttca cagcctggaa agttgcagaa gattggaagc taagaggaga gctctggtcg |
| 241 | ccgagggctc cttgaacggt acctaattgc cacctccctg gtccctgagc aaaggcctct |
| 301 | acaaatgggg cgcagcacgg cgagaggcgc aggatccagc tgttgagccc agggtgtctc |
| 361 | actgtctccg aactaccccc tgactttgtc ttccgttttg ctgagaaccc ttctcgcctc |
| 421 | cttgtagctt gggaaaagca agggcgctct atagtgtaca cacagtccct gagatctagt |
| 481 | ggaaggagcc attcaggacc aaggactatt tggagccctt tcctgtttgg gggagagtga |
| 541 | agggcgaggc tggaccagca agggaaggga gactagtgta aactcgccct ccagcgggat |
| 601 | gggagctgct gggttcctgt ggctgctgcc tccactgctt ttggcagcag cctcgtactc |
| 661 | cggagctgca accgatcagc gcgcgggttc cccagcctca gggcctcctc tgcagccccg |
| 721 | ggagccgctc agttattcgc gcctgcagag gaagagtctg gcagtggact tcgttgtacc |
| 781 | ctcgctcttc cgcgtctatg cccgagacct gctgctaccg cagccacggt ccccctcgga |
| 841 | gcccgaggct ggcgggctgg aggcgcgggg atcactggcc ctggattgtg agcctctgct |
| 901 | caggctgctg gggccactgc ctggaatctc ctgggcagat ggagccagtt ctcctagtcc |
| 961 | cgaggcgggt ccgacgctgt ccagggtgct gaagggaggc tcggtgcgca agctcaggcg |
| 1021 | tgccaaacag ctggtgctgg agctgggcga ggagacgatt cttgaaggct gtattggtcc |
| 1081 | cccagaggag gtagcggctg tggggatact ccagttcaac ctcagcgagc tgttcagctg |
| 1141 | gtggattctc cacggcgaag ggaggctgag gatccgcctg atgcctgaga agaaggcatc |
| 1201 | ggaagtgggc agggagggaa ggctatccag tgcgatccga gcctcccagc cccgccttct |
| 1261 | cttccagatc ttcgggacgg gacacagctc catggagtca ccctcagaaa cgccttctcc |
| 1321 | tcctggtacc ttcatgtgga atctgacctg gacgatgaaa gactccttcc ctttcctttc |
| 1381 | ccaccgcagt cgatatggtc tggagtgcag ctttgacttc ccctgtgagc tggaatattc |
| 1441 | tcctcccctg cacaaccacg ggaatcagag ctggtcctgg cgccatgtgc cctccgagga |
| 1501 | ggcctcgagg atgaacttgc tggatgggcc agaggcagag cattctcaag agatgcccag |
| 1561 | aggctccttc ctcctcctga acacctctgc agattccaag cataccattc tgagcccatg |
| 1621 | gatgaggagc agtagtgatc actgcacact ggctgtctcc gtgcacagac atctacagcc |
| 1681 | ttcggggaga tatgttgccc agctcctacc ccacaacgaa gctggaagag agattctttt |
| 1741 | ggtgcccacc ccagggaagc atggctggac agtgctgcag gggagagtcg ggcgcccagc |
| 1801 | aaacccattt cgagtggctc tggaatacat ctccagtggc aaccggagct tgtcggcggt |
| 1861 | ggatttcttt gccctgaaga actgcagtga agggacatcc ccaggctcca agatggcatt |
| 1921 | gcagagttcc ttcacttgtt ggaatgggac cgtcctccag ctcgggcaag cctgtgattt |
| 1981 | ccaccaggac tgtgcccaag gagaagatga gggccagctg tgcagtaaac ttcctgctgg |
| 2041 | attttactgt aactttgaaa atggcttctg tggctggacc caaagtccac tctcacccca |
| 2101 | tatgccccgg tggcaagtga ggaccctaag agatgcccat tcccagggcc accaaggccg |
| 2161 | tgccctgttg ctcagcacca ctgacatcct cgcttctgaa ggtgcaacag tgaccagtgc |
| 2221 | caccttccct gcaccaatga aaaattctcc ttgtgagctc cgcatgtcct ggctcatccg |
| 2281 | cggggttttg agaggaaacg tatctctggt gctggtggag aacaaaaccg gaaaggagca |
| 2341 | aagccggact gtctggcatg tcgccactga cgaaggctta agcctgtggc agcatacagt |
| 2401 | gctgtccctc ctcgatgtga ctgacaggtt ctggctgcag atagtcacat ggtggggtcc |
| 2461 | aggatccagg gcaaccgtgg gatttgacaa catttccatc agcctcgact gctaccttac |
| 2521 | catcagtgga gaggagaaaa tgtccctgaa ttcagtaccc aaatctagaa atctgtttga |
| 2581 | gaaaaaccca aacaaggagt caaaatcctg ggcaaacata tcaggaccaa ctcccatctt |
| 2641 | cgaccctaca gttcactggc tgttcaccac gtgtggggcc agtggacctc atggccccac |
| 2701 | ccaggcacag tgcaacaacg cctaccagaa ttccaacttg agcgtggtgg tgggaagtga |
| 2761 | agggcccttg aagggagtcc agatttggaa agtgccagct actgacacct acagtatctc |
| 2821 | gggctacgga gcagctggcg ggaaaggtgg gaaaaacacc atgatgcggt cccatggcgt |
| 2881 | gtctgtcctg ggcatcttca atctggagaa aggtgacaca ctctacatcc ttgtcggtca |
| 2941 | gcaaggggag gatgcctgtc ccagggcaaa ccaactaatc cagaaagtct gtgtgggtga |
| 3001 | gaacaatgtc atagaagaag agatccgagt gaacagaagc gtgcacgagt gggcaggagg |
| 3061 | aggaggaggt gggggtggag ccacctacgt gtttaagatg aaagatggcg tgcctgtacc |
| 3121 | cctgatcatt gcagctggtg gtggtggcag ggcctatggg gccaagacag aaacgttcca |
| 3181 | cccagagaga ctggagagta actcctcggt tctagggctg aacggcaatt ccggagccgc |
| 3241 | aggtggtgga ggcggctgga atgataacac ttccttgctc tgggccggaa agtctttgct |
| 3301 | ggagggtgcc gccggaggac attcctgccc ccaggccatg aagaagtggg ggtgggagac |
| 3361 | aagagggggt ttcggagggg gtggaggggg gtgctcctca ggtggaggag gcggaggata |
| 3421 | tataggtggc aacgcagcat caaacaatga ccccgaaatg gatggggaag atggggtttc |
| 3481 | cttcatcagt ccattgggta tcctgtacac cccggcctta aaagtgatgg agggccacgg |
| 3541 | ggaagtgaat atcaagcatt atctaaactg cagtcactgc gaggtagacg aatgtcacat |
| 3601 | ggaccccgag agccacaaag tcatctgctt ctgtgatcat gggaccgtgc tggctgatga |
| 3661 | tggtgtctcc tgcattgtgt cacccacccc ggagccccac ctgccgctct cattgatcct |
| 3721 | ctccgtcgtg acctctgccc tggtggctgc ccttgttctg gcattctccg gcatcatgat |
| 3781 | tgtgtaccgt cggaagcacc aggagttgca ggctatgcag atggaactgc agagccccga |
| 3841 | gtataagctg agcaagctac ggacctcgac catcatgacc gactacaacc ccaactactg |
| 3901 | cttcgctggc aagacttcct ccatcagtga cctgaaagaa gtgccacgga aaaacatcac |
| 3961 | actcatccgg ggcctaggcc atggcgcatt tggggaggtg tatgaaggcc aggtgtctgg |
| 4021 | aatgcccaat gacccaagcc ctctacaagt ggctgtaaag acgctgccag aagtgtgttc |
| 4081 | agaacaagat gagctggact ttctcatgga agctctgatc atcagcaaat tcaaccacca |
| 4141 | gaatattgtt cgctgcatcg gggtgagtct acaagccctg ccccgcttca tcctgctgga |
| 4201 | actcatggct ggcggagacc tcaagtcctt cctcagggag acacgccctc gcccgaacca |
| 4261 | acccacctcc ctggccatgc tggaccttct gcatgtggct cgggacattg cctgtggctg |
| 4321 | tcagtaccta gaggagaatc actttatcca ccgggatatt gctgctagaa actgtctgtt |
| 4381 | gacctgccca ggagctggaa gaatagcaaa gattggagac tttgggatgg cccgagatat |
| 4441 | ctacagggcc agctactacc gaaagggagg ctgcgccatg ctgccggtca agtggatgcc |
| 4501 | ccctgaagcc ttcatggaag ggatatttac ctctaaaaca gacacatggt cttttggagt |
| 4561 | gttgctatgg gaaatatttt ctcttggata tatgccgtac cccagcaaga gcaaccagga |
| 4621 | agttctggag tttgtcacca gcggaggacg gatggacccg cctaagaact gccccgggcc |
| 4681 | tgtataccgg ataatgacgc agtgctggca gcatcagcct gaagacagac ccaacttcgc |
| 4741 | catcattttg gagaggatcg aatactgcac ccaggacccc gatgtgatca acacagctct |
| 4801 | gcccatcgaa tacggtccag tagtagaaga ggaggagaaa gtgcccatgc gccccaaaga |
| 4861 | ccccgagggg atgccacctt tgctggtgtc tccccagcct gcgaagcacg aggaggcgtc |
| 4921 | cgcagctccc cagcccgcag ccctgacggc accaggccca tcggtgaaga agcccccggg |
| 4981 | tgcgggtgcg ggcgcgggcg cgggtgcggg tgccggcccg gtgccccgag gtgcggccga |
| 5041 | tcggggccac gtgaacatgg ctttctctca gcccaaccct cccccggagc tgcacaaagg |
| 5101 | cccgggatcc agaaacaagc cgaccagcct gtggaacccc acctacggct cgtggttcac |
| 5161 | cgagaagcct gccaaaaaga cccatcctcc gccaggcgcc gagccgcagg cgcgggcagg |
| 5221 | agcggccgag ggtggctgga ccgggccggg cgcggggccc cgcagagccg aggcagcgct |
| 5281 | gctgctagag ccatcggcgc tgagcgccac catgaaggag gtgccgctgt tcaggctgcg |
| 5341 | ccacttcccc tgcggcaatg tcaactatgg ttaccagcaa cagggtctcc ccttggaagc |
| 5401 | cacagccgcg ccaggggaca ccatgctgaa aagcaagaat aaggtcaccc agccggggcc |
| 5461 | ctgagccctg tactccacta gcttctcctc ctggcggagc cggagcccac ccagagggag |
| 5521 | atggacagga tggctccacc acaaacccaa gaccaaaact ttcatttttg tgccaacttg |
| 5581 | ttttgaagtg ccacatttta aaaaaaggaa acttgtgttt ttaagatgtg ttagaaggtt |
| 5641 | ttttgagcat gggttcatct atcctctcaa aagaagaaaa tgccattctt taaaaaagaa |
| 5701 | aaaaaagcaa tcagtgcaag gcccagattg gttgcgccaa gttttcgtgc atggtctgct |
| 5761 | gtacagtccc ctaaggcttc tttccgattt ttgtgtgcgc tctgcttccg cgtagtcaga |
| 5821 | aatagctgct tccatgtctc atagggggag tcctaggtgt ttcctttgcc ttatgaatat |
| 5881 | gaaccactcg aggggcgggc gagggaacag aaataaag |

By “alteration” is meant a change in the structure, such as a chromosomal tranlocation or a gene rearrangement or gene amplification, or the expression levels or activity of a gene or polypeptide as detected by standard art known methods such as those described herein. An alteration may be an increase or decrease. As used herein, an alteration includes a 5% change in expression levels, a 10% change in expression levels, preferably a 25% change, more preferably a 40% change, and most preferably a 50% or greater change in expression levels.

By “ameliorate” is meant decrease, reduce, delay diminish, suppress, attenuate, arrest, or stabilize the development or progression of a disease or pathological condition.

By “antibody” is meant an immunoglobulin polypeptide having immunogen binding ability. Antibodies are evoked or elicited in subjects (humans or other animals or mammals) following exposure to a specific antigen (immunogen). A subject capable of generating antibodies/immunoglobulins (i.e., an immune response) directed against a specific antigen/immunogen is said to be immunocompetent. Antibodies are characterized by reacting specifically with (e.g., binding to) an antigen or immunogen in some demonstrable way, antibody, and antigen/immunogen each being defined in terms of the other.

By “amphiphile” is meant a chemical compound possessing both hydrophilic and lipophilic properties. Such a compound is called amphiphilic or amphipathic. The amphiphile may be conjugated or linked to an antigen or adjuvant cargo by a solubility-promoting polar polymer chain. In some embodiments, the amphiphile is conjugated or linked to an adjuvant. In some embodiments, the adjuvant is Freund's adjuvant. In some embodiments, the amphiphile is conjugated or linked to an ALK antigen or immunogen. In some embodiments, the amphiphile is a lipophilic albumin-binding tail. In some embodiments, the amphiphile is N-hydroxy succinimidyl ester-end-functionalized poly(ethylene glycol)-lipid (NHS-PEG2KDa-DSPE).

By “antigen” is meant an agent that can stimulate an immune response in an animal. An antigen reacts with the products of specific humoral or cellular immunity, including those induced by heterologous immunogens. In some embodiments of the disclosed compositions and methods, the antigen is an ALK protein or an antibody-binding portion thereof.

By “chimeric antigen receptor T cell” or “CAR-T cell” is meant a T cell expressing a CAR that has antigen specificity determined by the antibody-derived targeting domain of the CAR. As used herein, “CAR-T cells” includes T cells or NK cells. As used herein, “CAR-T cells” includes cells engineered to express a CAR or a T cell receptor (TCR). In some embodiments, CAR-T cells can be T helper CD4+ and/or T effector CD8+ cells, optionally in defined proportions. In some embodiments, CAR-T cells may comprise total CD3+ cells. Methods of making CARS (e.g., for treatment of cancer) are publicly available (see, e.g., Park et al., Trends Biotechnol., 29:550-557, 2011: Grupp et al., N Engl J Med., 368:1509-1518, 2013; Han et al., J. Hematol Oncol. 6:47, 2013; Haso et al., (2013) Blood, 121, 1165-1174; PCT Pubs. WO2012/079000, WO2013/059593; and U.S. Pub. 2012/0213783, each of which is incorporated by reference herein in its entirety). In some embodiments, the CAR-T cell expresses an ALK CAR.

A “codon-optimized” nucleic acid (polynucleotide) refers to a nucleic acid sequence that has been altered such that the codons are optimal for expression in a particular system (such as a particular species of group of species). For example, a nucleic acid sequence can be optimized for expression in mammalian cells. Codon optimization does not alter the amino acid sequence of the encoded protein.

In this disclosure, “comprises,” “comprising,” “containing” and “having” and the like can have the meaning ascribed to them in U.S. patent law and can mean “includes,” “including,” and the like: “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of an analyte, compound, agent, or substance to be detected.

By “detectable label” is meant a composition that, when linked to a molecule of interest, renders the latter detectable, e.g., via spectroscopic, photochemical, biochemical, immunochemical, or chemical means. Nonlimiting examples of useful detectable labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include those caused by oncogenic ALK gene fusions, rearrangements, duplications, or mutations (e.g., ALK-positive cancers). In some embodiments, the cancer is an ALK-positive cancer.

By “ALK positive” is meant having detectable ALK polypeptide or polynucleotide expression. Methods for measuring ALK expression are described, for example, in Vernersson, et al. “Characterization of the expression of the ALK receptor tyrosine kinase in mice,” Gene Expr Patterns, 6:448-461 (2005) and in Dirks, et al. “Expression and functional analysis of the anaplastic lymphoma kinase (ALK) gene in tumor cell lines,” Int. J. Cancer, 100:49-56 (2002), the disclosures of which are incorporated herein by reference in their entirities for all purposes. In embodiments, an ALK positive cell contains a change to the structure of the ALK gene. In some cases, an ALK positive cell expresses ALK at higher levels than a reference cell (e.g., a healthy non-neoplastic cell). Nonlimiting examples of ALK-positive cancers include non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, and melanoma. The ALK-positive cancer may be caused by an oncogenic ALK gene that either forms a fusion gene with other genes, gains additional gene copies, or is genetically mutated. In some embodiments, the ALK-positive cancer is caused by an ALK fusion gene encoding an ALK fusion protein. In some embodiments, the ALK-positive cancer is caused by a fusion between the ALK gene and the nucleophosmin (NPM) gene encoding a NPM-ALK fusion protein. In some embodiments, the ALK-positive cancer is caused by a fusion between the ALK gene and the echinoderm microtubule-associated protein-like 4 (EML4) gene encoding an ELM4-ALK fusion protein.

By “effective amount” is meant the amount of an agent required to ameliorate, reduce, delay, improve, abrogate, diminish, or eliminate the symptoms and/or effects of a disease, condition, or pathology relative to an untreated patient. In some embodiments, an effective amount of a cell expressing a T cell receptor is the amount required to reduce the size of a tumor, stabilize tumor cell growth, eliminate a tumor, reduce or eliminate metastasis in a subject. In some embodiments, an effective amount of an ALK peptide is the amount required to induce an ALK-specific immune response in a subject immunized with the peptide. The effective amount of an immunogen or a composition comprising an immunogen, as used to practice the methods of therapeutic treatment of a disease, condition, or pathology, varies depending upon the manner of administration, the age, body weight, and general health of the subject. Ultimately, the attending physician or veterinarian will decide the appropriate amount and dosage regimen. Such amount is referred to as an “effective” amount.

By “epitope,” as used herein, means an antigenic determinant. An epitope is the part of an antigen molecule that by its structure determines the immunoglobulin that will recognize and specifically bind to elicit a specific immune response. In some embodiments, a disclosed engineered TCR or immunoglobulin specifically binds to an epitope on an ALK polypeptide.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 3, 4, 5, 6, 7, 8, 9, 10, 20, 30, 40, 50, 60, 70, 80, 90, or 100, 200, 300, 400, 500, 600, 700, 800, 900, or 1000 nucleotides or amino acids. A portion or fragment of a polypeptide may be a peptide. In the case of an antibody or immunoglobulin fragment, the fragment typically binds to the target antigen.

By “fusion protein” is meant a protein generated by expression of a nucleic acid (polynucleotide) sequence engineered from nucleic acid sequences encoding at least a portion of two different (heterologous) proteins or peptides. To create a fusion protein, the nucleic acid sequences must be in the same open reading frame and contain no internal stop codons. One protein can be located at the amino-terminal (N-terminal) portion of the fusion protein or at the carboxy-terminal (C-terminal) protein thus forming an amino-terminal fusion protein or a carboxy-terminal fusion protein, respectively.

For example, a fusion protein includes an ALK protein fused to a heterologous protein. In some embodiments, the fusion protein is an ALK protein fused to a nucleophosmin (NPM) protein. In some embodiments, the NPM-ALK fusion protein is at least 85%, at least 90%, at least 95%, at least 98%, at least 99%, or 100% identical to a NPM-ALK fusion protein in Homo sapiens. In some embodiments, the NPM-ALK fusion protein is at least 85%, at least 90%, at least 95%, at least 98%, at least 99%, or 100% identical to an exemplary NPM-ALK fusion protein amino acid sequence from Homo sapiens as provided below (see GenBank Accession Nos. BAA08343.1, AAA58698.1):

>BAA08343.1 p80 protein [Homo sapiens]
(ALK cytoplasmic portion in bold font)
(SEQ ID NO: 32975)
MEDSMDMDMSPLRPQNYLFGCELKADKDYHFKVDNDENEHQLSLRTVSLGAGAKDELHIVEAEA
MNYEGSPIKVTLATLKMSVQPTVSLGGFEITPPVVLRLKCGSGPVHISGQHLVVYRRKHQELQA
MQMELQSPEYKLSKLRTSTIMTDYNPNYCFAGKTSSISDLKEVPRKNITLIRGLGHGAFGEVYE
GQVSGMPNDPSPLQVAVKTLPEVCSEQDELDFLMEALIISKFNHQNIVRCIGVSLQSLPRFILL
ELMAGGDLKSFLRETRPRPSQPSSLAMLDLLHVARDIACGCQYLEENHFIHRDIAARNCLLTCP
GPGRVAKIGDFGMARDIYRASYYRKGGCAMLPVKWMPPEAFMEGIFTSKTDTWSFGVLLWEIFS
LGYMPYPSKSNQEVLEFVTSGGRMDPPKNCPGPVYRIMTQCWQHQPEDRPNFAIILERIEYCTQ
DPDVINTALPIEYGPLVEEEEKVPVRPKDPEGVPPLLVSQQAKREEERSPAAPPPLPTTSSGKA
AKKPTAAEVSVRVPRGPAVEGGHVNMAFSQSNPPSELHRVHGSRNKPTSLWNPTYGSWFTEKPT
KKNNPIAKKEPHERGNLGLEGSCTVPPNVATGRLPGASLLLEPSSLTANMKEVPLFRLRHFPCG
NVNYGYQQQGLPLEAATAPGAGHYEDTILKSKNSMNQPGP.
>AAA58698.1 nucleophosmin-anaplastic lymphoma
kinase fusion protein [Homo sapiens]
(SEQ ID NO: 32976)
MEDSMDMDMSPLRPQNYLFGCELKADKDYHFKVDNDENEHQLSLRTVSLGAGAKDELHIVEAEA
MNYEGSPIKVTLATLKMSVQPTVSLGGFEITPPVVLRLKCGSGPVHISGQHLVVYRRKHQELQA
MQMELQSPEYKLSKLRTSTIMTDYNPNYCFAGKTSSISDLKEVPRKNITLIRGLGHGAFGEVYE
GQVSGMPNDPSPLQVAVKTLPEVCSEQDELDFLMEALIISKFNHQNIVRCIGVSLQSLPRFILL
ELMAGGDLKSFLRETRPRPSQPSSLAMLDLLHVARDIACGCQYLEENHFIHRDIAARNCLLTCP
GPGRVAKIGDFGMARDIYRASYYRKGGCAMLPVKWMPPEAFMEGIFTSKTDTWSFGVLLWEIFS
LGYMPYPSKSNQEVLEFVTSGGRMDPPKNCPGPVYRIMTQCWQHQPEDRPNFAIILERIEYCTQ
DPDVINTALPIEYGPLVEEEEKVPVRPKDPEGVPPLLVSQQAKREEERSPAAPPPLPTTSSGKA
AKKPTAAEVSVRVPRGPAVEGGHVNMAFSQSNPPSELHKVHGSRNKPTSLWNPTYGSWFTEKPT
KKNNPIAKKEPHDRGNLGLEGSCTVPPNVATGRLPGASLLLEPSSLTANMKEVPLFRLRHFPCG
NVNYGYQQQGLPLEAATAPGAGHYEDTILKSKNSMNQPGP

In some embodiments, the NPM-ALK fusion protein is encoded by a nucleic acid sequence that is at least 85%, at least 90%, at least 95%, at least 98%, at least 99%, or 100% identical to an exemplary nucleic acid sequence from Homo sapiens as provided below (see GenBank Accessions No. D45915.1 and U04946.1):

>D45915.1: 90-2132 Homo sapiens mRNA for p80 protein, complete cds
(SEQ ID NO: 32977)
ATGGAAGATTCGATGGACATGGACATGAGCCCCCTGAGGCCCCAGAACTATCTTTTCGGTTGTG
AACTAAAGGCCGACAAAGATTATCACTTTAAGGTGGATAATGATGAAAATGAGCACCAGTTATC
TTTAAGAACGGTCAGTTTAGGGGCTGGTGCAAAGGATGAGTTGCACATTGTTGAAGCAGAGGCA
ATGAATTACGAAGGCAGTCCAATTAAAGTAACACTGGCAACTTTGAAAATGTCTGTACAGCCAA
CGGTTTCCCTTGGGGGCTTTGAAATAACACCACCAGTGGTCTTAAGGTTGAAGTGTGGTTCAGG
GCCAGTGCATATTAGTGGACAGCACTTAGTAGTGTACCGCCGGAAGCACCAGGAGCTGCAAGCC
ATGCAGATGGAGCTGCAGAGCCCTGAGTACAAGCTGAGCAAGCTCCGCACCTCGACCATCATGA
CCGACTACAACCCCAACTACTGCTTTGCTGGCAAGACCTCCTCCATCAGTGACCTGAAGGAGGT
GCCACGGAAAAACATCACCCTCATTCGGGGTCTGGGCCATGGAGCCTTTGGGGAGGTGTATGAA
GGCCAGGTGTCCGGAATGCCCAACGACCCAAGCCCCCTGCAAGTGGCTGTGAAGACGCTGCCTG
AAGTGTGCTCTGAACAGGACGAACTGGATTTCCTCATGGAAGCCCTGATCATCAGCAAATTCAA
CCACCAGAACATTGTTCGCTGCATTGGGGTGAGCCTGCAATCCCTGCCCCGGTTCATCCTGCTG
GAGCTCATGGCGGGGGGAGACCTCAAGTCCTTCCTCCGAGAGACCCGCCCTCGCCCGAGCCAGC
CCTCCTCCCTGGCCATGCTGGACCTTCTGCACGTGGCTCGGGACATTGCCTGTGGCTGTCAGTA
TTTGGAGGAAAACCACTTCATCCACCGAGACATTGCTGCCAGAAACTGCCTCTTGACCTGTCCA
GGCCCTGGAAGAGTGGCCAAGATTGGAGACTTCGGGATGGCCCGAGACATCTACAGGGCGAGCT
ACTATAGAAAGGGAGGCTGTGCCATGCTGCCAGTTAAGTGGATGCCCCCAGAGGCCTTCATGGA
AGGAATATTCACTTCTAAAACAGACACATGGTCCTTTGGAGTGCTGCTATGGGAAATCTTTTCT
CTTGGATATATGCCATACCCCAGCAAAAGCAACCAGGAAGTTCTGGAGTTTGTCACCAGTGGAG
GCCGGATGGACCCACCCAAGAACTGCCCTGGGCCTGTATACCGGATAATGACTCAGTGCTGGCA
ACATCAGCCTGAAGACAGGCCCAACTTTGCCATCATTTTGGAGAGGATTGAATACTGCACCCAG
GACCCGGATGTAATCAACACCGCTTTGCCGATAGAATATGGTCCACTTGTGGAAGAGGAAGAGA
AAGTGCCTGTGAGGCCCAAGGACCCTGAGGGGGTTCCTCCTCTCCTGGTCTCTCAACAGGCAAA
ACGGGAGGAGGAGCGCAGCCCAGCTGCCCCACCACCTCTGCCTACCACCTCCTCTGGCAAGGCT
GCAAAGAAACCCACAGCTGCAGAGGTCTCTGTTCGAGTCCCTAGAGGGCCGGCCGTGGAAGGGG
GACACGTGAATATGGCATTCTCTCAGTCCAACCCTCCTTCGGAGTTGCACAGGGTCCACGGATC
CAGAAATAAGCCCACCAGCTTGTGGAACCCAACGTACGGCTCCTGGTTTACAGAGAAACCCACC
AAAAAGAATAATCCTATAGCAAAGAAGGAGCCACACGAGAGGGGTAACCTGGGGCTGGAGGGAA
GCTGTACTGTCCCACCTAACGTTGCAACTGGGAGACTTCCGGGGGCCTCACTGCTCCTAGAGCC
CTCTTCGCTGACTGCCAATATGAAGGAGGTACCTCTGTTCAGGCTACGTCACTTCCCTTGTGGG
AATGTCAATTACGGCTACCAGCAACAGGGCTTGCCCTTAGAAGCCGCTACTGCCCCTGGAGCTG
GTCATTACGAGGATACCATTCTGAAAAGCAAGAATAGCATGAACCAGCCTGGGCCCTGA.
>U04946.1 Human nucleophosmin-anaplastic lymphoma kinase
fusion protein (NPM/ALK) mRNA, complete cds
(SEQ ID NO: 32978)
ATGGAAGATTCGATGGACATGGACATGAGCCCCCTGAGGCCCCAGAACTATCTTTTCGGTTGTG
AACTAAAGGCCGACAAAGATTATCACTTTAAGGTGGATAATGATGAAAATGAGCACCAGTTATC
TTTAAGAACGGTCAGTTTAGGGGCTGGTGCAAAGGATGAGTTGCACATTGTTGAAGCAGAGGCA
ATGAATTACGAAGGCAGTCCAATTAAAGTAACACTGGCAACTTTGAAAATGTCTGTACAGCCAA
CGGTTTCCCTTGGGGGCTTTGAAATAACACCACCAGTGGTCTTAAGGTTGAAGTGTGGTTCAGG
GCCAGTGCATATTAGTGGACAGCACTTAGTAGTGTACCGCCGGAAGCACCAGGAGCTGCAAGCC
ATGCAGATGGAGCTGCAGAGCCCTGAGTACAAGCTGAGCAAGCTCCGCACCTCGACCATCATGA
CCGACTACAACCCCAACTACTGCTTTGCTGGCAAGACCTCCTCCATCAGTGACCTGAAGGAGGT
GCCGCGGAAAAACATCACCCTCATTCGGGGTCTGGGCCATGGCGCCTTTGGGGAGGTGTATGAA
GGCCAGGTGTCCGGAATGCCCAACGACCCAAGCCCCCTGCAAGTGGCTGTGAAGACGCTGCCTG
AAGTGTGCTCTGAACAGGACGAACTGGATTTCCTCATGGAAGCCCTGATCATCAGCAAATTCAA
CCACCAGAACATTGTTCGCTGCATTGGGGTGAGCCTGCAATCCCTGCCCCGGTTCATCCTGCTG
GAGCTCATGGCGGGGGGAGACCTCAAGTCCTTCCTCCGAGAGACCCGCCCTCGCCCGAGCCAGC
CCTCCTCCCTGGCCATGCTGGACCTTCTGCACGTGGCTCGGGACATTGCCTGTGGCTGTCAGTA
TTTGGAGGAAAACCACTTCATCCACCGAGACATTGCTGCCAGAAACTGCCTCTTGACCTGTCCA
GGCCCTGGAAGAGTGGCCAAGATTGGAGACTTCGGGATGGCCCGAGACATCTACAGGGCGAGCT
ACTATAGAAAGGGAGGCTGTGCCATGCTGCCAGTTAAGTGGATGCCCCCAGAGGCCTTCATGGA
AGGAATATTCACTTCTAAAACAGACACATGGTCCTTTGGAGTGCTGCTATGGGAAATCTTTTCT
CTTGGATATATGCCATACCCCAGCAAAAGCAACCAGGAAGTTCTGGAGTTTGTCACCAGTGGAG
GCCGGATGGACCCACCCAAGAACTGCCCTGGGCCTGTATACCGGATAATGACTCAGTGCTGGCA
ACATCAGCCTGAAGACAGGCCCAACTTTGCCATCATTTTGGAGAGGATTGAATACTGCACCCAG
GACCCGGATGTAATCAACACCGCTTTGCCGATAGAATATGGTCCACTTGTGGAAGAGGAAGAGA
AAGTGCCTGTGAGGCCCAAGGACCCTGAGGGGGTTCCTCCTCTCCTGGTCTCTCAACAGGCAAA
ACGGGAGGAGGAGCGCAGCCCAGCTGCCCCACCACCTCTGCCTACCACCTCCTCTGGCAAGGCT
GCAAAGAAACCCACAGCTGCAGAGGTCTCTGTTCGAGTCCCTAGAGGGCCGGCCGTGGAAGGGG
GACACGTGAATATGGCATTCTCTCAGTCCAACCCTCCTTCGGAGTTGCACAAGGTCCACGGATC
CAGAAACAAGCCCACCAGCTTGTGGAACCCAACGTACGGCTCCTGGTTTACAGAGAAACCCACC
AAAAAGAATAATCCTATAGCAAAGAAGGAGCCACACGACAGGGGTAACCTGGGGCTGGAGGGAA
GCTGTACTGTCCCACCTAACGTTGCAACTGGGAGACTTCCGGGGGCCTCACTGCTCCTAGAGCC
CTCTTCGCTGACTGCCAATATGAAGGAGGTACCTCTGTTCAGGCTACGTCACTTCCCTTGTGGG
AATGTCAATTACGGCTACCAGCAACAGGGCTTGCCCTTAGAAGCCGCTACTGCCCCTGGAGCTG
GTCATTACGAGGATACCATTCTGAAAAGCAAGAATAGCATGAACCAGCCTGGGCCCTGA.

In some embodiments, the fusion protein is an ALK protein fused to an echinoderm microtubule-associated protein-like 4 (EML4) protein. In some embodiments, the ELM4-ALK fusion protein is at least 85%, at least 90%, at least 95%, at least 98%, at least 99%, or 100% identical to a ELM4-ALK fusion protein in Homo sapiens or a variant thereof. In some embodiments, the ELM4-ALK fusion protein is at least 85%, at least 90%, at least 95%, at least 98%, at least 99%, or 100% identical to an exemplary ELM4-ALK fusion protein amino acid sequence from Homo sapiens as provided below (see GenBank Accessions No. BAM37627.1 and BAF73611.1);

>BAM37627.1 EML4-ALK fusion protein [Homo sapiens]
(SEQ ID NO: 32979)
MDGFAGSLDDSISAASTSDVQDRLSALESRVQQQEDEITVLKAALADVLRRLAISEDHVASVKK
SVSSKGQPSPRAVIPMSCITNGSGANRKPSHTSAVSIAGKETLSSAAKSGTEKKKEKPQGQREK
KEESHSNDQSPQIRASPSPQPSSQPLQIHRQTPESKNATPTKSIKRPSPAEKSHNSWENSDDSR
NKLSKIPSTPKLIPKVTKTADKHKDVIINQEGEYIKMFMRGRPITMFIPSDVDNYDDIRTELPP
EKLKLEWAYGYRGKDCRANVYLLPTGEIVYFIASVVVLFNYEERTQRHYLGHTDCVKCLAIHPD
KIRIATGQIAGVDKDGRPLQPHVRVWDSVTLSTLQIIGLGTFERGVGCLDFSKADSGVHLCVID
DSNEHMLTVWDWQRKAKGAEIKTTNEVVLAVEFHPTDANTIITCGKSHIFFWTWSGNSLTRKQG
IFGKYEKPKFVQCLAFLGNGDVLTGDSGGVMLIWSKTTVEPTPGKGPKGVYQISKQIKAHDGSV
FTLCQMRNGMLLTGGGKDRKIILWDHDLNPEREIEFSASRARLPGHVAADHPPAVYRRKHQELQ
AMQMELQSPEYKLSKLRTSTIMTDYNPNYCFAGKTSSISDLKEVPRKNITLIRGLGHGAFGEVY
EGQVSGMPNDPSPLQVAVKTLPEVCSEQDELDFLMEALIISKFNHQNIVRCIGVSLQSLPRFIL
LELMAGGDLKSFLRETRPRPSQPSSLAMLDLLHVARDIACGCQYLEENHFIHRDIAARNCLLTC
PGPGRVAKIGDFGMARDIYRASYYRKGGCAMLPVKWMPPEAFMEGIFTSKTDTWSFGVLLWEIF
SLGYMPYPSKSNQEVLEFVTSGGRMDPPKNCPGPVYRIMTQCWQHQPEDRPNFAIILERIEYCT
QDPDVINTALPIEYGPLVEEEEKVPVRPKDPEGVPPLLVSQQAKREEERSPAAPPPLPTTSSGK
AAKKPTAAEISVRVPRGPAVEGGHVNMAFSQSNPPSELHKVHGSRNKPTSLWNPTYGSWFTEKP
TKKNNPIAKKEPHDRGNLGLEGSCTVPPNVATGRLPGASLLLEPSSLTANMKEVPLFRLRHFPC
GNVNYGYQQQGLPLEAATAPGAGHYEDTILKSKNSMNQPGP.
>BAF73611.1 fusion protein EML4-ALK variant 1 [Homo sapiens]
(SEQ ID NO: 32980)
MDGFAGSLDDSISAASTSDVQDRLSALESRVQQQEDEITVLKAALADVLRRLAISEDHVASVKK
SVSSKGQPSPRAVIPMSCITNGSGANRKPSHTSAVSIAGKETLSSAAKSGTEKKKEKPQGQREK
KEESHSNDQSPQIRASPSPQPSSQPLQIHRQTPESKNATPTKSIKRPSPAEKSHNSWENSDDSR
NKLSKIPSTPKLIPKVTKTADKHKDVIINQEGEYIKMFMRGRPITMFIPSDVDNYDDIRTELPP
EKLKLEWAYGYRGKDCRANVYLLPTGEIVYFIASVVVLFNYEERTQRHYLGHTDCVKCLAIHPD
KIRIATGQIAGVDKDGRPLQPHVRVWDSVTLSTLQIIGLGTFERGVGCLDFSKADSGVHLCVID
DSNEHMLTVWDWQKKAKGAEIKTTNEVVLAVEFHPTDANTIITCGKSHIFFWTWSGNSLTRKQG
IFGKYEKPKFVQCLAFLGNGDVLTGDSGGVMLIWSKTTVEPTPGKGPKVYRRKHQELQAMQMEL
QSPEYKLSKLRTSTIMTDYNPNYCFAGKTSSISDLKEVPRKNITLIRGLGHGAFGEVYEGQVSG
MPNDPSPLQVAVKTLPEVCSEQDELDFLMEALIISKFNHQNIVRCIGVSLQSLPRFILLELMAG
GDLKSFLRETRPRPSQPSSLAMLDLLHVARDIACGCQYLEENHFIHRDIAARNCLLTCPGPGRV
AKIGDFGMARDIYRASYYRKGGCAMLPVKWMPPEAFMEGIFTSKTDTWSFGVLLWEIFSLGYMP
YPSKSNQEVLEFVTSGGRMDPPKNCPGPVYRIMTQCWQHQPEDRPNFAIILERIEYCTQDPDVI
NTALPIEYGPLVEEEEKVPVRPKDPEGVPPLLVSQQAKREEERSPAAPPPLPTTSSGKAAKKPT
AAEVSVRVPRGPAVEGGHVNMAFSQSNPPSELHRVHGSRNKPTSLWNPTYGSWFTEKPTKKNNP
IAKKEPHERGNLGLEGSCTVPPNVATGRLPGASLLLEPSSLTANMKEVPLFRLRHFPCGNVNYG
YQQQGLPLEAATAPGAGHYEDTILKSKNSMNQPGP.

In some embodiments, the ELM4-ALK fusion protein is encoded by a nucleic acid sequence that is at least 85%, at least 90%, at least 95%, at least 98%, at least 99%, or 100% identical to an exemplary nucleic acid sequence from Homo sapiens as provided below (see GenBank Accessions No. AB663645.1 and AB274722.1):

>AB663645.1:15-3404 Homo sapiens EML4-ALK mRNA for EML4-ALK
fusion protein, complete cds
(SEQ ID NO: 32981)
ATGGACGGTTTCGCCGGCAGTCTCGATGATAGTATTTCTGCTGCAAGTACTTCTGATGTTCAAG
ATCGCCTGTCAGCTCTTGAGTCACGAGTTCAGCAACAAGAAGATGAAATCACTGTGCTAAAGGC
GGCTTTGGCTGATGTTTTGAGGCGTCTTGCAATCTCTGAAGATCATGTGGCCTCAGTGAAAAAA
TCAGTCTCAAGTAAAGGCCAACCAAGCCCTCGAGCAGTTATTCCCATGTCCTGTATAACCAATG
GAAGTGGTGCAAACAGAAAACCAAGTCATACCAGTGCTGTCTCAATTGCAGGAAAAGAAACTCT
TTCATCTGCTGCTAAAAGTGGTACAGAAAAAAAGAAAGAAAAACCACAAGGACAGAGAGAAAAA
AAAGAGGAATCTCATTCTAATGATCAAAGTCCACAAATTCGAGCATCACCTTCTCCCCAGCCCT
CTTCACAACCTCTCCAAATACACAGACAAACTCCAGAAAGCAAGAATGCTACTCCCACCAAAAG
CATAAAACGACCATCACCAGCTGAAAAGTCACATAATTCTTGGGAAAATTCAGATGATAGCCGT
AATAAATTGTCGAAAATACCTTCAACACCCAAATTAATACCAAAAGTTACCAAAACTGCAGACA
AGCATAAAGATGTCATCATCAACCAAGAAGGAGAATATATTAAAATGTTTATGCGCGGTCGGCC
AATTACCATGTTCATTCCTTCCGATGTTGACAACTATGATGACATCAGAACGGAACTGCCTCCT
GAGAAGCTCAAACTGGAGTGGGCATATGGTTATCGAGGAAAGGACTGTAGAGCTAATGTTTACC
TTCTTCCGACCGGGGAAATAGTTTATTTCATTGCATCAGTAGTAGTACTATTTAATTATGAGGA
GAGAACTCAGCGACACTACCTGGGCCATACAGACTGTGTGAAATGCCTTGCTATACATCCTGAC
AAAATTAGGATTGCAACTGGACAGATAGCTGGCGTGGATAAAGATGGAAGGCCTCTACAACCCC
ACGTCAGAGTGTGGGATTCTGTTACTCTATCCACACTGCAGATTATTGGACTTGGCACTTTTGA
GCGTGGAGTAGGATGCCTGGATTTTTCAAAAGCAGATTCAGGTGTTCATTTATGTGTTATTGAT
GACTCCAATGAGCATATGCTTACTGTATGGGACTGGCAGAGGAAAGCAAAAGGAGCAGAAATAA
AGACAACAAATGAAGTTGTTTTGGCTGTGGAGTTTCACCCAACAGATGCAAATACCATAATTAC
ATGCGGTAAATCTCATATTTTCTTCTGGACCTGGAGCGGCAATTCACTAACAAGAAAACAGGGA
ATTTTTGGGAAATATGAAAAGCCAAAATTTGTGCAGTGTTTAGCATTCTTGGGGAATGGAGATG
TTCTTACTGGAGACTCAGGTGGAGTCATGCTTATATGGAGCAAAACTACTGTAGAGCCCACACC
TGGGAAAGGACCTAAAGGTGTATATCAAATCAGCAAACAAATCAAAGCTCATGATGGCAGTGTG
TTCACACTTTGTCAGATGAGAAATGGGATGTTATTAACTGGAGGAGGGAAAGACAGAAAAATAA
TTCTGTGGGATCATGATCTGAATCCTGAAAGAGAAATAGAGTTTAGTGCTTCAAGGGCCAGGCT
GCCAGGCCATGTTGCAGCTGACCACCCACCTGCAGTGTACCGCCGGAAGCACCAGGAGCTGCAA
GCCATGCAGATGGAGCTGCAGAGCCCTGAGTACAAGCTGAGCAAGCTCCGCACCTCGACCATCA
TGACCGACTACAACCCCAACTACTGCTTTGCTGGCAAGACCTCCTCCATCAGTGACCTGAAGGA
GGTGCCGCGGAAAAACATCACCCTCATTCGGGGTCTGGGCCATGGCGCCTTTGGGGAGGTGTAT
GAAGGCCAGGTGTCCGGAATGCCCAACGACCCAAGCCCCCTGCAAGTGGCTGTGAAGACGCTGC
CTGAAGTGTGCTCTGAACAGGACGAACTGGATTTCCTCATGGAAGCCCTGATCATCAGCAAATT
CAACCACCAGAACATTGTTCGCTGCATTGGGGTGAGCCTGCAATCCCTGCCCCGGTTCATCCTG
CTGGAGCTCATGGCGGGGGGAGACCTCAAGTCCTTCCTCCGAGAGACCCGCCCTCGCCCGAGCC
AGCCCTCCTCCCTGGCCATGCTGGACCTTCTGCACGTGGCTCGGGACATTGCCTGTGGCTGTCA
GTATTTGGAGGAAAACCACTTCATCCACCGAGACATTGCTGCCAGAAACTGCCTCTTGACCTGT
CCAGGCCCTGGAAGAGTGGCCAAGATTGGAGACTTCGGGATGGCCCGAGACATCTACAGGGCGA
GCTACTATAGAAAGGGAGGCTGTGCCATGCTGCCAGTTAAGTGGATGCCCCCAGAGGCCTTCAT
GGAAGGAATATTCACTTCTAAAACAGACACATGGTCCTTTGGAGTGCTGCTATGGGAAATCTTT
TCTCTTGGATATATGCCATACCCCAGCAAAAGCAACCAGGAAGTTCTGGAGTTTGTCACCAGTG
GAGGCCGGATGGACCCACCCAAGAACTGCCCTGGGCCTGTATACCGGATAATGACTCAGTGCTG
GCAACATCAGCCTGAAGACAGGCCCAACTTTGCCATCATTTTGGAGAGGATTGAATACTGCACC
CAGGACCCGGATGTAATCAACACCGCTTTGCCGATAGAATATGGTCCACTTGTGGAAGAGGAAG
AGAAAGTGCCTGTGAGGCCCAAGGACCCTGAGGGGGTTCCTCCTCTCCTGGTCTCTCAACAGGC
AAAACGGGAGGAGGAGCGCAGCCCAGCTGCCCCACCACCTCTGCCTACCACCTCCTCTGGCAAG
GCTGCAAAGAAACCCACAGCTGCAGAGATCTCTGTTCGAGTCCCTAGAGGGCCGGCCGTGGAAG
GGGGACACGTGAATATGGCATTCTCTCAGTCCAACCCTCCTTCGGAGTTGCACAAGGTCCACGG
ATCCAGAAACAAGCCCACCAGCTTGTGGAACCCAACGTACGGCTCCTGGTTTACAGAGAAACCC
ACCAAAAAGAATAATCCTATAGCAAAGAAGGAGCCACACGACAGGGGTAACCTGGGGCTGGAGG
GAAGCTGTACTGTCCCACCTAACGTTGCAACTGGGAGACTTCCGGGGGCCTCACTGCTCCTAGA
GCCCTCTTCGCTGACTGCCAATATGAAGGAGGTACCTCTGTTCAGGCTACGTCACTTCCCTTGT
GGGAATGTCAATTACGGCTACCAGCAACAGGGCTTGCCCTTAGAAGCCGCTACTGCCCCTGGAG
CTGGTCATTACGAGGATACCATTCTGAAAAGCAAGAATAGCATGAACCAGCCTGGGCCCTGA.
>AB274722.1:271-3450 Homo sapiens mRNA for fusion protein
EML4-ALK variant 1, complete cds
(SEQ ID NO: 32982)
ATGGACGGTTTCGCCGGCAGTCTCGATGATAGTATTTCTGCTGCAAGTACTTCTGATGTTCAAG
ATCGCCTGTCAGCTCTTGAGTCACGAGTTCAGCAACAAGAAGATGAAATCACTGTGCTAAAGGC
GGCTTTGGCTGATGTTTTGAGGCGTCTTGCAATCTCTGAAGATCATGTGGCCTCAGTGAAAAAA
TCAGTCTCAAGTAAAGGCCAACCAAGCCCTCGAGCAGTTATTCCCATGTCCTGTATAACCAATG
GAAGTGGTGCAAACAGAAAACCAAGTCATACCAGTGCTGTCTCAATTGCAGGAAAAGAAACTCT
TTCATCTGCTGCTAAAAGTGGTACAGAAAAAAAGAAAGAAAAACCACAAGGACAGAGAGAAAAA
AAAGAGGAATCTCATTCTAATGATCAAAGTCCACAAATTCGAGCATCACCTTCTCCCCAGCCCT
CTTCACAACCTCTCCAAATACACAGACAAACTCCAGAAAGCAAGAATGCTACTCCCACCAAAAG
CATAAAACGACCATCACCAGCTGAAAAGTCACATAATTCTTGGGAAAATTCAGATGATAGCCGT
AATAAATTGTCGAAAATACCTTCAACACCCAAATTAATACCAAAAGTTACCAAAACTGCAGACA
AGCATAAAGATGTCATCATCAACCAAGAAGGAGAATATATTAAAATGTTTATGCGCGGTCGGCC
AATTACCATGTTCATTCCTTCCGATGTTGACAACTATGATGACATCAGAACGGAACTGCCTCCT
GAGAAGCTCAAACTGGAGTGGGCATATGGTTATCGAGGAAAGGACTGTAGAGCTAATGTTTACC
TTCTTCCGACCGGGGAAATAGTTTATTTCATTGCATCAGTAGTAGTACTATTTAATTATGAGGA
GAGAACTCAGCGACACTACCTGGGCCATACAGACTGTGTGAAATGCCTTGCTATACATCCTGAC
AAAATTAGGATTGCAACTGGACAGATAGCTGGCGTGGATAAAGATGGAAGGCCTCTACAACCCC
ACGTCAGAGTGTGGGATTCTGTTACTCTATCCACACTGCAGATTATTGGACTTGGCACTTTTGA
GCGTGGAGTAGGATGCCTGGATTTTTCAAAAGCAGATTCAGGTGTTCATTTATGTGTTATTGAT
GACTCCAATGAGCATATGCTTACTGTATGGGACTGGCAGAAGAAAGCAAAAGGAGCAGAAATAA
AGACAACAAATGAAGTTGTTTTGGCTGTGGAGTTTCACCCAACAGATGCAAATACCATAATTAC
ATGCGGTAAATCTCATATTTTCTTCTGGACCTGGAGCGGCAATTCACTAACAAGAAAACAGGGA
ATTTTTGGGAAATATGAAAAGCCAAAATTTGTGCAGTGTTTAGCATTCTTGGGGAATGGAGATG
TTCTTACTGGAGACTCAGGTGGAGTCATGCTTATATGGAGCAAAACTACTGTAGAGCCCACACC
TGGGAAAGGACCTAAAGTGTACCGCCGGAAGCACCAGGAGCTGCAAGCCATGCAGATGGAGCTG
CAGAGCCCTGAGTACAAGCTGAGCAAGCTCCGCACCTCGACCATCATGACCGACTACAACCCCA
ACTACTGCTTTGCTGGCAAGACCTCCTCCATCAGTGACCTGAAGGAGGTGCCGCGGAAAAACAT
CACCCTCATTCGGGGTCTGGGCCATGGAGCCTTTGGGGAGGTGTATGAAGGCCAGGTGTCCGGA
ATGCCCAACGACCCAAGCCCCCTGCAAGTGGCTGTGAAGACGCTGCCTGAAGTGTGCTCTGAAC
AGGACGAACTGGATTTCCTCATGGAAGCCCTGATCATCAGCAAATTCAACCACCAGAACATTGT
TCGCTGCATTGGGGTGAGCCTGCAATCCCTGCCCCGGTTCATCCTGCTGGAGCTCATGGCGGGG
GGAGACCTCAAGTCCTTCCTCCGAGAGACCCGCCCTCGCCCGAGCCAGCCCTCCTCCCTGGCCA
TGCTGGACCTTCTGCACGTGGCTCGGGACATTGCCTGTGGCTGTCAGTATTTGGAGGAAAACCA
CTTCATCCACCGAGACATTGCTGCCAGAAACTGCCTCTTGACCTGTCCAGGCCCTGGAAGAGTG
GCCAAGATTGGAGACTTCGGGATGGCCCGAGACATCTACAGGGCGAGCTACTATAGAAAGGGAG
GCTGTGCCATGCTGCCAGTTAAGTGGATGCCCCCAGAGGCCTTCATGGAAGGAATATTCACTTC
TAAAACAGACACATGGTCCTTTGGAGTGCTGCTATGGGAAATCTTTTCTCTTGGATATATGCCA
TACCCCAGCAAAAGCAACCAGGAAGTTCTGGAGTTTGTCACCAGTGGAGGCCGGATGGACCCAC
CCAAGAACTGCCCTGGGCCTGTATACCGGATAATGACTCAGTGCTGGCAACATCAGCCTGAAGA
CAGGCCCAACTTTGCCATCATTTTGGAGAGGATTGAATACTGCACCCAGGACCCGGATGTAATC
AACACCGCTTTGCCGATAGAATATGGTCCACTTGTGGAAGAGGAAGAGAAAGTGCCTGTGAGGC
CCAAGGACCCTGAGGGGGTTCCTCCTCTCCTGGTCTCTCAACAGGCAAAACGGGAGGAGGAGCG
CAGCCCAGCTGCCCCACCACCTCTGCCTACCACCTCCTCTGGCAAGGCTGCAAAGAAACCCACA
GCTGCAGAGGTCTCTGTTCGAGTCCCTAGAGGGCCGGCCGTGGAAGGGGGACACGTGAATATGG
CATTCTCTCAGTCCAACCCTCCTTCGGAGTTGCACAGGGTCCACGGATCCAGAAACAAGCCCAC
CAGCTTGTGGAACCCAACGTACGGCTCCTGGTTTACAGAGAAACCCACCAAAAAGAATAATCCT
ATAGCAAAGAAGGAGCCACACGAGAGGGGTAACCTGGGGCTGGAGGGAAGCTGTACTGTCCCAC
CTAACGTTGCAACTGGGAGACTTCCGGGGGCCTCACTGCTCCTAGAGCCCTCTTCGCTGACTGC
CAATATGAAGGAGGTACCTCTGTTCAGGCTACGTCACTTCCCTTGTGGGAATGTCAATTACGGC
TACCAGCAACAGGGCTTGCCCTTAGAAGCCGCTACTGCCCCTGGAGCTGGTCATTACGAGGATA
CCATTCTGAAAAGCAAGAATAGCATGAACCAGCCTGGGCCCTGA.

By “genetic vaccine” is meant an immunogenic composition comprising a polynucleotide encoding an antigen. In embodiments, the antigen is an ALK antigen.

By “HLA” is meant a human leukocyte antigen protein, or fragment thereof. As used herein “HLA class I” includes HLA-A, HLA-B, and HLA-C.

By “HLA-A*02” is meant the *02 allele group or serotype, or any member of such allele group or serotype, or fragments thereof, of MHC Class I human leukocyte antigen proteins having an α-chain encoded in the A locus. Members of the HLA-A*02 serotype or subgroup include, but are not limited to, HLA-A*02:01, HLA-A*02:02, HLA-A*02:03, HLA-A*02:05, HLA-A*02:06, HLA-A*02:07, HLA-A*02:11.

By “HLA-B*07” is meant the *07 allele group or serotype, or any member of such allele group or serotype, or fragments thereof, of MHC Class I human leukocyte antigen proteins having an α-chain encoded in the B locus. Members of the HLA-B*07 serotype or subgroup include, but are not limited to, HLA-B*07:02, HLA-B*07:04, HLA-B*07:05, HLA-B*07:07.

By “HLA-A*02:01 polypeptide” is meant a human leukocyte antigen protein, or fragment thereof, having an α-chain having at least about 85% amino acid sequence identity to GenBank Accession No. 8EB2_A. An exemplary HLA-A*02:01 α-chain amino acid sequence is provided below (GenBank Accession No. 8EB2_A):

(SEQ ID NO: 32983)
GSHSMRYFFTSVSRPGRGEPRFIAVGYVDDTQFVRFDSDAASQRM
EPRAPWIEQEGPEYWDGETRKVKAHSQTHRVDLGTLRGYYNQSEA
GSHTVQRMYGCDVGSDWRFLRGYHQYAYDGKDYIALKEDLRSWTA
ADMAAQTTKHKWEAAHVAEQLRAYLEGTCVEWLRRYLENGKETLQ
RTDAPKTHMTHHAVSDHEATLRCWALSFYPAEITLTWQRDGEDQT
QDTELVETRPAGDGTFQKWAAVVVPSGQEQRYTCHVQHEGLPKPL
TLRWE.

By “HLA-B*07:02” is meant a human leukocyte antigen protein, or fragment thereof, having an α-chain having at least about 85% amino acid sequence identity to GenBank Accession No. 7S8F_A. An exemplary HLA-B*07:02 α-chain amino acid sequence is provided below (GenBank Accession No. 7S8F_A):

(SEQ ID NO: 32984)
GSHSMRYFYTSVSRPGRGEPRFISVGYVDDTQFVRFDSDAASPRE
EPRAPWIEQEGPEYWDRNTQIYKAQAQTDRESLRNLRGYYNQSEA
GSHTLQSMYGCDVGPDGRLLRGHDQYAYDGKDYIALNEDLRSWTA
ADTAAQITQRKWEAAREAEQRRAYLEGECVEWLRRYLENGKDKLE
RADPPKTHVTHHPISDHEATLRCWALGFYPAEITLTWQRDGEDQT
QDTELVETRPAGDRTFQKWAAVVVPSGEEQRYTCHVQHEGLPKPL
TLRWE

“Hybridization” means hydrogen bonding, which may be Watson-Crick. Hoogsteen, or reversed Hoogsteen hydrogen bonding, between complementary nucleobases. For example, in DNA, adenine and thymine, and cytosine and guanine, are, respectively, complementary nucleobases that pair through the formation of hydrogen bonds. By “hybridize” is meant pairing to form a double-stranded molecule between complementary polynucleotide sequences (e.g., a gene), or portions thereof, under various conditions of stringency (e.g., Wahl, G. M. and S. L. Berger, (1987), Methods Enzymol., 152:399; Kimmel, A. R., (1987), Methods Enzymol. 152:507).

By way of example, stringent salt concentration will ordinarily be less than about 750) mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl. 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “immune effector cell” is meant a lymphocyte, once activated, capable of effecting an immune response upon a target cell. In some embodiments, immune effector cells are effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4 CD8 T cell or a CD4 CD8-T cell. In some embodiments the immune effector cell is a T helper cell. In some embodiments the T helper cell is a T helper 1 (Th1), a T helper 2 (Th2) cell, or a helper T cell expressing CD4 (CD4+ T cell).

By “immunogen” is meant agent which is capable, under appropriate conditions, of eliciting or stimulating an immune response. In an embodiment, an immune response includes a T-cell response. As used herein, an “immunogenic composition” is a composition comprising an immunogen (such as an ALK polypeptide) or a vaccine comprising an immunogen (such as an ALK polypeptide). As will be appreciated by the skilled person in the art, if administered to a subject in need prior to the subject's contracting disease or experiencing full-blown disease, an immunogenic composition can be prophylactic and result in the subject's eliciting an immune response, e.g., a cellular immune response, to protect against disease, or to prevent more severe disease or condition, and/or the symptoms thereof. If administered to a subject in need following the subject's contracting disease, an immunogenic composition can be therapeutic and result in the subject's eliciting an immune response, e.g., a cellular immune response, to treat the disease, e.g., by reducing, diminishing, abrogating, ameliorating, or eliminating the disease, and/or the symptoms thereof. In some embodiments, the immune response is a B-cell response, which results in the production of antibodies, e.g., neutralizing antibodies, directed against the immunogen or immunogenic composition comprising the antigen or antigen sequence. In some embodiments, the immune response is a T-cell response, which results in the production of T-lymphocytes. In a manner similar to the foregoing, in some embodiments, an immunogenic composition or vaccine can be prophylactic. In some embodiments, an immunogenic composition or vaccine can be therapeutic. In some embodiments, the disease is caused by oncogenic ALK gene fusions, rearrangements, duplications, or mutations (e.g., ALK-positive cancers). In some embodiments, the cancer is an ALK-positive cancer. In some embodiments, the ALK-positive cancer is non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, melanoma, or a combination thereof.

The term “immune response” is meant any response mediated by an immunoresponsive cell. In one example of an immune response, leukocytes are recruited to carry out a variety of different specific functions in response to exposure to an antigen (e.g., a foreign entity). Immune responses are multifactorial processes that differ depending on the type of cells involved. Immune responses include cell-mediated responses (e.g., T-cell responses), humoral responses (B-cell/antibody responses), innate responses and combinations thereof.

By “immunogenic composition” is meant a composition that elicits an immune response in a subject. In some instances, the subject is an immunized subject.

The term “immunize” refers to the process of rendering a subject protected from a disease or pathology, or the symptoms thereof, such as by vaccination. In an embodiment, the term “immunize” relates to injecting a polypeptide comprising an oncogenic ALK gene fusions, rearrangements, duplications, or mutations (e.g., ALK-positive cancers), or fragments thereof.

By “increases” is meant a positive alteration of at least 5%, 10%, 25%, 30%, 40%, 50%, 75%, 80%, 85%, 90%, 95%, 98%, or 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, protein, or peptide is purified if it is substantially free of cellular material, debris, non-relevant viral material, or culture medium when produced by recombinant DNA techniques, or of chemical precursors or other chemicals when chemically synthesized. Purity and homogeneity are typically determined using standard purification methods and analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids or proteins, as well as chemically synthesized nucleic acids or peptides.

By “isolated polynucleotide” is meant a nucleic acid molecule that is free of the genes which flank the gene, in the naturally-occurring genome of the organism from which the nucleic acid molecule of the invention is derived. In some instances the nucleic acid molecule is a DNA molecule or an RNA molecule. The term includes, for example, a recombinant DNA that is incorporated into a vector: into an autonomously replicating plasmid or virus: into the genomic DNA of a prokaryote or eukaryote; or that exists as a separate molecule independent of other sequences (for example, a cDNA or a genomic or cDNA fragment produced by PCR or restriction endonuclease digestion). In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 40%, by weight, at least 50%, by weight, at least 60%, by weight, free from the proteins and naturally-occurring organic molecules with which it is naturally associated. Preferably, an isolated polypeptide preparation is at least 75%, more preferably at least 90%, and most preferably at least 99%, by weight, free from the proteins and naturally-occurring organic molecules with which it is naturally associated. An isolated polypeptide may be obtained, for example, by extraction from a natural source; by expression of a recombinant nucleic acid encoding such a polypeptide; or by chemically synthesizing the protein. Purity can be measured by any standard, appropriate method, for example, column chromatography, polyacrylamide gel electrophoresis, or by HPLC analysis. An isolated polypeptide can refer to an ALK antigen or immunogen polypeptide generated by the methods described herein.

By “linker” is meant one or more amino acids that serve as a spacer between two polypeptides or peptides of a fusion protein.

By “marker” is meant any analyte having an alteration in expression level or activity that is associated with or used to characterize a disease, condition, pathology, or disorder.

As used herein, “obtaining” as in “obtaining an agent” includes synthesizing, isolating, purchasing, or otherwise acquiring the agent.

“Neoplasia” refers to cells or tissues exhibiting abnormal growth or proliferation. The term neoplasia encompasses cancer and solid tumors.

“Neuroblastoma” refers to a solid cancerous tumor that usually originates in the abdomen in adrenal gland tissue, but can also originate from nerve tissue in the neck, chest, abdomen, and pelvis. Neuroblastoma is derived from the neural crest and is characterized by a marked clinical heterogeneity (aggressive, unremitting growth to spontaneous remission). Neuroblastoma may metastasize to the lymph nodes, liver, lungs, bones and bone marrow. Neuroblastoma is the most common heterogenous and malignant tumor of early childhood, and two thirds of individuals with neuroblastoma are diagnosed when they are younger than 5 years. About 10% of neuroblastoma cases have activating point mutation in the ALK protein (e.g., ALK^{F1174L}).

By “operably linked” is meant that a first polynucleotide is positioned adjacent to a second polynucleotide that directs transcription of the first polynucleotide when appropriate molecules are bound to the second polynucleotide. By way of example, a first nucleic acid sequence is operably linked to a second nucleic acid sequence when the first nucleic acid sequence is placed in a functional relationship with the second nucleic acid sequence. For instance, a promoter is operably linked to a coding sequence if the promoter affects (allows) the transcription or expression of the coding sequence. Generally, operably linked DNA sequences are contiguous and, where necessary to join two protein-coding regions, are in the same open reading frame.

The nucleic acid sequence encoding an ALK peptide (antigen peptide) generated by the described methods can be optimized for expression in mammalian cells via codon-optimization and RNA optimization (such as to increase RNA stability) using procedures and techniques practiced in the art.

The term “pharmaceutically acceptable vehicle” refers to conventional carriers and excipients that are physiologically and pharmaceutically acceptable for use, particularly in mammalian subjects. A non-limiting example of a mammalian subject is a human subject. Pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic or immunogenic compositions, such as one or more vaccines, and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol, or the like as a vehicle. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

By “plasmid” is meant a circular nucleic acid molecule capable of autonomous replication in a host cell.

The terms “protein.” “peptide.” “polypeptide,” and their grammatical equivalents are used interchangeably herein, and refer to a polymer of amino acid residues linked together by peptide bonds. The terms refer to a protein, peptide, or polypeptide of any size, structure, or function. Typically, a protein, peptide, or polypeptide will be at least three (3) amino acids long. A protein, peptide, or polypeptide can refer to an individual protein or a collection of proteins. One or more of the amino acids in a protein, peptide, or polypeptide can be modified, such as glycoproteins, for example, by the addition of a chemical entity such as a carbohydrate group, a hydroxyl group, a phosphate group, a farnesyl group, an isofarnesyl group, a fatty acid group, a linker for conjugation, functionalization, or other modifications, etc. A protein, peptide, or polypeptide can also be a single molecule or can be a multi-molecular complex. A protein, peptide, or polypeptide can be just a fragment of a naturally occurring protein or peptide. A protein, peptide, or polypeptide can be naturally occurring, recombinant, or synthetic, or any combination thereof.

In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA or DNA. Any of the proteins provided herein can be produced by any method known in the art. For example, the proteins provided herein can be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

Conservative amino acid substitutions are those substitutions that, when made, least interfere with the properties of the original protein, that is, the structure and especially the function of the protein is conserved and is not significantly changed by such substitutions. Examples of conservative amino acid substitutions are known in the art, e.g., as set forth in, for example, U.S. Publication No. 2015/0030628. Conservative substitutions generally maintain (a) the structure of the polypeptide backbone in the area of the substitution, for example, as a sheet or helical conformation; (b) the charge or hydrophobicity of the molecule at the target site; and/or (c) the bulk of the side chain

The substitutions that are generally expected to produce the greatest changes in protein properties are non-conservative, for instance, changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

By “promoter” is meant a polynucleotide sufficient to direct transcription. A promoter includes necessary nucleic acid sequences near the start site of transcription. A promoter also optionally includes distal enhancer or repressor sequence elements. A “constitutive promoter” is a promoter that is continuously active and is not subject to regulation by external signals or molecules. In contrast, the activity of an “inducible promoter” is regulated by an external signal or molecule (for example, a transcription factor). By way of example, a promoter may be a CMV promoter.

As will be appreciated by the skilled practitioner in the art, the term “purified” does not require absolute purity: rather, it is intended as a relative term. Thus, for example, a purified peptide, protein, or other active compound is one that is isolated in whole or in part from naturally associated proteins and other contaminants. In certain embodiments, the term “substantially purified” refers to a peptide, protein, or other active compound that has been isolated from a cell, cell culture medium, or other crude preparation and subjected to routine methods, such as fractionation, chromatography, or electrophoresis, to remove various components of the initial preparation, such as proteins, cellular debris, and other components.

A “recombinant” nucleic acid or protein is one that has a sequence that is not naturally occurring or that has a sequence that is made by an artificial combination of two otherwise separated segments of sequence. Such an artificial combination is often accomplished by chemical synthesis or by the artificial manipulation of isolated segments of nucleic acids, for example, by genetic engineering techniques. A “non-naturally occurring” nucleic acid or protein is one that may be made via recombinant technology, artificial manipulation, or genetic or molecular biological engineering procedures and techniques, such as those commonly practiced in the art.

By “reduces” is meant a negative alteration of at least 5%, 10%, 25%, 30%, 40%, 50%, 75%, 80%, 85%, 90%, 95%, 98%, or 100%.

By “reference” is meant a standard or control condition. In some cases, the reference is a healthy cell or a healthy subject, or the reference is a cell or subject that does not have or is not associated with a cancer or tumor (e.g., an ALK-positive neoplasia, such as a non-small cell lung cancer (NSCLC)). In some instances, the reference is a subject or cell prior to being administered a composition or being treated for a disease or a subject or cell that has not been administered a composition or treatment. In some instances, the reference is a subject or cell prior to a change in a treatment.

A “reference sequence” is a defined sequence used as a basis for sequence comparison. The reference sequence can be an ALK antigen nucleotide or polypeptide sequence. A reference sequence may be a subset of or the entirety of a specified sequence: for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant an agent that recognizes and binds a polypeptide (e.g., an ALK polypeptide), but which does not substantially recognize and bind other molecules in a sample, for example, a biological sample, which naturally includes a polypeptide of the invention, such as an ALK polypeptide. For example, an engineered T-cell receptor that specifically binds an antigen (e.g., an ALK polypeptide or fragment thereof) expressed on the surface of a cell, but which does not substantially bind to other polypeptides, carbohydrates, lipids, or other element on the surface of the cell.

Nucleic acid molecules useful in the methods described herein include any nucleic acid molecule that encodes a polypeptide as described, or a fragment thereof. Such nucleic acid molecules need not be 100% identical with an endogenous nucleic acid sequence, but will typically exhibit substantial identity.

By “substantially identical” is meant a polypeptide or nucleic acid molecule exhibiting at least 50% identity to a reference amino acid sequence or nucleic acid sequence. Examples of reference amino acid sequences and nucleic acid sequences include any of those provided herein. In embodiments, such a sequence is at least 60%, or at least 80% or 85%, or at least or equal to 90%, 95%, 98% or even 99% identical at the amino acid level or nucleic acid to the sequence used for comparison. Polynucleotides having “substantial identity” to an endogenous sequence are in some instances capable of hybridizing with at least one strand of a double-stranded nucleic acid molecule.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J. Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “subject” is meant an animal. Non-limiting examples of animals include a mammal, including, but not limited to, a human, a non-human primate, or a non-human mammal, such as a bovine, equine, canine, ovine, or feline mammal, or a sheep, goat, llama, camel, or a rodent (e.g., rat, mouse), gerbil, or hamster. In a nonlimiting example, a subject is one who has, is at risk of developing, or who is susceptible to a disease caused by oncogenic ALK gene fusions, rearrangements, duplications, or mutations (e.g., ALK-positive cancers). In particular aspects as described herein, the subject is a human subject, such as a patient.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater, consecutively, such as to 100 or greater.

By “T Cell” is meant a type of white blood cell having a T cell receptor on the cell surface that functions in an adaptive immune response. T cells include, but are not limited to, CD4+ T cells and CD8+ T cells. A CD4+T lymphocyte is an immune cell that carries a marker on its surface known as “cluster of differentiation 4 (CD4).” These cells, also known as helper T cells, help orchestrate the immune response, including antibody responses as well as killer T cell responses. A subset of CD4+ T cells has regulatory functions (Treg cells). Various other subsets of CD4+ T cells are defined by their pattern of cytokine production (e.g. Th17 cells that produce IL-17, Th (cells that produce IL-9, etc). CD8+ T cells carry the “cluster of differentiation 8” (CD8) marker. In one embodiment, a CD8+ T cell is a cytotoxic T lymphocyte. In another embodiment, a CD8+ cell is a suppressor T cell. An effector function of a T cell is a specialized function of the T cell, such as cytolytic activity or helper activity including the secretion of cytokines.

By “T cell receptor (TCR)” is meant a polypeptide comprising an alpha locus, a beta locus or fragment thereof that binds a polypeptide of interest (e.g., ALK) or a fragment thereof. In some embodiments, the TCR is expressed as part of a complex with a CD3 polypeptide. In some embodiments, the T cell receptor binds an ALK polypeptide or fragment thereof. Is some embodiments, the TCR is membrane bound. In other embodiments, the TCR is soluble. In some embodiments, the ALK-specific TCRs recognize human ALK peptides presented by HLA-B*0) 7:02 human HLA allele. In some embodiments, a TCR comprises are listed in Tables 2 and 3.

By “TCR polynucleotide” is meant a nucleic acid molecule encoding a TCR polypeptide.

As used herein, the terms “treat,” “treating,” “treatment,” and the like refer to reducing, diminishing, decreasing, delaying, abrogating, ameliorating, or eliminating, a disease, condition, disorder, or pathology, and/or symptoms associated therewith. While not intending to be limiting. “treating” typically relates to a therapeutic intervention that occurs after a disease, condition, disorder, or pathology, and/or symptoms associated therewith, have begun to develop to reduce the severity of the disease, etc., and the associated signs and symptoms. It will be appreciated that, although not precluded, treating a disorder or condition does not require that the disease, condition, disorder, pathology, or the symptoms associated therewith, be completely eliminated.

As referred to herein, a “transformed” or “transfected” cell is a cell into which a nucleic acid molecule or polynucleotide sequence has been introduced by molecular biology techniques. As used herein, the term “transfection” encompasses all techniques by which a nucleic acid molecule or polynucleotide may be introduced into such a cell, including transfection with viral vectors, transformation with plasmid vectors, and introduction of naked nucleic acid (DNA or RNA) by electroporation, lipofection, and particle gun acceleration.

By “vaccine” is meant a preparation of immunogenic material capable of eliciting an immune response. In embodiments, a vaccine is administered to a subject to treat a disease, condition, or pathology, or to prevent a disease, condition, or pathology. In some instances, the disease is caused by oncogenic ALK gene fusions, rearrangements, duplications, or mutations (e.g., ALK-positive cancers). In embodiments, the immunogenic materials is a protein or nucleic acid molecule. The immunogenic material may include, for example, antigenic proteins, peptides, or DNA derived from ALK-expressing tumors or cell lines. Vaccines may elicit a prophylactic (preventative) immune response in the subject: they may also elicit a therapeutic response immune response in a subject. As mentioned above, methods of vaccine administration vary according to the vaccine, and can include routes or means, such as inoculation (intravenous or subcutaneous injection), ingestion, inhalation, or other forms of administration. Inoculations can be delivered by any number of routes, including parenteral, such as intravenous, subcutaneous, or intramuscular. Vaccines may also be administered with an adjuvant to boost the immune response.

As used herein, a “vector” refers to a nucleic acid molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. In some embodiments of the present disclosure, the vector encodes an ALK protein. In some embodiments, the vector is the pTR600 expression vector (U.S. Patent Application Publication No. 2002/0106798; Ross et al., 2000, Nat Immunol. 1(2):102-103; and Green et al., 2001, Vaccine 20:242-248).

Unless specifically stated or obvious from context, as used herein, the term “or” is understood to be inclusive. Unless specifically stated or obvious from context, as used herein, the terms “a,” “an,” and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of some embodiments for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides the results of Fluorescence Activated Cell Sorting (FACs) analysis showing isolation of murine CD8+ T cells reactive against ALK. CD8⁺ T cells reactive against the ALK PGPGRVAKI (SEQ ID NO: 32985) peptide were identified and sorted for their binding to the PGPGRVAKI (SEQ ID NO: 32985) DEXTRAMER made with ALK PGPGRVAKI (SEQ ID NO: 32985) peptide bound to H-2D^{d} MHC-I molecules. In this figure, FSC is forward scatter, the x-axis is labeled in increments of 200, and the y-axis is labeled on a logarithmic base 10 scale. The plots provide a comparison between mice vaccinated with the PGPGRVAKI (SEQ ID NO: 32985) peptide (VAX) versus unvaccinated (Naïve) mice. Figure discloses “PGPGRVAKI” as SEQ ID NO: 32985.

FIG. 2 provides a series of graphs showing cloning of murine TCRs against ALK. The paired sequence of α and β chains were cloned into a retroviral vector and transduced into BW5147.3 cells. The efficiency of transduction was evaluated by analyzing CD3 and TCRβ chain expression by flow cytometry and the specificity of the TCRs was demonstrated by selective binding to the ALK PGPGRVAKI (SEQ ID NO: 32985) DEXTRAMER.

FIG. 3 provides a graph showing in vitro validation of murine ALK TCR-T cell lines 1-5. IFN-γ production by murine ALK TCR-T cell lines was evaluated with ELISA; supernatants were collected 24 hours after incubation of ALK TCR-T cells with BALB/c splenocytes pulsed with increasing concentrations of ALK PGPGRVAKI (SEQ ID NO: 32985) peptide. Figure discloses “PGPGRVAKI” as SEQ ID NO: 32985.

FIG. 4 provides a graph showing in vitro validation of murine ALK TCR-T cells. IL2 production by murine ALK TCR-T cells was evaluated with ELISA; supernatants were collected 24 hours after incubation of ALK TCR-T cells with BALB/c splenocytes that were pulsed with increasing concentrations of ALK PGPGRVAKI (SEQ ID NO: 32985) peptide. Figure discloses “PGPGRVAKI” as SEQ ID NO: 32985.

FIG. 5 provides graphs showing in vitro validation of murine ALK TCR-T cells. IL2 production by murine ALK TCR-T cells was evaluated using ELISA; supernatants were collected 24 hours after incubation of ALK TCR-T cells with murine H2-D^{d} lung cancer cell lines, including cell lines expressing human EML4-ALK, an ALK translocation, Effector: Target ratio 1:1. For each set of four bars in the bar graph, the first bar is ASB-XIV, the second bar is ASB-XIV^{Eml4-Alk}, the third bar is Eml4-Alk, and the fourth bar is Eml4-Alk^{PGPGRVAKI} (“PGPGRVAKI” disclosed as SEQ ID NO: 32985). The panel at the right describes the efficiency of transduction of the H2-D^{d} cancer cell lines with human EML4-ALK, which was evaluated by analyzing H2-D^{d} expression by flow cytometry. Figure discloses “PGPGRVAKI” as SEQ ID NO: 32985.

FIG. 6 provides graphs showing in vitro validation of murine ALK-specific TCR-T cells. IL2 production by murine ALK-specific TCR-T cells was evaluated with ELISA; supernatants were collected 24 h after incubation of ALK-specific TCR-T cells with lymphoma H2-D^{d} lung cancer cell lines, E:T ratio 1:1. For each pair of bars in the bar graph, the left bar is Npm-Alk-1, and the right bar is Npm-Alk-2. The panel at the right describes the efficiency of transduction of the H2-D^{d} cancer cell lines with Npm-Alk-1 or Npm-Alk-2, which was evaluated by analyzing H2-D^{d} expression by flow cytometry.

FIG. 7 provides a bar graph showing murine ALK-specific TCR expression in primary murine CD8⁺ T cells. The percentage of expression of the murine ALK-specific TCR on the surface of primary murine CD8⁺ T cells was calculated two days after retroviral infection by dividing the number of cells binding an ALK MHC-I multimer optimized to accommodate multiple binding sites and numerous fluorophores to boost avidity in bind antigen-specific CD8+ T cells by the number of infected cells (GFP-positive cells). The Multimer is commercially available (Dextramer® reagent, Immudex).

FIG. 8 provides a bar graph showing Mean Fluorescence Intensity (MFI) of the ALK Dextramer®. The MFI of the ALK Dextramer® in primary murine CD8⁺ T cells was calculated 2 days after transduction with the ALK TCR.

FIG. 9 provides a bar graph showing in vitro evaluation of murine ALK-specific TCR-T cells killing activity. Murine ALK-specific TCR-T cell killing activity was evaluated by measuring the residual bioluminescence of luciferase-positive (FFUC⁺) lung tumor cells 3 days post-coculture (E:T ratio 1:1). Figure discloses “PGPGRVAKI” as SEQ ID NO: 32985.

FIG. 10 provides a series of plots showing the expression of IFN-γ and TNF-α, detected by intracellular staining, in a first cohort of HLA-B*0) 7:02 mice vaccinated with RPRPSQPSSL (SEQ ID NO: 32957) peptide. Peripheral blood of one naïve mouse and of HLA-B*07:02 mice vaccinated with the RPRPSQPSSL (SEQ ID NO: 32957) peptide was collected 7 days after the first vaccination boost and T cells were pulsed overnight with the RPRPSQPSSL (SEQ ID NO: 32957) peptide and with a mock peptide. Data shown are gated on CD8 T cells. Figure discloses “RPRPSQPSSL” as SEQ ID NO: 32957.

FIG. 11 provides a series of plots showing the expression of IFN-γ and TNF-α, detected by intracellular staining, in a first cohort of HLA-B*0) 7:02 mice vaccinated with IVRCIGVSL (SEQ ID NO: 32958) peptide. Peripheral blood of one naïve mouse and of HLA-B*07:02 mice vaccinated with IVRCIGVSL (SEQ ID NO: 32958) peptide was collected 7 days after the first vaccination boost and T cells were pulsed overnight with the IVRCIGVSL (SEQ ID NO: 32958) peptide and with a mock peptide. Data shown are gated on CD8 T cells. Figure discloses “IVRCIGVSL” as SEQ ID NO: 32958.

FIG. 12 provides a series of plots showing the expression of IFN-γ and TNF-α, detected by intracellular staining, in a second cohort of HLA-B*07:02 mice vaccinated with RPRPSQPSSL (SEQ ID NO: 32957) peptide. Peripheral blood of one naïve mouse and of HLA-B*0) 7:02 mice vaccinated with the RPRPSQPSSL (SEQ ID NO: 32957) peptide was collected 7 days after the first vaccination boost and T cells were pulsed overnight with the RPRPSQPSSL (SEQ ID NO: 32957) peptide and with a mock peptide. Data shown are gated on CD8⁺ T cells. Figure discloses “RPRPSQPSSL” as SEQ ID NO: 32957.

FIG. 13 provides a series of plots showing the expression of IFN-γ and TNF-α, detected by intracellular staining, in a second cohort of HLA-B*07:02 mice vaccinated with IVRCIGVSL (SEQ ID NO: 32958) peptide. Peripheral blood of one naïve mouse and of HLA-B*07:02 mice vaccinated with IVRCIGVSL (SEQ ID NO: 32958) peptide was collected 7 days after the first vaccination boost and T cells were pulsed overnight with the IVRCIGVSL (SEQ ID NO: 32958) peptide and with a mock peptide. Data shown are gated on CD8 T cells. Figure discloses “IVRCIGVSL” as SEQ ID NO: 32958.

FIG. 14 provides plots showing the isolation of CD8⁺ T cells reactive against RPRPSQPSSL (SEQ ID NO: 32957) peptide in a first cohort. Splenocytes and inguinal lymph-node cells of a HLA-B*07:02 naïve control mouse and of one HLA-B*0) 7:02 mouse vaccinated with RPRPSQPSSL (SEQ ID NO: 32957) peptide were collected 7 days after the last vaccination booster and incubated overnight with the RPRPSQPSSL (SEQ ID NO: 32957) peptide. Twelve hours after, untouched CD8⁺ T cells were purified and CD8⁺/CD137 (4-1BB)⁺ T cells were sorted and subjected to single cell sequencing. Figure discloses “RPRPSQPSSL” as 30) SEQ ID NO: 32957.

FIG. 15 provides plots showing the isolation of CD8+ T cells reactive against RPRPSQPSSL (SEQ ID NO: 32957) peptide in a first cohort. Splenocytes and inguinal lymph-node cells of a HLA-B*07:02 naïve control mouse and of three HLA-B*07:02 mice vaccinated with RPRPSQPSSL (SEQ ID NO: 32957) peptide were collected 7 days after the last vaccination booster and incubated overnight with the RPRPSQPSSL (SEQ ID NO: 32957) peptide. Twelve hours after, untouched CD8⁺ T cells were purified and CD8⁺/CD137(4-1BB)⁺ T cells were sorted and subjected to single cell sequencing. Figure discloses “RPRPSQPSSL” as SEQ ID NO: 32957.

FIG. 16 provides plots showing the isolation of CD8 T cells reactive against IVRCIGVSL (SEQ ID NO: 32958) peptide in a first cohort. Splenocytes and inguinal lymph-node cells of a HLA-B*07:02 naïve control mouse and of three HLA-B*07:02 mice vaccinated with IVRCIGVSL (SEQ ID NO: 32958) peptide were collected 7 days after the last vaccination booster and incubated overnight with the IVRCIGVSL (SEQ ID NO: 32958) peptide. Twelve hours after, untouched CD8 T cells were purified and CD8⁺/CD137(4-1BB)⁺ T cells were sorted and subjected to single cell sequencing. Figure discloses “IVRCIGVSL” as SEQ ID NO: 32958.

FIG. 17 provides FACS analyses showing the isolation of CD8⁺ T cells reactive against RPRPSQPSSL (SEQ ID NO: 32957) peptide in a second cohort. Splenocytes and inguinal lymph-node cells of a HLA-B*07:02 naïve control mouse and of a HLA-B*07:02 moused vaccinated with RPRPSQPSSL (SEQ ID NO: 32957) peptide were collected 7 days after the last vaccination booster and incubated overnight with the RPRPSQPSSL (SEQ ID NO: 32957) peptide. Twelve hours after, untouched CD8⁺ T cells were purified and CD8⁺/CD137(4-1BB)⁺ T cells were sorted and subjected to single cell sequencing. Figure discloses “RPRPSQPSSL” as SEQ ID NO: 32957.

FIG. 18 provides FACS analyses showing the isolation of CD8⁺ T cells reactive against IVRCIGVSL (SEQ ID NO: 32958) peptide in a second cohort. Splenocytes and inguinal lymph-node cells of a HLA-B*07:02 naïve control mouse and of four HLA-B*07:02 mice vaccinated with IVRCIGVSL (SEQ ID NO: 32958) peptide were collected 7 days after the last vaccination booster and incubated overnight with the IVRCIGVSL (SEQ ID NO: 32958) peptide. Twelve hours after, untouched CD8⁺ T cells were purified and CD8⁺/CD137(4-1BB)⁺ T cells were sorted and subjected to single cell sequencing. Figure discloses “IVRCIGVSL” as SEQ ID NO: 32958.

FIG. 19 provides a bar chart showing the relative frequency of the expanded TCR clonotypes identified from the first and the second cohorts of HLA*B07:02 mice vaccinated with RPRPSQPSSL (SEQ ID NO: 32957) peptide. TCR clonotypes were classified as expanded if their Variable-Diversity Joining rearrangement (VDJ) count was ≥4; TCR clonotypes were classified as not expanded if their VDJ count was <4. Figure discloses “RPRPSQPSSL” as SEQ ID NO: 32957.

FIG. 20 provides a bar chart showing the relative frequency of the expanded TCR clonotypes identified from the first and the second cohorts of HLA*B07:02 mice vaccinated with IVRCIGVSL (SEQ ID NO: 32958) peptide. TCR clonotypes were classified as expanded if their VDJ count was ≥4; TCR clonotypes were classified as not expanded if their VDJ count was <4. Figure discloses “IVRCIGVSL” as SEQ ID NO: 32958.

FIG. 21 provides a volcano plot showing differentially expressed genes between expanded (VDJ count≥4) and not expanded (VDJ count≤4) ALK-specific TCR clonotypes in one HLA-B*0) 7:02 mouse from a first cohort vaccinated with RPRPSQPSSL (SEQ ID NO: 32957). Right of right dotted line=upregulated genes (LFG>1.5), Left of left dotted line=downregulated genes (LFG<−1.5), Between the dotted lines=unchanged genes (p<0.05).

FIG. 22 provides a volcano plot showing differentially expressed genes between expanded (VDJ count≥4) and not expanded (VDJ count≤4) ALK-specific TCR clonotypes in three HLA-B*0) 7:02 mice from a first cohort vaccinated with RPRPSQPSSL (SEQ ID NO: 32957). Right of right dotted line=upregulated genes (LFG>1.5), Left of left dotted line=downregulated genes (LFG<−1.5), Between the dotted lines=unchanged genes (p<0.05).

FIG. 23 provides a volcano plot showing differentially expressed genes between expanded (VDJ count≥4) and not expanded (VDJ count≤4) ALK-specific TCR clonotypes in three HLA-B*07:02 mice from a first cohort vaccinated with IVRCIGVSL (SEQ ID NO: 32958). Right of right dotted line=upregulated genes (LFG>1.5), Left of left dotted line=downregulated genes (LFG<−1.5), Between the dotted lines=unchanged genes (p<0.05).

FIG. 24 provides a bubble plot showing pathways from Gene Set Enrichment Analysis (GSEA) that are enriched in expanded versus non-expanded ALK-specific TCR clonotypes sequenced from one HLA*B07:02 mouse from a first cohort vaccinated with RPRPSQPSSL (SEQ ID NO: 32957).

FIG. 25 provides a bubble plot showing pathways from Gene Set Enrichment Analysis (GSEA) that are enriched in expanded versus non-expanded ALK-specific TCR clonotypes sequenced from three HLA*B07:02 mice from a first cohort vaccinated with RPRPSQPSSL (SEQ ID NO: 32957).

FIG. 26 provides a bar graphs showing the percentage of infection of human ALK TCR-T cells (Cβ1) in CD8⁺ T and cells CD4⁺ T of two healthy donors. Untouched CD3⁺ T cells were purified from the Peripheral Blood Mononuclear Cells (PBMCs) of two healthy donors and activated in vitro with anti-CD3 anti-CD28 beads for 2 days before retroviral transduction with the ALK TCR constructs (Cβ1). The percentage of infection was calculated by evaluating GFP expression 3 days post-infection. Data shown are mean±SEM.

FIG. 27 provides a bar graph showing the percentage of infection of human ALK TCR-T cells (Cβ1 and Cβ2) in CD8⁺ T and cells CD4⁺ T cells of one healthy donor. Untouched CD3⁺ T cells were purified from the Peripheral Blood Mononuclear Cells (PBMCs) of one healthy donor and activated in vitro with anti-CD3 anti-CD28 beads for 2 days before retroviral transduction with the ALK TCR constructs (Cβ1 and Cβ2). The percentage of infection was calculated by evaluating GFP expression 3 days post-infection.

FIG. 28 provides a bar graph showing human ALK TCR-T expression (Cβ1) in CD8⁺ T and cells CD4⁺ T cells of two healthy donors. The expression of the ALK TCR-T cells on the surface of CD8⁺ T and cells CD4⁺ T cells of two healthy donors was calculated by evaluating the expression of murine TCRβ three days post-infection. Data shown are mean±SEM.

FIG. 29 provides a bar graph showing human ALK TCR-T expression (Cβ1 and Cβ2) in CD8⁺ T cells of one healthy donor. The expression of the ALK TCR-T cells on the CD8⁺ T cell surface of one healthy donor was calculated by evaluating the expression of murine TCRβ three days post-infection.

FIG. 30 provides a bar graph showing murine TCRβ mean fluoresce intensity (MFI). The MFI of murine TCRβ was calculated in both CD8⁺ T cells and CD4⁺ T cells of two healthy donors transduced with the human ALK TCR-T cells (Cβ1).

FIG. 31 provides a bar graph showing Dextramer® mean fluoresce intensity (MFI). The MFI of the ALK dextramer was evaluated in both CD8⁺ and CD4+ T cells of two healthy donors transduced with the human ALK TCR-T cells (Cβ1).

FIG. 32 provides a bar graph showing murine TCRβ and ALK Dextramer® mean fluorescence intensity (MFI). The MFI of the murine TCRβ was calculated in CD8⁺ T cells of one healthy donor transduced with the human ALK TCR-T cell (Cβ1 and Cβ2); the MFI of the ALK dextramer was evaluated in both CD8⁺ and CD4⁺ T cells of the same healthy donor transduced with the human ALK TCR-T cell (Cβ1 and Cβ2).

FIG. 33 provides a series of graphs showing a summary of human ALK TCR-T cells (Cβ1) percentage of infection, murine TCRβ expression, and Dextramer® binding in CD8⁺ T cells of one healthy donor.

FIG. 34 provides a series of graphs showing a summary of human ALK TCR-T cells (Cβ1) percentage of infection, murine TCRβ expression, and Dextramer® binding among CD4⁺ T cells of one healthy donor.

FIG. 35 provides a series of graphs showing a summary of human ALK TCR-T cells (Cβ1) percentage of infection, murine TCRβ expression, and Dextramer® binding among CD8⁺ T cells of healthy donor 2.

FIG. 36 provides a series of graphs showing a summary of human ALK TCR-T cells (Cβ1) percentage of infection, murine TCRβ expression, and dextramer binding among CD4⁺ T cells of healthy donor 2.

FIG. 37 provides a series of graphs showing a summary of human ALK TCR-T cells (Cβ2) percentage of infection, murine TCRβ expression, and dextramer binding among CD8⁺ T cells of healthy donor 1.

FIG. 38 provides a series of graphs showing a summary of human ALK TCR-T cells (Cβ1 and Cβ2) percentage of infection, murine TCRβ expression, and dextramer binding among CD8⁺ T cells of healthy donor 1.

FIG. 39 provides a series of graphs showing a summary of human ALK TCR-T cells (Cβ2) percentage of infection, murine TCRβ expression, and dextramer binding among CD4⁺ T cells of healthy donor 1.

FIG. 40 provides a series of graphs showing a summary of human ALK TCR-T cells (Cβ2) percentage of infection, murine TCRβ expression, and dextramer binding among CD4⁺ T cells of healthy donor 1.

FIG. 41 provides a series of graphs showing HLA*B07 expression in the human tumor cell lines NCI H2228, NCI H3122, NCI H226, Karpas 299, and JB6.

FIG. 42 provides bar graphs showing an in vitro evaluation of the human ALK TCR-T cells (Cβ1) killing activity. Human ALK TCR-T cells (Cβ1) killing activity was evaluated by measuring the residual bioluminescence of luciferase-positive (FFUC⁺) tumor cells NCI H2228, NCI H3122, NCI H226, and CaSki 3 days post-coculture (E:T ratio 1:1). Figure discloses “RPRPSQPSSL” as SEQ ID NO: 32957.

FIG. 43 provides bar graphs showing an in vitro evaluation of human ALK TCR-T cells (Cβ1) killing activity. Human ALK TCR-T cells (Cβ1) killing activity was evaluated by measuring the residual bioluminescence of luciferase-positive (FFUC⁺) tumor cells NCI H2228, NCI H3122, NCI H226, and CaSki 5 days post-coculture (E:T ratio 1:1). Figure discloses “RPRPSQPSSL” as SEQ ID NO: 32957.

FIG. 44 provides bar graphs showing an in vitro evaluation of human ALK TCR-T cells (Cβ1) killing activity. Human ALK TCR-T cells (Cβ1) killing activity was evaluated by measuring the residual bioluminescence of luciferase-positive (FFUC⁺) tumor cells Karpas 299 and JB6 3 and 5 days post-coculture (E:T ratio 1:1). Figure discloses “RPRPSQPSSL” as SEQ ID NO: 32957.

FIG. 45 provides bar graphs showing an in vitro evaluation of human ALK TCR-T cells (Cβ2) killing activity. Human ALK TCR-T cells (Cβ2) killing activity was evaluated by measuring the residual bioluminescence of luciferase-positive (FFUC⁺) tumor cells NCI H2228, NCI H3122, NCI H226, and CaSki 1 day post-coculture (E:T ratio 1:1). Figure discloses “RPRPSQPSSL” as SEQ ID NO: 32957.

FIG. 46 provides a bar graph showing an in vitro evaluation of human ALK TCR-T cells (Cβ2) killing activity. Human ALK TCR-T cells (Cβ2) killing activity was evaluated by measuring the residual bioluminescence of luciferase-positive (FFUC⁺) tumor cells Karpas 299 and JB6 1 day post-coculture (E:T ratio 1:1). Figure discloses “RPRPSQPSSL” as SEQ ID NO: 32957.

FIG. 47 provides bar graphs showing an in vitro evaluation of human ALK TCR-T cells (Cβ2) killing activity. Human ALK TCR-T cells (Cβ2) killing activity was evaluated by measuring the residual bioluminescence of luciferase-positive (FFUC⁺) tumor cells NCI H2228, NCI H3122, NCI H226, and CaSki 2 days post-coculture (E:T ratio 1:1). Figure discloses “RPRPSQPSSL” as SEQ ID NO: 32957.

FIG. 48 provides a bar graph showing an in vitro evaluation of human ALK TCR-T cells (Cβ2) killing activity. Human ALK TCR-T cells (Cβ2) killing activity was evaluated by measuring the residual bioluminescence of luciferase-positive (FFUC⁺) tumor cells Karpas 299 and JB6 2 days post-coculture (E:T ratio 1:1). Figure discloses “RPRPSQPSSL” as SEQ ID NO: 32957.

FIG. 49 provides bar graphs showing an in vitro evaluation of human ALK TCR-T cells (Cβ2) killing activity. Human ALK TCR-T cells (Cβ2) killing activity was evaluated by measuring the residual bioluminescence of luciferase-positive (FFUC⁺) tumor cells NCI H2228, NCI H3122, NCI H226, and CaSki 3 days post-coculture (E:T ratio 1:1). Figure discloses “RPRPSQPSSL” as SEQ ID NO: 32957.

FIG. 50 provides a bar graph showing an in vitro evaluation of human ALK TCR-T cells (Cβ2) killing activity. Human ALK TCR-T cells (Cβ2) killing activity was evaluated by measuring the residual bioluminescence of luciferase-positive (FFUC⁺) tumor cells Karpas 299 and JB6 3 days post-coculture (E:T ratio 1:1). Figure discloses “RPRPSQPSSL” as SEQ ID NO: 32957.

FIG. 51 provides bar graphs showing an in vitro evaluation of human ALK TCR-T cells (Cβ2) killing activity. Human ALK TCR-T cells (Cβ2) killing activity was evaluated by measuring the residual bioluminescence of luciferase-positive (FFUC⁺) tumor cells NCI H2228, NCI H3122, NCI H226, and CaSki 5 days post-coculture (E:T ratio 1:1). Figure discloses “RPRPSQPSSL” as SEQ ID NO: 32957.

FIG. 52 provides a bar graph showing an in vitro evaluation of human ALK TCR-T cells (Cβ2) killing activity. Human ALK TCR-T cells (Cβ2) killing activity was evaluated by measuring the residual bioluminescence of luciferase-positive (FFUC⁺) tumor cells Karpas 299 and JB6 3 days post-coculture (E:T ratio 1:1). Figure discloses “RPRPSQPSSL” as SEQ ID NO: 32957.

FIG. 53 provides an illustration showing a schematic workflow of ALK-specific TCRs isolation from HLA-A*02:01 and HLA-B*07:02 Peripheral Blood MonoCytes (PBMCs) of healthy donors.

FIG. 54 provides a series of plots showing the identification of CD8⁺ T cells reactive against RPRPSQPSSL (SEQ ID NO: 32957) peptide from Peripheral Blood MonoCytes (PBMCs) of an HLA-B*07:02 healthy donor. CD8⁺ T cells reactive against RPRPSQPSSL (SEQ ID NO: 32957) peptide were identified by staining with two RPRPSQPSSL (SEQ ID NO: 32957) DEXTRAMERS 10 days after stimulation of naïve CD8⁺ T cells with irradiated autologous mature dentritic cells (DCs) in presence of IL2, IL7, IL15 and RPRPSQPSSL (SEQ ID NO: 32957) peptide. Figure discloses “RPRPSQPSSL” as SEQ ID NO: 32957.

### DETAILED DESCRIPTION OF THE INVENTION

The disclosure features compositions and methods that are useful for treating anaplastic lymphoma kinase (ALK) neoplasias.

The invention is based, at least in part, upon the discovery that T cell receptors (TCRs) expressed as heterologous proteins in an immune effector cell specifically recognize ALK sequences presented by major histocompatibility complexes (MHCs) or human leukocyte antigens (HLAs) and were effective in targeting ALK positive tumor cells. In particular, TCRs of the present disclosure, when transduced into T cells, transformed such T cells, particularly CD8⁺ T cells, into T cells capable of specifically, selectively and strongly recognizing ALK positive tumor cells becoming effective killers of ALK positive tumor cells.

Provided herein are also multiple methods of producing engineered TCRs specific to ALK peptide sequences presented by MHCs or HLAs. Such methods include, but are not limited to, isolation of ALK specific TCRs from one or more of the following sources: 1) transgenic mice expressing humanized MHCs and injected with specific ALK peptide sequences; 2) peripheral blood of subjects having ALK positive tumors; and/or 3) tumor infiltrating lymphocytes (TILs) of subjects having ALK positive tumors. Not intending to be bound by theory, the data provided herein pave the way for the development of engineered TCRs specific to ALK peptide sequences for use in treating ALK positive tumors.

### T Cell Receptors

The TCR evolved to detect subtle changes in cellular proteins that can include viral peptides or mutated oncogenic proteins. Thus, TCR-based agents can be directed to the vast majority of truly tumor-specific antigens. Peptides derived from proteins of any subcellular location are presented on the cell surface in the context of major histocompatibility complexes (MHC), known as human leukocyte antigens (HLA) when referring to human MHC, where they are recognized by T cells through their TCRs. TCR-based therapies are able to recognize and react to cells expressing these mutated or differentially expressed, cancer-associated proteins presented on MHC. The exploitation of this powerful modality to treat cancer and possibly other serious diseases. Numerous approaches have been employed to generate a more potent anti-tumor T cell response. Tumor-reactive T cells expressing native TCRs can be stimulated in vivo through administration of vaccines, checkpoint blockade inhibitors, or cytokines. Alternatively, reactive T cells can be expanded ex vivo and reinfused for adoptive cell therapy

Several biomarkers, including PD-1, 4-1BB and cytokine production, such as of IFNγ, indicate the T cell has interacted with its cognate antigen and can be used to isolate T cells. Not all T cells that are multimer positive, and therefore peptide-MHC specific, are able to exert cytotoxic effects against tumor cells expressing tumor antigens. Conversely, multimer staining may not detect all antigen-reactive T cells. This may be due to decreased TCR surface density or expression of TCRs with low affinity. Multimer staining can be enhanced to detect low-affinity TCRs by staining with multimers with more peptide-MHC sites, i.e. tetramers, dextramers or dodecamers.

T cell receptor therapies include the heterologous expression of T-cell receptors, as well as the expression of soluble anti-ALK T Cell receptors, soluble anti-ALK antibodies, or soluble anti-ALK bispecific or drug conjugate antibodies, all of which recognize ALK peptides in the context of HLAs.

### TCR Heterologous Expression

Individual reactive TCRs can be sequenced and subsequently expressed exogenously in T cells prior to reinfusion. Isolated individual reactive TCRs can be transduced and expressed into other T cells, known as TCR-T cells, to broaden therapy to additional patients. Immunization of mice expressing human HLA molecules or mice expressing the human TCR repertoire can be used for immunization and isolation of high affinity TCRs. High affinity human TCRs with increased activity also can be isolated when human T cells are stimulated ex vivo with tumor antigens on HLA mismatched antigen presenting cells.

### ALK Positive Neoplasias

Lung cancer is the most common cause of cancer-related death worldwide, and the annual incidence of anaplastic lymphoma kinase (ALK) expressing non-small cell lung cancer (NSCLC) in the U.S. is about 8,000 cases. In these patients, treatment with ALK tyrosine kinase inhibitors (TKIs) fails to induce durable remissions. In this context, a successful engineered TCR based treatment could lead to durable responses and greatly improve survival and quality of life for NSCLC patients. ALK represents an attractive target because of its oncogenicity, its immunogenicity, and its restricted expression to tumor tissue rather than healthy adult tissue. Importantly, use of therapeutic compositions including engineered TCRs could potentially be extended to many other cancer types which are driven by ALK rearrangements or activating mutations (i.e., ALK-positive cancers), such as anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, and melanoma. Therefore, the engineered TCRs described can more effectively treat patients diagnosed with ALK-positive tumors.

As described below, the present invention features engineered TCRs which specifically recognize ALK peptide sequences presented by MHCs or HLAs, derived from ALK-positive cell lines and immune cells from patients with ALK-positive cancers. Such immunogenic antigens are also referred to as “immunogens” herein. The ALK-specific immunogenic antigens elicit a potent immune response, e.g., in the form of reactive T-lymphocytes, following administration or delivery to, or introduction into, a subject, particularly, a human subject. The engineered TCRs may be used in methods to treat and/or reduce disease caused by oncogenic ALK gene fusions, rearrangements, duplications, or mutations.

The immunogenic ALK antigens described herein may be used in immunogenic compositions (e.g., ALK-specific vaccines) that treat ALK-positive cancers caused by oncogenic ALK gene fusions, rearrangements, duplications, or mutations in a subject, particularly a human subject, to whom the immunogenic composition or vaccine, is administered. The vaccine elicits a potent ALK protein-specific T cell response that treats and/or protects against ALK-positive cancers in a subject. The antigens, immunogens, immunogenic compositions and vaccines, and pharmaceutical compositions thereof, of the invention provide an additional treatment option for patients that have either become resistant to or have failed to respond to prior and traditional therapies for ALK-positive cancers.

### Heterologous T Cell Receptors (TCRs)

The present disclosure features the isolation, identification, and recombinant expression of T cell receptors (TCRs) having specificity to ALK antigens or peptide sequences presented by major histocompatibility complexes (MHCs) and/or human leukocyte antigens (HLAs) in immune effector cells. In some embodiments, the TCRs of the present invention are used to effectively treat a disease and its symptoms, either prophylactically or therapeutically, caused by oncogenic ALK gene fusions, rearrangements, duplications, or mutations (e.g., ALK-positive cancers or tumors) following administration and delivery to a susceptible subject. The disclosure provides pharmaceutical compositions comprising a vector encoding the TCRs, as well as cells expressing the heterologous TCRs.

In some embodiments, the TCRs are able to recognize at least one, more than one, or all types of ALK-positive cancers. In some embodiments, when the TCRs provided herein are expressed by an immune cell (e.g., a CD8+ T cell), the immune cells expressing the TCRs (i.e., engineered immune cells) are capable of killing or inhibiting the growth of ALK positive cancers or tumors.

The present invention also provides the specific complementarity determining regions (CDRs) which allow the TCRs to specifically recognize and/or bind to ALK antigens or peptide sequences presented by major histocompatibility complexes (MHCs) and/or human leukocyte antigens (HLAs). Without intending to be bound by theory, since CDRs are found throughout the immunoglobulin superfamily, proteins, peptides, and fragments thereof incorporating the provided CDRs are also provided by the present invention (e.g., polypeptides such as antibodies incorporating the CDR sequences provided herein). In some embodiments, the TCRs or polypeptides of the present disclosure include one or more of the CDR sequences listed in Tables 2 or 3 provided herein.

In embodiments, the TCRs provided herein may be humanized or non-humanized. Humanized TCRs are TCRs in which at least part of the sequence has been altered from its initial form to render it more like human TCRs. In embodiments, the TCRs are non-humanized. Without intending to be bound by theory, non-humanized heterologous TCRs do not pair with endogenous TCRs in a transduced T cell, and accordingly the use of non-humanized TCRs may have the advantage of preventing endogenous TCR/heterologous TCR mismatch pairings.

In some embodiments, the disease caused by oncogenic ALK gene fusions, rearrangements, duplications, or mutations is an ALK-positive cancer. Nonlimiting examples of ALK-positive cancers include non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, melanoma, or a combination thereof. In some embodiments, the ALK-positive cancer is non-small cell lung cancer (NSCLC). In some embodiments, the ALK-positive cancer is anaplastic large cell lymphoma (ALCL).

### ALK Antigens and Immunogens

The present invention provides herein ALK antigens and peptide sequences recognizable by the TCRs of the present invention. In some embodiments, such ALK antigens represent oncogenic ALK gene fusions, rearrangements, duplications, or mutations (e.g., ALK-positive cancers). An ALK antigen or peptide sequence as described herein is a polypeptide, peptide, or antigen receptor-binding portion thereof. In some embodiments, the ALK antigen or peptide sequence is an ALK polypeptide or fragment thereof.

In some embodiments, the ALK antigen or peptide amino acid sequence comprises an amino acid sequence that is at least 80%, at least 85%, at least 90%, at least 95%, at least 98%, at least 99%, or 100% identical to any of the following amino acid sequences: RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); AMLDLLHVA (SEQ ID NO: 32961); GGDLKSFLRETRPRPSQPSSLAMLDLLHVA (SEQ ID NO: 32962); FNHQNIVRCIGVSL (SEQ ID NO: 32963); and/or GGDLKSFLRETRPRPSQPSSLAM (SEQ ID NO: 32964). In some embodiments, the ALK antigen or peptide comprises an amino acid sequence that is at least 90%, at least 95%, at least 98%, at least 99%, or 100% identical to: RPRPSQPSSL (SEQ ID NO: 32957). In some embodiments, the ALK antigen or peptide comprises an amino acid sequence that is at least 90%, at least 95%, at least 98%, at least 99%, or 100% identical to: IVRCIGVSL (SEQ ID NO: 32958). In some embodiments, the ALK antigen or peptide comprises an amino acid sequence that is at least 90%, at least 95%, at least 98%, at least 99%, or 100% identical to: VPRKNITLI (SEQ ID NO: 32959). In some embodiments, the ALK antigen or peptide comprises an amino acid sequence that is at least 90%, at least 95%, at least 98%, at least 99%, or 100% identical to: TAAEVSVRV (SEQ ID NO: 32960). In some embodiments, the ALK antigen or peptide comprises an amino acid sequence that is at least 90%, at least 95%, at least 98%, at least 99%, or 100% identical to: AMLDLLHVA (SEQ ID NO: 32961). In some embodiments, the ALK antigen or peptide comprises an amino acid sequence that is at least 80%, at least 85%, at least 90%, at least 95%, at least 98%, at least 99%, or 100% identical to: GGDLKSFLRETRPRPSQPSSLAMLDLLHVA (SEQ ID NO: 32962). In some embodiments, the ALK antigen or peptide comprises an amino acid sequence that is at least 90%, at least 95%, at least 98%, at least 99%, or 100% identical to: FNHQNIVRCIGVSL (SEQ ID NO: 32963). In some embodiments, the ALK antigen or peptide comprises an amino acid sequence that is at least 80%, at least 85%, at least 90%, at least 95%, at least 98%, at least 99%, or 100% identical to: GGDLKSFLRETRPRPSQPSSLAM (SEQ ID NO: 32964).

In some embodiments, the ALK antigen or peptide is conjugated to an amphiphile or amphiphilic tail. In some embodiments, the amphiphile is N-hydroxy succinimidyl ester-end-functionalized poly(ethylene glycol)-lipid (NHS-PEG2KDa-DSPE). ALK amph-peptides may significantly increase T-cell expansion and greatly enhance anti-tumor efficacy. ALK amph-peptides may be generated as taught in H. Liu et al., Structure-based programming of lymph-node targeting in molecular vaccines. Nature 507, 5199522 (2014), which is incorporated herein in its entirety.

In some embodiments, the ALK antigen or peptide, optionally conjugated to an amphiphile or amphiphilic tail, comprises an amino acid sequence selected from the following amino acid sequences: RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); AMLDLLHVA (SEQ ID NO: 32961); GGDLKSFLRETRPRPSQPSSLAMLDLLHVA (SEQ ID NO: 32962); FNHQNIVRCIGVSL (SEQ ID NO: 32963); and/or GGDLKSFLRETRPRPSQPSSLAM (SEQ ID NO: 32964).

In some embodiments, the ALK antigen or peptide, optionally conjugated to an amphiphile or amphiphilic tail, comprises flanking amino acid sequences. In some embodiments, the flanking amino acid sequences are on either side or on both sides of the ALK antigen or peptide sequence. In some embodiments, the ALK antigen or peptide sequence is a central core amino acid sequence with flanking amino acid sequences on both sides of the core. In some embodiments, the core amino acid sequence is about 9 to 10 amino acids in length. In some embodiments, the flanking amino acid sequences are between 5 to 15 amino acids. In some embodiments, the ALK antigen or peptide conjugated to an amphiphile or amphiphilic tail comprises an amino acid sequence that is about 9 to about 30 amino acids in length.

In some embodiments, the ALK antigen or peptide is a polynucleotide molecule. In some embodiments, the ALK antigen or peptide has a polynucleotide sequence that encodes a polypeptide or peptide antigen or fragment thereof as described herein.

In some embodiments, ALK polynucleotide sequences encode ALK antigen or peptide amino acid sequences that are at least 90%, 95%, at least 98%, at least 99%, or 100% identical to any of the following amino acid sequences: RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); AMLDLLHVA (SEQ ID NO: 32961); GGDLKSFLRETRPRPSQPSSLAMLDLLHVA (SEQ ID NO: 32962); FNHQNIVRCIGVSL (SEQ ID NO: 32963); and/or GGDLKSFLRETRPRPSQPSSLAM (SEQ ID NO: 32964). In some embodiments, the ALK polynucleotide sequence encodes the ALK antigen or peptide amino acid sequence that is at least 90%, at least 95%, at least 98%, at least 99%, or 100% identical to: RPRPSQPSSL (SEQ ID NO: 32957). In some embodiments, the ALK polynucleotide sequence encodes the ALK antigen or peptide amino acid sequence that is at least 90%, at least 95%, at least 98%, at least 99%, or 100% identical to: IVRCIGVSL (SEQ ID NO: 32958). In some embodiments, the ALK polynucleotide sequence encodes the ALK antigen or peptide amino acid sequence that is at least 90%, at least 95%, at least 98%, at least 99%, or 100% identical to: VPRKNITLI (SEQ ID NO: 32959). In some embodiments, the ALK polynucleotide sequence encodes the ALK antigen or peptide amino acid sequence that is at least 90%, at least 95%, at least 98%, at least 99%, or 100% identical to: TAAEVSVRV (SEQ ID NO: 32960). In some embodiments, the ALK polynucleotide sequence encodes the ALK antigen or peptide amino acid sequence that is at least 90%, at least 95%, at least 98%, at least 99%, or 100% identical to: AMLDLLHVA (SEQ ID NO: 32961). In some embodiments, the ALK polynucleotide sequence encodes the ALK antigen or peptide amino acid sequence that is at least 90%, at least 95%, at least 98%, at least 99%, or 100% identical to: GGDLKSFLRETRPRPSQPSSLAMLDLLHVA (SEQ ID NO: 32962). In some embodiments, the ALK polynucleotide sequence encodes the ALK antigen or peptide amino acid sequence that is at least 90%, at least 95%, at least 98%, at least 99%, or 100% identical to: FNHQNIVRCIGVSL (SEQ ID NO: 32963). In some embodiments, the ALK polynucleotide sequence encodes the ALK antigen or peptide amino acid sequence that is at least 90%, at least 95%, at least 98%, at least 99%, or 100% identical to:

(SEQ ID NO: 32964)
GGDLKSFLRETRPRPSQPSSLAM.

In some embodiments, the amino acid sequence of the antigen or peptide, e.g., the ALK protein, is reverse translated and optimized for expression in mammalian cells. As will be appreciated by a skilled practitioner in the art, optimization of the nucleic acid sequence includes optimization of the codons for expression of a sequence in mammalian cells and RNA optimization (such as RNA stability).

In some embodiments, fusion proteins comprising the ALK antigen polypeptides are as described herein. In some embodiments, the ALK polypeptide can be fused to any heterologous amino acid sequence to form the fusion protein. By way of example, peptide components of ALK polypeptides may be generated independently and then fused together to produce an intact ALK polypeptide antigen.

### ALK Immunogenic Compositions and Vaccines

The ALK antigens or immunogens may be used in immunogenic compositions or vaccines to elicit an immune response, e.g., a T-cell response, against disease caused by oncogenic ALK gene fusions, rearrangements, duplications, or mutations (e.g., ALK-positive cancers). In some embodiments, the immune response includes producing T-lymphocytes.

In particular embodiments, the ALK polypeptides of the immunogenic compositions or vaccines contain antigenic determinants that serve to elicit an immune response in a subject (e.g., the production of activated T-cells) that can treat and/or protect a subject against disease caused by oncogenic ALK gene fusions, rearrangements, duplications, or mutations (e.g., ALK-positive cancers) and symptoms thereof.

In some embodiments, such immunogenic compositions or vaccines as described herein contain at least one ALK antigen or immunogen and are effective in treating, reducing, delaying, or preventing at least one disease caused by oncogenic ALK gene fusions, rearrangements, duplications, or mutations (e.g., ALK-positive cancers). In some embodiments, such immunogenic compositions or vaccines as described herein contain two or more ALK antigens or immunogens and are effective in treating, reducing, or preventing at least one disease caused by oncogenic ALK gene fusions, rearrangements, duplications, or mutations (e.g., ALK-positive cancers). In some embodiments, the two or more ALK antigens or immunogens comprise one, two, or more amino acid sequences selected from the following: AMLDLLHVA (SEQ ID NO: 32961); RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); GGDLKSFLRETRPRPSQPSSLAMLDLLHVA (SEQ ID NO: 32962); FNHQNIVRCIGVSL (SEQ ID NO: 32963); and/or GGDLKSFLRETRPRPSQPSSLAM (SEQ ID NO: 32964). In some embodiments, the immunogenic compositions or vaccines contain at least one ALK antigen or immunogen conjugated to an amphiphile or amphiphilic tail. In some embodiments, at least one of the two or more ALK antigens or immunogens in an immunogenic composition or vaccine is conjugated to an amphiphile or amphiphilic tail. In some embodiments, the amphiphile is N-hydroxy succinimidyl ester-end-functionalized poly(ethylene glycol)-lipid (NHS-PEG2Kda-DSPE). In some embodiments, the two or more ALK antigens or immunogens are provided in equal concentration ratios in an immunogenic composition or vaccine.

Because the ALK antigens or immunogens and the sequences thereof as described herein and used as immunogenic compositions or vaccines elicit an immune response in an immunocompetent subject, they provide a superior vaccine against which an immune response (e.g., producing T-lymphocytes) is generated.

In some embodiments, an immunogenic composition or a vaccine is provided that elicits an immune response (e.g., producing T-lymphocytes) in a subject following introduction, administration, or delivery of the antigen or immunogen to the subject. The route of introduction, administration, or delivery is not limited and may include, for example, intravenous, subcutaneous, intramuscular, oral, or other routes. The immunogenic composition or vaccine may be therapeutic (e.g., administered to a subject following a symptom of disease caused by oncogenic ALK gene fusions, rearrangements, duplications, or mutations (e.g., ALK-positive cancers)) or prophylactic (e.g., administered to a subject prior to the subject having or expressing a symptom of disease, or full-blown disease, caused by oncogenic ALK gene fusions, rearrangements, duplications, or mutations (e.g., ALK-positive cancers)).

### Vectors

Vectors containing a nucleotide sequence encoding the TCRs described herein are provided. In some embodiments, the vector further includes a promoter operably linked to the nucleotide sequence encoding the TCR. In embodiments, the promoter is a cytomegalovirus (CMV) promoter. In embodiments, the promoter is a murine stem cell virus (MSCV) promoter.

The vectors used to express an TCR as described herein may be any suitable expression vector known and used in the art. In some embodiments, the vector is a prokaryotic or eukaryotic vector. In some embodiments, the vector is an expression vector, such as a eukaryotic (e.g., mammalian) expression vector. In another embodiment, the vector is a plasmid (prokaryotic or bacterial) vector. In another embodiment, the vector is a viral vector (e.g., a retroviral vector, lentiviral vector or an adeno associated viral vector). In some embodiments, the vector is an RNA polynucleotide suitable for translation in a cell. In embodiments, the vector is a MSCV vector.

In some embodiments, the vectors comprise a nucleotide sequence encoding a TCR amino acid sequence that is at least 85%, at least 90%, at least 95%, at least 96%, at least 97%, at least 98%, at least 99%, or 100% identical to a TCR sequence comprising an amino acid sequence provided in SEQ ID Nos 1-1484, 2969-4452, 5937-7420, 8905-10388, 11873-13356, 14841-17808, 19293-19901, 20513-21122, 21733-22342, 22953-23562, 24173-24782, 25393-26002, 26613-27222, 27833-27968, 28105-28240, 28377-28512, 28649-28784, 28921-29056, 29193-29328, 29465-29600, 29737-29764, 29793-29820, 29849-29876, 29905-29932, 29961-29988, 30018-30045, 30074-30101, 30130-30225, 30322-30417, 30514-30609, 30706-30801, 30898-30993, 31090-31185, or 31282-31377, herein. In some embodiments, the vectors comprise a nucleotide sequence encoding a CDR I amino acid sequence that is at least 85%, at least 90%, at least 95%, at least 96%, at least 97%, at least 98%, at least 99%, or 100% identical to a CDR 1 sequence provided in SEQ ID Nos 2969-4452, 20513-21122, 28105-28240, 29793-29820, or 30321-30416, herein. In some embodiments, the vectors comprise a nucleotide sequence encoding a CDR 2 amino acid sequence that is at least 85%, at least 90%, at least 95%, at least 96%, at least 97%, at least 98%, at least 99%, or 100% identical to a CDR 2 sequence provided in SEQ ID Nos 8905-10388, 22953-23562, 28649-28784, 29905-29932, or 30705-30800, herein. In some embodiments, the vectors comprise a nucleotide sequence encoding a CDR 3 amino acid sequence that is at least 85%, at least 90%, at least 95%, at least 96%, at least 97%, at least 98%, at least 99%, or 100% identical to a CDR 3 sequence provided in SEQ ID Nos 14841-16324, 25393-26002, 29193-29328, 30017-30044, or 31089-31184, herein.

Provided are isolated, non-naturally occurring TCRs, e.g., TCRs selected for specificity to ALK antigens or peptides sequences presented by MHCs or HLAs, produced by transfecting a host cell with an expression vector as known and used in the art under conditions sufficient to allow for expression of the TCR, e.g., a TCR, in the cell. Isolated cells including the vectors are also provided.

Collections of vectors are also contemplated. In certain embodiments, the collection of vectors includes plasmid encoding an TCR as described herein.

### Transgenic Effector Cells

Provided herein are effector cells expressing the TCRs specific to ALK antigens or peptide sequences provided herein (e.g., TCRs including one or more of the CDRs listed in Tables 2 or 3). In some embodiments, the transgenic effector cells include polynucleotide sequences encoding the TCRs specific to ALK antigens or peptide sequences provided herein. In some embodiments, the transgenic effector cells are produced by transforming or transfecting a host or naïve effector cell (e.g., a CD8+ and/or CD4+ cell, T cell, natural killer (NK) cell, cytotoxic T lymphocyte (CTL) cell, or regulatory T cell) with an expression vector containing a polynucleotide encoding an TCR, as described herein, as known and used in the art under conditions sufficient to allow for expression of the TCR, thereby producing the transgenic effector cell. Isolated transgenic effector cells (e.g., T cells, NK cells, CTL cells, or regulatory T cells) containing the vectors are also provided. In embodiments, the effector cell is isolated from a host and transduced ex vivo before infusion into a subject. The host may be the same as the subject, or different. Accordingly, in embodiments, the transgenic effector cell may be autologous, allogeneic, or heterologous.

In embodiments, T cells transduced with the heterologous TCRs of the present disclosure also include the knockout of endogenous TCRs in the T cells to prevent expression of the endogenous TCRs. In embodiments, one or more of the endogenous T cell receptor alpha constant (TRAC), T cell receptor beta constant 1 (TRBC1), and/or T cell receptor beta constant 2 (TRBC2) genes are knocked out. In embodiments, the endogenous TCRs are knocked out using gene editing techniques known in the art (e.g., CRISPR). In embodiments, knockout of the TRAC gene involves the use of the crRNA guide AGAGATCTCCCACACCCAAA (SEQ ID NO: 32986). In embodiments, knockout of the TRBC1 and/or TRBC2 genes involves the use of the crRNA guide AGAGATCTCCCACACCCAAA (SEQ ID NO: 32986).

### Polypeptides Specific to ALK

Provided herein are polypeptides having specificity to the ALK antigens or peptide sequences provided herein. Such polypeptides are characterized by their specificity and ability to bind to the ALK antigens or peptide sequences provided herein. In some embodiments, polypeptides of the present disclosure are members of the immunoglobulin superfamily, and may include, but are not limited to, T cell receptors, B cell receptors, antibodies (e.g., antibodies specific to the ALK antigens or peptides sequences provided herein, bispecific antibodies, drug conjugated antibodies, etc.), and variants, derivatives, or fragments thereof.

In some embodiments, the polypeptides have a complementarity determining region (CDR) 1 amino acid sequence that is at least 85%, at least 90%, at least 95%, at least 96%, at least 97%, at least 98%, at least 99%, or 100% identical to a CDR 1 sequence provided in SEQ ID Nos 2969-4452, 20513-21122, 28105-28240, 29793-29820, or 30321-30416, herein. In some embodiments, the polypeptides have a CDR 2 amino acid sequence that is at least 85%, at least 90%, at least 95%, at least 96%, at least 97%, at least 98%, at least 99%, or 100% identical to a CDR 2 sequence provided in SEQ ID Nos 8905-10388, 22953-23562, 28649-28784, 29905-29932, or 30705-30800, herein. In some embodiments, the polypeptides have a CDR 3 amino acid sequence that is at least 85%, at least 90%, at least 95%, at least 96%, at least 97%, at least 98%, at least 99%, or 100% identical to a CDR 3 sequence provided in SEQ ID Nos 14841-16324, 25393-26002, 29193-29328, 30017-30044, or 31089-31184, herein.

### Antibodies Specific to ALK

Provided herein are antibodies that selectively bind ALK antigens or peptide sequences. In some embodiments, the antibodies of the present disclosure are characterized by their ability to bind those ALK antigens or peptide sequences provided herein.

Methods of preparing antibodies are well known to those of ordinary skill in the science of immunology. As used herein, the term “antibody” means not only intact antibody molecules, but also fragments of antibody molecules that retain immunogen-binding ability, particularly ALK binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

In one embodiment, an antibody that binds an ALK antigen or peptide sequence is monoclonal. Alternatively, the antibody that binds an ALK antigen or peptide sequence is a polyclonal antibody. The preparation and use of polyclonal antibodies are known to the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing ALK peptide sequences or antigens disclosed herein, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding an ALK peptide sequence or antigen or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding an ALK peptide sequence or antigen, or immunogenic fragments thereof, can be expressed in cells in vitro, followed by isolation of the ALK peptide sequence or antigen and administration of the ALK peptide sequence or antigen to a suitable host in which antibodies are raised.

Alternatively, antibodies against an ALK peptide sequence or antigen may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

The disclosed ALK specific antibodies can be recombinantly fused or chemically conjugated (including both covalently and non-covalently conjugations) to a heterologous molecule (i.e., an unrelated molecule). The fusion does not necessarily need to be direct, but may occur through linker sequences.

In some embodiments, the disclosed ALK specific antibodies may be fused or conjugated to therapeutic compounds, preferably therapeutic compounds useful in treating neoplasias. In exemplary embodiments, the disclosed ALK specific antibodies may be fused or conjugated to compounds which effectively treat a disease and its symptoms, either prophylactically or therapeutically, caused by oncogenic ALK gene fusions, rearrangements, duplications, or mutations (e.g., ALK-positive cancers or tumors) following administration and delivery to a susceptible subject.

In some embodiments the antibody is conjugated to an agent. The agent can be, for example, a cytotoxic agent such as a chemotherapeutic drug, or radioactive isotype. In some embodiments, the agent is a toxin. Upon binding to ALK on a target tumor cell, the antibody and its payload can be internalized by the target cell. In this way, the antibody-drug conjugate can carry out selective destruction of the target cells. Suitable drug conjugates as well as methods of making and using antibody-drug conjugates in the treatment of immune disorders and cancer are known in the art. See for example, U.S. Published Application No. 2012/0288512, which describes anti-CD70-drug conjugates, is specifically incorporated by reference in its entirety.

Techniques for conjugating therapeutic compounds to antibodies are well known; see, e.g., Amon et al., “Monoclonal Antibodies For Immunotargeting Of Drugs In Cancer Therapy”, in MONOCLONAL ANTIBODIES AND CANCER THERAPY, Reisfeld et al. (eds.), 1985, pp. 243-56, Alan R. Liss, Inc.); Hellstrom et al., “Antibodies For Drug Delivery”, in CONTROLLED DRUG DELIVERY (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in MONOCLONAL ANTIBODIES '84: BIOLOGICAL AND CLINICAL APPLICATIONS, Pinchera et al. (eds.), 1985, pp. 475-506); “Analysis, Results, And Future Prospective Of The Therapeutic Use Of Radiolabeled Antibody In Cancer Therapy”, in MONOCLONAL ANTIBODIES FOR CANCER DETECTION AND THERAPY, Baldwin et al. (eds.), 1985, pp. 303-16, Academic Press; and Thorpe et al. (1982) “The Preparation And Cytotoxic Properties Of Antibody-Toxin Conjugates,” Immunol. Rev. 62:119-158.

### Compositions and Pharmaceutical Compositions for Administration

Compositions comprising one or more T cell receptors, vectors encoding T cell receptors, polypeptides (e.g., polypeptides specific to ALK peptide sequences and/or antigens), and/or transgenic cells expressing such receptors, as described herein are provided. In some embodiments, the compositions further comprise a pharmaceutically acceptable carrier, diluent, excipient, or vehicle. In some embodiments, an adjuvant (a pharmacological or immunological agent that modifies or boosts an immune response, e.g., to produce more antibodies that are longer-lasting) is also employed. For example, without limitation, the adjuvant can be an inorganic compound, such as alum, aluminum hydroxide, or aluminum phosphate; mineral or paraffin oil; squalene; detergents such as Quil A; plant saponins; Freund's complete or incomplete adjuvant, a biological adjuvant (e.g., cytokines such as IL-1, IL-2, or IL-12); bacterial products such as killed Bordetella pertussis, or toxoids; or immunostimulatory oligonucleotides (such as CpG oligonucleotides). In some embodiments, the adjuvant is conjugated to an amphiphile as previously described (H. Liu et al., Structure-based programming of lymph-node targeting in molecular vaccines. Nature 507, 5199522 (2014)). In some embodiments, the amphiphile is N-hydroxy succinimidyl ester-end-functionalized poly(ethylene glycol)-lipid (NHS-PEG2Kda-DSPE)

Compositions and preparations (e.g., physiologically or pharmaceutically acceptable compositions) containing T cell receptors, vectors encoding T cell receptors, polypeptides, and/or engineered cells expressing such receptors for parenteral administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions, or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases, and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include a therapeutically effective amount of an isolated T cell receptor, vector encoding T cell receptors, polypeptide, and/or engineered cell, as described herein, alone, or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives, and the like. Other media that can be used in the compositions and administration methods as described are normal saline and sesame oil.

### Methods of Treatment, Administration and Delivery

Methods of treating a disease (e.g., ALK-positive cancers (e.g., lung cancer, lymphoma or neuroblastoma)), or symptoms thereof, are provided. The methods comprise administering a therapeutically effective amount of an T cell receptor, vector encoding T cell receptors, polypeptide, and/or engineered cell, as described herein, or a pharmaceutical composition thereof, as described herein, to a subject (e.g., a mammal), in particular, a human subject. The invention provides methods of treating a subject suffering from, or at risk of, or susceptible to disease, or a symptom thereof, or delaying the progression of a disease (e.g., ALK-positive cancer (e.g., lung cancer, lymphoma or neuroblastoma)). In some embodiments, the method includes administering to the subject (e.g., a mammalian subject), an effective amount or a therapeutic amount of a T cell receptor, vector encoding a T cell receptor, polypeptide, and/or engineered cell, as described herein, or pharmaceutical composition thereof, sufficient to treat the disease, delay the growth of, or treat the symptoms thereof (e.g., ALK-positive cancers (e.g., lung cancer, lymphoma or neuroblastoma)). In embodiments, an effective amount of a T cell receptor, vector encoding a T cell receptor, polypeptide, and/or engineered cell, as described herein, or pharmaceutical composition thereof, is an amount sufficient to induce partial remission (PR) of a tumor in a subject, complete remission (CR) of a tumor in a subject, or stable disease (SD) in a subject.

In some embodiments, the methods herein include administering to the subject (including a human subject identified as in need of such treatment) an effective amount of an T cell receptor, vector encoding T cell receptors, polypeptide, and/or engineered cell, or a pharmaceutical composition thereof, as described herein to produce such effect. The treatment methods are suitably administered to subjects, particularly humans, suffering from, are susceptible to, or at risk of having a disease, or symptoms thereof, namely, cancer (e.g., ALK-positive cancers (e.g., lung cancer, lymphoma or neuroblastoma)). Nonlimiting examples of ALK-positive cancers include non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, melanoma, or a combination thereof. In some embodiments, the ALK-positive cancer is neuroblastoma.

The ALK-positive cancer may be caused by an oncogenic ALK gene that either forms a fusion gene with other genes, gains additional gene copies, or is genetically mutated. In some embodiments, the ALK-positive cancer is caused by an ALK fusion gene encoding an ALK fusion protein. In some embodiments, the ALK-positive cancer is caused by a fusion between the ALK gene and the nucleophosmin (NPM) gene encoding a NPM-ALK fusion protein. In some embodiments, the ALK-positive cancer is caused by a fusion between the ALK gene and the echinoderm microtubule-associated protein-like 4 (EML4) gene encoding an ELM4-ALK fusion protein. In some embodiments, the ALK-positive cancer is caused by a point mutation. In some embodiments, the point mutation is F1174L (ALK^{F1174L}).

Identifying a subject in need of such treatment can be based on the judgment of the subject or of a health care professional and can be subjective (e.g., opinion) or objective (e.g., measurable by a test or diagnostic method). Briefly, the determination of those subjects who are in need of treatment or who are “at risk” or “susceptible” can be made by any objective or subjective determination by a diagnostic test (e.g., blood sample, biopsy, genetic test, enzyme or protein marker assay), marker analysis, family history, and the like, including an opinion of the subject or a health care provider. The T cell receptor, vector encoding T cell receptors, polypeptide, and/or engineered cell, or pharmaceutical compositions thereof, as described herein, may also be used in the treatment of any other disorders in which disease caused by oncogenic ALK gene fusions, rearrangements, duplications or mutations may be implicated. A subject undergoing treatment can be a non-human mammal, such as a veterinary subject, or a human subject (also referred to as a “patient”). In some embodiments, the disease caused by oncogenic ALK-gene fusions, rearrangements, duplications, or mutations may be one resistant to treatment with TKIs (e.g., an anaplastic lymphoma kinase (ALK)-rearranged and/or ALK-positive neoplasia that is resistant to ALK tyrosine kinase inhibitor (TKI) therapy).

In addition, prophylactic methods of preventing or protecting against a disease (e.g., ALK-positive cancers (e.g., lung cancer, lymphoma or neuroblastoma)), or symptoms thereof, are provided. Such methods comprise administering a therapeutically effective amount of a pharmaceutical composition comprising an T cell receptor, vector encoding T cell receptors, polypeptide, and/or engineered cell as described herein to a subject (e.g., a mammal, such as a human), in particular, prior to development or onset of a disease (e.g., ALK-positive cancers (e.g., lung cancer, lymphoma or neuroblastoma)).

In another embodiment, a method of monitoring the progress of a disease (e.g., ALK-positive cancers (e.g., lung cancer, lymphoma or neuroblastoma)), or monitoring treatment of the disease is provided. The method includes a diagnostic measurement (e.g., CT scan, screening assay or detection assay) in a subject suffering from or susceptible to disease or symptoms thereof (e.g., ALK-positive cancers (e.g., lung cancer, lymphoma or neuroblastoma)), in which the subject has been administered an amount (e.g., a therapeutic amount) of an T cell receptor, vector encoding T cell receptors, polypeptide, and/or engineered cell, or a pharmaceutical composition thereof, as described herein, sufficient to treat the disease or symptoms thereof. The diagnostic measurement in the method can be compared to samples from healthy, normal controls; in a pre-disease sample of the subject; or in other afflicted/diseased patients to establish the treated subject's disease status. For monitoring, a second diagnostic measurement may be obtained from the subject at a time point later than the determination of the first diagnostic measurement, and the two measurements can be compared to monitor the course of disease or the efficacy of the therapy/treatment. In certain embodiments, a pre-treatment measurement in the subject (e.g., in a sample or biopsy obtained from the subject or CT scan) is determined prior to beginning treatment as described: this measurement can then be compared to a measurement in the subject after the treatment commences and/or during the course of treatment to determine the efficacy of (monitor the efficacy of) the disease treatment.

The T cell receptor, vector encoding T cell receptors, polypeptide, and/or engineered cell, or pharmaceutical compositions thereof, can be administered to a subject by any of the routes normally used for introducing a recombinant protein or composition containing the recombinant protein into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

The T cell receptor, vector encoding T cell receptors, polypeptide, and/or engineered cell, or pharmaceutical compositions thereof, can be administered in any suitable manner, such as with pharmaceutically acceptable carriers, diluents, or excipients as described supra. Pharmaceutically acceptable carriers are determined in part by the particular composition being administered, as well as by the particular method used to administer the composition. Accordingly, a pharmaceutical composition comprising the T cell receptor, vector encoding T cell receptors, polypeptide, and/or engineered cell, can be prepared using a wide variety of suitable and physiologically and pharmaceutically acceptable formulations. In some embodiments, the disclosed methods include isolating T cells from a subject, transducing the T cells with an expression vector (e.g., a lentiviral vector) encoding the TCRs, and administering the engineered TCR-expressing T cells to the subject for treatment of a disease ((e.g., ALK-positive cancers (e.g., lung cancer, lymphoma or neuroblastoma)) in the subject.

Administration of the T cell receptor, vector encoding T cell receptors, polypeptide, and/or engineered cell, or pharmaceutical compositions thereof, can be accomplished by single or multiple doses. The dose administered to a subject should be sufficient to induce a beneficial therapeutic response in a subject over time, such as to inhibit, block, reduce, ameliorate, protect against, or prevent disease (e.g., ALK-positive cancers (e.g., lung cancer, lymphoma or neuroblastoma)). The dose required will vary from subject to subject depending on the species, age, weight and general condition of the subject, by the severity of the cancer being treated, by the particular composition being used and by the mode of administration. An appropriate dose can be determined by a person skilled in the art, such as a clinician or medical practitioner, using only routine experimentation. One of skill in the art is capable of determining therapeutically effective amounts of T cell receptor, vector encoding T cell receptors, polypeptide, and/or engineered cell, or pharmaceutical compositions, that provide a therapeutic effect or protection against disease (e.g., ALK-positive cancers (e.g., lung cancer, lymphoma or neuroblastoma)) suitable for administering to a subject in need of treatment or protection.

In some embodiments, a T cell receptor, vector encoding T cell receptors, polypeptide, and/or engineered cell, or a pharmaceutical composition thereof, is administered as a maxium-tolerated dose (MTD). In some embodiments, MTD is the dose with estimated probability of dose limiting toxicity (DLT) closest to the target toxicity rate of 20%. In some embodiments, a T cell receptor, vector encoding T cell receptors, polypeptide, and/or engineered cell, or a pharmaceutical composition thereof, is administered in a therapeutically effective dose for a mammal. In some embodiments, the mammal is a mouse. In some embodiments, a mouse is administered a dose of 0.5 million to 15 million cells. In some embodiments, the mammal is a human. In some embodiments, a human is administered a dose of at least about 0.25×10⁶ engineered cells/kg, at least about 0.5×10⁶ engineered cells/kg, at least about 1×10⁶ engineered cells/kg, or at least about 1.5×10⁶ engineered cells/kg.

### Combination Therapies

The T cell receptor, vector encoding T cell receptors, polypeptide, and/or engineered cells expressing such receptors as described herein can be administered alone or in combination with other therapeutic agents in a subject for the treatment of cancer (e.g., ALK-positive cancer (e.g., neuroblastoma)). For example, the T cell receptor, vector encoding T cell receptors, polypeptide, and/or engineered cell can be administered with an adjuvant, such as alum, Freund's incomplete adjuvant, Freund's complete adjuvant, biological adjuvant, or immunostimulatory oligonucleotides (such as CpG oligonucleotides). The adjuvant may be conjugated to an amphiphile as previously described (H. Liu et al., Structure-based programming of lymph-node targeting in molecular vaccines. Nature 507, 5199522 (2014)). In some embodiments, the amphiphile conjugated to the adjuvant is N-hydroxy succinimidyl ester-end-functionalized poly(ethylene glycol)-lipid (NHS-PEG2KDa-DSPE).

One or more cytokines, such as interleukin-1 (IL-2), interleukin-6 (IL-6), interleukin-12 (IL-12), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), or interferon-gamma (IFN-γ): a stimulator of interferon genes (STING) agonist (e.g., ADU-S100); one or more growth factors, such as GM-CSF or granulocyte-colony stimulation factor (G-CSF): one or more molecules such as the TNF ligand superfamily member 4 ligand (OX40L) or the type 2 transmembrane glycoprotein receptor belonging to the TNF superfamily (4-1BBL), or combinations of these molecules, can be used as biological adjuvants, if desired or warranted (see, e.g., Salgaller et al., 1998, J. Surg. Oncol. 68 (2); 122-38; Lotze et al., 2000, Cancer J. Sci. Am. 6 (Suppl 1); S61-6; Cao et al., 1998, Stem Cells 16 (Suppl 1); 251-60; Kuiper et al., 2000, Adv. Exp. Med. Biol. 465:381-90). These molecules can be administered systemically (or locally) to a subject.

The T cell receptor, vector encoding T cell receptors, polypeptide, and/or engineered cell can also be administered as a combination therapy with one or more other therapeutic agents, such as an ALK peptide or fusion protein. ALK peptide vaccine, ALK inhibitors, tyrosine kinase inhibitors (TKIs), and/or immune checkpoint inhibitors. Non-limiting examples of ALK inhibitors include lorlatinib (Lobrena®), ceritinib, alectinib, and brigatinib (Gristina et al., Pharmaceuticals 13: Issue 12 10.3390/ph13120474. In other embodiments, an ALK inhibitor is an agent that degrades ALK (e.g., an ALK proteolysis-targeting chimera (PROTAC). In other embodiments, the ALK inhibitor is a an ALK covalent inhibitor, preferably an ALK covelent inhibitor designed to bind an amino acid in ALK, such as cysteine (e.g., Con B-1 as described in Yan et al., J Med Chem. 2021 Feb. 11; 64 (3):1558-1569; BNP7787 as described in Parker et al., Onco Targets Ther. 2015 Feb. 4; 8:375-83; Non-limiting examples of checkpoint inhibitors include programmed cell death protein 1 (PD-1) inhibitors, programmed death-ligand 1 (PD-L1) inhibitors, cytotoxic T-lymphocyte-associated antigen-4 (CTLA-4) inhibitors, T-cell immunoglobulin and mucin domain 3 (TIM3) inhibitors, lymphocyte-activation gene 3 (LAG3) inhibitors, T-cell immunoglobulin and ITIM domain (TIGIT) inhibitors, V-domain immunoglobulin suppressor of T cell activation (VISTA) inhibitors, immunoglobulin-like transcript 2 (ILT2) inhibitors, immunoglobulin-like transcript 4 (ILT4) inhibitors, and killer cell immunoglobulin-like receptor, three immunoglobulin domains and long cytoplasmic tail (KIR3DL3) inhibitors. Nonlimiting examples of PD-1 inhibitors include pembrolizumab (Keytruda®) and nivolumab (Opdivo®). Nonlimiting examples of CTLA-4 inhibitors include ipilimumab (Yervoy®). Non-limiting examples of PD-L1 inhibitors include atezolizumab (TECENTRIQ®), avelumab (BAVENCIO®), and durvalumab (IMFINZI®). Non-limiting examples of TIM3 inhibitors include sabatolimab and cobolimab. Non-limiting examples of LAG3 inhibitors include relatimab. Non-limiting examples of TIGIT inhibitors include vibostolimab, ociperlimab, domvanalimab, and etigilimab. Non-limiting examples of VISTA inhibitors include onvatilimab. Non-limiting examples of ILT2 inhibitors include BND-22. Non-limiting examples of ILT4 inhibitors include MK-4830 and JTX-8064. Non-limiting examples of KIR3DL3 inhibitors include NPX-267. Non-limiting examples of TKI inhibitors include crizotinib, ceritinib, alectinib, brigatinib, and lorlatinib. In some embodiments, one or more ALK peptides or fusion proteins, ALK peptide vaccines, ALK inhibitors, immune checkpoint inhibitors, and/or TKI inhibitors is/are administered simultaneously or sequentially with T cell receptors, vector encoding T cell receptors, polypeptide, and/or engineered cells, as described herein, to a subject (e.g., human). In embodiments, combination therapies disclosed herein are administered in an amount effective to induce partial remission (PR) of a tumor in a subject, complete remission (CR) of a tumor in a subject, or stable disease (SD) in a subject.

In some embodiments, the T cell receptor, vector encoding T cell receptors, polypeptide, and/or engineered cell is administered simultaneously or sequentially with an ALK peptide vaccine. In particular embodiments, the ALK peptide vaccine contains antigenic determinants that serve to elicit an immune response in a subject (e.g., the production of activated T-cells) that can treat and/or protect a subject against disease caused by oncogenic ALK gene fusions, rearrangements, duplications or mutations (e.g., ALK-positive cancers) and symptoms thereof. In some embodiments, the immune response includes producing T-lymphocytes. In some embodiments, the ALK peptide vaccine contains at least one ALK antigen or peptide or fragment thereof. In some embodiments, the ALK peptide vaccine contains two or more ALK peptides or antigens or fragments thereof. In some embodiments, the ALK peptides or antigens or fragments thereof are fragments of the cytoplasmic portion of an ALK protein, which bind a human leukocyte antigen (HLA). In some embodiments the ALK peptides or antigens or fragments thereof are modified with an amphiphilic conjugate to increase T-cell expansion and greatly enhance anti-tumor efficacy. In some embodiments, the amphiphile is N-hydroxy succinimidyl ester-end-functionalized poly(ethylene glycol)-lipid (NHS-PEG2KDa-DSPE).

### Kits

Also provided are kits containing the T cell receptor, vector encoding T cell receptors, polypeptide, and/or engineered cell, or a pharmaceutically acceptable composition thereof, as described herein, and a pharmaceutically acceptable carrier, diluent, or excipient, for administering to a subject, for example. In some embodiments, the kit is provided for treating cancer (e.g., ALK-positive cancer (e.g., lung cancer, lymphoma or neuroblastoma)) in a subject (e.g., human). In some embodiments, the kit is provided for making an T cell receptor, vector encoding T cell receptors, polypeptide, and/or engineered cell as provided herein. In some embodiments, the kit will contain one or more of an ALK antibody or antigen binding fragment thereof, nucleic acid molecule encoding for an ALK peptide, T cell receptor, vector encoding T cell receptors, polypeptide, and/or engineered cell, as disclosed herein. The TCR may be in the form of a polypeptide or a polynucleotide encoding a TCR, as described herein. In some embodiments, the kit comprises a vector containing a nucleotide sequence encoding an TCR as disclosed herein. As will be appreciated by the skilled practitioner in the art, such a kit may contain one or more containers, labels, carriers, diluents or excipients, as necessary, and instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Useful techniques for particular embodiments will be discussed in the sections that follow.

The following examples are put forth to provide those of ordinary skill in the art with a complete disclosure and description of how to make and use the assay, screening, and therapeutic methods of the invention, and are not intended to limit the scope of what the inventors regard as their invention.

### EXAMPLES

The following examples are provided to illustrate certain particular features and/or embodiments. The examples should not be construed to limit the disclosure to the particular features or embodiments described.

### Example 1: Isolation of ALK-Specific TCRs from HLA-A*02:01 and HLA-B*07:02 Transgenic Mice Vaccinated with ALK Peptides

Alpha Beta T cell receptors form a site that recognizes molecular complexes containing peptides bound to MHC class I or MHC class II (MHC-I, II) molecules.

Data supporting the feasibility of isolating and cloning ALK-specific T Cell Receptors (TCRs) was generated by vaccinating BALB/c mice with a peptide fragment of anaplastic lymphoma kinase (ALK), ALK peptide PGPGRVAKI (SEQ ID NO: 32985), which was emulsified with adjuvant cyclic diguanylate monophosphate (c-di-GMP). BALB/c mice received a priming vaccine containing the ALK peptide and adjuvant at day 0 followed by two booster vaccines at day 14 and day 21. BALB/c splenocytes were isolated from the mice seven days after the last vaccination booster. The splenocytes were then stimulated overnight in vitro with the ALK peptide PGPGRVAKI (SEQ ID NO: 32985).

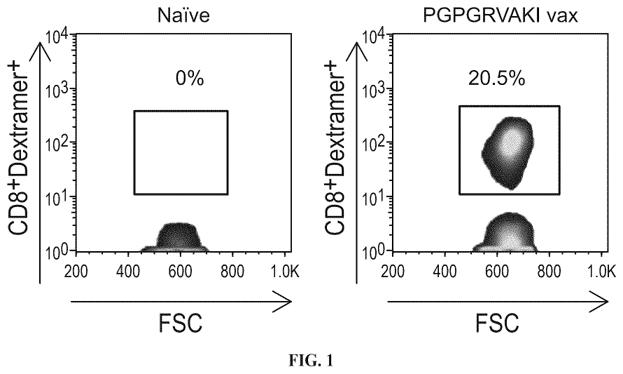

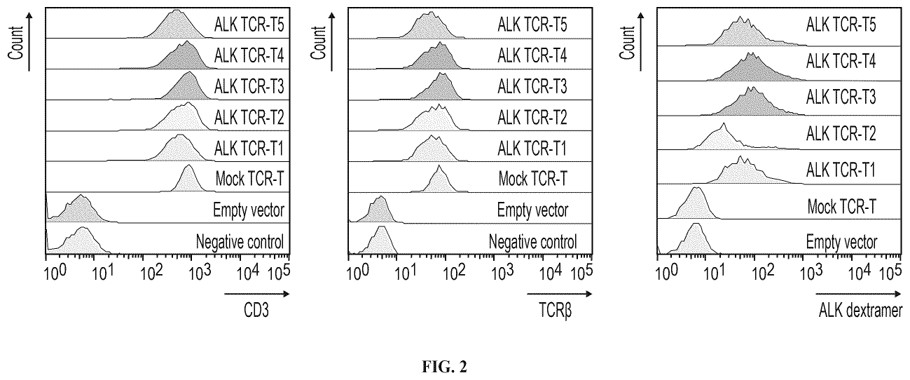

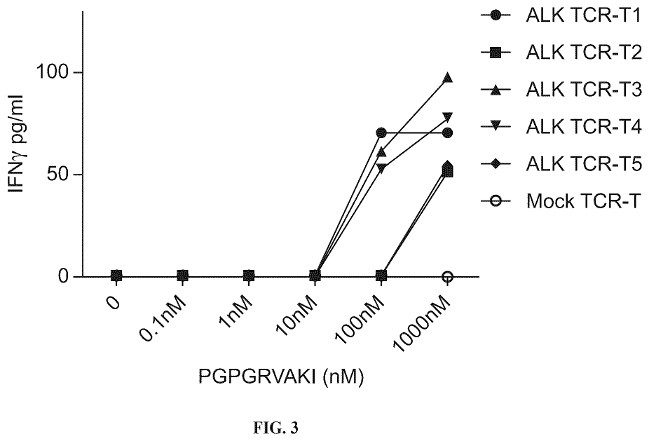

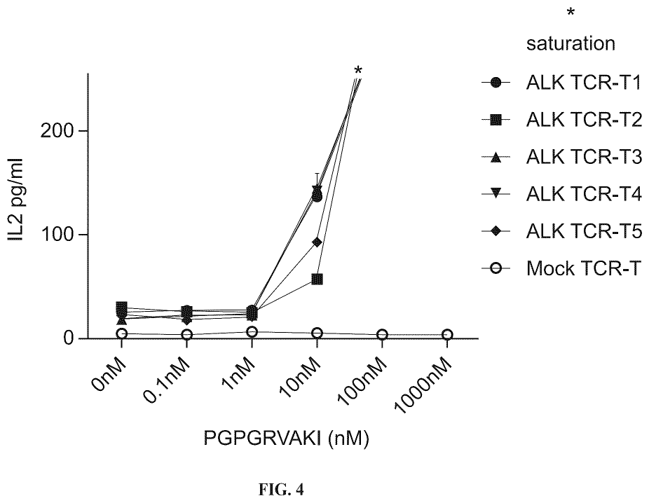

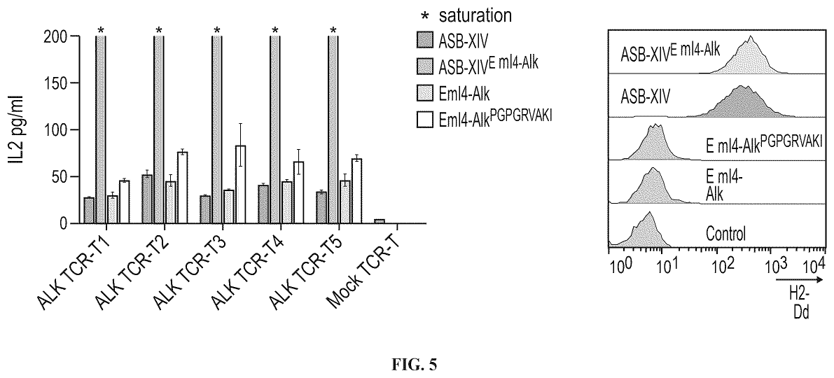

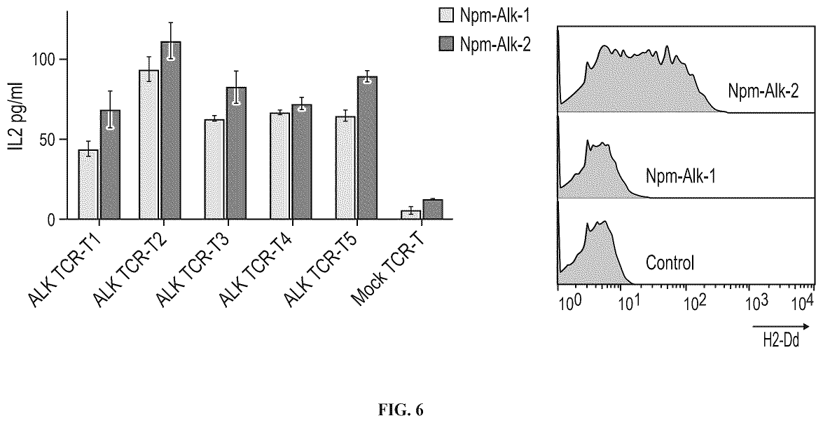

ALK-specific CD8+ T cells were sorted via staining with an MHC-I multimer optimized to accommodate multiple binding sites and numerous fluorophores to boost avidity in bind antigen-specific CD8+ T cells, which is commercially available (Dextramer® reagent, Immudex), that binds the PGPGRVAKI (SEQ ID NO: 32985) peptide with the mouse H-2D^{d} MHC-I (FIG. 1). Sorted T cells were subjected to single cell sequencing using 10× genomics technology. Among 5044 single cells sequenced, the five most expanded clonotypes were identified. The paired α and β chains of these five ALK-specific TCRs were cloned into a retroviral vector that efficiently transduced and reconstituted TCR expression in the TCR-deficient BW5147.3 lymphoma cell line. Remarkably, all five murine ALK-specific TCR-T cell lines generated recognized the PGPGRVAKI (SEQ ID NO: 32985) Dextramer®, demonstrating their specificity for the PGPGRVAKI (SEQ ID NO: 32985) peptide (FIG. 2). To test the functional activity of the murine ALK TCR-T cells, the release of IFN-γ (FIG. 3) and IL2 (FIG. 4) was measured following incubation of ALK TCR-T cells with 1) BALB/c splenocytes loaded with increasing concentrations of the ALK peptide PGPGRVAKI (SEQ ID NO: 32985) and 2) H2-Dd murine lung cancer cells in which the human EML4-ALK translocation was introduced with a retroviral infection (FIGS. 5 and 6). The results demonstrated that these five ALK-specific TCR-T cells are functional, as evidenced by the production of IFN-γ and IL-2.

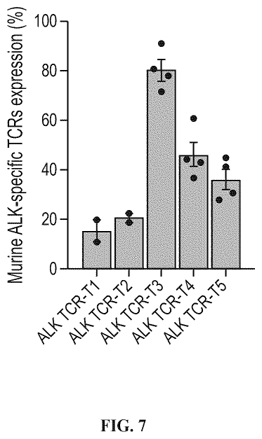

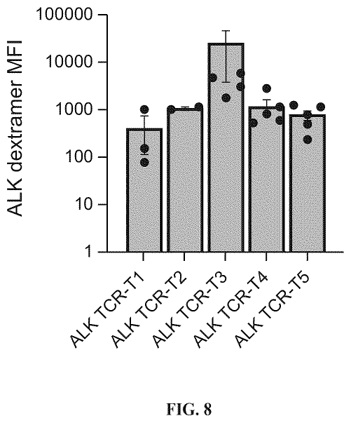

The killing activity of the murine ALK TCR-T cells was investigated in vitro by transducing primary CD8⁺ T cells with the murine ALK specific constructs. Untouched CD8⁺ T cells were purified from BALB/c splenocytes and activated in vitro for 24 h with 30 U/ml IL2 and anti-CD3/CD28 beads (T cell:beads ratio 1:1) before retroviral infection. The expression of the ALK-specific TCRs on the surface of the primary murine CD8⁺ T cells was analyzed two days post-infection by evaluating the expression of the green fluorescence protein expression (GFP) and the binding to the PGPGRVAKI (SEQ ID NO: 32985) Dextramer®). The level of expression of the ALK-specific TCRs on primary murine CD8⁺ T cells was construct-dependent with levels ranging from 15% to 80% (FIGS. 7 and 8); this difference reflected a variable level of mispairing of each ALK-specific TCR with the endogenous TCR repertoire.

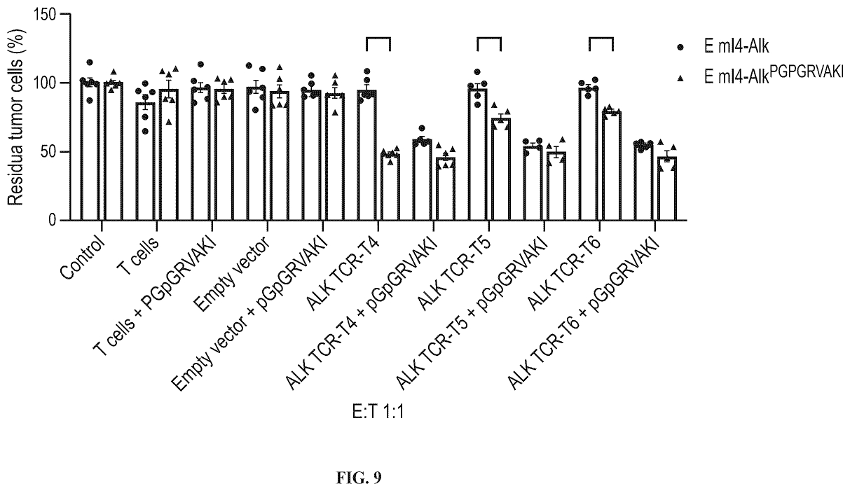

The ALK TCR-T3, ALK TCR-T4, and ALK TCR-T5 cells were co-cultured with luciferase-positive (FFLUC*) H2-Dd murine ALK-positive lung tumor cells and the residual bioluminescence of the tumor cells was measured three days post co-culture. ALK TCR-T3 killed more than 50% of H2-Dd ALK-positive lung tumor cells expressing the ALK peptide PGPGRVAKI (SEQ ID NO: 32985). No killing of H2-Dd positive and ALK-positive tumor cells that did not express the ALK peptide PGPGRVAKI (SEQ ID NO: 32985) was observed. Both ALK TCR-T4 and ALK TCR-T5 killed 30% of ALK-positive tumor cells expressing the PGPGRVAKI (SEQ ID NO: 32985), peptide (FIG. 9). The cloned sequences of the ALK-specific TCRs that recognize mouse ALK peptides presented by mouse MHC-I complex (BALB/c) are listed in Table 1.

TABLE 1
Sequences for ALK-specific TCRs that recognize mouse ALK peptides

| # | chain | fwr1 | cdr1 | fwr2 | cdr2 | fwr3 | cdr3 | fwr4 | Freqeuncy | proportion |
|---|---|---|---|---|---|---|---|---|---|---|
| Clonotype_1 | TRB | ETAVF | LGHDT | MYWYK | YNNKQ | IVNET | CASSQ | GPGTR | 826 | 0.16375892 |
| | | QTPNY | (SEQ | QDSKK | L | VPRRF | DWDEQ | LTVL | | |
| | | HVTQV | ID | LLKIM | (SEQ | SPQSS | YF | (SEQ | | |
| | | GNEVS | NO: | FS | ID | DKAHL | (SEQ | ID | | |
| | | FNCKQ | 3067) | (SEQ | NO: | NLRIK | ID | NO: | | |
| | | T | | ID | 9003) | SVEPE | NO: | 16327) | | |
| | | (SEQ | | NO: | | DSAVY | 32989) | | | |
| | | ID | | 6035) | | L | | | | |
| | | NO: | | | | (SEQ | | | | |
| | | 99) | | | | ID | | | | |
| | | | | | | NO: | | | | |
| | | | | | | 11971) | | | | |
| Clonotype_1 | TRB | GAGAC | CTGGG | ATGTA | TACAA | ATTGT | TGTGC | GGTCC | | |
| Nucleotide | | GGCTG | CCACG | TTGGT | TAATA | AAACG | CAGCA | CGGCA | | |
| sequence | | TTTTC | ATACT | ACAAG | AGCAA | AAACA | GCCAG | CCAGG | | |
| | | CAGAC | (SEQ | CAAGA | CTC | GTTCC | GACTG | CTCAC | | |
| | | TCCAA | ID | CTCTA | (SEQ | AAGGC | GGATG | GGTTT | | |
| | | ACTAT | NO: | AGAAA | ID | GCTTC | AACAG | TAG | | |
| | | CATGT | 4551) | TTGCT | NO: | TCACC | TACTT | (SEQ | | |
| | | CACAC | | GAAGA | 10487) | TCAGT | C | ID | | |
| | | AGGTG | | TTATG | | CTTCA | (SEQ | NO: | | |
| | | GGAAA | | TTTAG | | GATAA | ID | 17811) | | |
| | | TGAAG | | C | | AGCTC | NO: | | | |
| | | TGTCT | | (SEQ | | ATTTG | 32990) | | | |
| | | TTCAA | | ID | | AATCT | | | | |
| | | TTGTA | | NO: | | TCGAA | | | | |
| | | AGCAA | | 7519) | | TCAAG | | | | |
| | | ACT | | | | TCTGT | | | | |
| | | (SEQ | | | | AGAGC | | | | |
| | | ID | | | | CGGAG | | | | |
| | | NO: | | | | GACTC | | | | |
| | | 1583) | | | | TGCTG | | | | |
| | | | | | | TGTAT | | | | |
| | | | | | | CTC | | | | |
| | | | | | | (SEQ | | | | |
| | | | | | | ID | | | | |
| | | | | | | NO: | | | | |
| | | | | | | 13455) | | | | |
| Clonotype_1 | TRA | QQKVQ | DRNFQ | FWWYR | IFSDG | KKEGR | CAASE | GDGTQ | | |
| | | QSPES | Y | QHSGE | D | FTAHL | HGNNR | LVVKP | | |
| | | LSVPE | (SEQ | GPKAL | (SEQ | NKASL | IFF | (SEQ | | |
| | | GGMAS | ID | MS | ID | HVSLH | (SEQ | ID | | |
| | | FNCTS | NO: | (SEQ | NO: | IRDSQ | ID | NO: | | |
| | | S | 2970) | ID | 8906) | PSDSA | NO: | 16328) | | |
| | | (SEQ | | NO: | | LYF | 32991) | | | |
| | | ID | | 5938) | | (SEQ | | | | |
| | | NO: | | | | ID | | | | |
| | | 2) | | | | NO: | | | | |
| | | | | | | 11874) | | | | |
| Clonotype_1 | TRA | CAGCA | GATCG | TTCTG | ATCTT | AAGAA | TGTGC | GGTGA | | |
| Nucleotide | | GAAGG | TAATT | GTGGT | CTCTG | AGAAG | AGCTA | TGGGA | | |
| sequence | | TGCAG | TTCAG | ACAGA | ATGGT | GCAGA | GTGAG | CGCAG | | |
| | | CAGAG | TAC | CAGCA | GAC | TTCAC | CATGG | CTGGT | | |
| | | CCCAG | (SEQ | TTCTG | (SEQ | AGCTC | GAATA | GGTGA | | |
| | | AATCC | ID | GAGAA | ID | ACCTC | ACAGA | AGCCC | | |
| | | CTCAG | NO: | GGCCC | NO: | AATAA | ATCTT | A | | |
| | | TGTCC | 4454) | CAAGG | 10390) | GGCCA | CTTT | (SEQ | | |
| | | CAGAG | | CACTG | | GCCTG | (SEQ | ID | | |
| | | GGAGG | | ATGTC | | CATGT | ID | NO: | | |
| | | CATGG | | A | | TTCCC | NO: | 17812) | | |
| | | CCTCT | | (SEQ | | TGCAC | 32992) | | | |
| | | TTCAA | | ID | | ATCAG | | | | |
| | | CTGCA | | NO: | | AGACT | | | | |
| | | CTTCA | | 7422) | | CCCAG | | | | |
| | | AGT | | | | CCCAG | | | | |
| | | (SEQ | | | | TGACT | | | | |
| | | ID | | | | CCGCT | | | | |
| | | NO: | | | | CTCTA | | | | |
| | | 1486) | | | | CTTC | | | | |
| | | | | | | (SEQ | | | | |
| | | | | | | ID | | | | |
| | | | | | | NO: | | | | |
| | | | | | | 13358) | | | | |
| Clonotype_2 | TRB | DMKVT | MSHET | MYWYR | SYDVD | NSEGD | CASSL | GPGTR | 485 | 0.09615385 |
| | | QMPRY | (SEQ | QDPGL | S | IPKGY | GDSPD | LLVL | | |
| | | LIKRM | ID | GLQLI | (SEQ | RVSRK | TQYF | (SEQ | | |
| | | GENVL | NO: | YI | ID | KREHF | (SEQ | ID | | |
| | | LECGQ | 3131) | (SEQ | NO: | SLILD | ID | NO: | | |
| | | D | | ID | 9067) | SAKTN | NO: | 16341) | | |
| | | (SEQ | | NO: | | OTSVY | 32993) | | | |
| | | ID | | 6099) | | F | | | | |
| | | NO: | | | | (SEQ | | | | |
| | | 163) | | | | ID | | | | |
| | | | | | | NO: | | | | |
| | | | | | | 12035) | | | | |
| Clonotype_2 | TRB | GACAT | ATGAG | ATGTA | TCATA | AACAG | TGTGC | GGGCC | | |
| Nucleotide | | GAAAG | CCATG | CTGGT | CGATG | CGAAG | TAGCA | AGGCA | | |
| sequence | | TAACC | AAACA | ATCGA | TTGAT | GAGAC | GTTTA | CTCGG | | |
| | | CAGAT | (SEQ | CAAGA | AGT | ATCCC | GGGGA | CTCCT | | |
| | | GCCAA | ID | CCCTG | (SEQ | TAAAG | CAGCC | CGTGT | | |
| | | GATAC | NO: | GTCTG | ID | GATAC | CTGAC | TAG | | |
| | | CTGAT | 4615) | GGGCT | NO: | AGGGT | ACCCA | (SEQ | | |
| | | CAAAA | | ACAGC | 10551) | CTCAC | GTACT | ID | | |
| | | GAATG | | TGATT | | GGAAG | TT | NO: | | |
| | | GGAGA | | TATAT | | AAGCG | (SEQ | 17825) | | |
| | | GAATG | | C | | GGAGC | ID | | | |
| | | TTTTG | | (SEQ | | ATTTC | NO: | | | |
| | | CTGGA | | ID | | TCCCT | 32994) | | | |
| | | ATGTG | | NO: | | GATTC | | | | |
| | | GACAG | | 7583) | | TGGAT | | | | |
| | | GAC | | | | TCTGC | | | | |
| | | (SEQ | | | | TAAAA | | | | |
| | | ID | | | | CAAAC | | | | |
| | | NO: | | | | CAGAC | | | | |
| | | 1647) | | | | ATCTG | | | | |
| | | | | | | TGTAC | | | | |
| | | | | | | TTC | | | | |
| | | | | | | (SEQ | | | | |
| | | | | | | ID | | | | |
| | | | | | | NO: | | | | |
| | | | | | | 13519) | | | | |
| Clonotype_2 | TRA | QQKVQ | DRNFQ | FWWYR | IFSDG | KKEGR | CAASE | GQGTS | | |
| | | QSPES | Y | QHSGE | D | FTAHL | HGASS | LSVVP | | |
| | | LSVPE | (SEQ | GPKAL | (SEQ | NKASL | SFSKL | (SEQ | | |
| | | GGMAS | ID | MS | ID | HVSLH | VF | ID | | |
| | | LNCTS | NO: | (SEQ | NO: | IRDSQ | (SEQ | NO: | | |
| | | S | 2970) | ID | 8906) | PSDSA | ID | 16422) | | |
| | | (SEQ | | NO: | | LYF | NO: | | | |
| | | ID | | 5938) | | (SEQ | 32995) | | | |
| | | NO: | | | | ID | | | | |
| | | 64) | | | | NO: | | | | |
| | | | | | | 11874) | | | | |
| Clonotype_2 | TRA | CAGCA | GATCG | TTCTG | ATCTT | AAGAA | TGTGC | GGGCA | | |
| Nucleotide | | GAAGG | TAATT | GTGGT | CTCTG | AGAAG | AGCTA | GGGGA | | |
| sequence | | TGCAG | TTCAG | ACAGA | ATGGT | GCAGA | GTGAG | CATCC | | |
| | | CAGAG | TAC | CAGCA | GAC | TTCAC | CATGG | TTATC | | |
| | | CCCAG | (SEQ | TTCTG | (SEQ | AGCTC | GGCAT | AGTCG | | |
| | | AATCC | ID | GAGAA | ID | ACCTC | CCTCC | TTCCA | | |
| | | CTCAG | NO: | GGCCC | NO: | AATAA | TCCTT | A | | |
| | | TGTCC | 4454) | CAAGG | 10390) | GGCCA | CAGCA | (SEQ | | |
| | | CAGAG | | CACTG | | GCCTG | AGCTG | ID | | |
| | | GGAGG | | ATGTC | | CATGT | GTGTT | NO: | | |
| | | CATGG | | C | | TTCCC | T | 17906) | | |
| | | CCTCT | | (SEQ | | TGCAC | (SEQ | | | |
| | | CTCAA | | ID | | ATCAG | ID | | | |
| | | CTGCA | | NO: | | AGACT | NO: | | | |
| | | CTTCA | | 7944) | | CCCAG | 32996) | | | |
| | | AGT | | | | CCCAG | | | | |
| | | (SEQ | | | | TGACT | | | | |
| | | ID | | | | CCGCT | | | | |
| | | NO: | | | | CTCTA | | | | |
| | | 1548) | | | | CTTC | | | | |
| | | | | | | (SEQ | | | | |
| | | | | | | ID | | | | |
| | | | | | | NO: | | | | |
| | | | | | | 13358) | | | | |
| Clonotype_3 | TRB | EAAVT | NNHDY | MYWYR | SYVAD | TEKGD | CASSE | GPGTR | 263 | 0.05214116 |
| | | QSPRS | (SEQ | QDTGH | S | IPDGY | GSSYE | LTVL | | |
| | | KVAVT | ID | GLRLI | (SEQ | KASRP | QYF | (SEQ | | |
| | | GGKVT | NO: | HY | ID | SQENF | (SEQ | ID | | |
| | | LSCHO | 2975) | (SEQ | NO: | SLILE | ID | NO: | | |
| | | T | | ID | 8911) | LASLS | NO: | 16327) | | |
| | | (SEQ | | NO: | | QTAVY | 32997) | | | |
| | | ID | | 5939) | | F | | | | |
| | | NO: | | | | (SEQ | | | | |
| | | 7) | | | | ID | | | | |
| | | | | | | NO: | | | | |
| | | | | | | 11879) | | | | |
| Clonotype_3 | TRB | GAGGC | AATAA | ATGTA | TCATA | ACGGA | TGTGC | GGTCC | | |
| Nucleotide | | TGCAG | CCATG | CTGGT | TGTCG | GAAAG | CAGCA | CGGCA | | |
| sequence | | TCACC | ACTAT | ATCGG | CTGAC | GAGAT | GTGAG | CCAGG | | |
| | | CAAAG | (SEQ | CAGGA | AGC | ATCCC | GGGAG | CTCAC | | |
| | | TCCAA | ID | CACGG | (SEQ | TGATG | CTCCT | GGTTT | | |
| | | GAAGC | NO: | GGCAT | ID | GGTAC | ATGAA | TAG | | |
| | | AAGGT | 4459) | GGGCT | NO: | AAGGC | CAGTA | (SEQ | | |
| | | GGCAG | | GAGGC | 10395) | CTCCA | CTTC | ID | | |
| | | TAACA | | TGATC | | GACCA | (SEQ | NO: | | |
| | | GGAGG | | CATTA | | AGCCA | ID | 17811) | | |
| | | AAAGG | | C | | AGAGA | NO: | | | |
| | | TGACA | | (SEQ | | ATTTC | 32998) | | | |
| | | TTGAG | | ID | | TCTCT | | | | |
| | | CTGTC | | NO: | | CATTC | | | | |
| | | ACCAG | | 7427) | | TGGAG | | | | |
| | | ACT | | | | TTGGC | | | | |
| | | (SEQ | | | | TTCCC | | | | |
| | | ID | | | | TTTCT | | | | |
| | | NO: | | | | CAGAC | | | | |
| | | 1491) | | | | AGCTG | | | | |
| | | | | | | TATAT | | | | |
| | | | | | | TTC | | | | |
| | | | | | | (SEQ | | | | |
| | | | | | | ID | | | | |
| | | | | | | NO: | | | | |
| | | | | | | 13363) | | | | |
| Clonotype_3 | TRA | QQKVQ | DRNFQ | FWWYR | IFSDG | KKEGR | CAASE | GTGTK | | |
| | | QSPES | Y | QHSGE | D | FTAHL | HGNSG | LQVVP | | |
| | | LSVPE | (SEQ | GPKAL | (SEQ | NKASL | TYQRF | (SEQ | | |
| | | GGMAS | ID | MS | ID | HVSLH | (SEQ | ID | | |
| | | FNCTS | NO: | (SEQ | NO: | IRDSQ | ID | NO: | | |
| | | S | 2970) | ID | 8906) | PSDSA | NO: | 16432) | | |
| | | (SEQ | | NO: | | LYF | 32999) | | | |
| | | ID | | 5938) | | (SEQ | | | | |
| | | NO: | | | | ID | | | | |
| | | 2) | | | | NO: | | | | |
| | | | | | | 11874) | | | | |
| Clonotype_3 | TRA | CAGCA | GATCG | TTCTG | ATCTT | AAGAA | TGTGC | GGAAC | | |
| Nucleotide | | GAAGG | TAATT | GTGGT | CTCTG | AGAAG | AGCTA | TGGGA | | |
| sequence | | TGCAG | TTCAG | ACAGA | ATGGT | GCAGA | GTGAG | CAAAA | | |
| | | CAGAG | TAC | CAGCA | GAC | TTCAC | CATGG | CTCCA | | |
| | | CCCAG | (SEQ | TTCTG | (SEQ | AGCTC | AAATT | AGTCG | | |
| | | AATCC | ID | GAGAA | ID | ACCTC | CTGGG | TTCCA | | |
| | | CTCAG | NO: | GGCCC | NO: | AATAA | ACTTA | A | | |
| | | TGTCC | 4454) | CAAGG | 10390) | GGCCA | CCAGA | (SEQ | | |
| | | CAGAG | | CACTG | | GCCTG | GGTTT | ID | | |
| | | GGAGG | | ATGTC | | CATGT | (SEQ | NO: | | |
| | | CATGG | | A | | TTCCC | ID | 17916) | | |
| | | CCTCT | | (SEQ | | TGCAC | NO: | | | |
| | | TTCAA | | ID | | ATCAG | 33000) | | | |
| | | CTGCA | | NO: | | AGACT | | | | |
| | | CTTCA | | 7422) | | CCCAG | | | | |
| | | AGT | | | | CCCAG | | | | |
| | | (SEQ | | | | TGACT | | | | |
| | | ID | | | | CCGCT | | | | |
| | | NO: | | | | CTCTA | | | | |
| | | 1486) | | | | CTTC | | | | |
| | | | | | | (SEQ | | | | |
| | | | | | | ID | | | | |
| | | | | | | NO: | | | | |
| | | | | | | 13358) | | | | |
| Clonotype_4 | TRB | DAGVT | SGHND | LFWYR | FRSKS | MEDGG | CASSF | GPGTR | 183 | 0.03628073 |
| | | QTPRH | (SEQ | QTKIQ | L | AFKDR | SDWGD | LTVL | | |
| | | EVAEK | ID | GLELL | (SEQ | FKAEM | EQYF | (SEQ | | |
| | | GQTII | NO: | SY | ID | LNSSF | (SEQ | ID | | |
| | | LKCEP | 4207) | (SEQ | NO: | STLKI | ID | NO: | | |
| | | V | | ID | 10143) | QPTEP | NO: | 16327) | | |
| | | (SEQ | | NO: | | KDSAV | 33001) | | | |
| | | ID | | 7175) | | YL | | | | |
| | | NO: | | | | (SEQ | | | | |
| | | 1239) | | | | ID | | | | |
| | | | | | | NO: | | | | |
| | | | | | | 13111) | | | | |
| Clonotype_4 | TRB | GATGC | TCAGG | CTTTT | TTCCG | ATGGA | TGTGC | GGTCC | | |
| Nucleotide | | TGGAG | CCACA | CTGGT | CAGCA | AGATG | CAGCA | CGGCA | | |
| sequence | | TTACC | ATGAC | ACAGA | AGTCT | GTGGG | GTTTC | CCAGG | | |
| | | CAGAC | (SEQ | CAGAC | CTT | GCTTT | TCGGA | CTCAC | | |
| | | ACCCA | ID | CAAGA | (SEQ | CAAGG | CTGGG | GGTTT | | |
| | | GACAT | NO: | TACAG | ID | ATCGA | GGGAT | TAG | | |
| | | GAGGT | 5691) | GGACT | NO: | TTCAA | GAACA | (SEQ | | |
| | | GGCAG | | AGAGT | 11627) | AGCTG | GTACT | ID | | |
| | | AGAAA | | TGCTG | | AGATG | TC | NO: | | |
| | | GGACA | | AGCTA | | CTAAA | (SEQ | 17811) | | |
| | | AACAA | | C | | TTCAT | ID | | | |
| | | TAATC | | (SEQ | | CCTTC | NO: | | | |
| | | CTGAA | | ID | | TCCAC | 33002) | | | |
| | | GTGTG | | NO: | | TCTGA | | | | |
| | | AGCCA | | 8659) | | AGATT | | | | |
| | | GTT | | | | CAACC | | | | |
| | | (SEQ | | | | TACAG | | | | |
| | | ID | | | | AACCC | | | | |
| | | NO: | | | | AAGGA | | | | |
| | | 2723) | | | | CTCAG | | | | |
| | | | | | | CTGTG | | | | |
| | | | | | | TATCT | | | | |
| | | | | | | G | | | | |
| | | | | | | (SEQ | | | | |
| | | | | | | ID | | | | |
| | | | | | | NO: | | | | |
| | | | | | | 14595) | | | | |
| Clonotype_4 | TRA | QQKVQ | DRNFQ | FWWYR | IFSDG | KKEGR | CAASE | GKGTQ | | |
| | | QSPES | Y | QHSGE | D | FTAHL | HGNNR | VVVLP | | |
| | | LSVPE | (SEQ | GPKAL | (SEQ | NKASL | LTL | (SEQ | | |
| | | GGMAS | ID | MS | ID | HVSLH | (SEQ | ID | | |
| | | LNCTS | NO: | (SEQ | NO: | IRDSQ | ID | NO: | | |
| | | S | 2970) | ID | 8906) | PSDSA | NO: | 16644) | | |
| | | (SEQ | | NO: | | LYF | 33003) | | | |
| | | ID | | 5938) | | (SEQ | | | | |
| | | NO: | | | | ID | | | | |
| | | 64) | | | | NO: | | | | |
| | | | | | | 11874) | | | | |
| Clonotype_4 | TRA | CAGCA | GATCG | TTCTG | ATCTT | AAGAA | TGTGC | GGGAA | | |
| Nucleotide | | GAAGG | TAATT | GTGGT | CTCTG | AGAAG | AGCTA | GGGAA | | |
| sequence | | TGCAG | TTCAG | ACAGA | ATGGT | GCAGA | GTGAG | CCCAG | | |
| | | CAGAG | TAC | CAGCA | GAC | TTCAC | CACGG | GTGGT | | |
| | | CCCAG | (SEQ | TTCTG | (SEQ | AGCTC | CAACA | GGTGT | | |
| | | AATCC | ID | GAGAA | ID | ACCTC | ACAGA | TACCA | | |
| | | CTCAG | NO: | GGCCC | NO: | AATAA | CTTAC | A | | |
| | | TGTCC | 4454) | CAAGG | 10390) | GGCCA | TTTG | (SEQ | | |
| | | CAGAG | | CACTG | | GCCTG | (SEQ | ID | | |
| | | GGAGG | | ATGTC | | CATGT | ID | NO: | | |
| | | CATGG | | C | | TTCCC | NO: | 18128) | | |
| | | CCTCT | | (SEQ | | TGCAC | 33004) | | | |
| | | CTCAA | | ID | | ATCAG | | | | |
| | | CTGCA | | NO: | | AGACT | | | | |
| | | CTTCA | | 7944) | | CCCAG | | | | |
| | | AGT | | | | CCCAG | | | | |
| | | (SEQ | | | | TGACT | | | | |
| | | ID | | | | CCGCT | | | | |
| | | NO: | | | | CTCTA | | | | |
| | | 1548) | | | | CTTC | | | | |
| | | | | | | (SEQ | | | | |
| | | | | | | ID | | | | |
| | | | | | | NO: | | | | |
| | | | | | | 13358) | | | | |
| Clonotype_5 | TRB | GGIIT | FNHDT | MYWYR | SITEN | LQKGD | CASSI | GPGTR | 171 | 0.03390167 |
| | | QTPKF | (SEQ | QDSGK | D | LSEGY | SDWGD | LTVL | | |
| | | LIGQE | ID | GLRLI | (SEQ | DASRE | EQYF | (SEQ | | |
| | | GQKLT | NO: | YY | ID | KKSSF | (SEQ | ID | | |
| | | LKCQQ | 2995) | (SEQ | NO: | SLTVT | ID | NO: | | |
| | | N | | ID | 8931) | SAQKN | NO: | 16327) | | |
| | | (SEQ | | NO: | | EMAVF | 33005) | | | |
| | | ID | | 5963) | | L | | | | |
| | | NO: | | | | (SEQ | | | | |
| | | 27) | | | | ID | | | | |
| | | | | | | NO: | | | | |
| | | | | | | 11899) | | | | |
| Clonotype_5 | TRB | GGTGG | TTCAA | ATGTA | TCAAT | CTTCA | TGTGC | GGTCC | | |
| Nucleotide | | CATCA | TCATG | CTGGT | AACTG | AAAAG | CAGCA | CGGCA | | |
| sequence | | TTACT | ATACA | ACCGA | AAAAC | GCGAT | GTATA | CCAGG | | |
| | | CAGAC | (SEQ | CAGGA | GAT | CTATC | TCGGA | CTCAC | | |
| | | ACCCA | ID | TTCAG | (SEQ | TGAAG | CTGGG | GGTTT | | |
| | | AATTC | NO: | GGAAA | ID | GCTAT | GGGAT | TAG | | |
| | | CTGAT | 4479) | GGATT | NO: | GATGC | GAACA | (SEQ | | |
| | | TGGTC | | GAGAC | 10415) | GTCTC | GTACT | ID | | |
| | | AGGAA | | TGATC | | GAGAG | TC | NO: | | |
| | | GGGCA | | TACTA | | AAGAA | (SEQ | 17811) | | |
| | | AAAAC | | T | | GTCAT | ID | | | |
| | | TGACC | | (SEQ | | CTTTT | NO: | | | |
| | | TTGAA | | ID | | TCTCT | 33006) | | | |
| | | ATGTC | | NO: | | CACTG | | | | |
| | | AACAG | | 7447) | | TGACA | | | | |
| | | AAT | | | | TCTGC | | | | |
| | | (SEQ | | | | CCAGA | | | | |
| | | ID | | | | AGAAC | | | | |
| | | NO: | | | | GAGAT | | | | |
| | | 1511) | | | | GGCCG | | | | |
| | | | | | | TTTTT | | | | |
| | | | | | | CTC | | | | |
| | | | | | | (SEQ | | | | |
| | | | | | | ID | | | | |
| | | | | | | NO: | | | | |
| | | | | | | 13383) | | | | |
| Clonotype_5 | TRA | QQKVQ | DRNFQ | FWWYR | IFSDG | KKEGR | CAASE | GDGTQ | | |
| | | QSPES | Y | QHSGE | D | FTAHL | HGNNR | LVVKP | | |
| | | LSVPE | (SEQ | GPKAL | (SEQ | NKASL | IFF | (SEQ | | |
| | | GGMAS | ID | MS | ID | HVSLH | (SEQ | ID | | |
| | | FNCTS | NO: | (SEQ | NO: | IRDSQ | ID | NO: | | |
| | | S | 2970) | ID | 8906) | PSDSA | NO: | 16328) | | |
| | | (SEQ | | NO: | | LYF | 32991) | | | |
| | | ID | | 5938) | | (SEQ | | | | |
| | | NO: | | | | ID | | | | |
| | | 2) | | | | NO: | | | | |
| | | | | | | 11874) | | | | |
| Clonotype_5 | TRA | CAGCA | GATCG | TTCTG | ATCTT | AAGAA | TGTGC | GGTGA | | |
| Nucleotide | | GAAGG | TAATT | GTGGT | CTCTG | AGAAG | AGCTA | TGGGA | | |
| sequence | | TGCAG | TTCAG | ACAGA | ATGGT | GCAGA | GTGAG | CGCAG | | |
| | | CAGAG | TAC | CAGCA | GAC | TTCAC | CACGG | CTGGT | | |
| | | CCCAG | SEQ | TTCTG | SEQ | AGCTC | CAATA | GGTGA | | |
| | | AATCC | ID | GAGAA | ID | ACCTC | ACAGA | AGCCC | | |
| | | CTCAG | NO: | GGCCC | NO: | AATAA | ATCTT | A | | |
| | | TGTCC | 4454) | CAAGG | 10390) | GGCCA | CTTT | SEQ | | |
| | | CAGAG | | CACTG | | GCCTG | SEQ | ID | | |
| | | GGAGG | | ATGTC | | CATGT | ID | NO: | | |
| | | CATGG | | A | | TTCCC | NO: | 17812) | | |
| | | CCTCT | | SEQ | | TGCAC | 33007) | | | |
| | | TTCAA | | ID | | ATCAG | | | | |
| | | CTGCA | | NO: | | AGACT | | | | |
| | | CTTCA | | 7422) | | CCCAG | | | | |
| | | AGT | | | | CCCAG | | | | |
| | | SEQ | | | | TGACT | | | | |
| | | ID | | | | CCGCT | | | | |
| | | NO: | | | | CTCTA | | | | |
| | | 1486) | | | | CTTC | | | | |
| | | | | | | SEQ | | | | |
| | | | | | | ID | | | | |
| | | | | | | NO: | | | | |
| | | | | | | 13358) | | | | |

FWR=framework region; CDR=complementarity determining region

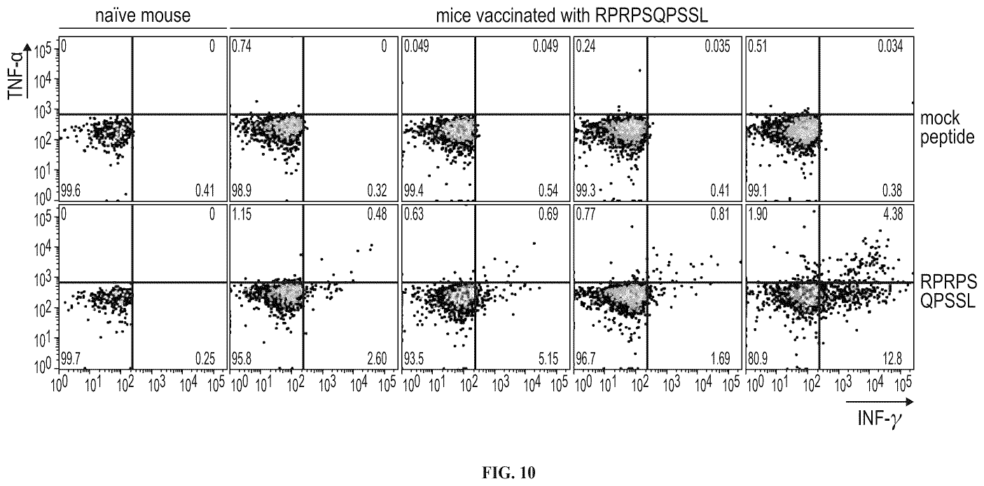

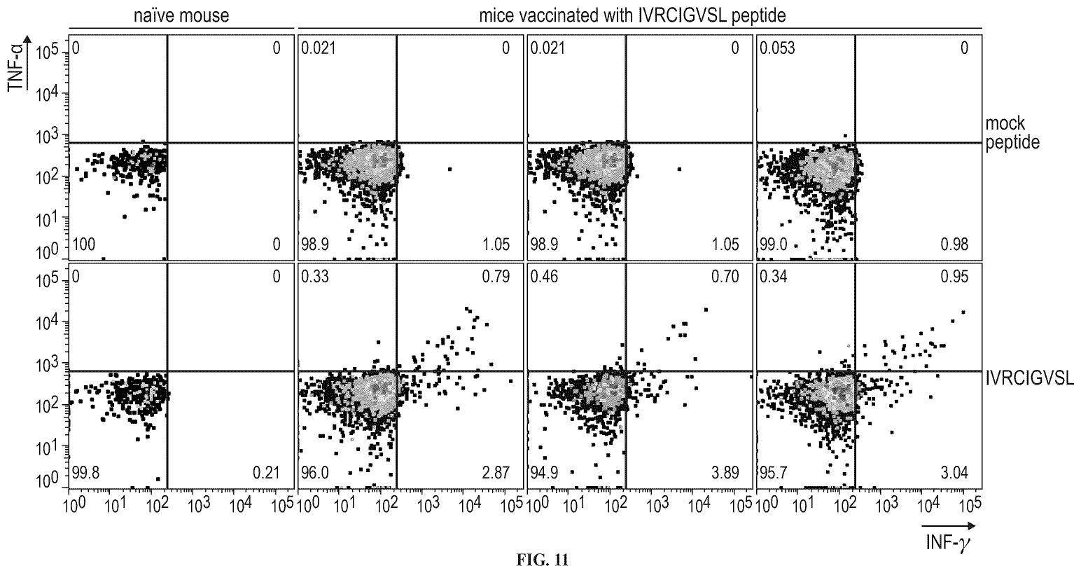

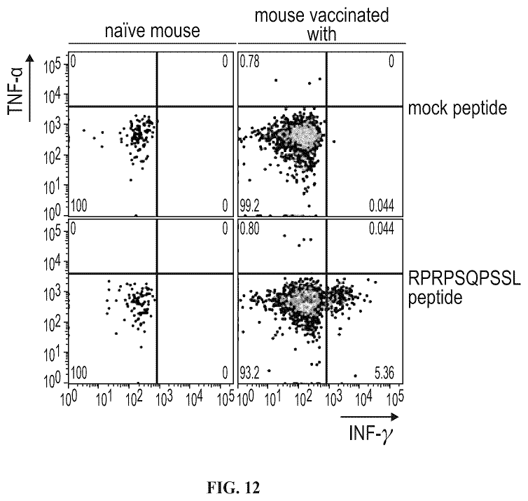

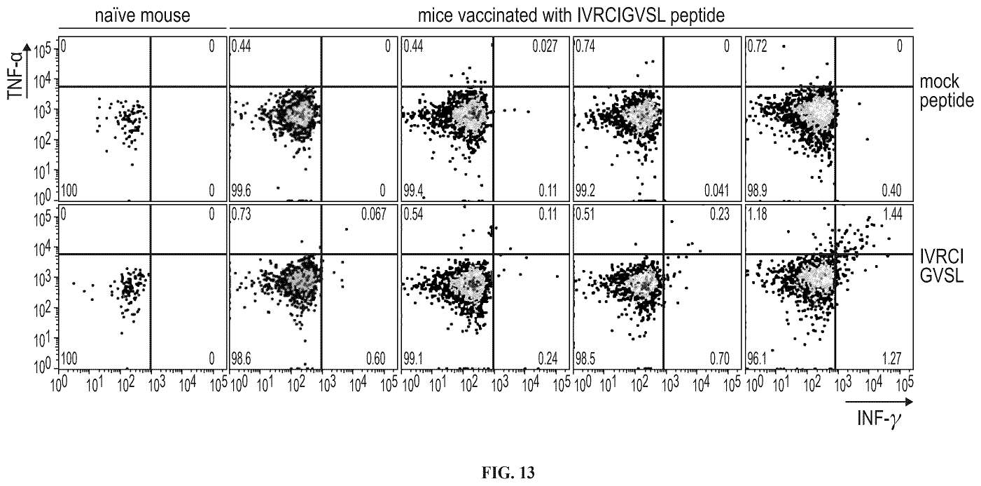

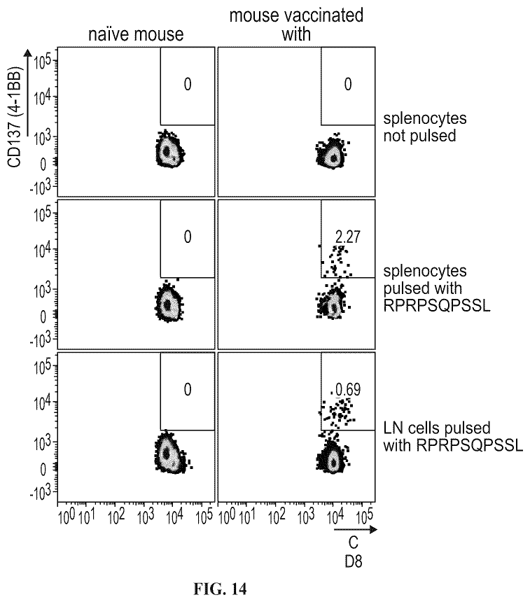

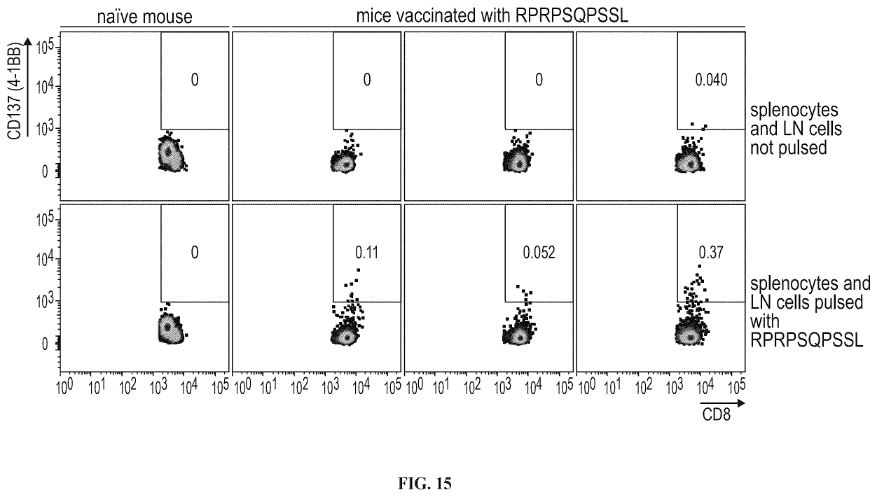

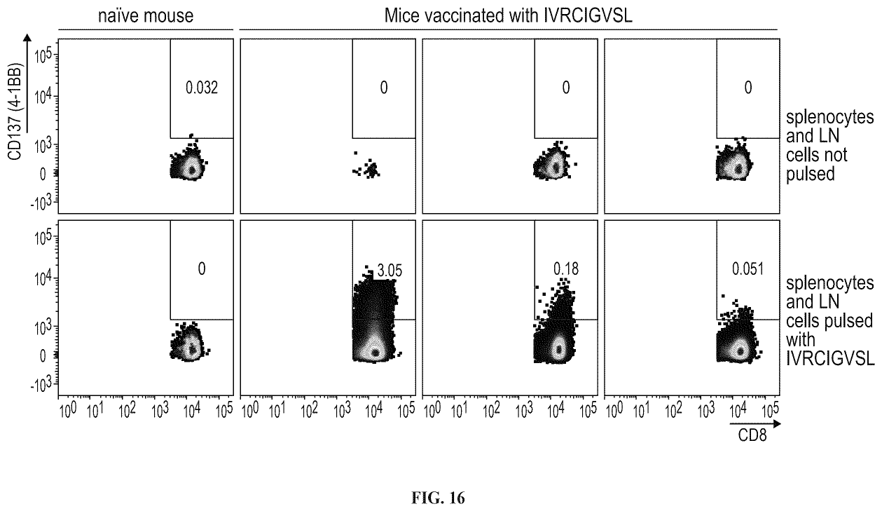

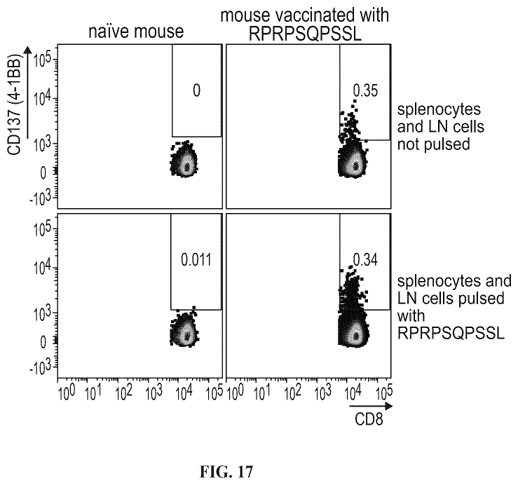

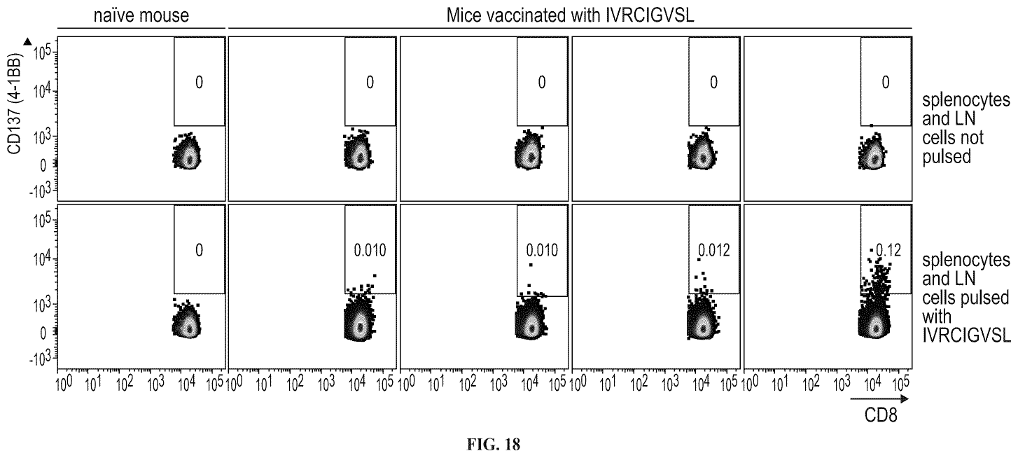

To isolate TCRs recognizing human ALK peptides, four cohorts of HLA-B*07:02 transgenic mice (CB6F1-B2mtm1Unc Tg(B2M)55Hpl Tg(HLA-B*0702/H2-Kb)B7.xx, Taconic®) were vaccinated with either RPRPSQPSSL (SEQ ID NO: 32957) or IVRCIGVSL (SEQ ID NO: 32958) peptides. Mice received two priming subcutis injections at the base of the tail of either RPRPSQPSSL (SEQ ID NO: 32957) or IVRCIGVSL (SEQ ID NO: 32958) peptide and adjuvant cyclic diguanylate monophosphate (c-di-GMP) at day 0 and day 14. Seven days after the first vaccination booster, intracellular staining (ICS) was performed from the peripheral blood of naïve mice and mice vaccinated with either RPRPSQPSSL (SEQ ID NO: 32957) or IVRCIGVSL (SEQ ID NO: 32958). The results showed that CD8 T cells of mice vaccinated with RPRPSQPSSL (SEQ ID NO: 32957) or IVRCIGVSL (SEQ ID NO: 32958) produced both IFN-γ and TNF-α (FIGS. 10-13). Seven days after the last vaccination booster, which occurred on day 56, splenocytes and inguinal lymph node cells of one naïve mouse and mice vaccinated with either RPRPSQPSSL (SEQ ID NO: 32957) or IVRCIGVSL (SEQ ID NO: 32958) were collected and stimulated overnight in vitro with either RPRPSQPSSL (SEQ ID NO: 32957) or IVRCIGVSL (SEQ ID NO: 32958) peptides, respectively. Next, untouched CD8⁺ T cells were purified and the expression of CD137 (4-1BB), a marker of T cell activation, was evaluated by flow cytometry. The results showed that CD137 (4-1BB) expression was detectable only in CD8⁺ T cells collected from mice vaccinated with RPRPSQPSS (SEQ ID NO: 32987) or IVRCIGVSL (SEQ ID NO: 32958) peptides that were also pulsed overnight in vitro with either RPRPSQPSS (SEQ ID NO: 32987) or IVRCIGVSL (SEQ ID NO: 32958) peptides (FIGS. 14-18).

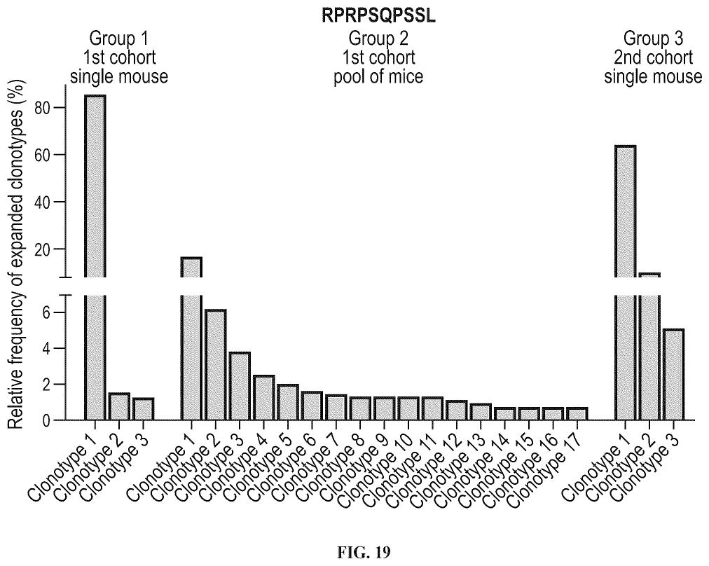

CD8⁺/CD137⁺ (4-1BB) T cells were sorted from the splenocytes, and inguinal lymph node cells of mice vaccinated with either RPRPSQPSSL (SEQ ID NO: 32957) or IVRCIGVSL (SEQ ID NO: 32958) peptides and subjected to single-cell sequencing (10× Genomics®). TCR pair-ended sequencing of 412 CD8/CD137 (4-1BB) T cells sorted from one mouse of the first cohort of HLA*B07:02 mice vaccinated with RPRPSQPSSL (SEQ ID NO: 32957), identified 48 unique clonotypes, whose relative frequency ranged from 85.44% to 0.24%, while 305 unique clonotypes whose relative frequency ranged from 16.61% to 0.18% were identified from 554 CD8⁺/CD137⁺ (4-1BB) sorted from three mice of the same cohort (FIG. 19). The cloned sequences of the ALK-specific TCRs that recognize human ALK peptides presented by HLA-B*07:02 human HLA from the one mouse of the first cohort of HLA*B07:02 mice vaccinated with RPRPSQPSSL (SEQ ID NO: 32957) are provided in SEQ ID NOs XXX. The cloned sequences of the ALK-specific TCRs that recognize human ALK peptides presented by HLA-B*07:02 human HLA from the three mice of the first cohort of HLA*B07:02 mice vaccinated with RPRPSQPSSL (SEQ ID NO: 32957) are provided in SEQ ID NOs XXX.

59 CD8⁺/CD137⁺ (4-1BB) T cells that were sorted from one mouse of the second cohort of HLA*B07:02 mice vaccinated with RPRPSQPSSL (SEQ ID NO: 32957) were also sequenced, resulting in 14 unique clonotypes, whose frequency ranged from 64.4% to 1.7% (FIG. 19). The cloned sequences of the ALK-specific TCRs that recognize human ALK peptides presented by HLA-B*07:02 human HLA from the one mouse of the second cohort of HLA*B07:02 mice vaccinated with RPRPSQPSSL (SEQ ID NO: 32957) are provided in SEQ ID NOs XXX.

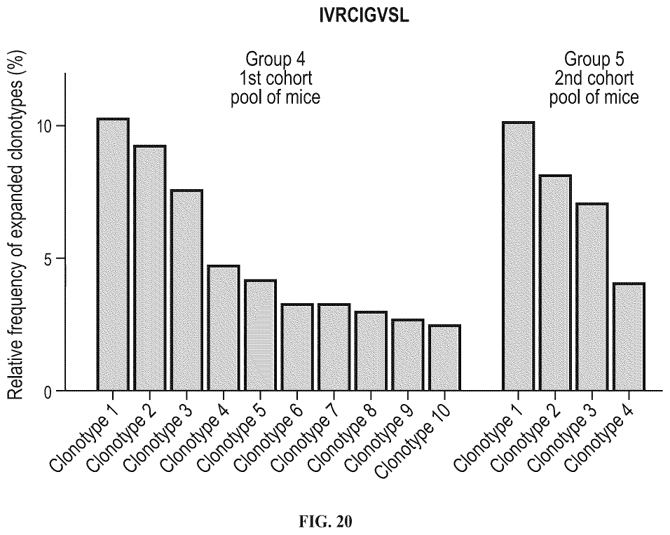

TCR pair-ended sequencing of 4398 CD8⁺/CD137⁺ (4-1BB) T cells sorted from the pool of three mice of the first cohort of HLA*B07:02 mice vaccinated with IVRCIGVSL (SEQ ID NO: 32958) identified 742 unique clonotypes, whose frequency ranged from 10.28% to 0.02%, while TCR pair-ended sequencing of 98 CD8⁺/CD137⁺ (4-1BB) T cells sorted from four mice of the second cohort of HLA*B07:02 mice vaccinated with IVRCIGVSL (SEQ ID NO: 32958) identified 68 unique clonotypes whose frequency ranged from 10.20% to 1.02% (FIG. 20). The cloned sequences of the ALK-specific TCRs that recognize human ALK peptides presented by HLA-B*0) 7:02 human HLA from the three mice of the first cohort of HLA*B07:02 mice vaccinated with IVRCIGVSL (SEQ ID NO: 32958) are provided in SEQ ID NOs XXX. The cloned sequences of the ALK-specific TCRs that recognize human ALK peptides presented by HLA-B*0) 7:02 human HLA from the four mice of the second cohort of HLA*B07:02 mice vaccinated with IVRCIGVSL (SEQ ID NO: 32958) are provided in SEQ ID NOs XXX.

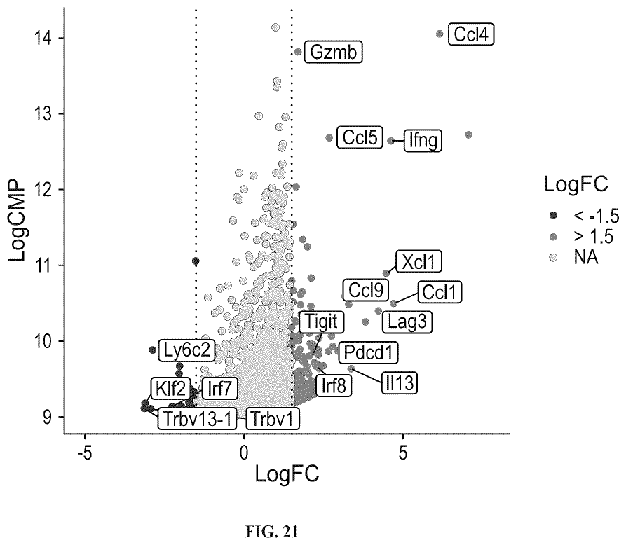

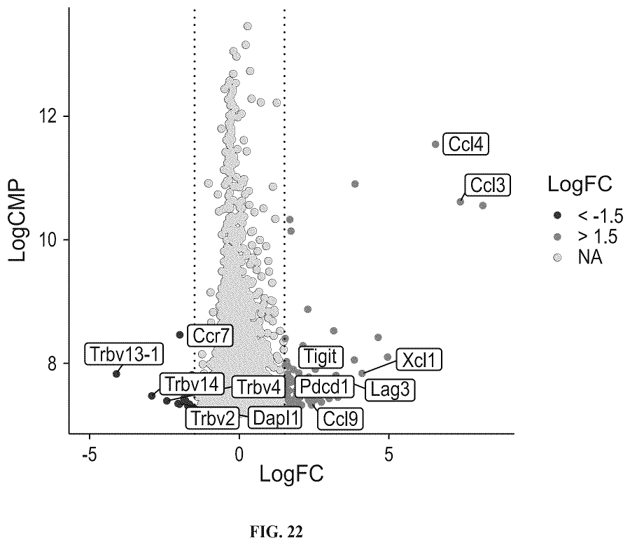

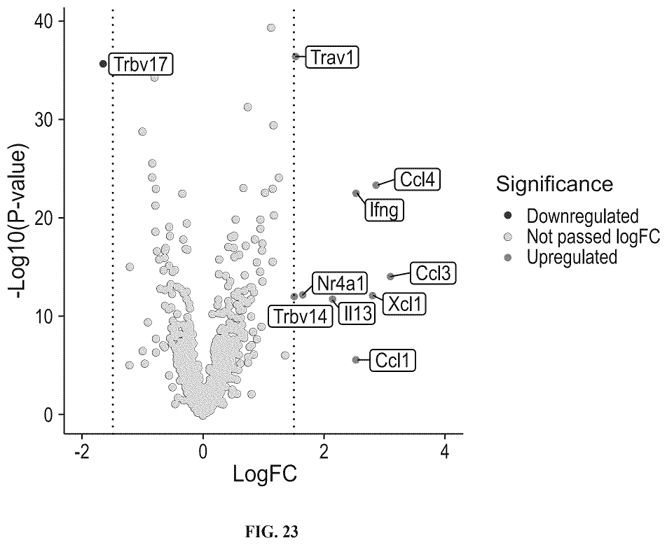

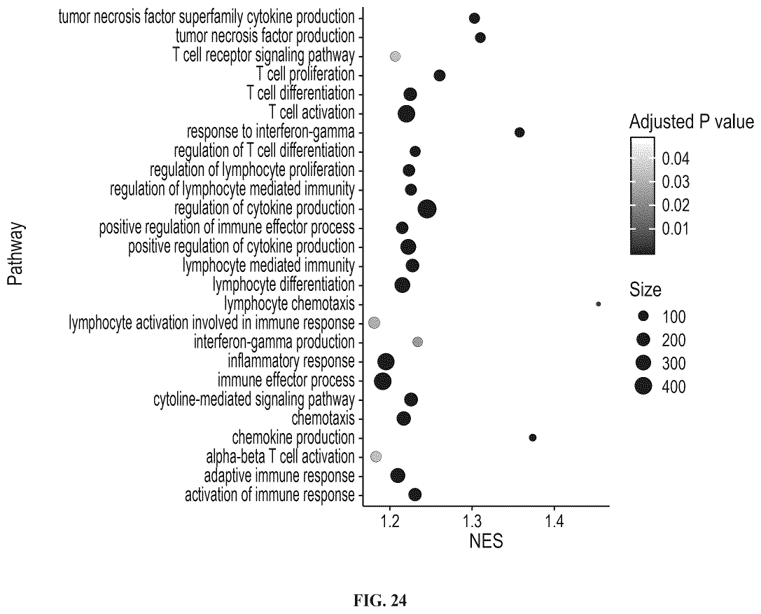

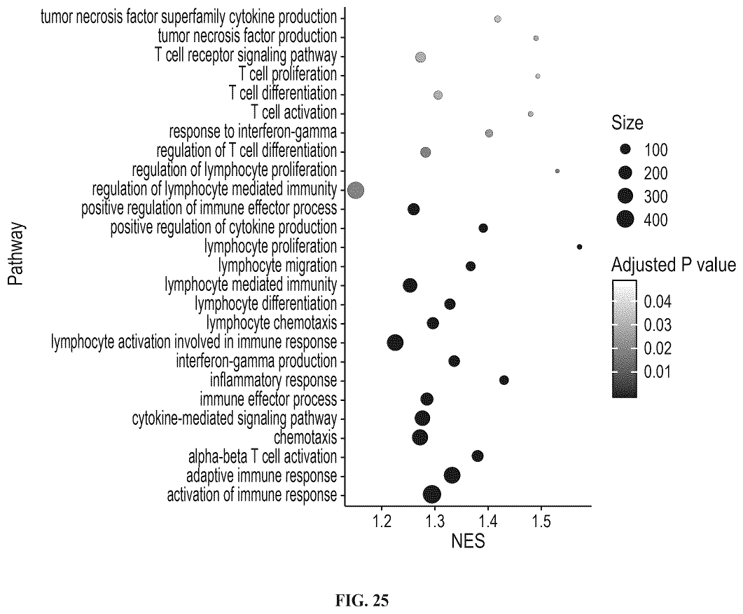

The transcriptomic profile of the expanded clonotypes (VDJ count≥4) was analyzed versus the not expanded clonotypes (VDJ count<4) identified in the first cohort of mice vaccinated with both RPRPSQPSSL (SEQ ID NO: 32957) and IVRCIGVSL (SEQ ID NO: 32958). It was found that the expanded clonotypes are characterized by a different gene expression profile compared with the not expanded clonotypes (FIGS. 21-23). Gene Set Enrichment Analysis was also performed between the expanded and the not expanded ALK clonotypes identified from the first cohort of HLA*B07:02 mice vaccinated with RPRPSQPSSL (SEQ ID NO: 32957). Significant upregulation of multiple pathways of the adaptive immune response was observed, including T-cell activation, IFN-γ signaling, IFN-γ production, cytokine release, and T-cell proliferation in the expanded clonotype group, suggesting functional activity of these clonotypes (FIGS. 24-25).

The paired α and β sequence of all the expanded ALK-specific clonotypes (VDJ count≥4) isolated from the first cohort of HLA*B07:02 mice vaccinated with RPRPSQPSSL (SEQ ID NO: 32957) was cloned into a retroviral vector. Two different retroviral vectors were generated for the three most expanded ALK clonotypes of group 1 and for the two most expanded clonotypes of group 2, one expressing the constant β chain 1 (Cβ1) of the murine TCR, and the other expressing the constant β chain 2 (Cβ2) of the murine TCR. All the other ALK expanded clonotypes were cloned with Cβ1 or Cβ2 based on the RNA sequencing data of these cells. Untouched CD3 T cells were purified from Peripheral Blood Mononuclear Cells of healthy donors (PMBCs) and were activated in vitro with anti-CD3 and anti-CD28 beads (ratio cells:beads cells 1:1). 10 ng/ml rhIL-7 and 5 ng/ml rhIL 15 for two days before retroviral transduction with the ALK TCR constructs (both Cβ1 and Cβ2). The transduction efficiency was evaluated three days post-retroviral infection by analyzing the expression of the GFP that was inserted as a reporter in the retroviral construct.

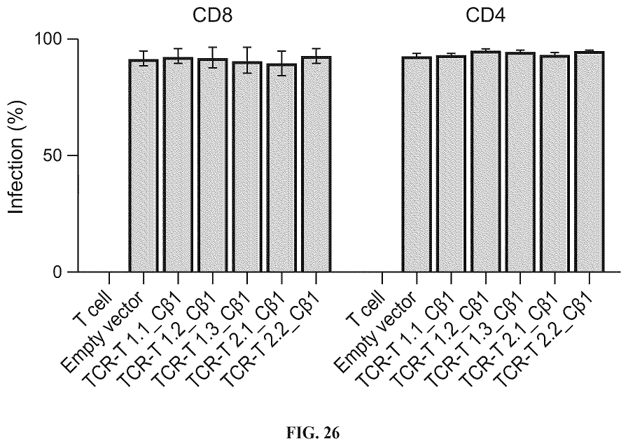

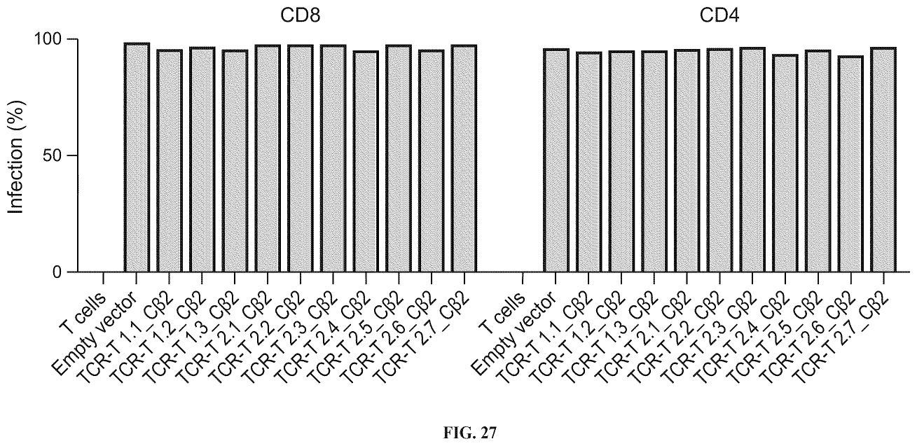

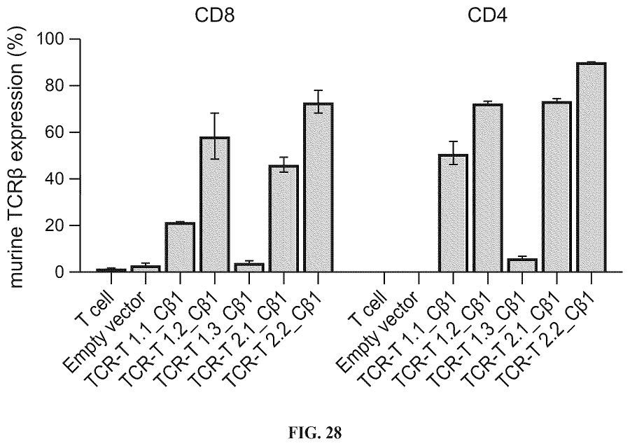

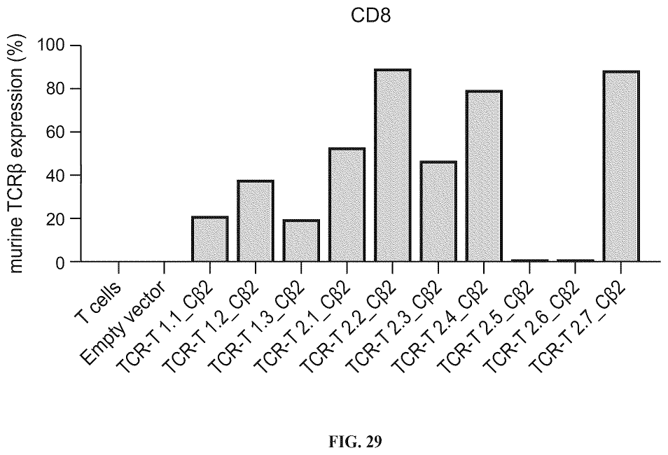

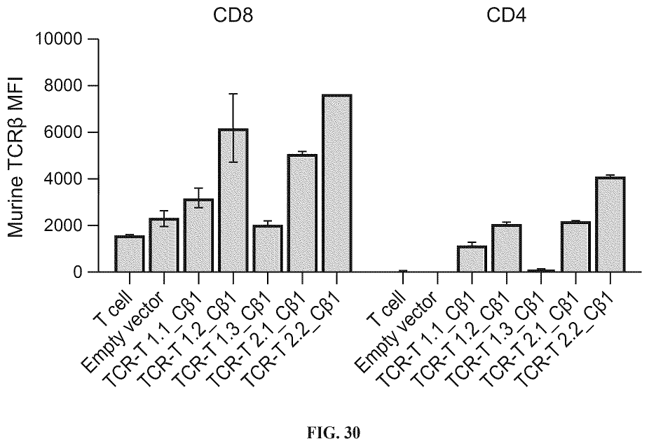

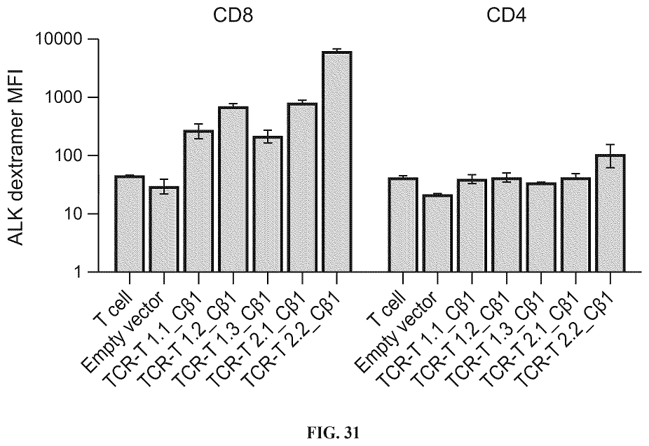

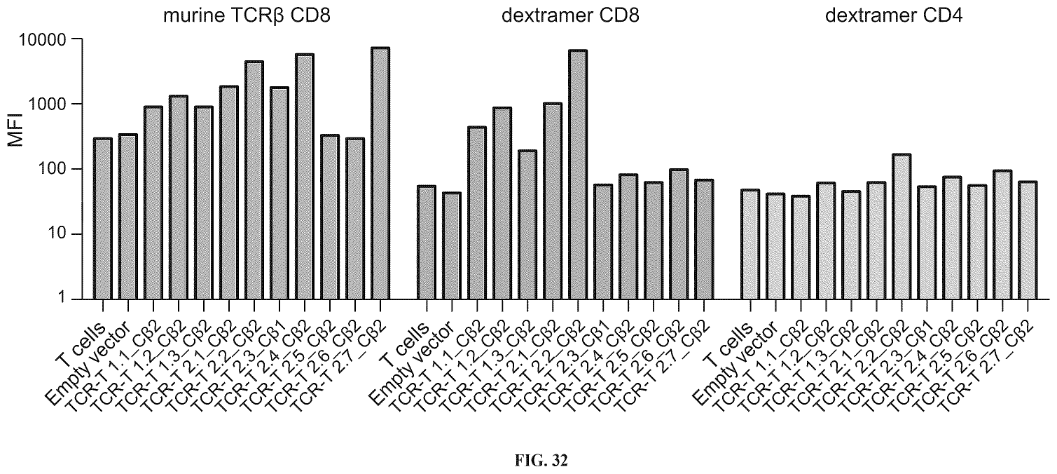

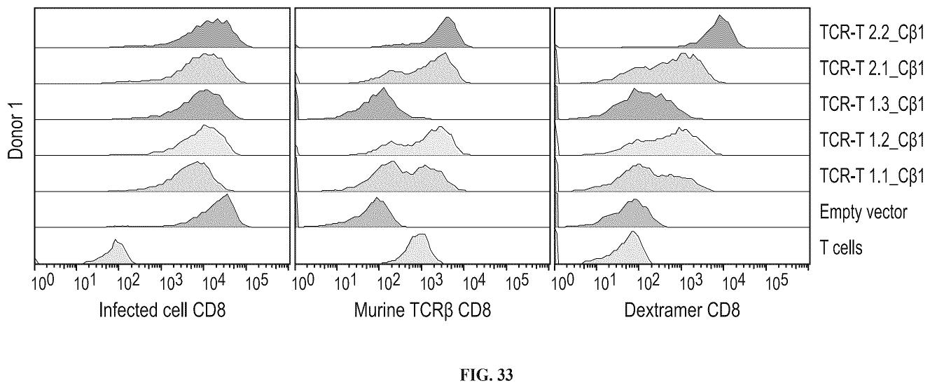

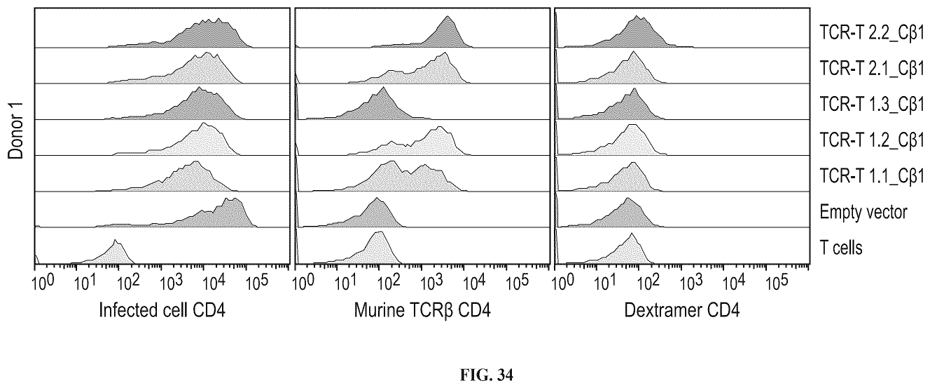

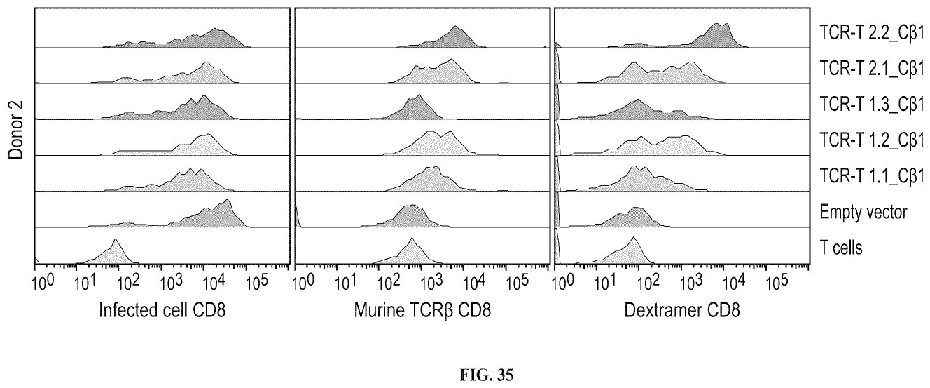

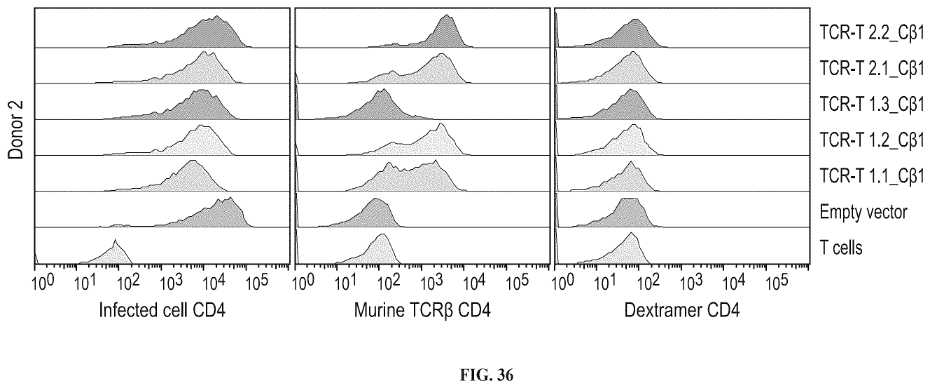

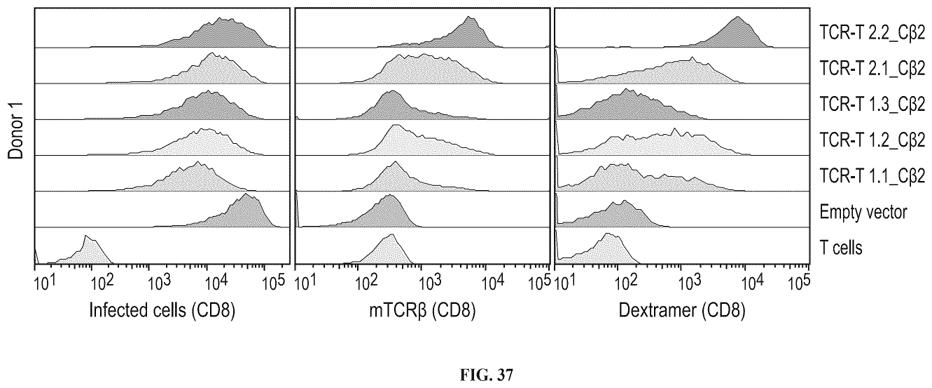

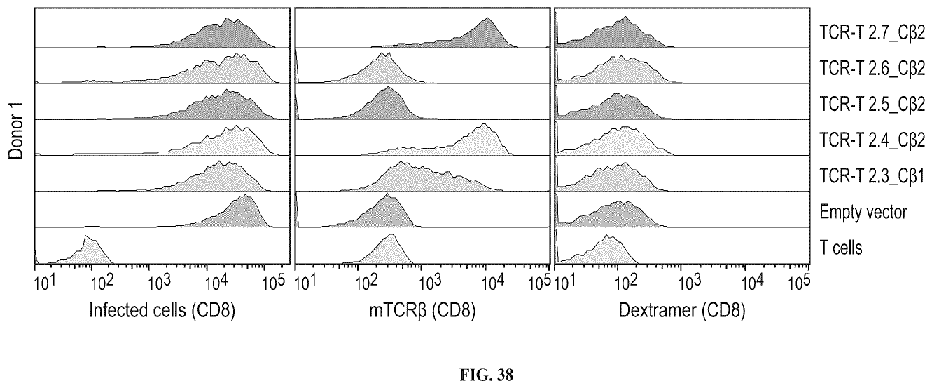

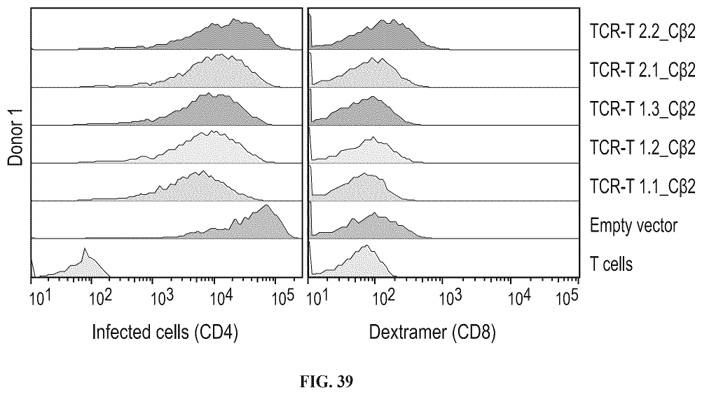

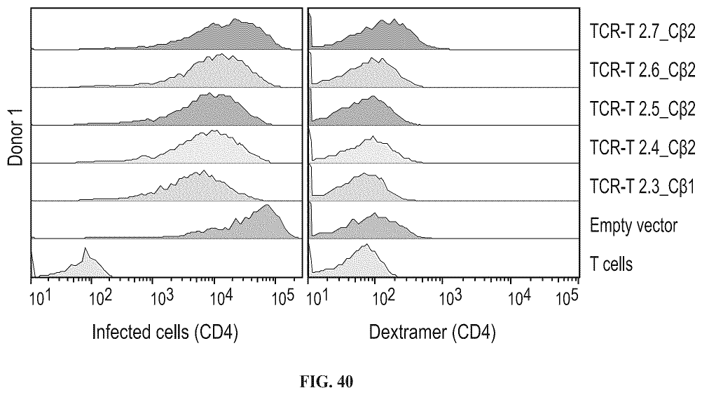

GFP expression revealed that more than 90% of the CD3⁺ T cells of two independent donors were infected with the ALK TCR constructs (both Cβ1 and Cβ2) and the infection level among CD8 T and CD4 T subpopulations was comparable (FIGS. 26-27). The percentage of expression of each ALK TCR (both Cβ1 and Cβ2) was calculated by evaluating the expression and the mean fluorescence intensity (MFI) of the murine TCRβ on the surface of both CD8 T cells and CD4⁺ T cells (FIGS. 28-30). Furthermore, the specificity of the ALK TCR constructs (both Cβ1 and Cβ2) was evaluated by levels of binding to an ALK Dextramer® that has the RPRPSQPSSL (SEQ ID NO: 32957) peptide bound to HLA*B07:02 molecules (FIGS. 31-32). The results demonstrated that despite the transduction efficiency being comparable among all the different ALK constructs tested in two healthy donors, the expression of the ALK TCRs on the surface of both CD8 T cells and CD4 T was construct-dependent, ranging from 2% to 90% and reflecting a different level of mispairing of each ALK TCR construct with the endogenous TCR repertoire. The level of binding to the ALK Dextramer® was variable among all the ALK TCR-T cells (both Cβ1 and Cβ2), with percentages spanning from 1% to 96% in both CD8⁺ T cells and CD4 T of two healthy donors (FIGS. 33-40).

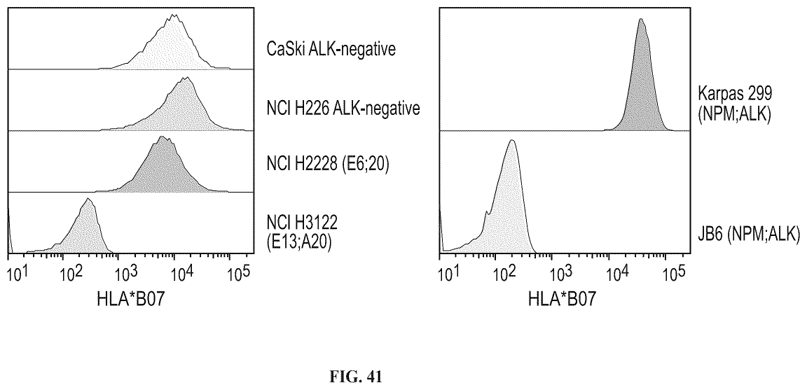

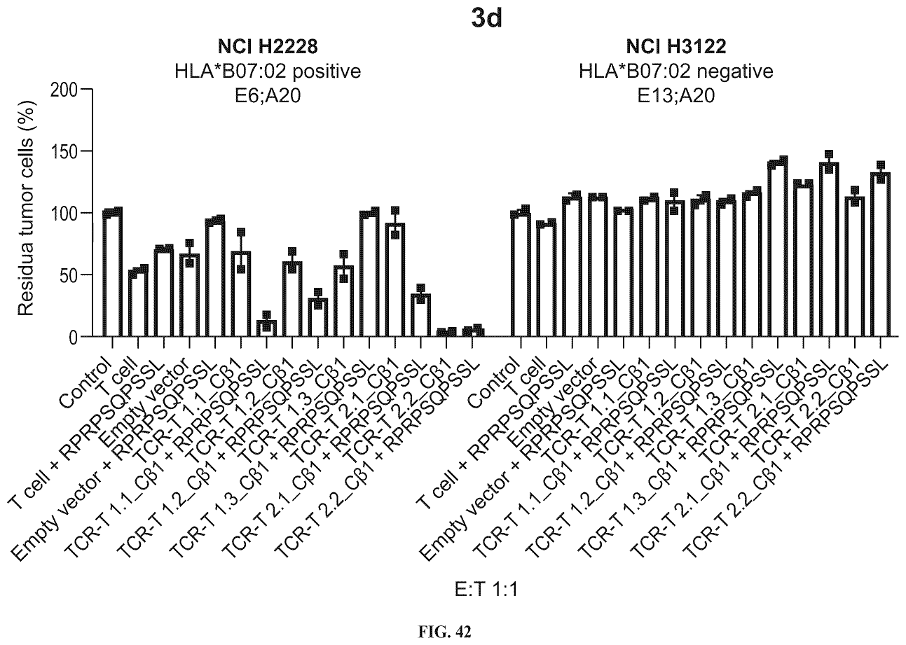

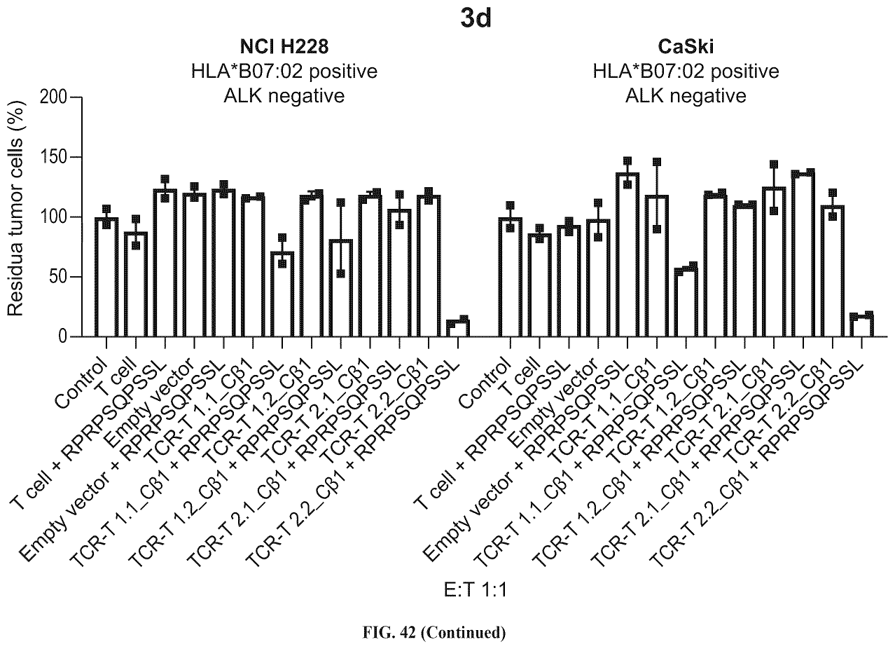

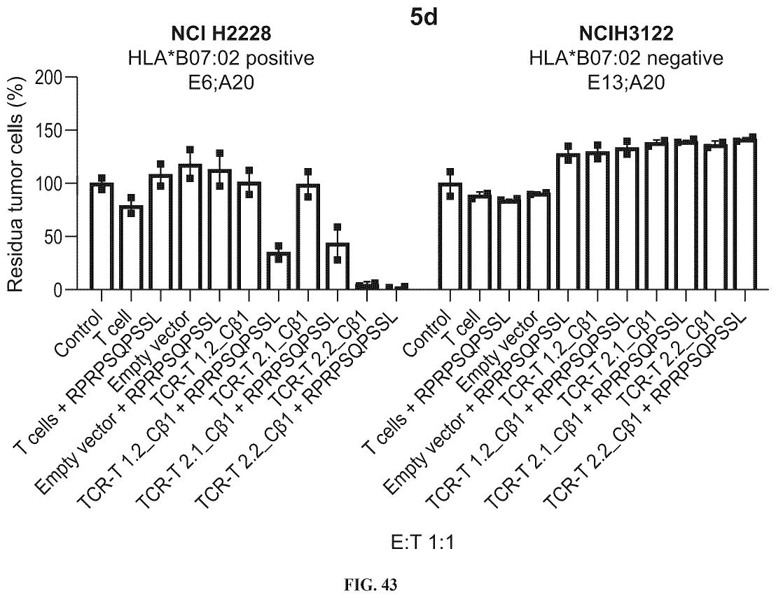

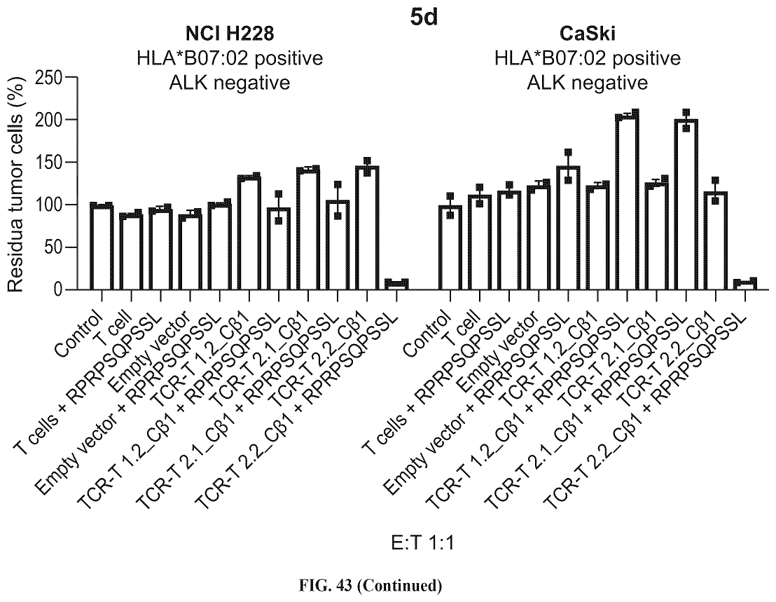

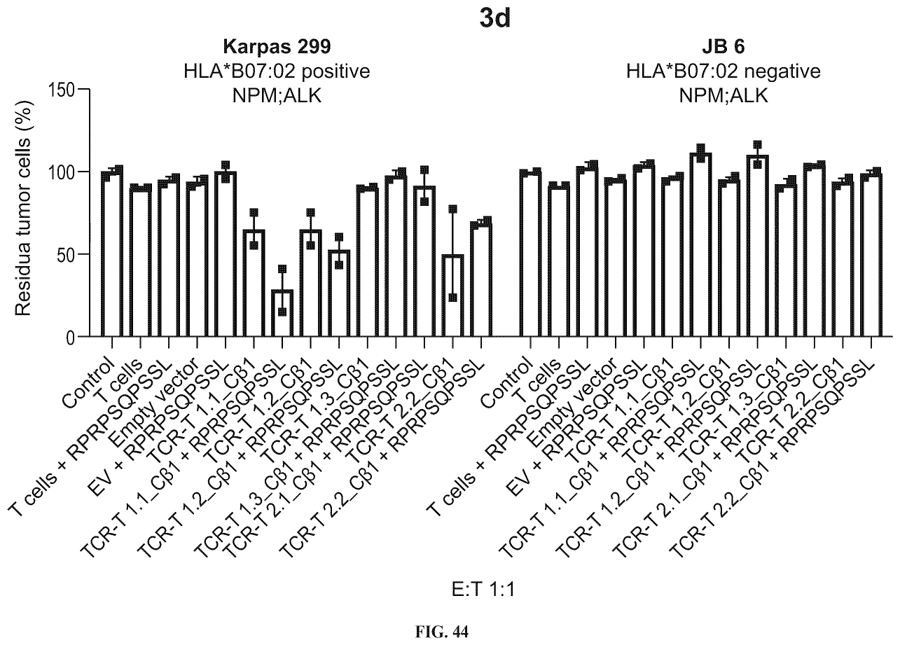

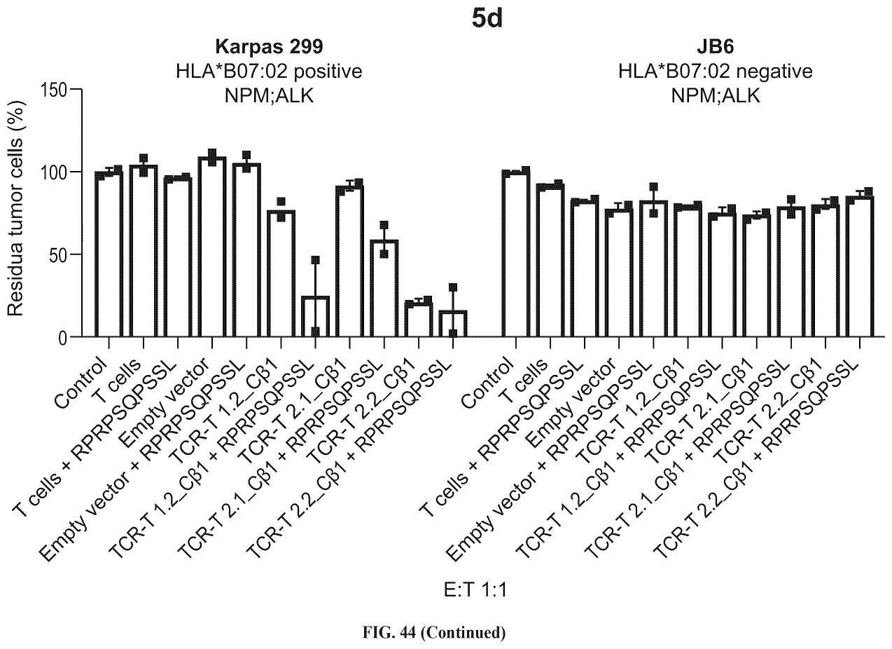

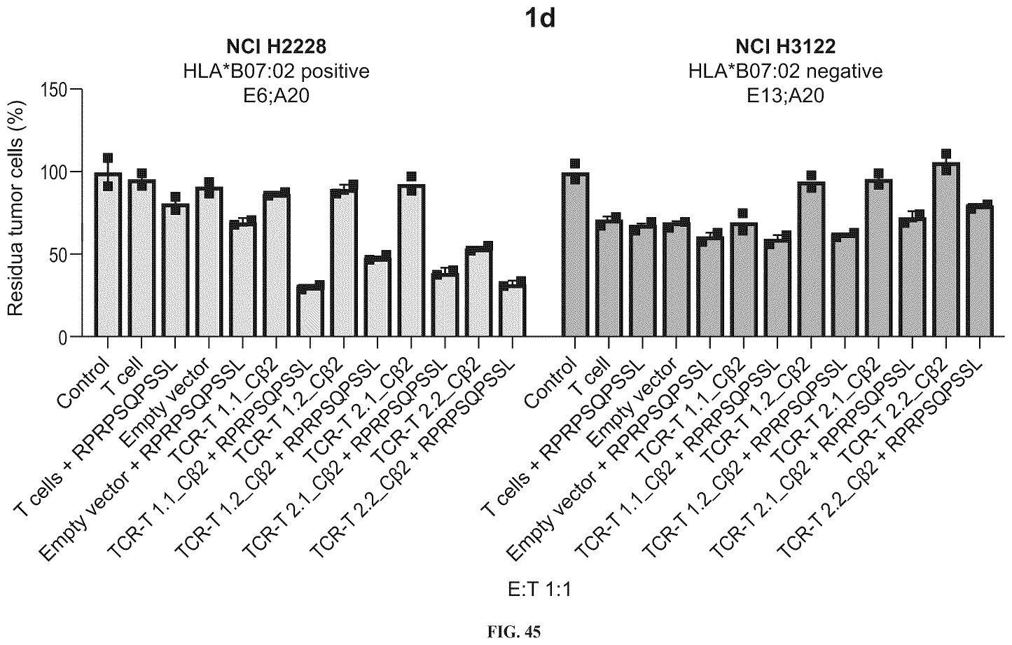

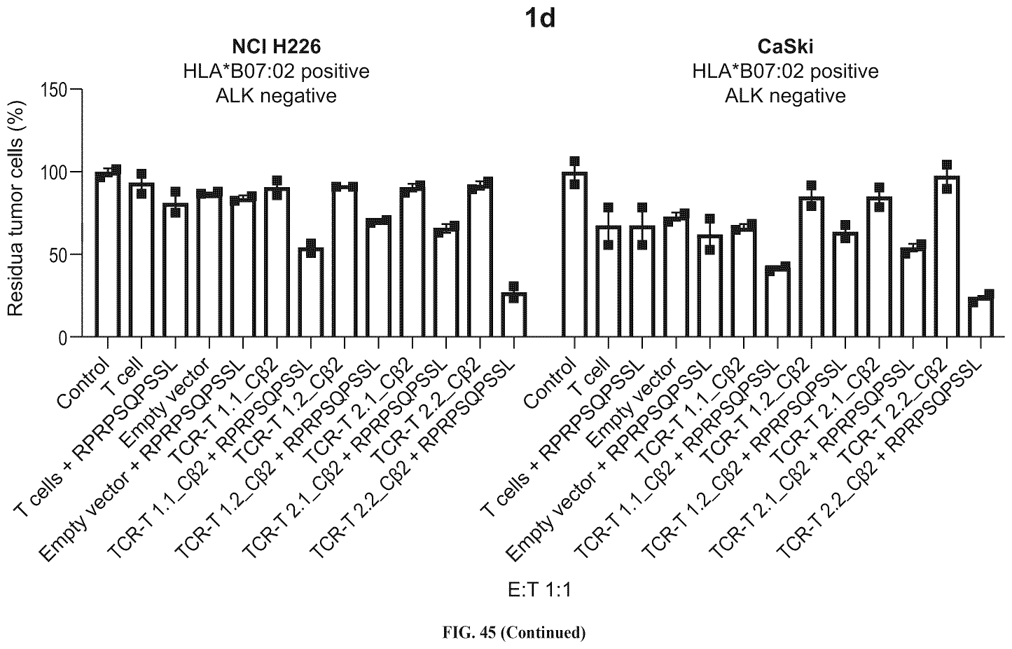

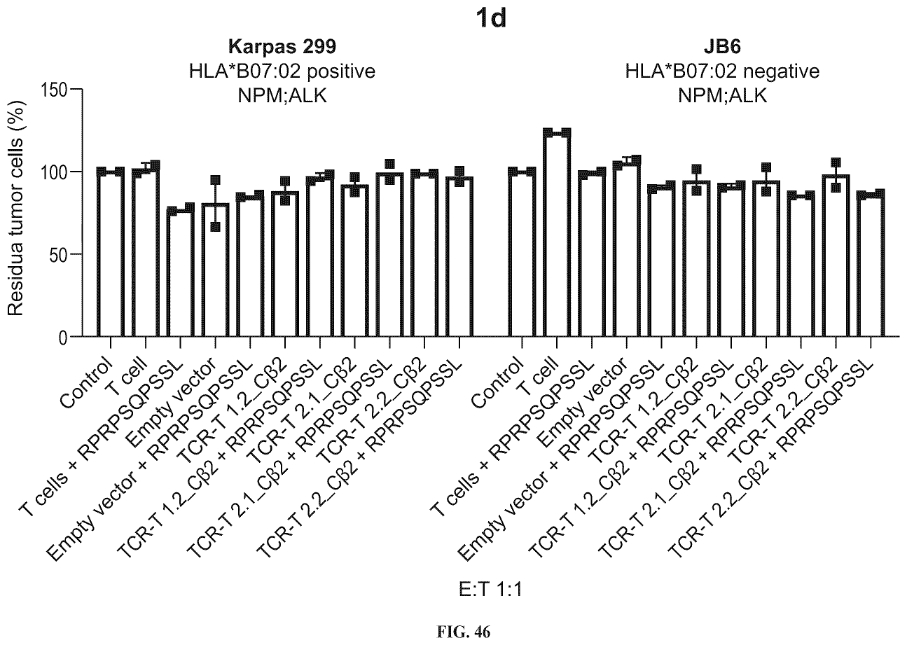

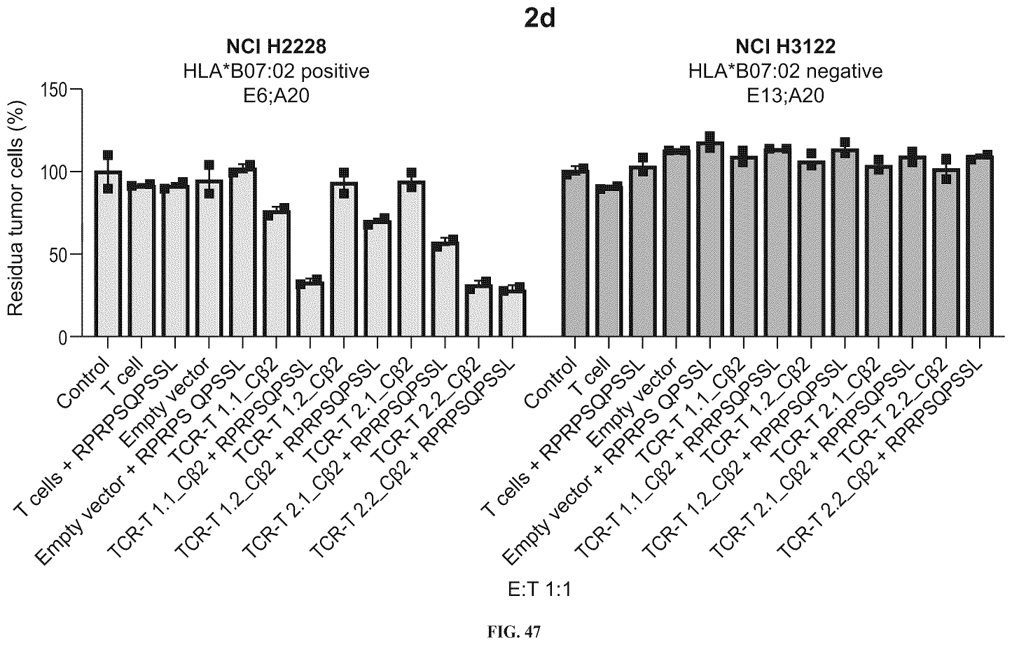

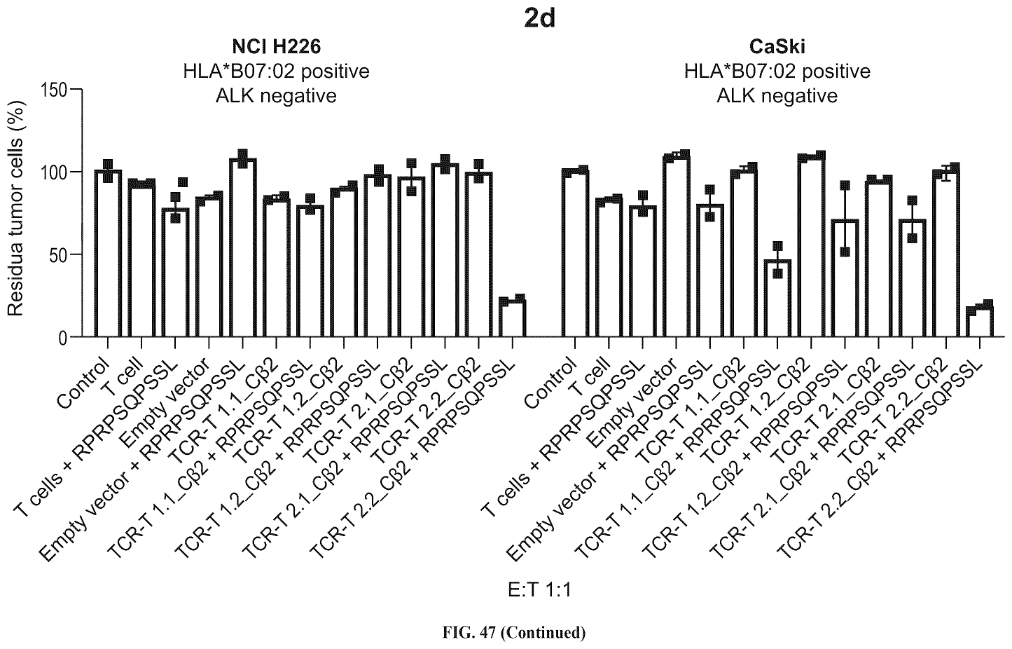

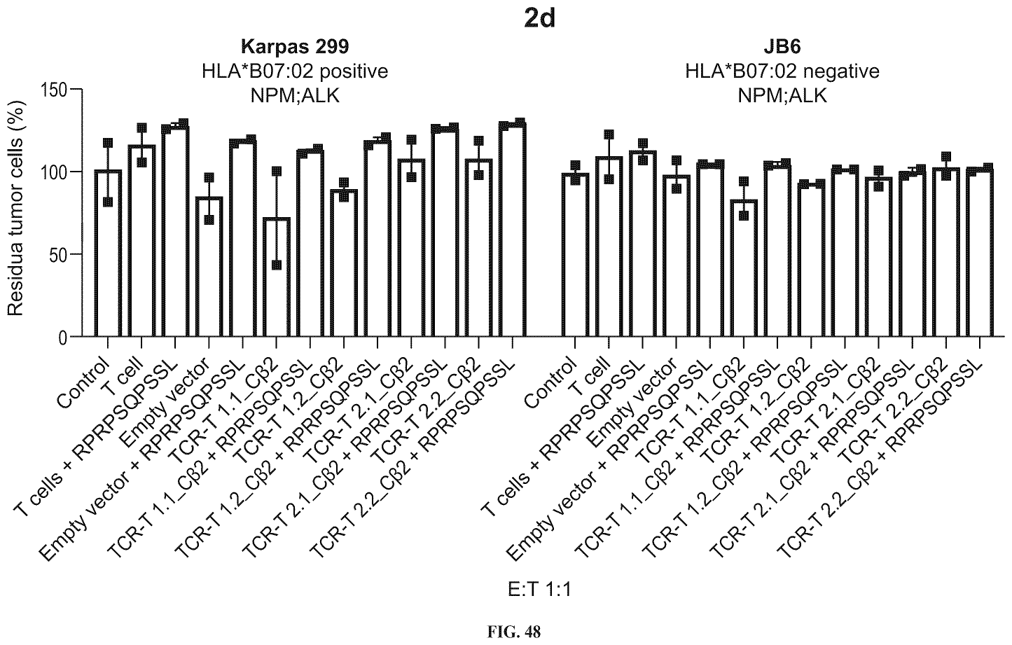

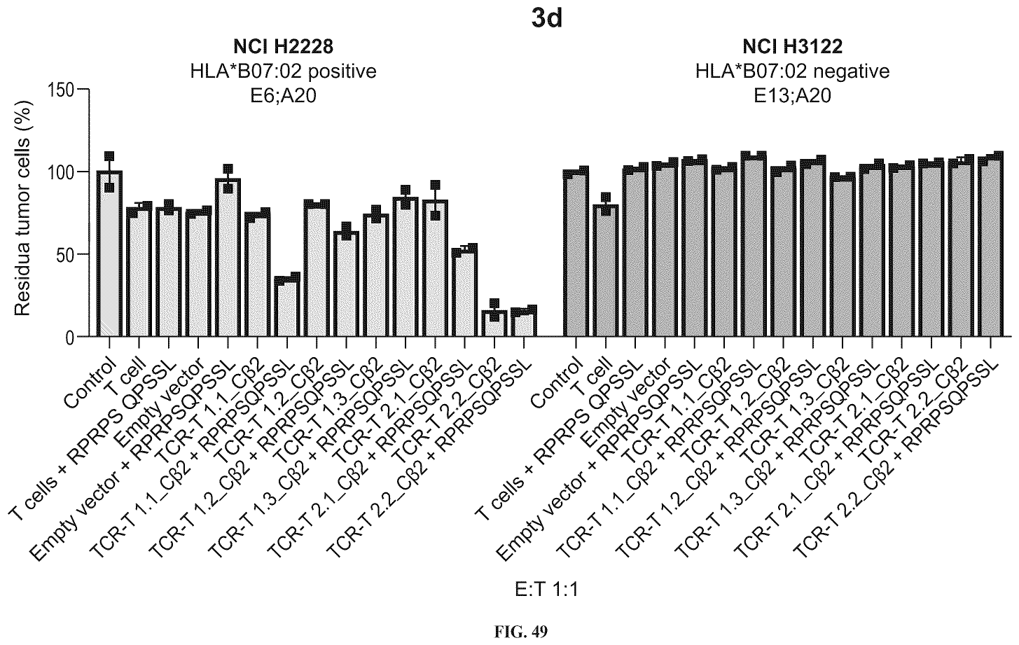

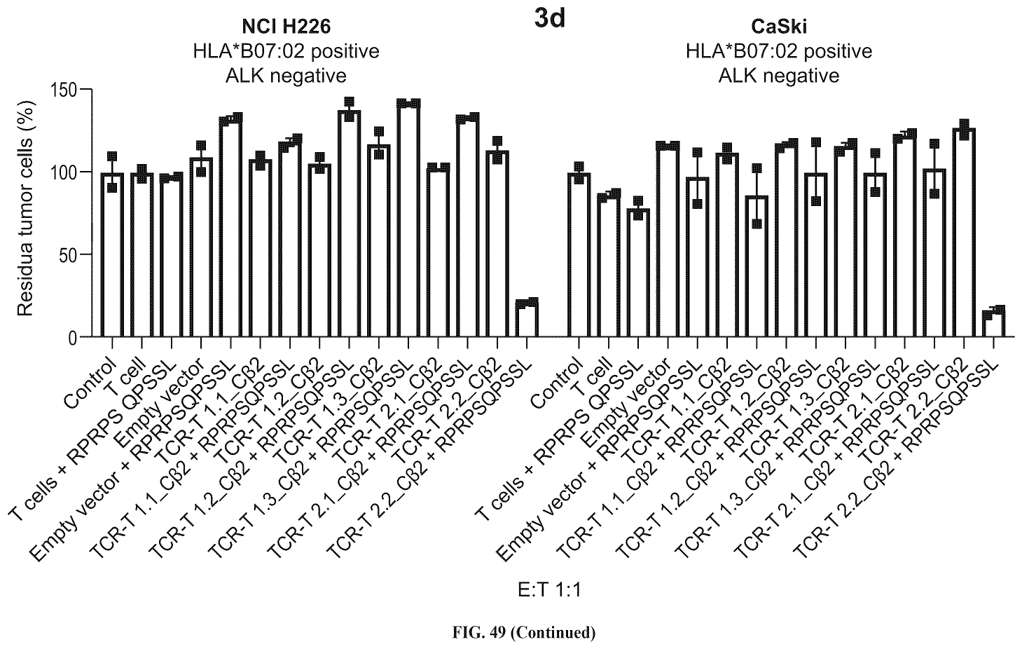

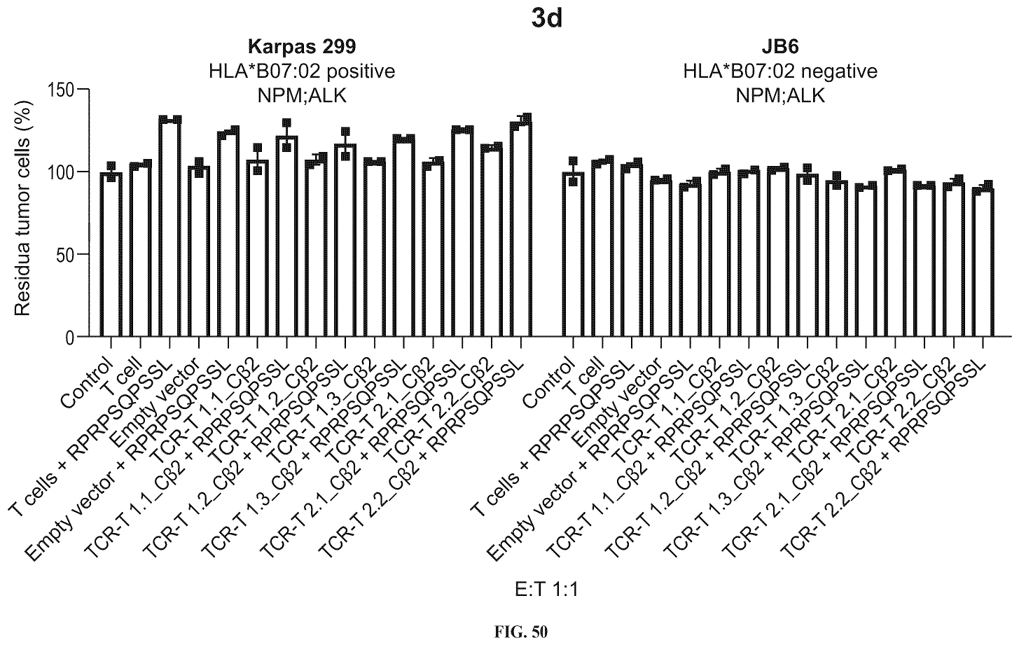

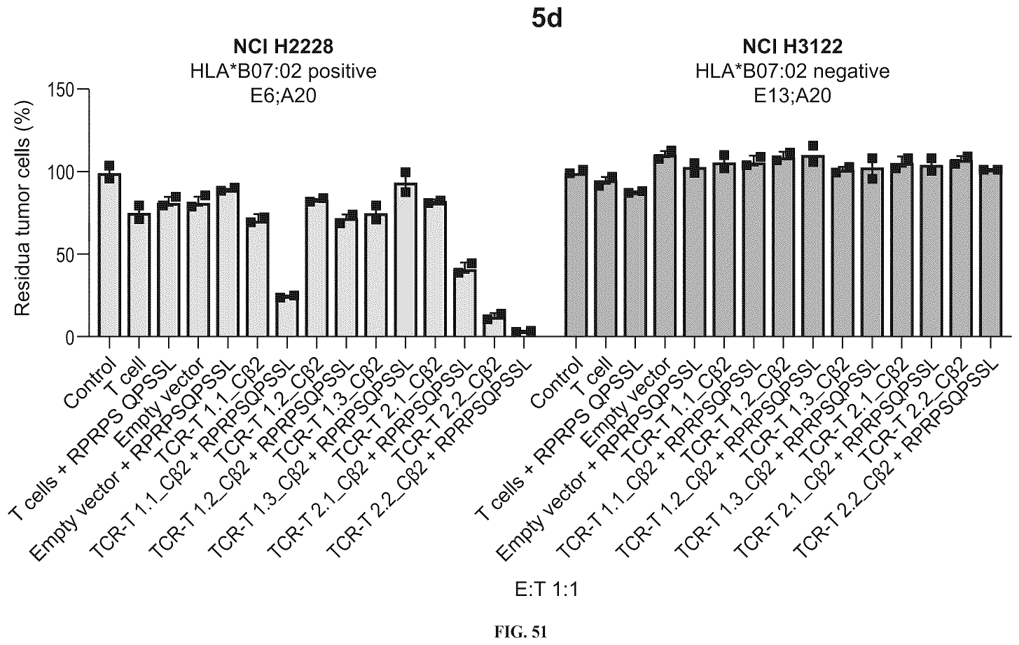

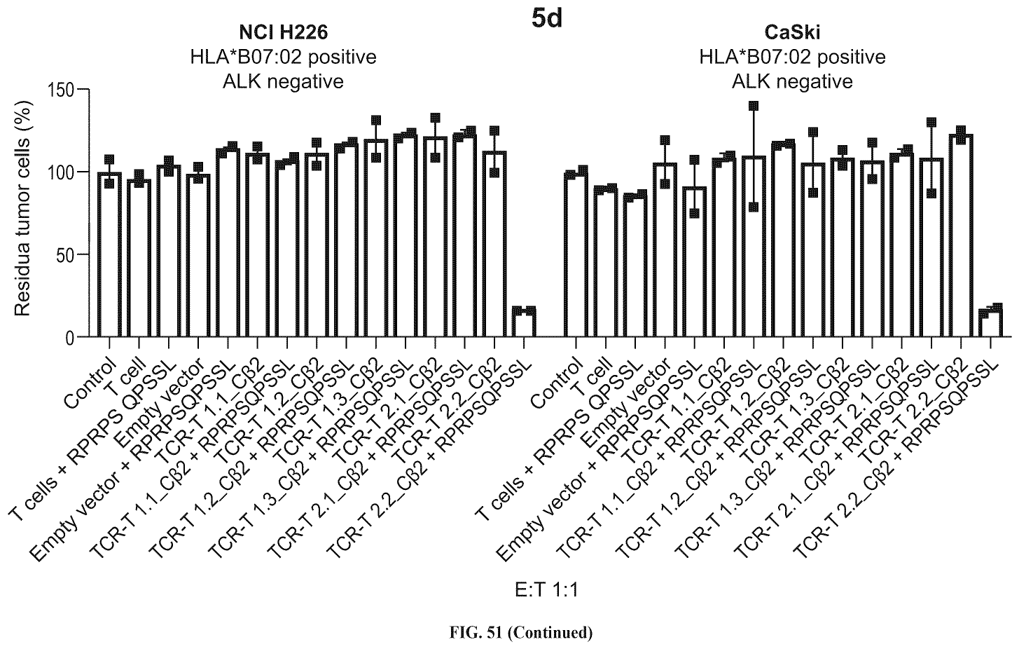

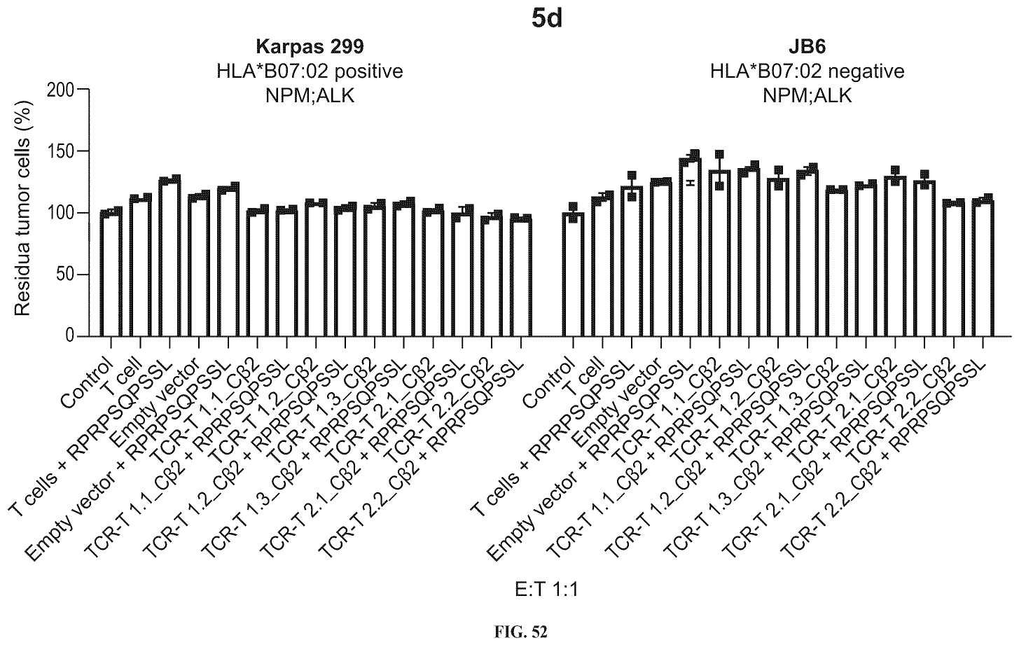

The ALK TCR-T cells were expanded in vitro for 11 days with replacement of 10 ng/ml rhIL-7 and 5 ng/ml rhIL 15 every 2 days. On day 11, untouched CD8⁺ T cells were purified and kept in culture for 2 more days in the absence of rhIL7 and rhIL15. On day 12, a panel of luciferase-positive (FFLUC) tumor cells: NCI H2228 (HLA*B07:02 positive. E6; A20). NCI H3122 (HLA*B07:02 negative E13; A20), NCI H226 (HLA*B07:02 positive. ALK-negative), CaSki (HLA-B07:02 positive, ALK-negative), Karpas 299 (HLA*B07:02 positive, NPM; ALK) and JB6 (HLA*B07:02 negative, NPM; ALK) (FIG. 41) were plated in black 96 well plates, and on day 13, ALK TCR-T cells were added to the tumor cells with a ratio of 1:1. The number of ALK TCR-T cells added was normalized based on the binding to the ALK Dextramer®. The percentage killing was calculated by evaluating the residual bioluminescence of the tumor cells with an IVIS machine at different time points after the co-culture started. The results demonstrated the specificity of the ALK TCR-T cells we generated for both ALK and HLA*B07:02, as no killing of both ALK-negative tumor cells that express HLA*B07:02 and ALK-positive tumor cells that do not express HLA*B07:02 was observed. To further confirm the specificity of the ALK TCR-T cells, the results showed that TCR-T 2.2_Cβ1 and TCR-T 2.2_Cβ2 were able to kill ALK-negative cells that express HLA*B07:02 only when ALK-negative cells were pulsed for two hours with the ALK peptide RPRPSQPSSL (SEQ ID NO: 32957) before the co-culture was started; no killing from any of the TCR-T cells generated when pulsed with the ALK peptide RPRPSQPSSL (SEQ ID NO: 32957) tumor cells that are negative for HLA*B07:02 expression was observed. Instead, very specific, potent, and time-dependent killing of the TCR-T 2.2_Cβ1 and Cβ2 in ALK-positive cells that express HLA*B07:02 was observed (FIGS. 42-52).

Table 2 provides TCR sequences of TCRs isolated from 3 mice of the first cohort of mice vaccinated with RPRPSQPSSL (SEQ ID NO: 32957). Table 3 provides sequences of TCRs isolated from one additional mouse of the first cohort of mice vaccinated with RPRPSQPSSL (SEQ ID NO: 32957).

TABLE 2
TCR sequences from 3 mice, cohort 1, RPRPSQPSSL (SEQ ID NO: 32957) vaccination

| # | Freq | Chain | fwr1 | cdr1 | fwr2 | cdr2 | fwr3 | cdr3 | fwr4 |
|---|---|---|---|---|---|---|---|---|---|
| Clonotype_ | 92 | TRB | GGIITQTPKFLIGQEGQK | FNHDT | MYWYRQDSGKGLR | SITEND | LQKGDLSEGYDASREKKSSFSLTVT | CASSIGTGDNQAPL | GEGTRLSV |
| 1 | | | LTLKCQQN (SEQ ID | (SEQ ID | LIYY (SEQ ID | (SEQ ID | SAQKNEMAVFL (SEQ ID NO: | F (SEQ ID NO: | L (SEQ ID |
| | | | NO: 27) | NO: 2995) | NO: 5963) | NO: 8931) | 11899) | 25393) | NO: 16441) |
| Clonotype_ | 92 | TRA | GEQVEQLPSSLIVQEGAS | DSASVY | FPWYKQEPGKRLQ | IRSNMER | KQNQRLTLLFDKKTKHLSLHITATQ | CSASNNYAQGLTF | GLGTRVSVF |
| 1 | | | VLINCSYT (SEQ ID | (SEQ ID | FIID (SEQ ID | (SEQ ID | PGDSAIYF (SEQ ID NO: | (SEQ ID NO: | P (SEQ ID |
| | | | NO: 160) | NO: | NO: 6096) | NO: | 12032) | 25394) | NO: |
| | | | | 3128) | | 8952) | | | 16344) |
| Clonotype_ | 34 | TRB | VTLLEQNPRWRLVPRGQA | NSQYPW | MSWYQQDLQKQLQ | LRSPGD | KEVKSLPGADYLATRVTDTELRLQV | CTCSADPGGYAEQF | GPGTRLTVL |
| 2 | | | VNLRCILK (SEQ ID | (SEQ ID | WLFT (SEQ ID | (SEQ ID | ANMSQGRTLY (SEQ ID NO: | F (SEQ ID NO: | (SEQ ID |
| | | | NO: 23) | NO: 2991) | NO: 5959) | NO: 8927) | 11895) | 25395) | NO: |
| | | | | | | | | | 16327) |
| Clonotype_ | 34 | TRA | GEQVEQLPSSLIVQEGAS | DSASVY | FPWYKQEPGKRLQ | IRSNMER | KQNQRLTLLFDKKTKHLSLHITATQ | CSASDNYAQGLTF | GLGTRVSVF |
| 2 | | | VLINCSYT (SEQ ID | (SEQ ID | FIID (SEQ ID | (SEQ ID | PGDSAIYF (SEQ ID NO: | (SEQ ID NO: | P (SEQ ID |
| | | | NO: 160) | NO: | NO: 6096) | NO: | 12032) | 25396) | NO: |
| | | | | 3128) | | 8952) | | | 16344) |
| Clonotype_ | 21 | TRB | GGIITQTPKFLIGQEGQK | FNHDT | MYWYRQDSGKGLR | SITEND | LQKGDLSEGYDASREKKSSFSLTVT | CASTDSGNTLYF | GEGSRLIVV |
| 3 | | | LTLKCQQN | (SEQ ID | LIYY (SEQ ID | (SEQ ID | SAQKNEMAVFL (SEQ ID NO: | (SEQ ID NO: | (SEQ ID |
| | | | (SEQ ID | NO: | NO: 5963) | NO: | 11899) | 25397) | NO: |
| | | | NO: 27) | 2995) | | 8931) | | | 16471) |
| Clonotype_ | 21 | TRA | AQSVTQPDARVTVSEGAS | YFGTPY | FWYVQYPRQGLQ | YYPGDPV | GVNGFEAEFSKSNSSFHLRKASVH | CAVTSSGQKLVF | GQGTILKVY |
| 3 | | | LQLRCKYS (SEQ ID | (SEQ ID | LLLK (SEQ ID | V (SEQ | WSDWAVYF (SEQ ID NO: | (SEQ ID NO: | L (SEQ ID |
| | | | NO: 12) | NO: | NO: 5948) | ID NO: | 11924) | 25398) | NO: |
| | | | | 3020) | | 8956) | | | 16414) |
| Clonotype_ | 14 | TRB | EAAVTQSPRSKVAVTGGK | NNHDY | MYWYRQDTGHGLR | SYVADS | TEKGDIPDGYKASRPSQENFSLILE | CASREGDNSAETLY | GSGTRLTVL |
| 4 | | | VTLSCHQT (SEQ ID | (SEQ ID | LIHY (SEQ ID | (SEQ ID | LASLSQTAVYF (SEQ ID NO: | F (SEQ ID NO: | (SEQ ID |
| | | | NO: 7) | NO: | NO: 5939) | NO: | 11879) | 25399) | NO: |
| | | | | 2975) | | 8911) | | | 16325) |
| Clonotype_ | 14 | TRA | GEQVEQRPPHLSVREGDS | DPNSYY | FFWYKQEPGAGLQ | VFSSTEI | NEGQGFTVLLNKKDKQLSLNLTAAH | CAVTPSSSFSKLVF | GQGTSLSVV |
| 4 | | | AVIICTYT (SEQ ID | (SEQ ID | LLMK (SEQ ID | (SEQ ID | PGDSAVYF (SEQ ID NO: | (SEQ ID NO: | P (SEQ ID |
| | | | NO: 108) | NO: | NO: 6044) | NO: | 11980) | 25400) | NO: |
| | | | | 3010) | | 8946) | | | 16422) |
| Clonotype_ | 11 | TRB | DTTVKQNPRYKLARVGKP | MNHDT | MYWYQKKPNQAPK | YYDKIL | NREADTFEKFQSSRPNNSFCSLYIG | CASSRDGHSNTGQL | GEGSKLTVL |
| 5 | | | VNLICSQT (SEQ ID | (SEQ ID | LLLF (SEQ ID | (SEQ ID | SAGLEYSAMYL (SEQ ID NO: | YF (SEQ ID NO: | (SEQ ID |
| | | | NO: 299) | NO: 3267) | NO: 6235) | NO: 9203) | 12171) | 25401) | NO: 16347) |
| Clonotype_ | 11 | TRA | TQTVSQSDAHVTVFEGDS | YGGSIY | LSWYIQHHGHGLQ | YYSGNPV | GVNGFEAEFSKSDSSFHLRKASVH | CAVRGSNAKLTF | GKGTKLSVK |
| 5 | | | VELRCNYS (SEQ ID | (SEQ ID | FLLK (SEQ ID | V (SEQ | WSDSAVYF (SEQ ID NO: | (SEQ ID NO: | S (SEQ ID |
| | | | NO: 110) | NO: | NO: 6518) | ID NO: | 11946) | 25402) | NO: |
| | | | | 3078) | | 9014) | | | 16342) |
| Clonotype_ | 9 | TRB | DMKVTQMPRYLIKRMGEN | MSHET | MYWYRQDPGLGLQ | SYDVDS | NSEGDIPKGYRVSRKKREHFSLILD | CASSFPLGDYAEQF | GPGTRLTVL |
| 6 | | | VLLECGQD (SEQ ID | (SEQ ID | LIYI (SEQ ID | (SEQ ID | SAKTNQTSVYF (SEQ ID NO: | F (SEQ ID NO: | (SEQ ID |
| | | | NO: 163) | NO: | NO: 6099) | NO: | 12035) | 25403) | NO: |
| | | | | 3131) | | 9067) | | | 16327) |
| Clonotype_ | 9 | TRA | QQKVQQSPESLSVPEGGM | DRNFQY | FWWYRQHSGEGPK | IFSDGD | KKEGRFTAHLNKASLHVSLHIRDSQ | CAASGSSGSWQLIF | GSGTQLTVM |
| 6 | | | ASLNCTSS (SEQ ID | (SEQ ID | ALMS (SEQ ID | (SEQ ID | PSDSALYF (SEQ ID NO: | (SEQ ID NO: | P (SEQ ID |
| | | | NO: 64) | NO: | NO: 5938) | NO: | 11874) | 25404) | NO: |
| | | | | 2970) | | 8906) | | | 16400) |
| Clonotype_ | 8 | TRB | EAAVTQSPRSKVAVTGGK | NNHDY | MYWYRQDTGHGLR | SYVADS | TEKGDIPDGYKASRPSQENFSLILE | CASSDTGEEVFF | GKGTRLTVV |
| 7 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25405) | NO: |
| | | | | 2975) | | 8911) | | | 16333) |
| Clonotype_ | 8 | TRA | SQWGEENLQALSIQEGED | TYTTV | VQWYRQKSGKGPA | IRSNERE | KRSGRLRATLDTSSQSSSLSITGTL | CATDNTGYQNFYF | GKGTSLTVI |
| 7 | | | VTMNCSYK (SEQ ID | (SEQ ID | QLIL (SEQ ID | (SEQ ID | ATDTAVYF (SEQ ID NO: | (SEQ ID NO: | P (SEQ ID |
| | | | NO: 80) | NO: 3048) | NO: 6586) | NO: 8928) | 12030) | 25406) | NO: 16366) |
| Clonotype_ | 7 | TRB | EAAVTQSPRNKVAVTGGK | NNHNN | MYWYRQDTGHGLR | SYGAGS | TEKGDIPDGYKASRPSQENFSLILE | CASGRDNSDYTF | GSGTRLLVI |
| 8 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25407) | NO: |
| | | | | | | | | | 16337) |
| Clonotype_ | 7 | TRA | GQGVEQPDNLMSVEGTFA | TSGFNG | LSWYQQREGHAPV | VVLDGL | KDSGHFSTFLSRSNGYSYLLLTELQ | CAVAGNTRKLIF | GLGTTLQVQ |
| 8 | | | RVNCTYS (SEQ ID NO: | (SEQ ID | FLSY (SEQ ID | (SEQ ID | IKDSASYL (SEQ ID NO: | (SEQ ID NO: | P (SEQ ID |
| | | | 4) | NO: | NO: 5940) | NO: | 11876) | 25408) | NO: |
| | | | | 2972) | | 8908) | | | 16326) |
| Clonotype_ | 7 | TRB | EAAVTQSPRNKVAVTGGK | NNHNN | MYWYRQDTGHGLR | SYGAGS | TEKGDIPDGYKASRPSQENFSLILE | CASGDGGAQDTQYF | GPGTRLLVL |
| 9 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25409) | NO: |
| | | | | | | | | | 16341) |
| Clonotype_ | 7 | TRA | GDSVTQKQGQVTLSEDDF | TTTYPT | LFWYVQYPGQGPQ | VTTANNK | GISRGFEATYDKGTTSFHLQKASVQ | CALVDRASSGQKLV | GQGTILKVY |
| 9 | | | LFINCTYS (SEQ ID | (SEQ ID | LLLK (SEQ ID | (SEQ ID | ESDSAVYF (SEQ ID NO: | F (SEQ ID NO: | L (SEQ ID |
| | | | NO: 510) | NO: | NO: 6446) | NO: | 12382) | 25410) | NO: |
| | | | | 3478) | | 9054) | | | 16414) |
| Clonotype_ | 7 | TRB | EAAVTQSPRSKVAVTGGK | INNHDY | MYWYRQDTGHGLR | SYVADS | TEKGDIPDGYKASRPSQENFSLILE | CASSDQGAYEQYF | GPGTRLTVL |
| 10 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: 2975) | NO: 5939) | NO: 8911) | 11879) | 25411) | NO: 16327) |
| Clonotype_ | 7 | TRA | GDSVTQTEGLVTLTKGLP | TTYSPF | LFWYVQHLNEAPK | SSTDNKR | TEHQGFYATLHKSSSSFHLQKSSVQ | CALRPNYNVLYF | GSGTKLTVE |
| 10 | | | VMLNCTYQ (SEQ ID | (SEQ ID | LLLK (SEQ ID | (SEQ ID | LSDSALYF (SEQ ID NO: | (SEQ ID NO: | P (SEQ ID |
| | | | NO: 8) | NO: 2976) | NO: 5944) | NO: 8912) | 11880) | 25412) | NO: 16336) |
| Clonotype_ | 6 | TRB | NTKITQSPRYLILGRANK | LGHNA | MYWYKQSAEKPPE | YNLKQL | IRNETVPSRFIPECPDSSKLLLHIS | CASSQDEGWGEAEQ | GPGTRLTVL |
| 11 | | | SLECEQH (SEQ ID NO: | (SEQ ID | LMFL (SEQ ID | (SEQ ID | AVDPEDSAVYF (SEQ ID NO: | FF (SEQ ID NO: | (SEQ ID |
| | | | 95) | NO: | NO: 6031) | NO: | 11967) | 25413) | NO: |
| | | | | 2983) | | 8999) | | | 16327) |
| Clonotype_ | 6 | TRA | GQQVKQSSPSLTVQEGGI | NDMFDY | FAWYKKYPDNSPT | VRSNVDK | REDGRFTVFLNKSGKHFSLHITASQ | CAAAGFASALTF | GSGTKVIVL |
| 11 | | | LILNCDYE (SEQ ID | (SEQ ID | LLIS (SEQ ID | (SEQ ID | PEDTAVYL (SEQ ID NO: | (SEQ ID NO: | PY (SEQ ID |
| | | | NO: 78) | NO: | | NO: | 11950) | 25414) | NO: |
| | | | | 3046) | | 8982) | | | 17004) |
| Clonotype_ | 5 | TRB | AQTIHQWPVAEIKAVGSP | GKSSPN | LYWYWQATGGTLQ | SITVG | VESVVQLNLSASRPKDDQFILSTE | CAWRTGGVNTGQLY | GEGSKLTVL |
| 12 | | | LSLGCTIK (SEQ ID | (SEQ ID | QLFY (SEQ ID | (SEQ ID | KLLLSHSGFYL (SEQ ID NO: | F (SEQ ID NO: | (SEQ ID |
| | | | NO: 35) | NO: 3003) | NO: 5971) | NO: 8939) | 11907) | 25415) | NO: 16347) |
| Clonotype_ | 5 | TRA | AQKVTQTQTSISVMEKTT | TQDSSYF | LFWYKQTASGEIV | QDSYKKE | ATVGHYSLNFQKPKSSIGLIITATQ | CAMREDSGGSNYKL | GKGTLLTVT |
| 12 | | | VTMDCVYE (SEQ ID | (SEQ ID | FLIR (SEQ ID | N (SEQ | IEDSAVYF (SEQ ID NO: | TF (SEQ ID NO: | P (SEQ ID |
| | | | NO: 44) | NO: | NO: 5942) | ID NO: | 11878) | 16046) | NO: |
| | | | | 3056) | | 8910) | | | 16642) |
| Clonotype_ | 4 | TRB | EAGVTQSPRYAVLQEGQA | SGHDT | LYWYQQPRDQGPQ | FRDEAV | IDNSQLPSDRFSAVRPKGINSTLKI | CASSHSSEQFF | GPGTRLTVL |
| 13 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25417) | NO: |
| | | | | 2969) | | 8905) | | | 16327) |
| Clonotype_ | 4 | TRA | GDSVTQTEGLVTLTKGLP | TTYSPF | LFWYVQHLNEAPK | SSTDNKR | TEHQGFYATLHKSSSSFHLQKSSVQ | CALINNAGAKLTF | GGGTRLTVR |
| 13 | | | VMLNCTYQ (SEQ ID | (SEQ ID | LLLK (SEQ ID | (SEQ ID | LSDSALYF (SEQ ID NO: | (SEQ ID NO: | P (SEQ ID |
| | | | NO: 8) | NO: | NO: 5944) | NO: | 11880) | 25418) | NO: |
| | | | | 2976) | | 8912) | | | 16338) |
| Clonotype_ | 4 | TRB | DPKIIQKPKYLVAVTGSE | LGHNA | MYWYRQSAKKPLE | YSYQKL | MDNQTASSRFQPQSSKKNHLDLQIT | CASSQDLGHPNERL | GHGTKLSVL |
| 14 | | | KILICEQY (SEQ ID | (SEQ ID | FMFS (SEQ ID | (SEQ ID | ALKPDDSATYF (SEQ ID NO: | FF (SEQ ID NO: | (SEQ ID |
| | | | NO: 15) | NO: | NO: 5951) | NO: | 11887) | 25419) | NO: |
| | | | | 2983) | | 8919) | | | 16401) |
| Clonotype_ | 4 | TRA | GDQVKQSPSALSLQEGTN | IATTT | VQWFLQNPRGSLM | LVPGT | KENGRLKSTFNSKESYSTLHIRDAQ | CAAHSGGSNAKLTF | GKGTKLSVK |
| 14 | | | SALRCNFS (SEQ ID | (SEQ ID | NLFY (SEQ ID | (SEQ ID | LEDSGTYF (SEQ ID NO: | (SEQ ID NO: | S (SEQ ID |
| | | | NO: 258) | NO: | NO: 6194) | NO: | 12130) | 25420) | NO: |
| | | | | 3226) | | 9162) | | | 16342) |
| Clonotype_ | 4 | TRB | EAAVTQSPRSKVAVTGGK | INNHDY | MYWYRQDTGHGLR | SYVADS | TEKGDIPDGYKASRPSQENFSLILE | CASRGLGVYAEQFF | GPGTRLTVL |
| 15 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25421) | NO: |
| | | | | 2975) | | 8911) | | | 16327) |
| Clonotype_ | 4 | TRA | AQKVTQTQTSISVMEKTT | TQDSSYF | FLIR (SEQ ID | QDSYKKE | ATVGHYSLNFQKPKSSIGLIITATQ | CAMREGPDYANKMI | GLGTILRVR |
| 15 | | | VTMDCVYE (SEQ ID | (SEQ ID | LFWYKQTASGEIV | N (SEQ | IEDSAVYF (SEQ ID NO: | F (SEQ ID NO: | P (SEQ ID |
| | | | NO: 44) | NO: | NO: 5942) | ID NO: | 11878) | 25422) | NO: |
| | | | | 3056) | | 8910) | | | 16416) |
| Clonotype_ | 4 | TRB | GGIITQTPKFLIGQEGQK | FNHDT | MYWYRQDSGKGLR | SITEND | LQKGDLSEGYDASREKKSSFSLTVT | CASSIGTGDNQAPL | GEGTRLSVL |
| 16 | | | LTLKCQQN (SEQ ID | (SEQ ID | LIYY (SEQ ID | (SEQ ID | SAQKNEMAVFL (SEQ ID NO: | F (SEQ ID NO: | (SEQ ID |
| | | | NO: 27) | NO: 2995) | NO: 5963) | NO: 8931) | 11899) | 25393) | NO: |
| | | | | | | | | | 16441) |
| Clonotype_ | 4 | TRA | GEQVEQLPSSLIVQEGAS | DSASVY | FIID (SEQ ID | IRSNMER | KQNQRLTLLFDKKTKHLSLHITATQ | CSASNNYAQGLTF | GLGTRVSVF |
| 16 | | | VLINCSYT (SEQ ID | (SEQ ID | NO: 6096) | (SEQ ID | PGDSAIYF (SEQ ID NO: | (SEQ ID NO: | P (SEQ ID |
| | | | NO: 160) | NO: | FPWYKQEPGKRLQ | NO: 8952) | 12032) | 25394) | NO: 16344) |
| | | | | 3128) | | | | | |
| Clonotype_ | 4 | TRB | GGIITQTPKFLIGQEGQK | FNHDT | MYWYRQDSGKGLR | SITEND | LQKGDLSEGYDASREKKSSFSLTVT | CASSIGQGAGNTLY | GEGSRLIVV |
| 17 | | | LTLKCQQN (SEQ ID | (SEQ ID | LIYY (SEQ ID | (SEQ ID | SAQKNEMAVFL (SEQ ID NO: | F (SEQ ID NO: | (SEQ ID |
| | | | NO: 27) | NO: | NO: 5963) | NO: | 11899) | 25425) | NO: |
| | | | | 2995) | | 8931) | | | 16471) |
| Clonotype_ | 4 | TRA | GDSVTQTEGLVTLTEGLP | TAYSTF | LFWYVQHLNEAPK | SSTDNKR | TEHQGFHATLHKSSSSFHLQKSSAQ | CALSVTGANTGKLT | GHGTILRVH |
| 17 | | | VMLNCTYQ (SEQ ID | (SEQ ID | LLLK (SEQ ID | (SEQ ID | LSDSALYY (SEQ ID NO: | F (SEQ ID NO: | P (SEQ ID |
| | | | NO: 46) | NO: | NO: 5944) | NO: | 11918) | 25426) | NO: |
| | | | | 3030) | | 8912) | | | 16356) |
| Clonotype_ | 3 | TRB | NTKITQSPRYLILGRANK | ILGHNA | MYWYKQSAEKPPE | YNLKQL | IRNETVPSRFIPECPDSSKLLLHIS | CASSPGTGGGLYNS | AAGTRLTVT |
| 18 | | | SLECEQH (SEQ ID NO: | (SEQ ID | LMFL (SEQ ID | (SEQ ID | AVDPEDSAVYF (SEQ ID NO: | PLYF (SEQ ID | (SEQ ID |
| | | | 95) | NO: | NO: 6031) | NO: | 11967) | NO: 25427) | NO: |
| | | | | 2983) | | 8999) | | | 16381) |
| Clonotype_ | 3 | TRA | GQQVQQSPASLVLQEGEN | TSLNS | MQWFYQRPGGSLV | NPSGT | KHSGRLTSTTVIKERRSSLHISSSQ | CALKRNNNNAPRF | GAGTKLSVK |
| 18 | | | AELQCNFS (SEQ ID | (SEQ ID | SLFY (SEQ ID | (SEQ ID | TTDSGTYL (SEQ ID NO: | (SEQ ID NO: | P (SEQ ID |
| | | | NO: 10) | NO: 2978) | NO: 5946) | NO: 8914) | 11928) | 25428) | NO: |
| | | | | | | | | | 16426) |
| Clonotype_ | 3 | TRB | DPKIIQKPKYLVAVTGSE | LGHNA | MYWYRQSAKKPLE | YSYQKL | MDNQTASSRFQPQSSKKNHLDLQIT | CASSQNRDRGNTEV | GKGTRLTVV |
| 19 | | | KILICEQY (SEQ ID | (SEQ ID | FMFS (SEQ ID | (SEQ ID | ALKPDDSATYF (SEQ ID NO: | FF (SEQ ID NO: | (SEQ ID |
| | | | NO: 15) | NO: 2983) | NO: 5951) | NO: 8919) | 11887) | 25429) | NO: 16333) |
| Clonotype_ | 3 | TRA | GDSVTQTEGQVTVSESKS | TTSIAYP | LFWYVRYPGEGLQ | VITAGQK | GSSRGFEATYNKETTSFHLQKASVQ | CALGAPHTDKVVF | GTGTRLQVS |
| 19 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 60) | ID NO: | NO: 5996) | NO: | 11932) | 25430) | NO: |
| | | | | 3028) | | 8964) | | | 16374) |
| Clonotype_ | 3 | TRB | EAAVTQSPRNKVAVTGGK | INNHNN | MYWYRQDTGHGLR | SYGAGS | TEKGDIPDGYKASRPSQENFSLILE | CASGDAGQPNTGQL | GEGSKLTVL |
| 20 | | | VTLSCNQT (SEQ ID | (SEQ ID | LIHY (SEQ ID | (SEQ ID | LATPSQTSVYF (SEQ ID NO: | YF (SEQ ID NO: | (SEQ ID |
| | | | NO: 3) | NO: | NO: 5939) | NO: | 11875) | 25431) | NO: |
| | | | | 2971) | | 8907) | | | 16347) |
| Clonotype_ | 3 | TRA | AQKVTQTQTSISVMEKTT | TQDSSYF | LFWYKQTASGEIV | QDSYKKE | ATVGHYSLNFQKPKSSIGLIITATQ | CAMTSNTDKVVF | GTGTRLQVS |
| 20 | | | VTMDCVYE (SEQ ID | (SEQ ID | FLIR (SEQ ID | N (SEQ | IEDSAVYF (SEQ ID NO: | (SEQ ID NO: | P (SEQ ID |
| | | | NO: 44) | NO: | NO: 5942) | ID NO: | 11878) | 25432) | NO: |
| | | | | 3056) | | 8910) | | | 16374) |
| Clonotype_ | 3 | TRB | EAAVTQSPRNKVAVTGGK | NNHNN | MYWYRQDTGHGLR | SYGAGS | TEKGDIPDGYKASRPSQENFSLILE | CASGDVRNERLFF | GHGTKLSVL |
| 21 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25433) | NO: |
| | | | | 2971) | | 8907) | | | 16401) |
| Clonotype_ | 3 | TRA | GEQVEQRPPHLSVREGDS | DSATAY | FSWYKQEAGAGLQ | VLSNVDR | KEEQGLTVLLNKKDKRLSLNLTAAH | CAVSPSSGSWQLIF | GSGTQLTVM |
| 21 | | | AIIICTYT (SEQ ID | (SEQ ID | LLMS (SEQ ID | (SEQ ID | PGDSAVYF (SEQ ID NO: | (SEQ ID NO: | P (SEQ ID |
| | | | NO: 22) | NO: | NO: 5958) | NO: | 11894) | 25434) | NO: |
| | | | | 2990) | | 8926) | | | 16400) |
| Clonotype_ | 3 | TRB | EAAVTQSPRSKVAVTGGK | NNHDY | MYWYRQDTGHGLR | SYVADS | TEKGDIPDGYKASRPSQENFSLILE | CASSEGISNERLFF | GHGTKLSVL |
| 22 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25435) | NO: |
| | | | | 2975) | | 8911) | | | 16401) |
| Clonotype_ | 3 | TRA | AQKVTQTQTSISVMEKTT | TRDSSYF | LFWYKQTASGEIV | QDSYKKE | ATEGHYSLNFQKPKSSIGLIITATQ | CAMREVNTGANTGK | GHGTILRVH |
| 22 | | | VTMDCVYE (SEQ ID | (SEQ ID | FLIR (SEQ ID | N (SEQ | IEDSAVYF (SEQ ID NO: | LTF (SEQ ID NO: | P (SEQ ID |
| | | | NO: 44) | NO: | NO: 5942) | ID NO: | 11916) | 25436) | NO: |
| | | | | 2974) | | 8910) | | | 16356) |
| Clonotype_ | 3 | TRB | EAAVTQSPRSKVAVTGGK | NNHDY | MYWYRQDTGHGLR | SYVADS | TEKGDIPDGYKASRPSQENFSLILE | CASSDGWGVYEQYF | GPGTRLTVL |
| 23 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: 2975) | NO: 5939) | NO: 8911) | 11879) | 25437) | NO: |
| | | | | | | | | | 16327) |
| Clonotype_ | 3 | TRA | QQKVQQSPESLTVSEGAM | DGTSNN | FRWYRQHSAKGLE | IFSDGE | KEEGRFTAHLNRANLHVSLHIREPQ | CAANTGANTGKLTF | GHGTILRVH |
| 23 | | | ASLNCTFS (SEQ ID | (SEQ ID | VLVS (SEQ ID | (SEQ ID | PSDSAVYL (SEQ ID NO: | (SEQ ID NO: | P (SEQ ID |
| | | | NO: 92) | NO: | NO: 6028) | NO: | 11964) | 25438) | NO: |
| | | | | 3060) | | 8996) | | | 16356) |
| Clonotype_ | 2 | TRB | NTKITQSPRYLILGRANK | LGHNA | MYWYKQSAEKPPE | YNLKQL | IRNETVPSRFIPECPDSSKLLLHIS | CASNRDRDTEVFF | GKGTRLTVV |
| 24 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25439) | NO: |
| | | | | 2983) | | 8999) | | | 16333) |
| Clonotype_ | | TRA | GDSVTQKQGQVTLSEDDF | TTTYPT | LFWYVQYPGQGPQ | VTTANNK | GISRGFEATYDKGTTSFHLQKASVQ | CALAASNTGYQNFY | GKGTSLTVI |
| 24 | | | LFINCTYS (SEQ ID | (SEQ ID | LLLK (SEQ ID | (SEQ ID | ESDSAVYF (SEQ ID NO: | F (SEQ ID NO: | P (SEQ ID |
| | | | NO: 510) | NO: | NO: 6446) | NO: | 12382) | 25440) | NO: |
| | | | | 3478) | | 9054) | | | 16366) |
| Clonotype_ | 2 | TRB | NTKITQSPRYLILGRANK | GHNA | MYWYKQSAEKPPE | YNLKQL | IRNETVPSRFIPECPDSSKLLLHIS | CASSQDYSSGNTLY | GEGSRLIVV |
| 25 | | | SLECEQH (SEQ ID NO: | (SEQ ID | LMFL (SEQ ID | (SEQ ID | AVDPEDSAVYF (SEQ ID NO: | F (SEQ ID NO: | (SEQ ID |
| | | | 95) | NO: | NO: 6031) | NO: | 11967) | 25441) | NO: |
| | | | | 2983) | | 8999) | | | 16471) |
| Clonotype_ | 2 | TRA | GQQVQQSPASLVLQEGEN | STATR | LQWFYQHPGGRLV | NPSGT | KHTGRLTSTTVTNERRGSLHISSSQ | CANYNVLYF (SEQ | GSGTKLTVE |
| 25 | | | AELQCNFS (SEQ ID | (SEQ ID | SLFY (SEQ ID | (SEQ ID | TTDSGTYF (SEQ ID NO: 11966) | ID NO: 25442) | P (SEQ ID |
| | | | NO: 10) | NO: 3062) | NO: 6030) | NO: 8914) | | | NO: |
| | | | | | | | | | 16336) |
| Clonotype_ | 2 | TRB | SVLLYQKPNRDICQSGTS | SQVVS | MFWYQQFQEQSLM | ANEGSEA | TYESGFTKDKFPISRPNLTFSTLTV | CSSRDGGAQDTQYF | GPGTRLLVL |
| 26 | | | LKIQCVAD (SEQ ID | (SEQ ID | LMAT (SEQ ID | (SEQ ID | NNARPGDSSIYF (SEQ ID NO: | (SEQ ID NO: | (SEQ ID |
| | | | NO: 567) | NO: 3535) | NO: 6503) | NO: 9471) | 12439) | 25443) | NO: 16341) |
| Clonotype_ | 2 | TRA | AQKVTQTQTSISVMEKTT | TRDSSYF | LFWYKQTASGEIV | QDSYKKE | ATEGHYSLNFQKPKSSIGLIITATQ | CAMGTGNYKYVF | GAGTRLKVI |
| 26 | | | VTMDCVYE (SEQ ID | (SEQ ID | FLIR (SEQ ID | N (SEQ | IEDSAVYF (SEQ ID NO: 11916) | (SEQ ID NO: | A (SEQ ID |
| | | | NO: 44) | NO: 2974) | NO: 5942) | ID NO: | | 25444) | NO: 16380) |
| | | | | | | 8910) | | | |
| Clonotype_ | 2 | TRB | ETAVFQTPNYHVTQVGNE | LGHDT | MYWYKQDSKKLLK | YNNKQL | IVNETVPRRFSPQSSDKAHLNLRIK | CASSQLGESSYEQY | GPGTRLTVL |
| 27 | | | VSFNCKQT (SEQ ID | (SEQ ID | IMFS (SEQ ID | (SEQ ID | SVEPEDSAVYL (SEQ ID NO: | F (SEQ ID NO: | (SEQ ID |
| | | | NO: 99) | NO: | NO: 6035) | NO: | 11971) | 25445) | NO: |
| | | | | 3067) | | 9003) | | | 16327) |
| Clonotype_ | 2 | TRA | GQQVQQSPASLVLQEGEN | STATQ | LQWFYQSPGGSLV | NPSGT | KHTGRLTSTTVTKERRSSLHISSSQ | CAIALGGYKLTF | GTGTSLLVD |
| 27 | | | AELQCNFS (SEQ ID | (SEQ ID | SLLS (SEQ ID | (SEQ ID | TTDSGTYL (SEQ ID NO: | (SEQ ID NO: | P (SEQ ID |
| | | | NO: 10) | NO: | NO: 6258) | NO: | 12194) | 25446) | NO: |
| | | | | 3290) | | 8914) | | | 16398) |
| Clonotype_ | 2 | TRB | ETAVFQTPNYHVTQVGNE | LGHDT | MYWYKQDSKKLLK | YNNKQL | IVNETVPRRFSPQSSDKAHLNLRIK | CASSSRGLDSYEQY | GPGTRLTVL |
| 28 | | | VSFNCKQT (SEQ ID | (SEQ ID | IMFS (SEQ ID | (SEQ ID | SVEPEDSAVYL (SEQ ID NO: | F (SEQ ID NO: | (SEQ ID |
| | | | NO: 99) | NO: | NO: 6035) | NO: | 11971) | 25447) | NO: |
| | | | | 3067) | | 9003) | | | 16327) |
| Clonotype_ | 2 | TRA | GQQVQQSPASLVLQEGEN | TSLNS | MQWFYQRPGGSLV | NPSGT | KQSGRLTSTTVIKERRSSLHISSSQ | CAMDSAGNKLTF | GIGTRVLVR |
| 28 | | | AELQCNFS (SEQ ID | (SEQ ID | SLFY (SEQ ID | (SEQ ID | TTDSGTYL (SEQ ID NO: 11882) | (SEQ ID NO: | P (SEQ ID |
| | | | NO: 10) | NO: 2978) | NO: 5946) | NO: 8914) | | 15096) | NO: 16580) |
| Clonotype_ | 2 | TRB | EAAVTQSPRNKVAVTGGK | NNHNN | MYWYRQDTGHGLR | SYGAGS | TEKGDIPDGYKASRPSQENFSLILE | CASGAPGLGDYEQY | GPGTRLTVL |
| 29 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: 2971) | NO: 5939) | NO: 8907) | 11875) | 25449) | NO: |
| | | | | | | | | | 16327) |
| Clonotype_ | 2 | TRA | GQQVQQSPASLVLQEGEN | STATR | LQWFYQHPGGRLV | NPSGT | KHTGRLTSTTVTNERRGSLHISSSQ | CAIDRGNYAQGLTF | GLGTRVSVF |
| 29 | | | AELQCNFS (SEQ ID | (SEQ ID | SLFY (SEQ ID | (SEQ ID | TTDSGTYF (SEQ ID NO: | (SEQ ID NO: | P (SEQ ID |
| | | | NO: 10) | NO: | NO: 6030) | NO: | 11966) | 25450) | NO: |
| | | | | 3062) | | 8914) | | | 16344) |
| Clonotype_ | 2 | TRB | EAAVTQSPRNKVAVTGGK | NNHNN | MYWYRQDTGHGLR | SYGAGS | TEKGDIPDGYKASRPSQENFSLILE | CASGDGAGGGAEQF | GPGTRLTVL |
| 30 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25451) | NO: |
| | | | | 2971) | | 8907) | | | 16327) |
| Clonotype_ | 2 | TRA | GDSVTQTEGQVTVSESKS | ATSIGYP | LFWYVRYPGEGLQ | VITAGQK | GSSRGFEATYNKEATSFHLQKASVQ | CALSNTGYQNFYF | GKGTSLTVI |
| 30 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 60) | ID NO: | NO: 5996) | NO: | 12094) | 25452) | NO: |
| | | | | 3330) | | 8964) | | | 16366) |
| Clonotype_ | 2 | TRB | EAAVTQSPRNKVAVTGGK | NNHNN | MYWYRQDTGHGLR | SYGAGS | TEKGDIPDGYKASRPSQENFSLILE | CASGGTGGGDTQYF | GPGTRLLVL |
| 31 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25453) | NO: |
| | | | | | | | | | 16341) |
| Clonotype_ | 2 | TRA | GDSVTQMQGQVTLSEDDF | TTWYPT | LFWYVQYPGEGPQ | VTTANNK | GISRGFEATYDKRTTSFHLQKASVQ | CVLGEGSNYQLIW | GSGTKLIIK |
| 31 | | | LFINCTYS (SEQ ID | (SEQ ID | LLLK (SEQ ID | (SEQ ID | ESDSAVYY (SEQ ID NO: | (SEQ ID NO: | P (SEQ ID |
| | | | NO: 150) | NO: | NO: 6086) | NO: | 12022) | 25454) | NO: |
| | | | | 3118) | | 9054) | | | 16372) |
| Clonotype_ | 2 | TRB | EAAVTQSPRSKVAVTGGK | NNHDY | MYWYRQDTGHGLR | SYVADS | TEKGDIPDGYKASRPSQENFSLILE | CASSRDNSYNSPLY | AAGTRLTVT |
| 32 | | | VTLSCHQT (SEQ ID | (SEQ ID | LIHY (SEQ ID | (SEQ ID | LASLSQTAVYF (SEQ ID NO: | F (SEQ ID NO: | (SEQ ID |
| | | | NO: 7) | NO: | NO: 5939) | NO: | 11879) | 25455) | NO: |
| | | | | 2975) | | 8911) | | | 16381) |
| Clonotype_ | 2 | TRA | AQSVDQPDAHVTLYEGAS | YSAAPY | LFWYVQYPGQSLQ | YITGDAV | KGTKGFEAEFRKSNSSFNLKKSPAH | CACVGDNSKLIW | GLGTSLVVN |
| 32 | | | LELRCSYS (SEQ ID | (SEQ ID | FLLK (SEQ ID | V (SEQ | WSDSAKYF (SEQ ID NO: | (SEQ ID NO: | P (SEQ ID |
| | | | NO: 398) | NO: | NO: 6216) | ID NO: | 12152) | 25456) | NO: |
| | | | | 3248) | | 9302) | | | 17292) |
| Clonotype_ | 2 | TRB | EAAVTQSPRSKVAVTGGK | NNHDY | MYWYRQDTGHGLR | SYVADS | TEKGDIPDGYKASRPSQENFSLILE | CASSDGTTLYF | GAGTRLSVL |
| 33 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25457) | NO: |
| | | | | 2975) | | 8911) | | | 16365) |
| Clonotype_ | 2 | TRA | AQSVDQPDAHVTLSEGAS | YSAAPY | LFWYVQYPGQSLQ | YITGDTV | KGTKGFEAEFRKSNSSFNLKKSPAH | CALEAGGGSNYKLT | GKGTLLTVT |
| 33 | | | LELRCSYS (SEQ ID | (SEQ ID | FLLK (SEQ ID | V (SEQ | WSDSAKYF (SEQ ID NO: | F (SEQ ID NO: | P (SEQ ID |
| | | | NO: 280) | NO: | NO: 6216) | ID NO: | 12152) | 25458) | NO: |
| | | | | 3248) | | 9184) | | | 16642) |
| Clonotype_ | 2 | TRB | DTTVKQNPRYKLARVGKP | MNHDT | MYWYQKKPNQAPK | YYDKIL | NREADTFEKFQSSRPNNSFCSLYIG | CASSWTGRDAEQFF | GPGTRLTVL |
| 34 | | | VNLICSQT (SEQ ID | (SEQ ID | LLLF (SEQ ID | (SEQ ID | SAGLEYSAMYL (SEQ ID NO: | (SEQ ID NO: | (SEQ ID |
| | | | NO: 299) | NO: | NO: 6235) | NO: | 12171) | 25459) | NO: |
| | | | | 3267) | | 9203) | | | 16327) |
| Clonotype_ | 2 | TRA | AQSVTQPDARVTVSEGAS | SSVTPY | LFWYVQYPRQGLQ | YYSGDPV | GVNGFEAEFSKSNSSFHLRKASVH | CAVSMANAPRF | GAGTKLTVK |
| 34 | | | LQLRCKYS (SEQ ID | (SEQ ID | LLLK (SEQ ID | V (SEQ | WSDSAVYF (SEQ ID NO: | (SEQ ID NO: | P (SEQ ID |
| | | | NO: 12) | NO: | NO: 5948) | ID NO: | 11892) | 25460) | NO: |
| | | | | 2988) | | 8916) | | | 16424) |
| Clonotype_ | 2 | TRB | AQTIHQWPVAEIKAVGSP | GKSSPN | LYWYWQATGGTLQ | SITVG | VESVVQLNLSASRPKDDQFILSTE | CAWSLPSRLSDEQY | GPGTRLTVL |
| 35 | | | LSLGCTIK (SEQ ID | (SEQ ID | QLFY (SEQ ID | (SEQ ID | KLLLSHSGFYL (SEQ ID NO: | F (SEQ ID NO: | (SEQ ID |
| | | | NO: 35) | NO: | NO: 5971) | NO: | 11907) | 25461) | NO: |
| | | | | 3003) | | 8939) | | | 16327) |
| Clonotype_ | 2 | TRA | GEQVEQRPPHLSVREGDS | DPNSYY | FFWYKQEPGAGLQ | VFSSTEI | NEGQGFTVLLNKKDKQLSLNLTAAH | CAASTNAYKVIF | GKGTHLHVL |
| 35 | | | AVIICTYT (SEQ ID | (SEQ ID | LLMK (SEQ ID | (SEQ ID | PGDSAVYF (SEQ ID NO: | (SEQ ID NO: | P (SEQ ID |
| | | | NO: 108) | NO: | NO: 6044) | NO: | 11980) | 25462) | NO: |
| | | | | 3010) | | 8946) | | | 16330) |
| Clonotype_ | 2 | TRB | GGIITQTPKFLIGQEGQK | FNHDT | MYWYRQDSGKGLR | SITEND | LQKGDLSEGYDASREKKSSFSLTVT | CASSLGQFTEVFF | GKGTRLTVV |
| 36 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: 2995) | NO: 5963) | NO: 8931) | 11899) | 25463) | NO: |
| | | | | | | | | | 16333) |
| Clonotype_ | 2 | TRA | AQSVDQPDAHVTLSEGAS | YSAAPY | LFWYVQYPGQSLQ | YITGDTV | KGTKGFEAEFRKSNSSFNLKKSPAH | CALEGRYGSSGNKL | GIGTLLSVK |
| 36 | | | LELRCSYS (SEQ ID | (SEQ ID | FLLK (SEQ ID | V (SEQ | WSDSAKYF (SEQ ID NO: | IF (SEQ ID NO: | P (SEQ ID |
| | | | NO: 280) | NO: | NO: 6216) | ID NO: | 12152) | 25464) | NO: |
| | | | | 3248) | | 9184) | | | 16456) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQRDSGEQYF | GPGTRLTVL |
| 37 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25465) | NO: |
| | | | | 2983) | | 8999) | | | 16327) |
| Clonotype_ | 1 | TRA | GDSVTQTEGQVALSEEDF | ASGYPA | LFWYVQYPGEGPQ | ASRDKEK | GSSRGFEATYNKEATSFHLQKASVQ | CALTANTGNYKYVF | GAGTRLKVI |
| 37 | | | LTIHCNYS (SEQ ID | (SEQ ID | FLFR (SEQ ID | (SEQ ID | ESDSAVYY (SEQ ID NO: | (SEQ ID NO: | A (SEQ ID |
| | | | NO: 14) | NO: | NO: 6050) | NO: | 12094) | 25466) | NO: |
| | | | | 3082) | | 8918) | | | 16380) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQEQNAETLYF | GSGTRLTVL |
| 38 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25467) | NO: |
| | | | | 2983) | | 8999) | | | 16325) |
| Clonotype_ | 1 | TRA | (SEQEKVQQSPESLIVPEGAM | NSASQS | IWWYQQHPGKGPE | IFSNGN | KKEGRLTVYLNRASLHVSLHIRDSQ | CAAATSSGQKLVF | GQGTILKVY |
| 38 | | | SSLNCTFS (SEQ ID | (SEQ ID | ALIS (SEQ ID | (SEQ ID | PSDSAVYL (SEQ ID NO: | (SEQ ID NO: | L (SEQ ID |
| | | | NO: 96) | NO: | NO: 6032) | NO: | 11968) | 25468) | NO: |
| | | | | 3064) | | 9000) | | | 16414) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQDRGFAEQFF | GPGTRLTVL |
| 39 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25469) | NO: |
| | | | | 2983) | | 8999) | | | 16327) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMRESSSSFSKLV | GQGTSLSVV |
| 39 | | | VTMDCVYE (SEQ ID | (SEQ ID | FLIR (SEQ ID | N (SEQ | IEDSAVYF (SEQ ID NO: | F (SEQ ID NO: | P (SEQ ID |
| | | | NO: 44) | NO: | NO: 5942) | ID NO: | 11878) | 25470) | NO: |
| | | | | 3056) | | 8910) | | | 16422) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQDWVSYNSPL | AAGTRLTVT |
| 40 | | | SLECEQH (SEQ ID NO: | (SEQ ID | LMFL (SEQ ID | (SEQ ID | AVDPEDSAVYF (SEQ ID NO: | YF (SEQ ID NO: | (SEQ ID |
| | | | 95) | NO: | NO: 6031) | NO: | 11967) | 25471) | NO: |
| | | | | 2983) | | 8999) | | | 16381) |
| Clonotype_ | 1 | TRA | GDSVTQTEGLVTLTEGLP | STYSPF | LFWYVQHLNEAPK | SFTDNKR | PEHQGFHATLHKSSSSFHLQKSSAQ | CARGDNSKLIW | GLGTSLVVN |
| 40 | | | VMLNCTYQ (SEQ ID | (SEQ ID | LLLK (SEQ ID | (SEQ ID | LSDSALYY (SEQ ID NO: | (SEQ ID NO: | P (SEQ ID |
| | | | NO: 46) | NO: | NO: 5944) | NO: | 11944) | 25472) | NO: |
| | | | | 3040) | | 8950) | | | 17292) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQDPGGLAETL | GSGTRLTVL |
| 41 | | | SLECEQH (SEQ ID NO: | (SEQ ID | LMFL (SEQ ID | (SEQ ID | AVDPEDSAVYF (SEQ ID NO: | YF (SEQ ID NO: | (SEQ ID |
| | | | 95) | NO: | NO: 6031) | NO: | 11967) | 25473) | NQ |
| | | | | 2983) | | 8999) | | | 16325) |
| Clonotype_ | 1 | TRA | GDSVIQMQGQVTLSENDF | TTGYPT | LFWYVQYSGEGPQ | VTTANNK | GSSRGFEATYDKGTTSFHLQKTSVQ | CAMSPNMGYKLTF | GTGTSLLVD |
| 41 | | | LFINCTYS (SEQ ID | (SEQ ID | LLLQ (SEQ ID | (SEQ ID | EIDSAVYY (SEQ ID NO: | (SEQ ID NO: | P (SEQ ID |
| | | | NO: 456) | NO: | NO: 6392) | NO: 9054) | 12328) | 25474) | NO: |
| | | | | 3220) | | | | | 16398) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQDAGDTLYF | GEGSRLIVV |
| 42 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25475) | NO: |
| | | | | 2983) | | 8999) | | | 16471) |
| Clonotype_ | 1 | TRA | AQKVTQTQTSISVMEKTT | TRDSSYF | LFWYKQTASGEIV | QDSYKKE | ATEGHYSLNFQKPKSSIGLIITATQ | CAMRGHSSSSFSKL | GQGTSLSVV |
| 42 | | | VTMDCVYE (SEQ ID | (SEQ ID | FLIR (SEQ ID | N (SEQ | IEDSAVYF (SEQ ID NO: | VF (SEQ ID NO: | P (SEQ ID |
| | | | NO: 44) | NO: | NO: 5942) | ID NO: | 11916) | 25476) | NO: |
| | | | | 2974) | | 8910) | | | 16422) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQDGGDAETLY | GSGTRLTVL |
| 43 | | | SLECEQH (SEQ ID NO: | (SEQ ID | LMFL (SEQ ID | (SEQ ID | AVDPEDSAVYF (SEQ ID NO: | F (SEQ ID NO: | (SEQ ID |
| | | | 95) | NO: | NO: 6031) | NO: | 11967) | 25477) | NO: |
| | | | | 2983) | | 8999) | | | 16325) |
| Clonotype_ | 1 | TRA | AQSVTQPDARVTVSEGAS | SSVTPY | LFWYVQYPRQGLQ | YYSGDPV | GVNGFEAEFSKSNSSFHLRKASVH | CAVSSLNVGDNSKL | GLGTSLVVN |
| 43 | | | LQLRCKYS (SEQ ID | (SEQ ID | LLLK (SEQ ID | V (SEQ | WSDSAVYF (SEQ ID NO: 11892) | IW (SEQ ID NO: | P (SEQ ID |
| | | | NO: 12) | NO: 2988) | NO: 5948) | ID NO: | | 25478) | NO: |
| | | | | | | 8916) | | | 17292) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQDGHYAEQFF | GPGTRLTVL |
| 44 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25479) | NO: |
| | | | | 2983) | | 8999) | | | 16327) |
| Clonotype_ | 1 | TRA | AQKVTQTQTSISVMEKTT | TRDSSYF | LFWYKQTASGEIV | QDSYKKE | ATEGHYSLNFQKPKSSIGLIITATQ | CAMRGQGTGSKLSF | GKGAKLTVS |
| 44 | | | VTMDCVYE (SEQ ID | (SEQ ID | FLIR (SEQ ID | N (SEQ | IEDSAVYF (SEQ ID NO: | (SEQ ID NO: | P (SEQ ID |
| | | | NO: 44) | NO: | NO: 5942) | ID NO: | 11916) | 25480) | NO: |
| | | | | 2974) | | 8910) | | | 16524) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQDWDSGNTLY | GEGSRLIVV |
| 45 | | | SLECEQH (SEQ ID NO: | (SEQ ID | LMFL (SEQ ID | (SEQ ID | AVDPEDSAVYF (SEQ ID NO: | F (SEQ ID NO: | (SEQ ID |
| | | | 95) | NO: | NO: 6031) | NO: 8999) | 11967) | 25481) | NO: |
| | | | | 2983) | | | | | 16471) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMRLTGNTRKLIF | GLGTTLQVQ |
| 45 | | | VTMDCVYE (SEQ ID | (SEQ ID | FLIR (SEQ ID | N (SEQ | IEDSAVYF (SEQ ID NO: | (SEQ ID NO: | P (SEQ ID |
| | | | NO: 44) | NO: | NO: 5942) | ID NO: | 11878) | 25482) | NO: |
| | | | | 3056) | | 8910) | | | 16326) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQAQSQNTLYF | GAGTRLSVL |
| 46 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25483) | NO: |
| | | | | 2983) | | 8999) | | | 16365) |
| Clonotype_ | 1 | TRA | GEQVEQRPPHLSVREGDS | DPNSYY | FFWYKQEPGAGLQ | VFSSTEI | NEGQGFTVLLNKKDKQLSLNLTAAH | CAVRASSGSWQLIF | GSGTQLTVM |
| 46 | | | AVIICTYT (SEQ ID | (SEQ ID | LLMK (SEQ ID | (SEQ ID | PGDSAVYF (SEQ ID NO: | (SEQ ID NO: | P (SEQ ID |
| | | | NO: 108) | NO: | NO: 6044) | NO: | 11980) | 25484) | NO: |
| | | | | 3010) | | 8946) | | | 16400) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQAGNYAEQFF | GPGTRLTVL |
| 47 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25485) | NO: |
| | | | | 2983) | | 8999) | | | 16327) |
| Clonotype_ | 1 | TRA | GDSVTQTEGQVALSEEDF | ASGYPA | LFWYVQYPGEGPQ | ASRDKEK | GSSRGFEATYNKEATSFHLQKASVQ | CALSAMTNSAGNKL | GIGTRVLVR |
| 47 | | | LTIHCNYS (SEQ ID | (SEQ ID | FLFR (SEQ ID | (SEQ ID | ESDSAVYY (SEQ ID NO: 12094) | TF (SEQ ID NO: | P (SEQ ID |
| | | | NO: 14) | NO: | NO: 6050) | NO: 8918) | | 25486) | NO: 16580) |
| | | | | 3082) | | | | | |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQAGTGDAEQF | GPGTRLTVL |
| 48 | | | SLECEQH (SEQ ID NO: | (SEQ ID | LMFL (SEQ ID | (SEQ ID | AVDPEDSAVYF (SEQ ID NO: | F (SEQ ID NO: | (SEQ ID |
| | | | 95) | NO: | NO: 6031) | NO: | 11967) | 25487) | NO: |
| | | | | 2983) | | 8999) | | | 16327) |
| Clonotype_ | 1 | TRA | GQQVQQSPASLVLQEGEN | TSLNS | MQWFYQRPEGSLV | NPSGT | KQSGRLTSTTVIKERRSSLHISSSQ | CASPF (SEQ ID | GDGTQLVVK |
| 48 | | | AELQCNFS (SEQ ID | (SEQ ID | SLFY (SEQ ID | (SEQ ID | ITDSGTYL (SEQ ID NO: | NO: 25488) | P (SEQ ID |
| | | | NO: 10) | NO: | NO: 6192) | NO: | 12038) | | NO: |
| | | | | 2978) | | 8914) | | | 16328) |
| Clonotype_ | 1 | TRB | NTKITQSPRYLILGRANK | LGHNA | MYWYKQSAEKPPE | YNLKQL | IRNETVPSRFIPECPDSSKILLHIS | CASSQGETLYF | GSGTRLTVL |
| 49 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25489) | NO: |
| | | | | 2983) | | 8999) | | | 16325) |
| Clonotype_ | 1 | TRA | GDSVTQTEGQVALSEEDF | ASGYPT | LFWYVQYPGEGPQ | ASRDKEK | GSSRGFEATYDKGTTSFHLRKASVQ | CALSGDYANKMIF | GLGTILRVR |
| 49 | | | LTIHCNYS (SEQ ID | (SEQ ID | LLFR (SEQ ID | (SEQ ID | ESDSAVYY (SEQ ID NO: | (SEQ ID NO: | P (SEQ ID |
| | | | NO: 14) | NO: | NO: 5950) | NO: | 11886) | 25490) | NO: |
| | | | | 2982) | | 8918) | | | 16416) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSQGGQAPLF | GEGTRLSVL |
| 50 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25491) | NO: |
| | | | | 2983) | | 8999) | | | 16441) |
| Clonotype_ | 1 | TRA | SQLAEENLWALSVHEGES | TSITA | LQWYRQKSGEGPA | IRSNERE | KRNGRLRATLDTSSQSSSLSITATR | CAIASSSFSKLVF | GQGTSLSVV |
| 50 | | | VTVNCSYK (SEQ ID | (SEQ ID | QLIL (SEQ ID | (SEQ ID | CEDTAVYF (SEQ ID NO: | (SEQ ID NO: | P (SEQ ID |
| | | | NO: 190) | NO: | NO: 5960) | NO: | 11896) | 15566) | NO: |
| | | | | 2992) | | 8928) | | | 16422) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSPTGISNERLF | GHGTKLSVL |
| 51 | | | SLECEQH (SEQ ID NO: | (SEQ ID | LMFL (SEQ ID | (SEQ ID | AVDPEDSAVYF (SEQ ID NO: | F (SEQ ID NO: | (SEQ ID |
| | | | 95) | NO: | NO: 6031) | NO: | 11967) | 25493) | NO: |
| | | | | 2983) | | 8999) | | | 16401) |
| Clonotype_ | 1 | TRA | GQQVQQSPASLVLQEGEN | TSLNS | MQWFYQRPGGSLV | NPSGT | KHSGRLTSTTVIKERRSSLHISSSQ | CALAPSNTNKVVF | GTGTRLQVL |
| 51 | | | AELQCNFS (SEQ ID | (SEQ ID | SLFY (SEQ ID | (SEQ ID | TTDSGTYL (SEQ ID NO: 11928) | (SEQ ID NO: | P (SEQ ID |
| | | | NO: 10) | NO: 2978) | NO: 5946) | NO: 8914) | | 25494) | NO: |
| | | | | | | | | | 16698) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSPRQTNTGQLY | GEGSKLTVL |
| 52 | | | SLECEQH (SEQ ID NO: | (SEQ ID | LMFL (SEQ ID | (SEQ ID | AVDPEDSAVYF (SEQ ID NO: | F (SEQ ID NO: | (SEQ ID |
| | | | 95) | NO: | NO: 6031) | NO: | 11967) | 25495) | NO: |
| | | | | 2983) | | 8999) | | | 16347) |
| Clonotype_ | 1 | TRA | GDSVTQTEGLVTLTEGLP | STYSPF | LFWYVQHLNEAPK | SFTDNKR | PEHQGFHATLHKSSSSFHLQKSSAQ | CALWGSGGKLTL | GAGTRLQVN |
| 52 | | | VMLNCTYQ (SEQ ID | (SEQ ID | LLLK (SEQ ID | (SEQ ID | LSDSALYY (SEQ ID NO: | (SEQ ID NO: | L (SEQ ID |
| | | | NO: 46) | NO: 3040) | NO: 5944) | NO: 8950) | 11944) | 25496) | NO: |
| | | | | | | | | | 17020) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSPGTGPNERLF | GHGTKLSVL |
| 53 | | | SLECEQH (SEQ ID NO: | (SEQ ID | LMFL (SEQ ID | (SEQ ID | AVDPEDSAVYF (SEQ ID NO: | F (SEQ ID NO: | (SEQ ID |
| | | | 95) | NO: | NO: 6031) | NO: | 11967) | 25497) | NO: |
| | | | | 2983) | | 8999) | | | 16401) |
| Clonotype_ | 1 | TRA | QQQVRQSPQSLTVWEGGT | DSTFNY | FPWYQQFPGEGPA | ILSVSDK | KEDGRFTTFFNKREKKLSLHIIDSQ | CAASAPGGYKVVF | GSGTRLLVS |
| 53 | | | TVLTCSYE (SEQ ID | (SEQ ID | LLIS (SEQ ID | (SEQ ID | PGDSATYF (SEQ ID NO: | (SEQ ID NO: | P (SEQ ID |
| | | | NO: 374) | NO: | NO: 6004) | NO: | 12246) | 25498) | NO: |
| | | | | 3036) | | 9200) | | | 16354) |
| Clonotype_ | 1 | TRB | NTKITQSPRYLILGRANK | LGHNA | MYWYKQSAEKPPE | YNLKQL | IRNETVPSRFIPECPDSSKLLLHIS | CASSPGLGDEQYF | GPGTRLTVL |
| 54 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: 8999) | 11967) | 25499) | NO: 16327) |
| | | | | 2983) | | | | | |
| Clonotype_ | 1 | TRA | AQSVTQPDARVTVSEGAS | SSVTPY | LFWYVQYPRQGLQ | YYSGDPV | GVNGFEAEFSKSNSSFHLRKASVH | CAAQQGTGSKLSF | GKGAKLTVS |
| 54 | | | LQLRCKYS (SEQ ID | (SEQ ID | LLLK (SEQ ID | V (SEQ | WSDSAVYF (SEQ ID NO: | (SEQ ID NO: | P (SEQ ID |
| | | | NO: 12) | NO: | NO: 5948) | ID NO: | 11892) | 25500) | NO: |
| | | | | 2988) | | 8916) | | | 16524) |
| Clonotype_ | 1 | TRB | NTKITQSPRYLILGRANK | LGHNA | MYWYKQSAEKPPE | YNLKQL | IRNETVPSRFIPECPDSSKILLHIS | CASGTGGVEQYF | GPGTRLTVL |
| 55 | | | SLECEQH (SEQ ID NO: | (SEQ ID | LMFL (SEQ ID | (SEQ ID | AVDPEDSAVYF (SEQ ID NO: | (SEQ ID NO: | (SEQ ID |
| | | | 95) | NO: | NO: 6031) | NO: | 11967) | 25501) | NO: |
| | | | | 2983) | | 8999) | | | 16327) |
| Clonotype_ | 1 | TRA | GQQVQQSPASLVLQEGEN | TSLNS | SLFY (SEQ ID | NPSGT | KQSGRLTSTTVIKERRSSLHISSSQ | CAMELGGADRLTF | GKGTQLIIQ |
| 55 | | | AELQCNFS (SEQ ID | (SEQ ID | NO: 5946) | (SEQ ID | TTDSGTYL (SEQ ID NO: 11882) | (SEQ ID NO: | P (SEQ ID |
| | | | NO: 10) | NO: | MQWFYQRPGGSLV | NO: | | 25502) | NO: |
| | | | | 2978) | | 8914) | | | 16360) |
| Clonotype_ | 1 | TRB | NAGVIQTPRHKVTGKGQE | SGHSA | VFWYRQTIVQGLE | FRNQAP | IDDSGMPKERFSAQMPNQSHSTLKI | CASSLDASQNTLYF | GAGTRLSVL |
| 56 | | | ATLWCEPI (SEQ ID | (SEQ ID | FLTY (SEQ ID | (SEQ ID | (SEQSTQPQDSAVYL (SEQ ID NO: | (SEQ ID NO: | (SEQ ID |
| | | | NO: 17) | NO: | NO: 5953) | NO: | 11889) | 25503) | NO: |
| | | | | 2985) | | 8921) | | | 16365) |
| Clonotype_ | 1 | TRA | GQQVQQSPSSLVLQEGEN | STATQ | LQWFYQSPGGSLV | NPSGT | KHTGRLTSTTVTKERRSSLHISSSQ | CAIDPITGSGGKLT | GTGTRLQVN |
| 56 | | | AELQCNFS (SEQ ID | (SEQ ID | SLLS (SEQ ID | (SEQ ID | TTDSGTYL (SEQ ID NO: | L (SEQ ID NO: | L (SEQ ID |
| | | | NO: 322) | NO: | NO: 6258) | NO: | 12194) | 25504) | NO: |
| | | | | 3290) | | 8914) | | | 16448) |
| Clonotype_ | 1 | TRB | NAGVIQTPRHKVTGKGQE | SGHSA | VFWYRQTIVQGLE | FRNQAP | IDDSGMPKERFSAQMPNQSHSTLKI | CASSLAGDEQYF | GPGTRLTVL |
| 57 | | | ATLWCEPI (SEQ ID | (SEQ ID | FLTY (SEQ ID | (SEQ ID | (SEQSTQPQDSAVYL (SEQ ID NO: | (SEQ ID NO: | (SEQ ID |
| | | | NO: 17) | NO: 2985) | NO: 5953) | NO: 8921) | 11889) | 25505) | NO: |
| | | | | | | | | | 16327) |
| Clonotype_ | 1 | TRA | AQSVTQPDARVTVSEGAS | YSATPY | ILFWYVQYPRQGLQ | YYSGDPV | GVNGFEAEFSKSNSSFHLRKASVH | CAVRDYANKMIF | GLGTILRVR |
| 57 | | | LQLRCKYS (SEQ ID | (SEQ ID | LLLK (SEQ ID | V (SEQ | WSDSAVYF (SEQ ID NO: 11892) | (SEQ ID NO: | P (SEQ ID |
| | | | NO: 12) | NO: | NO: 5948) | ID NO: | | 25506) | NO: 16416) |
| | | | | 2980) | | 8916) | | | |
| Clonotype_ | 1 | TRB | NAGVIQTPRHKVTGKGQE | SGHSA | VFWYRQTIVQGLE | FRNQAP | IDDSGMPKERFSAQMPNQSHSTLKI | CASSLAGGGSAETL | GSGTRLTVL |
| 58 | | | ATLWCEPI (SEQ ID | (SEQ ID | FLTY (SEQ ID | (SEQ ID | (SEQSTQPQDSAVYL (SEQ ID NO: | YF (SEQ ID NO: | (SEQ ID |
| | | | NO: 17) | NO: | NO: 5953) | NO: | 11889) | 25507) | NO: |
| | | | | 2985) | | 8921) | | | 16325) |
| Clonotype_ | 1 | TRA | GQQVQQSPASLVLQEGEN | TSLNS | MQWFYQRPEGSLV | NPSGT | KQSGRLTSTTVIKERRSSLHISSSQ | CAMDPGTGSNRLTF | GKGTKFSLI |
| 58 | | | AELQCNFS (SEQ ID | (SEQ ID | SLFY (SEQ ID | (SEQ ID | ITDSGTYL (SEQ ID NO: | (SEQ ID NO: | P (SEQ ID |
| | | | NO: 10) | NO: | NO: 6192) | NO: | 12038) | 25508) | NO: |
| | | | | 2978) | | 8914) | | | 16608) |
| Clonotype_ | 1 | TRB | NSKVIQTPRYLVKGQGQK | KGHPV | VFWYQQNKNNEFK | FQNQEV | ILQQIDMTEKRFSAECPSNSPCSLEI | CASSLRDDQDTQYF | GPGTRLLVL |
| 59 | | | AKMRCIPE (SEQ ID | (SEQ ID | FLIN (SEQ ID | (SEQ ID | (SEQSSEAGDSALYL (SEQ ID NO: | (SEQ ID NO: | (SEQ ID |
| | | | NO: 19409) | NO: | NO: 21849) | NO: | 24289) | 25509) | NO: |
| | | | | 20629) | | 23069) | | | 16341) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGSSNTDKVV | GTGTRLQVS |
| 59 | | | VTMDCVYE (SEQ ID | (SEQ ID | FLIR (SEQ ID | N (SEQ | IEDSAVYF (SEQ ID NO: | F (SEQ ID NO: | P (SEQ ID |
| | | | NO: 44) | NO: | NO: 5942) | ID NO: | 11878) | 25510) | NO: |
| | | | | 3056) | | 8910) | | | 16374) |
| Clonotype_ | 1 | TRB | SVLLYQKPNRDICQSGTS | SQVVS | MFWYQQFQEQSLM | ANEGSEA | TYESGFTKDKFPISRPNLTFSTLTV | CSSTGSYAEQFF | GPGTRLTVL |
| 60 | | | LKIQCVAD (SEQ ID | (SEQ ID | LMAT (SEQ ID | (SEQ ID | NNARPGDSSIYF (SEQ ID NO: | (SEQ ID NO: | (SEQ ID |
| | | | NO: 567) | NO: | NO: 6503) | NO: | 12439) | 25511) | NO: |
| | | | | 3535) | | 9471) | | | 16327) |
| Clonotype_ | 1 | TRA | GEQVEQRPPHLSVREGDS | DPNSYY | FFWYKQEPGAGLQ | VFSSTEI | NEGQGFTVLLNKKDKQLSLNLTAAH | CAVGLSNYNVLYF | GSGTKLTVE |
| 60 | | | AVIICTYT (SEQ ID | (SEQ ID | LLMK (SEQ ID | (SEQ ID | PGDSAVYF (SEQ ID NO: | (SEQ ID NO: | P (SEQ ID |
| | | | NO: 108) | NO: | NO: 6044) | NO: | 11980) | 25512) | NO: |
| | | | | 3010) | | 8946) | | | 16336) |
| Clonotype_ | 1 | TRB | SVLLYQKPNRDICQSGTS | SQVVS | LMAT (SEQ ID | ANEGSEA | TYESGFTKDKFPISRPNLTFSTLTV | CSSSEQFF (SEQ | GPGTRLTVL |
| 61 | | | LKIQCVAD (SEQ ID | (SEQ ID | NO: 6503) | (SEQ ID | NNARPGDSSIYF (SEQ ID NO: | ID NO: 25513) | (SEQ ID |
| | | | NO: 567) | NO: | MFWYQQFQEQSLM | NO: | 12439) | | NO: |
| | | | | 3535) | | 9471) | | | 16327) |
| Clonotype_ | 1 | TRA | GEQVEQRPPHLSVREGDS | DPNSYY | FFWYKQEPGASLQ | VFSSTEI | NEGQGFTVLINKKPTRLSLNLTAAH | CAVSGANTGKLTF | GHGTILRVH |
| 61 | | | AVITCTYT (SEQ ID | (SEQ ID | LLMK (SEQ ID | (SEQ ID | PGDSAAYF (SEQ ID NO: | (SEQ ID NO: | P (SEQ ID |
| | | | NO: 42) | NO: | NO: 5978) | NO: | 24294) | 25514) | NO: |
| | | | | 3010) | | 8946) | | | 16356) |
| Clonotype_ | 1 | TRB | SVLLYQKPNRDICQSGTS | SQVVS | MFWYQQFQEQSLM | ANEGSEA | TYESGFTKDKFPISRPNLTFSTLTV | CSSSEQFF (SEQ | GPGTRLTVL |
| 62 | | | LKIQCVAD (SEQ ID | (SEQ ID | LMAT (SEQ ID | (SEQ ID | NNARPGDSSIYF (SEQ ID NO: | ID NO: 25513) | (SEQ ID |
| | | | NO: 567) | NO: | NO: 6503) | NO: | 12439) | | NO: |
| | | | | 3535) | | 9471) | | | 16327) |
| Clonotype_ | 1 | TRA | GEQVEQRPPHLSVREGDS | DPNSYY | FFWYKQEPGASLQ | VFSSTEI | NEGQGFTVLLNKKDKRLSLNLTAAH | CAVSGANTGKLTF | GHGTILRVH |
| 62 | | | AVITCTYT (SEQ ID | (SEQ ID | LLMK (SEQ ID | (SEQ ID | PGDSAAYF (SEQ ID NO: | (SEQ ID NO: | P (SEQ ID |
| | | | NO: 42) | NO: | NO: 5978) | NO: | 11914) | 25514) | NO: |
| | | | | 3010) | | 8946) | | | 16356) |
| Clonotype_ | 1 | TRB | SVLLYQKPNRDICQSGTS | SQVVS | MFWYQQFQEQSLM | ANEGSEA | TYESGFTKDKFPISRPNLTFSTLTV | CSSSEGAEVFF | GKGTRLTVV |
| 63 | | | LKIQCVAD (SEQ ID | (SEQ ID | LMAT (SEQ ID | (SEQ ID | NNARPGDSSIYF (SEQ ID NO: | (SEQ ID NO: | (SEQ ID |
| | | | NO: 567) | NO: | NO: 6503) | NO: | 12439) | 25517) | NO: |
| | | | | 3535) | | 9471) | | | 16333) |
| Clonotype_ | 1 | TRA | AQSVTQPDARVTVSEGAS | YSGTPY | FWYVQYPRQGLQ | YYSGDPV | GVNGFEAEFSKSNSSFHLRKASVH | CVLSGGHKVVF | GTGTRLQVL |
| 63 | | | LQLRCKYS (SEQ ID | (SEQ ID | LLLK (SEQ ID | V (SEQ | WSDSAVYF (SEQ ID NO: | (SEQ ID NO: | P (SEQ ID |
| | | | NO: 12) | NO: | NO: 5948) | ID NO: | 11892) | 25518) | NO: |
| | | | | 3054) | | 8916) | | | 16698) |
| Clonotype_ | 1 | TRB | SVLLYQKPNRDICQSGTS | SQVVS | MFWYQQFQEQSLM | ANEGSEA | TYESGFTKDKFPISRPNLTFSTLTV | CSSSGGRDTLYF | GAGTRLSVL |
| 64 | | | LKIQCVAD (SEQ ID | (SEQ ID | LMAT (SEQ ID | (SEQ ID | NNARPGDSSIYF (SEQ ID NO: | (SEQ ID NO: | (SEQ ID |
| | | | NO: 567) | NO: | NO: 6503) | NO: | 12439) | 25519) | NO: |
| | | | | 3535) | | 9471) | | | 16365) |
| Clonotype_ | 1 | TRA | GEQVEQRPPHLSVREGDS | DPNSYY | FFWYKQEPGASLQ | VFSSTEI | NEGQGFTVLLNKKDKRLSLNLTAAH | CAVSNSAGNKLTF | GIGTRVLVR |
| 64 | | | AFITCTYT (SEQ ID | (SEQ ID | LLMK (SEQ ID | (SEQ ID | PGDSAAYF (SEQ ID NO: | (SEQ ID NO: | P (SEQ ID |
| | | | NO: 846) | NO: | NO: 5978) | NO: | 11914) | 25520) | NO: |
| | | | | 3010) | | 8946) | | | 16580) |
| Clonotype_ | 1 | TRB | SVLLYQKPNRDICQSGTS | SQVVS | MFWYQQFQEQSLM | ANEGSEA | TYESGFTKDKFPISRPNLTFSTLTV | CSSHRGEVFF (SEQ | GKGTRLTVV |
| 65 | | | LKIQCVAD (SEQ ID | (SEQ ID | LMAT (SEQ ID | (SEQ ID | NNARPGDSSIYF (SEQ ID NO: | ID NO: 25521) | (SEQ ID |
| | | | NO: 567) | NO: 3535) | NO: 6503) | NO: 9471) | 12439) | | NO: 16333) |
| Clonotype_ | 1 | TRA | GEQVEQRPPHLSVREGDS | DPNSYY | FFWYKQEPGASLQ | VFSSTEI | NEGQGFTVLLNKKDKRLSLNLTAAH | CAVSERYNQGKLIF | GQGTKLSIK |
| 65 | | | AVITCTYT (SEQ ID | (SEQ ID | LLMK (SEQ ID | (SEQ ID | PGDSAAYF (SEQ ID NO: | (SEQ ID NO: | P (SEQ ID |
| | | | NO: 42) | NO: | NO: 5978) | NO: | 11914) | 25522) | NO: |
| | | | | 3010) | | 8946) | | | 16332) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASTGGTRDTQYF | GPGTRLLVL |
| 66 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25523) | NO: |
| | | | | 2969) | | 8905) | | | 16341) |
| Clonotype_ | 1 | TRA | GDSVTQTEGLVTLTKGLP | TTYSPF | LFWYVQHLNEAPK | SSTDNKR | TEHQGFYATLHKSSSSFHLQKSSVQ | CALSENYNQGKLIF | GQGTKLSIK |
| 66 | | | VMLNCTYQ (SEQ ID | (SEQ ID | LLLK (SEQ ID | (SEQ ID | LSDSALYF (SEQ ID NO: | (SEQ ID NO: | P (SEQ ID |
| | | | NO: 8) | NO: | NO: 5944) | NO: | 11880) | 14848) | NO: |
| | | | | 2976) | | 8912) | | | 16332) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSPRDWGYEQYF | GPGTRLTVL |
| 67 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: 2969) | NO: 5937) | NO: 8905) | 11873) | 25525) | NO: |
| | | | | | | | | | 16327) |
| Clonotype_ | 1 | TRA | GDSVTQTEGQVTVSESKS | TTSIAYP | LLLK (SEQ ID | VITAGQK | GSSRGFEATYNKETTSFHLQKASVQ | CALVPMDYANKMIF | GLGTILRVR |
| 67 | | | LIINCTYS (SEQ ID | N (SEQ | NO: 5996) | (SEQ ID | ESDSAVYY (SEQ ID NO: | (SEQ ID NO: | P (SEQ ID |
| | | | NO: 60) | ID NO: | LFWYVRYPGEGLQ | NO: 8964) | 11932) | 25526) | NO: 16416) |
| | | | | 3028) | | | | | |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSPIEWGQGNTL | GEGSRLIVV |
| 68 | | | VSFWCDPI (SEQ ID | (SEQ ID | LLVY (SEQ ID | (SEQ ID | (SEQSAKQGDTATYL (SEQ ID NO: | YF (SEQ ID NO: | (SEQ ID |
| | | | NO: 1) | NO: | NO: 5937) | NO: | 11873) | 25527) | NO: |
| | | | | 2969) | | 8905) | | | 16471) |
| Clonotype_ | 1 | TRA | GDSVTQTEGPVTLSEGTS | TKQYPT | LFWYVQYPGEGPQ | VPKANEK | GSSRGFEATYNKEATSFHLQKASVQ | CALSEDSGYNKLTF | GKGTVLLVS |
| 68 | | | LTVNCSYE (SEQ ID | (SEQ ID | LLFK (SEQ ID | (SEQ ID | ESDSAVYY (SEQ ID NO: | (SEQ ID NO: | P (SEQ ID |
| | | | NO: 38) | NO: | NO: 5974) | NO: | 12094) | 25528) | NO: |
| | | | | 3006) | | 8942) | | | 16378) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSPPGQYYAEQF | GPGTRLTVL |
| 69 | | | VSFWCDPI (SEQ ID | (SEQ ID | LLVY (SEQ ID | (SEQ ID | (SEQSAKQGDTATYL (SEQ ID NO: | F (SEQ ID NO: | (SEQ ID |
| | | | NO: 1) | NO: | NO: 5937) | NO: | 11873) | 25529) | NO: |
| | | | | 2969) | | 8905) | | | 16327) |
| Clonotype_ | 1 | TRA | GQGVEQPDNLMSVEGTFA | TSGFNG | ILSWYQQREGHAPV | VVLDGL | KDSGHFSTFLSRSNGYSYLLLTELQ | CAVSTASLGKLQF | GTGTQVVVT |
| 69 | | | RVNCTYS (SEQ ID NO: | (SEQ ID | FLSY (SEQ ID | (SEQ ID | IKDSASYL (SEQ ID NO: 11876) | (SEQ ID NO: | P (SEQ ID |
| | | | 4) | NO: | NO: 5940) | NO: 8908) | | 25530) | NO: 16488) |
| | | | | 2972) | | | | | |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSLQGANTEVFF | GKGTRLTVV |
| 70 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25531) | NO: |
| | | | | 2969) | | 8905) | | | 16333) |
| Clonotype_ | 1 | TRA | GDSVTQTEGLVTLTEGLP | STYSPF | LFWYVQHLNEAPK | SFTDNKR | PEHQGFHATLHKSSSSFHLQKSSAQ | CALTHSSNTNKVVF | GTGTRLQVL |
| 70 | | | VMLNCTYQ (SEQ ID | (SEQ ID | LLLK (SEQ ID | (SEQ ID | LSDSALYY (SEQ ID NO: | (SEQ ID NO: | P (SEQ ID |
| | | | NO: 46) | NO: | NO: 5944) | NO: | 11944) | 25532) | NO: |
| | | | | 3040) | | 8950) | | | 16698) |
| Clonotype_ | 1 | TRB | JEAGVTQSPRYAVLQEGQA | SGHDT | LYWYQQPRDQGPQ | FRDEAV | IDNSQLPSDRFSAVRPKGTNSTLKI | CASSLPAETLYF | GSGTRLTVL |
| 71 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25533) | NO: |
| | | | | 2969) | | 8905) | | | 16325) |
| Clonotype_ | 1 | TRA | AQSVTQPDARVTVSEGAS | SSGTPY | ILFWYVQYPRQGLQ | YYSGDPV | GVNGFEAEFSKSNSSFHLRKASVH | CVLRASSSGQKLVF | GQGTILKVY |
| 71 | | | LQLRCKYS (SEQ ID | (SEQ ID | LLLK (SEQ ID | V (SEQ | WSDSAVYF (SEQ ID NO: | (SEQ ID NO: | L (SEQ ID |
| | | | NO: 12) | NO: | NO: 5948) | ID NO: | 11892) | 25534) | NO: |
| | | | | 2998) | | 8916) | | | 16414) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSDWGSYEQYF | GPGTRLTVL |
| 72 | | | VSFWCDPI (SEQ ID | (SEQ | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25535) | NO: |
| | | | | 2969) | | 8905) | | | 16327) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAASGYGSSGNKLI | GIGTLLSVK |
| 72 | | | ASLNCTSS (SEQ ID | (SEQ ID | ALMS (SEQ ID | (SEQ ID | PSDSALYF (SEQ ID NO: | F (SEQ ID NO: | P (SEQ ID |
| | | | NO: 64) | NO: 2970) | NO: 5938) | NO: 8906) | 11874) | 25536) | NO: |
| | | | | | | | | | 16456) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSPGTGRNTEVF | GKGTRLTVV |
| 73 | | | VSFWCDPI (SEQ ID | (SEQ ID | LLVY (SEQ ID | (SEQ ID | (SEQSAKQGDTATYL (SEQ ID NO: | F (SEQ ID NO: | (SEQ ID |
| | | | NO: 1) | NO: | NO: 5937) | NO: | 11873) | 25537) | NO: |
| | | | | 2969) | | 8905) | | | 16333) |
| Clonotype_ | 1 | TRA | GDSVTQTEGQVALSEEDF | ASGYPA | LFWYVQYPGEGPQ | ASRDKEK | GSSRGFEATYNKEATSFHLQKASVQ | CALSGSSSFSKLVF | GQGTSLSVV |
| 73 | | | LTIHCNYS (SEQ ID | (SEQ ID | FLFR (SEQ ID | (SEQ ID | ESDSAVYY (SEQ ID NO: 12094) | (SEQ ID NO: | P (SEQ ID |
| | | | NO: 14) | NO: 3082) | NO: 6050) | NO: 8918) | | 25538) | NO: 16422) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSSGTDSYEQYF | GPGTRLTVL |
| 74 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25539) | NO: |
| | | | | 2969) | | 8905) | | | 16327) |
| Clonotype_ | 1 | TRA | QQNVQQSPESLIVPEGAR | DSASQY | FWWYRQHSGKAPK | IFSNGE | KEEGRFTIHLNKASLHFSLHIRDSQ | CAVKAYQGGRALIF | GTGTTVSVS |
| 74 | | | TSLNCTFS (SEQ ID | (SEQ ID | ALMS (SEQ ID | (SEQ ID | PSDSALYL (SEQ ID NO: | (SEQ ID NO: | P (SEQ ID |
| | | | NO: 76) | NO: | NO: 6012) | NO: | 11948) | 25540) | NO: |
| | | | | 3044) | | 8932) | | | 16348) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSSGTANTEVFF | GKGTRLTVV |
| 75 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25541) | NO: |
| | | | | 2969) | | 8905) | | | 16333) |
| Clonotype_ | 1 | TRA | GDSVTQTEGLVTLTKGLP | TTYSPF | ILFWYVQHLNEAPK | SSTDNKR | TEHQGFYATLHKSSSSFHLQKSSVQ | CALRNGGSGNKLIF | GTGTLLSVK |
| 75 | | | VMLNCTYQ (SEQ ID | (SEQ ID | LLLK (SEQ ID | (SEQ ID | LSDSALYF (SEQ ID NO: | (SEQ ID NO: | P (SEQ ID |
| | | | NO: 8) | NO: | NO: 5944) | NO: | 11880) | 25542) | NO: |
| | | | | 2976) | | 8912) | | | 16350) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSLRSYEQYF | GPGTRLTVL |
| 76 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25543) | NO: |
| | | | | 2969) | | 8905) | | | 16327) |
| Clonotype_ | 1 | TRA | QQQVRQSPQSLTVWEGET | DSTFNY | FPWYQQFPGEGPA | IRSVSDK | KEDGRFTIFFNKREKKLSLHITDSQ | CAAASNYNVLYF | GSGTKLTVE |
| 76 | | | AILNCSYE (SEQ ID | (SEQ ID | LLIS (SEQ ID | (SEQ ID | PGDSATYF (SEQ ID NO: | (SEQ ID NO: | P (SEQ ID |
| | | | NO: 68) | NO: | NO: 6004) | NO: | 11940) | 25544) | NO: |
| | | | | 3036) | | 8988) | | | 16336) |
| Clonotype_ | | TRB | EAGVTQSPRYAVLQEGQA | SGHDT | LYWYQQPRDQGPQ | FRDEAV | IDNSQLPSDRFSAVRPKGTNSTLKI | CASSLWGASAETLY | GSGTRLTVL |
| 77 | | | VSFWCDPI (SEQ ID | (SEQ ID | LLVY (SEQ ID | (SEQ ID | (SEQSAKQGDTATYL (SEQ ID NO: | F (SEQ ID NO: | (SEQ ID |
| | | | NO: 1) | NO: | NO: 5937) | NO: | 11873) | 25545) | NO: |
| | | | | 2969) | | 8905) | | | 16325) |
| Clonotype_ | 1 | TRA | GDSVTQTEGQVALSEEDF | ASGYPA | LFWYVQYPGEGPQ | ASRDKEK | GSSRGFEATYDKGTTSFHLRKASVQ | CALGDYNQGKLIF | GQGTKLSIK |
| 77 | | | LTIHCNYS (SEQ ID | (SEQ ID | FLFR (SEQ ID | (SEQ ID | ESDSAVYY (SEQ ID NO: 11886) | (SEQ ID NO: | P (SEQ ID |
| | | | NO: 14) | NO: | NO: 6050) | NO: | | 15964) | NO: |
| | | | | 3082) | | 8918) | | | 16332) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSFDGANTEVFF | GKGTRLTVV |
| 78 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25547) | NO: |
| | | | | 2969) | | 8905) | | | 16333) |
| Clonotype_ | 1 | TRA | GDSVTQTEGLVTVTEGLP | TTYLTIA | FFWYVQYLNEAPQ | SSTDNKR | TEHQGFHATLHKSSSSFHLQKSSAQ | CALRSGGSNYKLTF | GKGTLLTVT |
| 78 | | | VKLNCTYQ (SEQ ID | (SEQ ID | VLLK (SEQ ID | (SEQ ID | LSDSALYY (SEQ ID NO: | (SEQ ID NO: | P (SEQ ID |
| | | | NO: 116) | NO: | NO: 6052) | NO: | 11918) | 25548) | NO: |
| | | | | 3084) | | 8912) | | | 16642) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSFGGRTNERLF | GHGTKLSVL |
| 79 | | | VSFWCDPI (SEQ ID | (SEQ ID | LLVY (SEQ ID | (SEQ ID | (SEQSAKQGDTATYL (SEQ ID NO: | F (SEQ ID NO: | (SEQ ID |
| | | | NO: 1) | NO: | NO: 5937) | NO: | 11873) | 25549) | NO: |
| | | | | 2969) | | 8905) | | | 16401) |
| Clonotype_ | 1 | TRA | QQQVRQSSQSLTVWEGET | NSAFDY | FPWYQQFPGEGPA | IRSVSDK | KEDGRFTIFFNKREKNLSLHIKDSQ | CAASGTNYGSSGNK | GIGTLLSVK |
| 79 | | | AILNCSYE (SEQ ID | (SEQ ID | LLIA (SEQ ID | (SEQ ID | PGDSATYF (SEQ ID NO: | LIF (SEQ ID NO: | P (SEQ ID |
| | | | NO: 84) | NO: | NO: 6020) | NO: | 11956) | 25550) | NO: |
| | | | | 3052) | | 8988) | | | 16456) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSFGDTGNTLYF | GEGSRLIVV |
| 80 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25551) | NO: |
| | | | | 2969) | | 8905) | | | 16471) |
| Clonotype_ | 1 | TRA | GDSVTQTEGQVALSEEDF | ASGYPA | LFWYVQYPGEGPQ | ASRDKEK | GSSRGFEATYNKEATSFHLQKASVQ | CALSGATNAYKVIF | GKGTHLHVL |
| 80 | | | LTIHCNYS (SEQ ID | (SEQ ID | FLFR (SEQ ID | (SEQ ID | ESDSAVYY (SEQ ID NO: | (SEQ ID NO: | P (SEQ ID |
| | | | NO: 14) | NO: | NO: 6050) | NO: | 12094) | 25552) | NO: |
| | | | | 3082) | | 8918) | | | 16330) |
| Clonotype_ | 1 | TRB | EAGVTQSPRYAVLQEGQA | SGHDT | LYWYQQPRDQGPQ | FRDEAV | IDNSQLPSDRFSAVRPKGTNSTLKI | CASSFGDTQYF | GPGTRLLVL |
| 81 | | | VSFWCDPI (SEQ ID | (SEQ ID | LLVY (SEQ ID | (SEQ ID | (SEQSAKQGDTATYL (SEQ ID NO: | (SEQ ID NO: | (SEQ ID |
| | | | NO: 1) | NO: | NO: 5937) | NO: | 11873) | 25553) | NO: |
| | | | | 2969) | | 8905) | | | 16341) |
| Clonotype_ | 1 | TRA | GDSVTQTEGPVTLSEGTS | TKQYPT | LFWYVQYPGEGPQ | VPKANEK | GSNRGFEATYNKEATSFHLQKASVQ | CALSDEYQGGRALI | GTGTTVSVS |
| 81 | | | LTVNCSYE (SEQ ID | (SEQ ID | LLFK (SEQ ID | (SEQ ID | ESDSAVYY (SEQ ID NO: | F (SEQ ID NO: | P (SEQ ID |
| | | | NO: 38) | NO: 3006) | NO: 5974) | NO: 8942) | 11910) | 25554) | NO: 16348) |
| Clonotype_ | | TRB | EAGVTQSPRYAVLQEGQA | SGHDT | LYWYQQPRDQGPQ | FRDEAV | IDNSQLPSDRFSAVRPKGTNSTLKI | CASSFWGSSAETLY | GSGTRLTVL |
| 82 | | | VSFWCDPI (SEQ ID | (SEQ ID | LLVY (SEQ ID | (SEQ ID | (SEQSAKQGDTATYL (SEQ ID NO: | F (SEQ ID NO: | (SEQ ID |
| | | | NO: 1) | NO: | NO: 5937) | NO: | 11873) | 25555) | NO: |
| | | | | 2969) | | 8905) | | | 16325) |
| Clonotype_ | 1 | TRA | GQQVQQSPASLVLQEGEN | STATQ | LQWFYQSPGGSLV | NPSGT | KHTGRLTSTTVTKERRSSLHISSSQ | CYSSGSWQLIF | GSGTQLTVM |
| 82 | | | AELQCNFS (SEQ ID | (SEQ ID | SLLS (SEQ ID | (SEQ ID | TTDSGTYL (SEQ ID NO: 12194) | (SEQ ID NO: | P (SEQ ID |
| | | | NO: 10) | NO: | NO: 6258) | NO: 8914) | | 25556) | NO: 16400) |
| | | | | 3290) | | | | | |
| Clonotype_ | 1 | TRB | DMKVTQMPRYLIKRMGEN | MSHET | MYWYRQDPGLGLQ | SYDVDS | NSEGDIPKGYRVSRKKREHFSLILD | CASSGTDYEQYF | GPGTRLTVL |
| 83 | | | VLLECGQD (SEQ ID | (SEQ ID | LIYI (SEQ ID | (SEQ ID | SAKTNQTSVYF (SEQ ID NO: | (SEQ ID NO: | (SEQ ID |
| | | | NO: 163) | NO: | NO: 6099) | NO: | 12035) | 25557) | NO: |
| | | | | 3131) | | 9067) | | | 16327) |
| Clonotype_ | 1 | TRA | GEQVEQRPPHLSVPEGDS | IDSATAY | FYWYKQEPGAGLQ | VFSNVDR | KEEQGLTVLLNKKDKQLSLNLTAAH | CAVSAGKTGNTRKL | GLGTTLQVQ |
| 83 | | | AVIICTYT (SEQ ID | (SEQ ID | LLMS (SEQ ID | (SEQ ID | PGDSAVYF (SEQ ID NO: | IF (SEQ ID NO: | P (SEQ ID |
| | | | NO: 100) | NO: | NO: 6036) | NO: | 11972) | 25558) | NO: |
| | | | | 2990) | | 9004) | | | 16326) |
| Clonotype_ | 1 | TRB | DMKVTQMPRYLIKRMGEN | MSHET | MYWYRQDPGLGLQ | SYDVDS | NSEGDIPKGYRVSRKKREHFSLILD | CASSLGEANQAPLF | GEGTRLSVL |
| 84 | | | VLLECGQD (SEQ ID | (SEQ ID | LIYI (SEQ ID | (SEQ ID | SAKTNQTSVYF (SEQ ID NO: | (SEQ ID NO: | (SEQ ID |
| | | | NO: 163) | NO: | NO: 6099) | NO: | 12035) | 25559) | NO: |
| | | | | 3131) | | 9067) | | | 16441) |
| Clonotype_ | 1 | TRA | GDSVTQTEGPVTLSEGTS | TKQYPT | LFWYVQYPGEGPQ | VPKANEK | GSNRGFEATYNKEATSFHLQKASVQ | CALSVASSGSWQLI | GSGTQLTVM |
| 84 | | | LTVNCSYE (SEQ ID | (SEQ ID | LLFK (SEQ ID | (SEQ ID | ESDSAVYY (SEQ ID NO: | F (SEQ ID NO: | P (SEQ ID |
| | | | NO: 38) | NO: | NO: 5974) | NO: | 11910) | 25560) | NO: |
| | | | | 3006) | | 8942) | | | 16400) |
| Clonotype_ | 1 | TRB | DMKVTQMPRYLIKRMGEN | MSHET | MYWYRQDPGLGLQ | SYDVDS | NSEGDIPKGYRVSRKKREHFSLILD | CASSGTGGVQDTQY | GPGTRLLVL |
| 85 | | | VLLECGQD (SEQ ID | (SEQ ID | LIYI (SEQ ID | (SEQ ID | SAKTNQTSVYF (SEQ ID NO: | F (SEQ ID NO: | (SEQ ID |
| | | | NO: 163) | NO: | NO: 6099) | NO: | 12035) | 25561) | NO: |
| | | | | 3131) | | 9067) | | | 16341) |
| Clonotype_ | 1 | TRA | SQWGEENLQALSIQEGED | TYTTV | VQWYRQKSGKGPA | IRSNERE | KRSGRLRATLDTSSQSSSLSITGTL | CASMDSNYQLIW | GSGTKLIIK |
| 85 | | | VTMNCSYK (SEQ ID | (SEQ ID | QLIL (SEQ ID | (SEQ ID | ATDTAVYF (SEQ ID NO: | (SEQ ID NO: | P (SEQ ID |
| | | | NO: 80) | NO: | NO: 6586) | NO: | 12030) | 25562) | NO: |
| | | | | 3048) | | 8928) | | | 16372) |
| Clonotype_ | 1 | TRB | DMKVTQMPRYLIKRMGEN | MSHET | MYWYRQDPGLGLQ | SYDVDS | NSEGDIPKGYRVSRKKREHFSLILD | CASSLGGNNERLFF | GHGTKLSVL |
| 86 | | | VLLECGQD (SEQ ID | (SEQ ID | LIYI (SEQ ID | (SEQ ID | SAKTNQTSVYF (SEQ ID NO: | (SEQ ID NO: | (SEQ ID |
| | | | NO: 163) | NO: | NO: 6099) | NO: | 12035) | 25563) | NO: |
| | | | | 3131) | | 9067) | | | 16401) |
| Clonotype_ | 1 | TRA | QQQVRQSPQSLTVWEGET | NSAFDY | FPWYQQFPGEGPA | ILSVSNK | KEDGRFTIFFNKREKKLSLHIADSQ | CAASGNTGYQNFYF | GKGTSLTVI |
| 86 | | | AILNCSYE (SEQ ID | (SEQ ID | LLIS (SEQ ID | (SEQ ID | PGDSATYF (SEQ ID NO: | (SEQ ID NO: | P (SEQ ID |
| | | | NO: 68) | NO: | NO: 6004) | NO: | 11996) | 15714) | NO: |
| | | | | 3052) | | 9028) | | | 16366) |
| Clonotype_ | 1 | TRB | DMKVTQMPRYLIKRMGEN | MSHET | MYWYRQDPGLGLQ | SYDVDS | NSEGDIPKGYRVSRKKREHFSLILD | CASSLDRNTEVFF | GKGTRLTVV |
| 87 | | | VLLECGQD (SEQ ID | (SEQ ID | LIYI (SEQ ID | (SEQ ID | SAKTNQTSVYF (SEQ ID NO: | (SEQ ID NO: | (SEQ ID |
| | | | NO: 163) | NO: | NO: 6099) | NO: | 12035) | 25565) | NO: |
| | | | | 3131) | | 9067) | | | 16333) |
| Clonotype_ | 1 | TRA | GQQVKQSSPSLTVQEGGI | NDMFDY | FAWYKKYPDNSPT | VRSNVDK | REDGRLTVFLNKSGKHFSLHITASQ | CAAGAQDQGGSAKL | GEGTKLTVS |
| 87 | | | SILNCDYE (SEQ ID | (SEQ ID | LLIS (SEQ ID | (SEQ ID | PEDTAVYL (SEQ ID NO: | IF (SEQ ID NO: | S (SEQ ID |
| | | | NO: 248) | NO: | NO: 6014) | NO: | 12120) | 25566) | NO: |
| | | | | 3046) | | 8982) | | | 16384) |
| Clonotype_ | 1 | TRB | DMKVTQMPRYLIKRMGEN | MSHET | MYWYRQDPGLGLQ | SYDVDS | NSEGDIPKGYRVSRKKREHFSLILD | CASSFGLEVFF | GKGTRLTVV |
| 88 | | | VLLECGQD (SEQ ID | (SEQ ID | LIYI (SEQ ID | (SEQ ID | SAKTNQTSVYF (SEQ ID NO: | (SEQ ID NO: | (SEQ ID |
| | | | NO: 163) | NO: | NO: 6099) | NO: | 12035) | 25567) | NO: |
| | | | | 3131) | | 9067) | | | 16333) |
| Clonotype_ | 1 | TRA | DAKTTQPDSMESTEGETV | TISGNEY | YVTH (SEQ ID | GLQQN | TTNSMAFLAIASDRKSSTLILPHVS | CILRVQGGSAKLIF | GEGTKLTVS |
| 88 | | | HLPCSHA (SEQ ID NO: | (SEQ ID | IYWYRQVPLQGPE | (SEQ ID | LRDAAVYH (SEQ ID NO: | (SEQ ID NO: | S (SEQ ID |
| | | | 104) | NO: | NO: 6040) | NO: | 12266) | 25568) | NO: |
| | | | | 3072) | | 9008) | | | 16384) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQTRDNYNSPL | AAGTRLTVT |
| 89 | | | KILICEQY (SEQ ID | (SEQ ID | FMFS (SEQ ID | (SEQ ID | ALKPDDSATYF (SEQ ID NO: | YF (SEQ ID NO: | (SEQ ID |
| | | | NO: 15) | NO: | NO: 5951) | NO: 8919) | 11887) | 25569) | NO: 16381) |
| | | | | 2983) | | | | | |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGTNTDKVVF | GTGTRLQVS |
| 89 | | | VTMDCVYE (SEQ ID | (SEQ ID | FLIR (SEQ ID | N (SEQ | IEDSAVYF (SEQ ID NO: | (SEQ ID NO: | P (SEQ ID |
| | | | NO: 44) | NO: | NO: 5942) | ID NO: | 11878) | 25570) | NO: |
| | | | | 3056) | | 8910) | | | 16374) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQEPGLGGIDT | GPGTRLLVL |
| 90 | | | KILICEQY (SEQ ID | (SEQ ID | FMFS (SEQ ID | (SEQ ID | ALKPDDSATYF (SEQ ID NO: | YF (SEQ ID NO: | (SEQ ID |
| | | | NO: 15) | NO: | NO: 5951) | NO: | 11887) | 25571) | NO: |
| | | | | 2983) | | 8919) | | | 16341) |
| Clonotype_ | 1 | TRA | GDSVTQTEGQVTVSESKS | TTSIAYP | LFWYVRYPGEGLQ | VITAGQK | GSSRGFEATYNKETTSFHLQKASVQ | CALGPSNNRIFF | GDGTQLVVK |
| 90 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 60) | ID NO: | NO: 5996) | NO: | 11932) | 25572) | NO: |
| | | | | 3028) | | 8964) | | | 16328) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQEWVNQDTQY | GPGTRLLVL |
| 91 | | | KILICEQY (SEQ ID | (SEQ ID | FMFS (SEQ ID | (SEQ ID | ALKPDDSATYF (SEQ ID NO: | F (SEQ ID NO: | (SEQ ID |
| | | | NO: 15) | NO: | NO: 5951) | NO: 8919) | 11887) | 25573) | NO: 16341) |
| | | | | 2983) | | | | | |
| Clonotype_ | 1 | TRA | GQQVQQSPTSLVLQEGEN | TSLNS | MQWFYQRPGGSLV | NPSGT | ITDSGTYL (SEQ ID NO: 12038) | CAMERFGTGYQNFY | GKGTSLTVI |
| 91 | | | AELQCNFS (SEQ ID | (SEQ ID | SVFY (SEQ ID | (SEQ ID | KQSGRLTSTTVIKERRSSLHISSSQ | F (SEQ ID NO: | P (SEQ ID |
| | | | NO: 528) | NO: 2978) | NO: 6464) | NO: 8914) | | 25574) | NO: |
| | | | | | | | | | 16366) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQDNSAETLYF | GSGTRLTVL |
| 92 | | | KILICEQY (SEQ ID | (SEQ ID | FMFS (SEQ ID | (SEQ ID | ALKPDDSATYF (SEQ ID NO: | (SEQ ID NO: | (SEQ ID |
| | | | NO: 15) | NO: | NO: 5951) | NO: | 11887) | 25575) | NO: |
| | | | | 2983) | | 8919) | | | 16325) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGGNAGAKLT | GGGTRLTVR |
| 92 | | | VTMDCVYE (SEQ ID | (SEQ ID | FLIR (SEQ ID | N (SEQ | IEDSAVYF (SEQ ID NO: 11878) | F (SEQ ID NO: | P (SEQ ID |
| | | | NO: 44) | NO: | NO: 5942) | ID NO: | | 25576) | NO: 16338) |
| | | | | 3056) | | 8910) | | | |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQDRGDTEVFF | GKGTRLTVV |
| 93 | | | KILICEQY (SEQ ID | (SEQ ID | FMFS (SEQ ID | (SEQ ID | ALKPDDSATYF (SEQ ID NO: | (SEQ ID NO: | (SEQ ID |
| | | | NO: 15) | NO: | NO: 5951) | NO: | 11887) | 25577) | NO: |
| | | | | 2983) | | 8919) | | | 16333) |
| Clonotype_ | 1 | TRA | SQWGEENLQALSIQEGED | TYTTV | VQWYRQKSGKGPA | IRSNERE | KRSGRLRATLDTSSQSSSLSITGTL | CACLSGSFNKLTF | GAGTRLAVC |
| 93 | | | VTMNCSYK (SEQ ID | (SEQ ID | QLIL (SEQ ID | (SEQ ID | ATDTAVYF (SEQ ID NO: | (SEQ ID NO: | P (SEQ ID |
| | | | NO: 80) | NO: | NO: 6586) | NO: | 12030) | 25578) | NO: |
| | | | | 3048) | | 8928) | | | 16692) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQDHISNERLF | GHGTKLSVL |
| 94 | | | KILICEQY (SEQ ID | (SEQ ID | FMFS (SEQ ID | (SEQ ID | ALKPDDSATYF (SEQ ID NO: | F (SEQ ID NO: | (SEQ ID |
| | | | NO: 15) | NO: | NO: 5951) | NO: | 11887) | 25579) | NO: |
| | | | | 2983) | | 8919) | | | 16401) |
| Clonotype_ | 1 | TRA | GDSVTQTEGPVTVSESES | ATSIAYP | LFWYVRYPGEGLQ | VITAGQK | GSSRGFEATYNKETTSFHLQKASVQ | CALGDRDYAQGLTF | GLGTRVSVF |
| 94 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 130) | ID NO: | NO: 5996) | NO: | 11932) | 25580) | NO: |
| | | | | 3098) | | 8964) | | | 16344) |
| Clonotype_ | 1 | TRB | DPKIIQKPKYLVAVTGSE | ILGHNA | MYWYRQSAKKPLE | YSYQKL | MDNQTASSRFQPQSSKKNHLDLQIT | CASSQDPFQDTQYF | GPGTRLLVL |
| 95 | | | KILICEQY (SEQ ID | (SEQ ID | FMFS (SEQ ID | (SEQ ID | ALKPDDSATYF (SEQ ID NO: | (SEQ ID NO: | (SEQ ID |
| | | | NO: 15) | NO: | NO: 5951) | NO: | 11887) | 25581) | NO: |
| | | | | 2983) | | 8919) | | | 16341) |
| Clonotype_ | 1 | TRA | GDSVTQKQGQVTLSEDDF | TTTYPT | LFWYVQYPGQGPQ | VTTANNK | GISRGFEATYDKGTTSFHLQKASVQ | CALVDDSGTYQRF | GTGTKLQVV |
| 95 | | | LFINCTYS (SEQ ID | (SEQ ID | LLLK (SEQ ID | (SEQ ID | ESDSAVYF (SEQ ID NO: 12382) | (SEQ ID NO: | P (SEQ ID |
| | | | NO: 510) | NO: | NO: 6446) | NO: 9054) | | 25582) | NO: 16432) |
| | | | | 3478) | | | | | |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQDGGARDSDY | GSGTRLLVI |
| 96 | | | KILICEQY (SEQ ID | (SEQ ID | FMFS (SEQ ID | (SEQ ID | ALKPDDSATYF (SEQ ID NO: | TF (SEQ ID NO: | (SEQ ID |
| | | | NO: 15) | NO: | NO: 5951) | NO: | 11887) | 25583) | NO: |
| | | | | 2983) | | 8919) | | | 16337) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAASDPMLSGSFNK | GAGTRLAVC |
| 96 | | | ASFNCTSS (SEQ ID | (SEQ ID | ALMS (SEQ ID | (SEQ ID | PSDSALYF (SEQ ID NO: | LTF (SEQ ID NO: | P (SEQ ID |
| | | | NO: 2) | NO: | NO: 5938) | NO: | 11874) | 25584) | NO: |
| | | | | 2970) | | 8906) | | | 16692) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQDQISNERLF | GHGTKLSVL |
| 97 | | | KILICEQY (SEQ ID | (SEQ ID | FMFS (SEQ ID | (SEQ ID | ALKPDDSATYF (SEQ ID NO: | F (SEQ ID NO: | (SEQ ID |
| | | | NO: 15) | NO: | NO: 5951) | NO: | 11887) | 25585) | NO: |
| | | | | 2983) | | 8919) | | | 16401) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAAFHDSGYNKLTF | GKGTVLLVS |
| 97 | | | ASLNCTSS (SEQ ID | (SEQ ID | ALMS (SEQ ID | (SEQ ID | PSDSALYF (SEQ ID NO: | (SEQ ID NO: | P (SEQ ID |
| | | | NO: 64) | NO: | NO: 5938) | NO: | 11874) | 25586) | NO: |
| | | | | 2970) | | 8906) | | | 16378) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQDVTGGYEQY | GPGTRLTVL |
| 98 | | | KILICEQY (SEQ ID | (SEQ ID | FMFS (SEQ ID | (SEQ ID | ALKPDDSATYF (SEQ ID NO: | F (SEQ ID NO: | (SEQ ID |
| | | | NO: 15) | NO: 2983) | NO: 5951) | NO: 8919) | 11887) | 25587) | NO: 16327) |
| Clonotype_ | | TRA | GDSVTQTEGQVTVSESKS | ATSIGYP | LFWYVRYPGEGLQ | VITAGQK | GSSRGFEATYNKEATSFHLQKASVQ | CALSWSSGSWQLIF | GSGTQLTVM |
| 98 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 60) | ID NO: | NO: 5996) | NO: 8964) | 12094) | 25588) | NO: 16400) |
| | | | | 3330) | | | | | |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQDWGQDAETL | GSGTRLTVL |
| 99 | | | KILICEQY (SEQ ID | (SEQ ID | FMFS (SEQ ID | (SEQ ID | ALKPDDSATYF (SEQ ID NO: | YF (SEQ ID NO: | (SEQ ID |
| | | | NO: 15) | NO: | NO: 5951) | NO: | 11887) | 25589) | NO: |
| | | | | 2983) | | 8919) | | | 16325) |
| Clonotype_ | 1 | TRA | GDSVTQTEGLVTLTEGLP | STYSPF | LFWYVQHLNEAPK | SFTDNKR | PEHQGFHATLHKSSSSFHLQKSSAQ | CALSDAGSGGKLTL | GAGTRLQVN |
| 99 | | | VMLNCTYQ (SEQ ID | (SEQ ID | LLLK (SEQ ID | (SEQ ID | LSDSALYY (SEQ ID NO: | (SEQ ID NO: | L (SEQ ID |
| | | | NO: 46) | NO: | NO: 5944) | NO: | 11944) | 25590) | NO: |
| | | | | 3040) | | 8950) | | | 17020) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQADWDNQDTQ | GPGTRLLVL |
| 100 | | | KILICEQY (SEQ ID | (SEQ ID | FMFS (SEQ ID | (SEQ ID | ALKPDDSATYF (SEQ ID NO: | YF (SEQ ID NO: | (SEQ ID |
| | | | NO: 15) | NO: 2983) | NO: 5951) | NO: 8919) | 11887) | 25591) | NO: 16341) |
| Clonotype_ | 1 | TRA | AQKVTQTQTSISVVEKTT | TRDSSYF | LFWYKQTASGEIV | QDSYKKE | ATVGHYSLNFQKPKSSIGLIITATQ | CAMRESSGGNYKPT | GKGTSLVVH |
| 100 | | | VTMDCVYE (SEQ ID | (SEQ ID | FLIR (SEQ ID | N (SEQ | IEDSAVYF (SEQ ID NO: | F (SEQ ID NO: | P (SEQ ID |
| | | | NO: 6) | NO: | NO: 5942) | ID NO: | 11878) | 25592) | NO: |
| | | | | 2974) | | 8910) | | | 16394) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSQGTNSDYTF | GSGTRLLVI |
| 101 | | | KILICEQY (SEQ ID | (SEQ ID | FMFS (SEQ ID | (SEQ ID | ALKPDDSATYF (SEQ ID NO: | (SEQ ID NO: | (SEQ ID |
| | | | NO: 15) | NO: | NO: 5951) | NO: | 11887) | 25593) | NO: |
| | | | | 2983) | | 8919) | | | 16337) |
| Clonotype_ | 1 | TRA | GEKVEQHESTLSVREGDS | DTASSY | FPWYKQEAGKSLH | IRSNVDR | KQSQRLTVLLDKKAKRFSLHITATQ | CAARSAGNKLTF | GIGTRVLVR |
| 101 | | | AVINCTYT (SEQ ID | ID NO: | FVID (SEQ ID | (SEQ ID | PEDSAIYF (SEQ ID NO: | (SEQ ID NO: | P (SEQ ID |
| | | | NO: 54) | 3022) | NO: 5990) | NO: | 12238) | 25594) | NO: |
| | | | | | | 8958) | | | 16580) |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASSPQIANTEVFF | GKGTRLTVV |
| 102 | | | KILICEQY (SEQ ID | (SEQ ID | FMFS (SEQ ID | (SEQ ID | ALKPDDSATYF (SEQ ID NO: | (SEQ ID NO: | (SEQ ID |
| | | | NO: 15) | NO: | NO: 5951) | NO: | 11887) | 25595) | NO: |
| | | | | 2983) | | 8919) | | | 16333) |
| Clonotype_ | 1 | TRA | AQSVTQPDARVTVSEGAS | YSATPY | ILFWYVQYPRQGLQ | YYSGDPV | GVNGFEAEFSKSNSSFHLRKASVH | CAVIGGGSAKLIF | GEGTKLTVS |
| 102 | | | LQLRCKYS (SEQ ID | (SEQ ID | LLLK (SEQ ID | V (SEQ | WSDSAVYF (SEQ ID NO: 11892) | (SEQ ID NO: | S (SEQ ID |
| | | | NO: 12) | NO: | NO: 5948) | ID NO: | | 25596) | NO: 16384) |
| | | | | 2980) | | 8916) | | | |
| Clonotype_ | 1 | TRB | DPKIIQKPKYLVAVTGSE | LGHNA | MYWYRQSAKKPLE | YSYQKL | MDNQTASSRFQPQSSKKNHLDLQIT | CASGLGVSAEQFF | GPGTRLTVL |
| 103 | | | KILICEQY (SEQ ID | (SEQ ID | FMFS (SEQ ID | (SEQ ID | ALKPDDSATYF (SEQ ID NO: | (SEQ ID NO: | (SEQ ID |
| | | | NO: 15) | NO: | NO: 5951) | NO: | 11887) | 25597) | NO: |
| | | | | 2983) | | 8919) | | | 16327) |
| Clonotype_ | 1 | TRA | AQKVTQTQTSISVMEKTT | TRDSSYF | LFWYKQTASGEIV | QDSYKKE | ATEGHYSLNFQKPKSSIGLIITATQ | CAMREGPGYQNFYF | GKGTSLTVI |
| 103 | | | VTMDCVYE (SEQ ID | (SEQ ID | FLIR (SEQ ID | N (SEQ | IEDSAVYF (SEQ ID NO: | (SEQ ID NO: | P (SEQ ID |
| | | | NO: 44) | NO: | NO: 5942) | ID NO: | 11916) | 25598) | NO: |
| | | | | 2974) | | 8910) | | | 16366) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASRQGAYAEQFF | GPGTRLTVL |
| 104 | | | VSFNCKQT (SEQ ID | (SEQ ID | IMFS (SEQ ID | (SEQ ID | SVEPEDSAVYL (SEQ ID NO: | (SEQ ID NO: | (SEQ ID |
| | | | NO: 99) | NO: | NO: 6035) | NO: | 11971) | 25599) | NO: |
| | | | | 3067) | | 9003) | | | 16327) |
| Clonotype_ | 1 | TRA | AQSVTQPDARVTVSEGAS | YFGTPY | LFWYVQYPRQGLQ | YYPGDPV | GVNGFEAEFSKSNSSFHLRKASVH | CAVSPLNQGGSAKL | GEGTKLTVS |
| 104 | | | LQLRCKYS (SEQ ID | (SEQ ID | LLLK (SEQ ID | V (SEQ | WSDWAVYF (SEQ ID NO: | IF (SEQ ID NO: | S (SEQ ID |
| | | | NO: 12) | NO: | NO: 5948) | ID NO: | 11924) | 25600) | NO: |
| | | | | 3020) | | 8956) | | | 16384) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSKQGYEQYF | GPGTRLTVL |
| 105 | | | VSFNCKQT (SEQ ID | (SEQ ID | IMFS (SEQ ID | (SEQ ID | SVEPEDSAVYL (SEQ ID NO: | (SEQ ID NO: | (SEQ ID |
| | | | NO: 99) | NO: | NO: 6035) | NO: | 11971) | 25601) | NO: |
| | | | | 3067) | | 9003) | | | 16327) |
| Clonotype_ | 1 | TRA | AQSVTQPDARVTVSEGAS | YSGTPY | LFWYVQYPRQGLQ | YYSGDPV | GVNGFEAEFIKSNSSFHLRKASVH | CAVSSYTGNYKYVF | GAGTRLKVI |
| 105 | | | LQLRCKYS (SEQ ID | (SEQ ID | LLLK (SEQ ID | V (SEQ | WSDSAVYF (SEQ ID NO: | (SEQ ID NO: | A (SEQ ID |
| | | | NO: 12) | NO: | NO: 5948) | ID NO: | 12016) | 25602) | NO: |
| | | | | 3054) | | 8916) | | | 16380) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSPTANSDYTF | GSGTRLLVI |
| 106 | | | VSFNCKQT (SEQ ID | (SEQ ID | IMFS (SEQ ID | (SEQ ID | SVEPEDSAVYL (SEQ ID NO: | (SEQ ID NO: | (SEQ ID |
| | | | NO: 99) | NO: | NO: 6035) | NO: | 11971) | 25603) | NO: |
| | | | | 3067) | | 9003) | | | 16337) |
| Clonotype_ | 1 | TRA | AQKVTQTQTSISVMEKTT | TQDSSYF | FLIR (SEQ ID | QDSYKKE | ATVGHYSLNFQKPKSSIGLIITATQ | CAMREGYAQGLTF | GLGTRVSVF |
| 106 | | | VTMDCVYE (SEQ ID | (SEQ ID | LFWYKQTASGEIV | N (SEQ | IEDSAVYF (SEQ ID NO: | (SEQ ID NO: | P (SEQ ID |
| | | | NO: 44) | NO: | NO: 5942) | ID NO: | 11878) | 15140) | NO: |
| | | | | 3056) | | 8910) | | | 16344) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSPWGGALDTQY | GPGTRLLVL |
| 107 | | | VSFNCKQT (SEQ ID | (SEQ ID | IMFS (SEQ ID | (SEQ ID | SVEPEDSAVYL (SEQ ID NO: | F (SEQ ID NO: | (SEQ ID |
| | | | NO: 99) | NO: | NO: 6035) | NO: | 11971) | 25605) | NO: |
| | | | | 3067) | | 9003) | | | 16341) |
| Clonotype_ | 1 | TRA | TQTVSQSDAHVTVFEGDS | YGGSIY | LSWYIQHHGRGLQ | YYSGNPV | GVNGFEAEFSKSDSSFHLRKASVH | CAVRSDTNAYKVIF | GKGTHLHVL |
| 107 | | | VELRCNYS (SEQ ID | (SEQ ID | FLLK (SEQ ID | V (SEQ | WSDSAVYF (SEQ ID NO: | (SEQ ID NO: | P (SEQ ID |
| | | | NO: 110) | NO: | NO: 6046) | ID NO: | 11946) | 25606) | NO: |
| | | | | 3078) | | 9014) | | | 16330) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSDYWGSSYEQY | GPGTRLTVL |
| 108 | | | VSFNCKQT (SEQ ID | (SEQ ID | IMFS (SEQ ID | (SEQ ID | SVEPEDSAVYL (SEQ ID NO: | F (SEQ ID NO: | (SEQ ID |
| | | | NO: 99) | NO: | NO: 6035) | NO: | 11971) | 25607) | NO: |
| | | | | 3067) | | 9003) | | | 16327) |
| Clonotype_ | 1 | TRA | GDSVTQKQGQVTLSEDDF | TTTYPT | LLWYVQYPGQGPQ | VTTANNK | GISRGFEATYDKGTTSFHLQKASVQ | CALVGGGNYKPTF | GKGTSLVVH |
| 108 | | | LFINCTYS (SEQ ID | (SEQ ID | LLLK (SEQ ID | (SEQ ID | ESDSAVYF (SEQ ID NO: | (SEQ ID NO: | P (SEQ ID |
| | | | NO: 510) | NO: | NO: 21948) | NO: | 12382) | 25608) | NO: |
| | | | | 3478) | | 9054) | | | 16394) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSADWGAGDTQY | GPGTRLLVL |
| 109 | | | VSFNCKQT (SEQ ID | (SEQ ID | IMFS (SEQ ID | (SEQ ID | SVEPEDSAVYL (SEQ ID NO: | F (SEQ ID NO: | (SEQ ID |
| | | | NO: 99) | NO: | NO: 6035) | NO: | 11971) | 25609) | NO: |
| | | | | 3067) | | 9003) | | | 16341) |
| Clonotype_ | 1 | TRA | GDSVTQKQGQVTLSEDDF | TTTYPT | ILFWYVQYPGQGPQ | VTTANNK | GISRGFEATYDKGTTSFHLQKASVQ | CALAQTSGGNYKPT | GKGTSLVVH |
| 109 | | | LFINCTYS (SEQ ID | (SEQ ID | LLLK (SEQ ID | (SEQ ID | ESDSAVYF (SEQ ID NO: | F (SEQ ID NO: | P (SEQ ID |
| | | | NO: 510) | NO: | NO: 6446) | NO: | 12382) | 25610) | NO: |
| | | | | 3478) | | 9054) | | | 16394) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSYTGGDSYEQY | GPGTRLTVL |
| 110 | | | VSFNCKQT (SEQ ID | (SEQ ID | IMFS (SEQ ID | (SEQ ID | SVEPEDSAVYL (SEQ ID NO: | F (SEQ ID NO: | (SEQ ID |
| | | | NO: 99) | NO: 3067) | NO: 6035) | NO: 9003) | 11971) | 25611) | NO: 16327) |
| Clonotype_ | 1 | TRA | GQQVQQSPASLVLQEGEN | TSLNS | MQWFYQRPGGSLV | NPSGT | KQSGRLTSTTVIKERRSSLHISSSQ | CAITSSGQKLVF | GQGTILKVY |
| 110 | | | AELQCNFS (SEQ ID | (SEQ ID | SLFY (SEQ ID | (SEQ ID | TTDSGTYL (SEQ ID NO: 11882) | (SEQ ID | L (SEQ ID |
| | | | NO: 10) | NO: | NO: 5946) | NO: | | NO: 25612) | NO: |
| | | | | 2978) | | 8914) | | | 16414) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSYRLGRDTGQL | GEGSKLTVL |
| 111 | | | VSFNCKQT (SEQ ID | (SEQ ID | IMFS (SEQ ID | (SEQ ID | SVEPEDSAVYL (SEQ ID NO: | YF (SEQ ID NO: | (SEQ ID |
| | | | NO: 99) | NO: | NO: 6035) | NO: | 11971) | 25613) | NO: |
| | | | | 3067) | | 9003) | | | 16347) |
| Clonotype_ | 1 | TRA | GDSVTQTEGLVTLTEGLP | TAYSTF | LFWYVQHLNEAPK | SSTDNKR | TEHQGFHATLHKSSSSFHLQKSSAQ | CALWTGNYKYVF | GAGTRLKVI |
| 111 | | | VMLNCTYQ (SEQ ID | (SEQ ID | LLLK (SEQ ID | (SEQ ID | LSDSALYY (SEQ ID NO: | (SEQ ID NO: | A (SEQ ID |
| | | | NO: 46) | NO: 3030) | NO: 5944) | NO: 8912) | 11918) | 25614) | NO: |
| | | | | | | | | | 16380) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSWTKDTQYF | GPGTRLLVL |
| 112 | | | VSFNCKQT (SEQ ID | (SEQ ID | IMFS (SEQ ID | (SEQ ID | SVEPEDSAVYL (SEQ ID NO: | (SEQ ID NO: | (SEQ ID |
| | | | NO: 99) | NO: | NO: 6035) | NO: | 11971) | 25615) | NO: |
| | | | | 3067) | | 9003) | | | 16341) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMRETSGGNYKPT | GKGTSLVVH |
| 112 | | | VTMDCVYE (SEQ ID | (SEQ ID | FLIR (SEQ ID | N (SEQ | IEDSAVYF (SEQ ID NO: 11878) | F (SEQ ID NO: | P (SEQ ID |
| | | | NO: 44) | NO: 3056) | NO: 5942) | ID NO: | | 25616) | NO: 16394) |
| | | | | | | 8910) | | | |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSFDWGAGDTQY | GPGTRLLVL |
| 113 | | | VSFNCKQT (SEQ ID | (SEQ ID | IMFS (SEQ ID | (SEQ ID | SVEPEDSAVYL (SEQ ID NO: | F (SEQ ID NO: | (SEQ ID |
| | | | NO: 99) | NO: | NO: 6035) | NO: | 11971) | 25617) | NO: |
| | | | | 3067) | | 9003) | | | 16341) |
| Clonotype_ | 1 | TRA | GQQVQQSPTSLVLQEGEN | TSLNS | MQWFYQRPGGSLI | NPSGT | KQSGRLTSTTVIKERRSSLHISSSQ | CAMEATSSGQKLVF | GQGTILKVY |
| 113 | | | AELQCNFS (SEQ ID | (SEQ ID | SVFY (SEQ ID | (SEQ ID | ITDSGTYL (SEQ ID NO: | (SEQ ID NO: | L (SEQ ID |
| | | | NO: 528) | NO: | NO: 6710) | NO: | 12038) | 25618) | NO: |
| | | | | 2978) | | 8914) | | | 16414) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASRTGGIGEQYF | GPGTRLTVL |
| 114 | | | VSFNCKQT (SEQ ID | (SEQ ID | IMFS (SEQ ID | (SEQ ID | SVEPEDSAVYL (SEQ ID NO: | (SEQ ID NO: | (SEQ ID |
| | | | NO: 99) | NO: | NO: 6035) | NO: | 11971) | 25619) | NO: |
| | | | | 3067) | | 9003) | | | 16327) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAASEHLWGNYGNE | GAGTKLTIK |
| 114 | | | ASFNCTSS (SEQ ID | (SEQ ID | ALMS (SEQ ID | (SEQ ID | PSDSALYF (SEQ ID NO: | KITF (SEQ ID | P (SEQ ID |
| | | | NO: 2) | NO: | NO: 5938) | NO: | 11874) | NO: 25620) | NO: |
| | | | | 2970) | | 8906) | | | 16512) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASSPGLGDYEQYF | GPGTRLTVL |
| 115 | | | VSFNCKQT (SEQ ID | (SEQ ID | IMFS (SEQ ID | (SEQ ID | SVEPEDSAVYL (SEQ ID NO: | (SEQ ID NO: | (SEQ ID |
| | | | NO: 99) | NO: | NO: 6035) | NO: | 11971) | 25621) | NO: |
| | | | | 3067) | | 9003) | | | 16327) |
| Clonotype_ | 1 | TRA | GEKVEQHESTLSVREGDS | DTASSY | FPWYKQEAGKGLH | IRSNVDR | KQSQRLIVLLDKKAKRFSLHITATQ | CAAPGGYKVVF | GSGTRLLVS |
| 115 | | | AVINCTYT (SEQ ID | (SEQ ID | FVID (SEQ ID | (SEQ ID | PEDSAIYF (SEQ ID NO: | (SEQ ID NO: | P (SEQ ID |
| | | | NO: 54) | NO: | NO: 5994) | NO: | 11926) | 25622) | NO: |
| | | | | 3022) | | 8958) | | | 16354) |
| Clonotype_ | 1 | TRB | ETAVFQTPNYHVTQVGNE | LGHDT | MYWYKQDSKKLLK | YNNKQL | IVNETVPRRFSPQSSDKAHLNLRIK | CASVGFSYEQYF | GPGTRLTVL |
| 116 | | | VSFNCKQT (SEQ ID | (SEQ ID | IMFS (SEQ ID | (SEQ ID | SVEPEDSAVYL (SEQ ID NO: | (SEQ ID NO: | (SEQ ID |
| | | | NO: 99) | NO: | NO: 6035) | NO: | 11971) | 25623) | NO: |
| | | | | 3067) | | 9003) | | | 16327) |
| Clonotype_ | 1 | TRA | GDSVTQTEGLVTLTEGLP | TAYSTF | LFWYVQHLNEAPK | SSTDNKR | TEHQGFHATLHKSSSSFHLQKSSAQ | CALSDRDNNAPRF | GAGTKLTVK |
| 116 | | | VMLNCTYQ (SEQ ID | (SEQ ID | LLLK (SEQ ID | (SEQ ID | LSDSALYY (SEQ ID NO: | (SEQ ID NO: | P (SEQ ID |
| | | | NO: 46) | NO: | NO: 5944) | NO: | 11918) | 25624) | NO: |
| | | | | 3030) | | 8912) | | | 16424) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASDWGANTGQLYF | GEGSKLTVL |
| 117 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25625) | NO: |
| | | | | 2971) | | 8907) | | | 16347) |
| Clonotype_ | 1 | TRA | GEQVEQLPSILRVQEGSS | DSASNY | FPWYKQEPGENPK | IRSNMER | KQIQELIVLLDKKAKRFSLHITDTQ | CAAPSSSFSKLVF | GQGTSLSVV |
| 117 | | | ASINCSYE (SEQ ID | (SEQ ID | LIID (SEQ ID | (SEQ ID | PGDSAMYF (SEQ ID NO: | (SEQ ID NO: | P (SEQ ID |
| | | | NO: 48) | NO: | NO: 5984) | NO: | 11920) | 25626) | NO: |
| | | | | 3016) | | 8952) | | | 16422) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDWGGSAETLY | GSGTRLTVL |
| 118 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25627) | NO: |
| | | | | 2971) | | 8907) | | | 16325) |
| Clonotype_ | 1 | TRA | QQQVRQSPQSLTVWEGET | DSTFDY | FPWYRQFPGKSPA | ISLVSNK | KEDGRFTIFFNKREKKLSLHITDSQ | CAASEDSGTYQRF | GTGTKLQVV |
| 118 | | | TILNCSYE (SEQ ID | (SEQ ID | LLIA (SEQ ID | (SEQ ID | PGDSATYF (SEQ ID NO: | (SEQ ID NO: | P (SEQ ID |
| | | | NO: 19528) | NO: | NO: 21968) | NO: | 11940) | 25628) | NO: |
| | | | | 3208) | | 23188) | | | 16432) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDPRTGANTEV | GKGTRLTVV |
| 119 | | | VTLSCNQT (SEQ ID | (SEQ ID | LIHY (SEQ ID | (SEQ ID | LATPSQTSVYF (SEQ ID NO: | FF (SEQ ID NO: | (SEQ ID |
| | | | NO: 3) | NO: 2971) | NO: 5939) | NO: 8907) | 11875) | 25629) | NO: 16333) |
| Clonotype_ | 1 | TRA | QQKVQQSPESLTVSEGAM | DGTSNN | FRWYRQHSAKGLE | IFSDGE | KEEGRFTAHLNRANLHVSLHIREPQ | CAVTGGYKVVF | GSGTRLLVS |
| 119 | | | ASLNCTFS (SEQ ID | (SEQ ID | VLVS (SEQ ID | (SEQ ID | PSDSAVYL (SEQ ID NO: | (SEQ ID NO: | P (SEQ ID |
| | | | NO: 92) | NO: | NO: 6028) | NO: | 11964) | 25630) | NO: |
| | | | | 3060) | | 8996) | | | 16354) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGTDEQYF (SEQ | GPGTRLTVL |
| 120 | | | VTLSCNQT (SEQ ID | (SEQ ID | LIHY (SEQ ID | (SEQ ID | LATPSQTSVYF (SEQ ID NO: | ID NO: 25631) | (SEQ ID |
| | | | NO: 3) | NO: | NO: 5939) | NO: | 11875) | | NO: |
| | | | | 2971) | | 8907) | | | 16327) |
| Clonotype_ | 1 | TRA | GEQVEQLPSILRVQEGSS | NSASNY | FPWYKQEPGENPK | IRSNMER | KQTQGLIVLLDKKAKRFSLHITDTQ | CAATSSGSWQLIF | GSGTQLTVM |
| 120 | | | ASINCTYE (SEQ ID | (SEQ ID | LIID (SEQ ID | (SEQ ID | PGDSAMYF (SEQ ID NO: | (SEQ ID NO: | P (SEQ ID |
| | | | NO: 170) | NO: 3138) | NO: 5984) | NO: 8952) | 12042) | 25632) | NO: |
| | | | | | | | | | 16400) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDRGGQDTQYF | GPGTRLLVL |
| 121 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25633) | NO: |
| | | | | 2971) | | 8907) | | | 16341) |
| Clonotype_ | 1 | TRA | GDSVTQTEGQVTVSESKS | TTSIAYP | LFWYVRYPGEGLQ | VITAGQK | GSSRGFEATYNKETTSFHLQKASVQ | CALGVVVGQLTF | GSGTRLQVY |
| 121 | | | LIINCTYS (SEQ ID | N (SEQ | LLLK (SEQ ID | (SEQ ID | ESDSAVYY (SEQ ID NO: | (SEQ ID NO: | A (SEQ ID |
| | | | NO: 60) | ID NO: | NO: 5996) | NO: | 11932) | 25634) | NO: |
| | | | | 3028) | | 8964) | | | 26854) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDYEQYF (SEQ | GPGTRLTVL |
| 122 | | | VTLSCNQT (SEQ ID | (SEQ ID | LIHY (SEQ ID | (SEQ ID | LATPSQTSVYF (SEQ ID NO: | ID NO: 25635) | (SEQ ID |
| | | | NO: 3) | NO: 2971) | NO: 5939) | NO: 8907) | 11875) | | NO: 16327) |
| Clonotype_ | 1 | TRA | QQKVQQSPESLTVSEGAM | DGTSDN | FRWYRQHSGKGLE | IFSDGE | KEEGRFTAHLNRASLHVSLHIREPQ | CAMMGGRALIF | GTGTTVSVS |
| 122 | | | ASLNCTFS (SEQ ID | (SEQ ID | VLVS (SEQ ID | (SEQ ID | PSDSAVYL (SEQ ID NO: | (SEQ ID NO: | P (SEQ ID |
| | | | NO: 92) | NO: | NO: 6592) | NO: | 12528) | 25636) | NO: |
| | | | | 3624) | | 8996) | | | 16348) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGTNNNQAPLF | GEGTRLSVL |
| 123 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25637) | NO: |
| | | | | 2971) | | 8907) | | | 16441) |
| Clonotype_ | 1 | TRA | QQKVQQSPESLTVSEGAM | DGTSNN | FRWYRQHSAKGLE | IFSDGE | KEEGRFTAHLNRANLHVSLHIREPQ | CAVMSNYNVLYF | GSGTKLTVE |
| 123 | | | ASLNCTFS (SEQ ID | (SEQ ID | VLVS (SEQ ID | (SEQ ID | PSDSAVYL (SEQ ID NO: 11964) | (SEQ ID NO: | P (SEQ ID |
| | | | NO: 92) | NO: 3060) | NO: 6028) | NO: 8996) | | 15660) | NO: |
| | | | | | | | | | 16336) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGPGGATEVFF | GKGTRLTVV |
| 124 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25639) | NO: 16333) |
| Clonotype_ | 1 | TRA | GEQVEQRPPHLSVREGDS | DPNSYY | FFWYKQEPGAGLQ | VFSSTEI | NEGQGFTVLLNKKDKQLSLNLTAAH | CAVSAGKLTF (SEQ | GTGTSLLVD |
| 124 | | | AVIICTYT (SEQ ID | (SEQ ID | LLMK (SEQ ID | (SEQ ID | PGDSAVYF (SEQ ID NO: | ID NO: 25640) | P (SEQ ID |
| | | | NO: 108) | NO: | NO: 6044) | NO: | 11980) | | NO: |
| | | | | 3010) | | 8946) | | | 16398) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGETGGSNERLF | GHGTKLSVL |
| 125 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25641) | NO: |
| | | | | 2971) | | 8907) | | | 16401) |
| Clonotype_ | 1 | TRA | QQQVRQSSQSLTVWEGET | NSAFDY | FPWYQQFPGEGPA | IRSVSDK | KEDGRFTIFFNKREKNLSLHIKDSQ | CAAVPSSGSWQLIF | GSGTQLTVM |
| 125 | | | AILNCSYE (SEQ ID | (SEQ ID | LLIA (SEQ ID | (SEQ ID | PGDSATYF (SEQ ID NO: | (SEQ ID NO: | P (SEQ ID |
| | | | NO: 84) | NO: | NO: 6020) | NO: 8988) | 11956) | 25642) | NO: 16400) |
| | | | | 3052) | | | | | |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGETGGYAEQFF | GPGTRLTVL |
| 126 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25643) | NO: |
| | | | | 2971) | | 8907) | | | 16327) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAARTGGYKVVF | GSGTRLLVS |
| 126 | | | ASLNCTSS (SEQ ID | (SEQ ID | ALMS (SEQ ID | (SEQ ID | PSDSALYF (SEQ ID NO: | (SEQ ID NO: | P (SEQ ID |
| | | | NO: 64) | NO: | NO: 5938) | NO: | 11874) | 25644) | NO: |
| | | | | 2970) | | 8906) | | | 16354) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGEGGGLYEQYF | GPGTRLTVL |
| 127 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25645) | NO: |
| | | | | 2971) | | 8907) | | | 16327) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAASDSGSWQLIF | GSGTQLTVM |
| 127 | | | ASFNCTSS (SEQ ID | (SEQ ID | ALMS (SEQ ID | (SEQ ID | PSDSALYF (SEQ ID NO: | (SEQ ID NO: | P (SEQ ID |
| | | | NO: 2) | NO: | NO: 5938) | NO: | 11874) | 25646) | NO: |
| | | | | 2970) | | 8906) | | | 16400) |
| Clonotype_ | 1 | TRB | EAAVTQSPRNKVAVTGGK | NNHNN | MYWYRQDTGHGLR | SYGAGS () | TEKGDIPDGYKASRPSQENFSLILE | CASGDRGRGEQYF | GPGTRLTVL |
| 128 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25647) | NO: |
| | | | | 2971) | | 8907) | | | 16327) |
| Clonotype_ | 1 | TRA | GDSVTQTEGPVTLSEGTS | TKQYPT | ILFWYVQYPGEGPQ | VPKANEK | GSSRGFEATYNKEATSFHLQKASVQ | CALSERAGANTGKL | GHGTILRVH |
| 128 | | | LTVNCSYE (SEQ ID | (SEQ ID | LLFK (SEQ ID | (SEQ ID | ESDSAVYY (SEQ ID NO: | TF (SEQ ID NO: | P (SEQ ID |
| | | | NO: 38) | NO: | NO: 5974) | NO: | 12094) | 25648) | NO: |
| | | | | 3006) | | 8942) | | | 16356) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDLGSSAETLY | GSGTRLTVL |
| 129 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25649) | NO: |
| | | | | 2971) | | 8907) | | | 16325) |
| Clonotype_ | 1 | TRA | SQLAEENSWALSVHEGES | TSITA | LQWYRQKSGKGPA | IRSNERE | KRNGRLRATLDTSSQSSSLSITATR | CATDLDYANKMIF | GLGTILRVR |
| 129 | | | VTVNCSYK (SEQ ID | (SEQ ID | QLIL (SEQ ID | (SEQ ID | CEDTAVYF (SEQ ID NO: | (SEQ ID NO: | P (SEQ ID |
| | | | NO: 188) | NO: | NO: 6124) | NO: | 11896) | 25650) | NO: |
| | | | | 2992) | | 8928) | | | 16416) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDWGAYEQYF | GPGTRLTVL |
| 130 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25651) | NO: |
| | | | | 2971) | | 8907) | | | 16327) |
| Clonotype_ | 1 | TRA | GQQVQQSPTSLVLQEGEN | TSLNS | MQWFYQRPGGSLV | NPSGT | KQSGRLTSTTVIKERRSSLHISSSQ | CAMWASSSFSKLVF | GQGTSLSVV |
| 130 | | | AELQCNFS (SEQ ID | (SEQ ID | SVFY (SEQ ID | (SEQ ID | ITDSGTYL (SEQ ID NO: 12038) | (SEQ ID NO: | P (SEQ ID |
| | | | NO: 528) | NO: 2978) | NO: 6464) | NO: 8914) | | 25652) | NO: |
| | | | | | | | | | 16422) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGETGASSDYTF | GSGTRLLVI |
| 131 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25653) | NO: |
| | | | | 2971) | | 8907) | | | 16337) |
| Clonotype_ | 1 | TRA | QQKVQQSPESLIVPEGGM | DRNVDY | FWWYRQHSGKSPK | IFSNGE | KEEGRFTVHLNKASLHTSLHIRDSQ | CAAWTGGYKVVF | GSGTRLLVS |
| 131 | | | ASLNCTSS (SEQ ID | (SEQ ID | MLMS (SEQ ID | (SEQ ID | PSDSALYL (SEQ ID NO: | (SEQ ID NO: | P (SEQ ID |
| | | | NO: 28) | NO: | NO: 5964) | NO: | 11900) | 25654) | NO: |
| | | | | 2996) | | 8932) | | | 16354) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGETGGDAETLY | GSGTRLTVL |
| 132 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25655) | NO: |
| | | | | 2971) | | 8907) | | | 16325) |
| Clonotype_ | 1 | TRA | QQQVRQSPQSLTVWEGET | NSAFDY | FPWYQQFPGEGPA | ILSVSNK | KEDGRFTIFFNKREKKLSLHIADSQ | CAASSNTGYQNFYF | GKGTSLTVI |
| 132 | | | AILNCSYE (SEQ ID | (SEQ ID | LLIS (SEQ ID | (SEQ ID | PGDSATYF (SEQ ID NO: | (SEQ ID NO: | P (SEQ ID |
| | | | NO: 68) | NO: | NO: 6004) | NO: | 11996) | 25656) | NO: |
| | | | | 3052) | | 9028) | | | 16366) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGERTVQDTQYF | GPGTRLLVL |
| 133 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25657) | NO: |
| | | | | 2971) | | 8907) | | | 16341) |
| Clonotype_ | 1 | TRA | GDSVTQTEGLVTLTEGLP | TIYSNAF | LFWYVHYLNESPW | SSTDNKR | TEHQGFHATLHKSSSSFHLQKSSAQ | CALSDNNAGAKLTF | GGGTRLTVR |
| 133 | | | VMLNCTYQ (SEQ ID | (SEQ ID | LLLR (SEQ ID | (SEQ ID | LSDSALYY (SEQ ID NO: | (SEQ ID NO: | P (SEQ ID |
| | | | NO: 46) | NO: | NO: 21998) | NO: | 11918) | 25658) | NO: |
| | | | | 3858) | | 8912) | | | 16338) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGESQNTLYF | GAGTRLSVL |
| 134 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: 8907) | 11875) | 25659) | NO: 16365) |
| | | | | 2971) | | | | | |
| Clonotype_ | 1 | TRA | GDSVTQTEGPVTLSEGTS | TKQYPT | LFWYVQYPGEGPQ | VPKANEK | GSSRGFEATYNKEATSFHLQKASVQ | CALSDGTGYQNFYF | GKGTSLTVI |
| 134 | | | LTVNCSYE (SEQ ID | (SEQ ID | LLFK (SEQ ID | (SEQ ID | ESDSAVYY (SEQ ID NO: | (SEQ ID NO: | P (SEQ ID |
| | | | NO: 38) | NO: | NO: 5974) | NO: | 12094) | 25660) | NO: |
| | | | | 3006) | | 8942) | | | 16366) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGEGLGGRETLY | GSGTRLTVL |
| 135 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25661) | NO: |
| | | | | 2971) | | 8907) | | | 16325) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGEGGRALIF | GTGTTVSVS |
| 135 | | | VTMDCVYE (SEQ ID | (SEQ ID | FLIR (SEQ ID | N (SEQ | IEDSAVYF (SEQ ID NO: | (SEQ ID NO: | P (SEQ ID |
| | | | NO: 44) | NO: | NO: 5942) | ID NO: | 11878) | 25662) | NO: |
| | | | | 3056) | | 8910) | | | 16348) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGEGLGGRGDTQ | GPGTRLLVL |
| 136 | | | VTLSCNQT (SEQ ID | (SEQ ID | LIHY (SEQ ID | (SEQ ID | LATPSQTSVYF (SEQ ID NO: | YF (SEQ ID NO: | (SEQ ID |
| | | | NO: 3) | NO: 2971) | NO: 5939) | NO: 8907) | 11875) | 25663) | NO: |
| | | | | | | | | | 16341) |
| Clonotype_ | 1 | TRA | LAKTTQPPSMEAYEGQEV | NIATSEY | IYWYRQVPHQGPQ | GYKDYVV | NEVASLFISADRKLSTLSLPWVSLR | CIVTDPWGSNTNKV | GTGTRLQVL |
| 136 | | | NVSCSHT (SEQ ID NO: | (SEQ ID | FIIQ (SEQ ID | (SEQ ID | DAAVYY (SEQ ID NO: 11978) | VF (SEQ ID NO: | P (SEQ ID |
| | | | 106) | NO: | NO: 6042) | NO: | | 25664) | NO: |
| | | | | 3074) | | 9010) | | | 16698) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGEGQGNTEVFF | GKGTRLTVV |
| 137 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25665) | NO: |
| | | | | 2971) | | 8907) | | | 16333) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGDYSNNRLT | GKGTQVVVL |
| 137 | | | VTMDCVYE (SEQ ID | (SEQ ID | FLIR (SEQ ID | N (SEQ | IEDSAVYF (SEQ ID NO: | L (SEQ ID NO: | P (SEQ ID |
| | | | NO: 44) | NO: | NO: 5942) | ID NO: | 11878) | 25666) | NO: |
| | | | | 3056) | | 8910) | | | 16644) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGEGETTEVFF | GKGTRLTVV |
| 138 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25667) | NO: |
| | | | | 2971) | | 8907) | | | 16333) |
| Clonotype_ | 1 | TRA | GEQVEQLPSSLIVQEGAS | DSASVY | FPWYKQEPGKRLQ | IRSNMER | KQNQRLTLLFDKKTKHLSLHITATQ | CSAPGAPNSAGNKL | GIGTRVLVR |
| 138 | | | VLINCSYT (SEQ ID | (SEQ ID | FIID (SEQ ID | (SEQ ID | PGDSAIYF (SEQ ID NO: | TF (SEQ ID NO: | P (SEQ ID |
| | | | NO: 160) | NO: | NO: 6096) | NO: | 12032) | 25668) | NO: |
| | | | | 3128) | | 8952) | | | 16580) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGEGAANTEVFF | GKGTRLTVV |
| 139 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25669) | NO: |
| | | | | 2971) | | 8907) | | | 16333) |
| Clonotype_ | 1 | TRA | GDSVTQTEGPVTLSEGTS | TKQYPT | LFWYVQYPGEGPQ | VPKANEK | GSNRGFEATYNKEATSFHLQKASVQ | CALSDGTSGSWQLI | GSGTQLTVM |
| 139 | | | LTVNCSYE (SEQ ID | (SEQ ID | LLFK (SEQ ID | (SEQ ID | ESDSAVYY (SEQ ID NO: | F (SEQ ID NO: | P (SEQ ID |
| | | | NO: 38) | NO: | NO: 5974) | NO: | 11910) | 25670) | NO: |
| | | | | 3006) | | 8942) | | | 16400) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDNWGDTQYF | GPGTRLLVL |
| 140 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25671) | NO: |
| | | | | | | | | | 16341) |
| Clonotype_ | 1 | TRA | GDSVTQTEGLVTLTEGLP | STYSPF | LFWYVQHLNEAPK | SFTDNKR | PEHQGFHATLHKSSSSFHLQKSSAQ | CALSDQGGSAKLIF | GEGTKLTVS |
| 140 | | | VMLNCTYQ (SEQ ID | (SEQ ID | LLLK (SEQ ID | (SEQ ID | LSDSALYY (SEQ ID NO: | (SEQ ID NO: | S (SEQ ID |
| | | | NO: 46) | NO: | NO: 5944) | NO: | 11944) | 25672) | NO: |
| | | | | 3040) | | 8950) | | | 16384) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDTGDSDYTF | GSGTRLLVI |
| 141 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25673) | NO: |
| | | | | 2971) | | 8907) | | | 16337) |
| Clonotype_ | 1 | TRA | GDSVTQKQGQVTLSEDDF | TTTYPT | ILLWYVQYPGQGPQ | VTTANNK | GISRGFEATYDKGTTSFHLQKASVQ | CALVSYYGSSGNKL | GIGTLLSVK |
| 141 | | | LFINCTYS (SEQ ID | (SEQ ID | LLLK (SEQ ID | (SEQ ID | ESDSAVYF (SEQ ID NO: | IF (SEQ ID NO: | P (SEQ ID |
| | | | NO: 510) | NO: | NO: 21948) | NO: | 12382) | 25674) | NO: |
| | | | | 3478) | | 9054) | | | 16456) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDPGDAEQFF | GPGTRLTVL |
| 142 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25675) | NO: |
| | | | | 2971) | | 8907) | | | 16327) |
| Clonotype_ | 1 | TRA | GQQVQQSPASLVLQEGEN | TSLNS | MQWFYQRPEGSLV | NPSGT | KQSGRLTSTTVIKERRSSLHISSSQ | CAMEPGTGNYKYVF | GAGTRLKVI |
| 142 | | | AELQCNFS (SEQ ID | (SEQ ID | SLFY (SEQ ID | (SEQ ID | ITDSGTYL (SEQ ID NO: | (SEQ ID NO: | A (SEQ ID |
| | | | NO: 10) | NO: | NO: 6192) | NO: | 12038) | 25676) | NO: |
| | | | | 2978) | | 8914) | | | 16380) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDPGNSDYTF | GSGTRLLVI |
| 143 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25677) | NO: |
| | | | | | | | | | 16337) |
| Clonotype_ | 1 | TRA | AQSVTQPDARVTVSEGAS | YSGTPY | LFWYVQYPRQGLQ | YYSGDPV | GVNGFEAEFSKSNSSFHLRKASVH | CVLSVTNTGKLTF | GDGTVLTVK |
| 143 | | | LQLRCKYS (SEQ ID | (SEQ ID | LLLK (SEQ ID | V (SEQ | WSDSAVYF (SEQ ID NO: | (SEQ ID NO: | P (SEQ ID |
| | | | NO: 12) | NO: | NO: 5948) | ID NO: | 11892) | 25678) | NO: |
| | | | | 3054) | | 8916) | | | 16516) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDLGGHYEQYF | GPGTRLTVL |
| 144 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: 8907) | 11875) | 25679) | NO: 16327) |
| | | | | 2971) | | | | | |
| Clonotype_ | 1 | TRA | SQWGEENLQALSIQEGED | TYTTV | VHWYRQDSGRGPA | IRSNERE | KRSGRLRATLDTSSQSSSLSITAAQ | CAPASSGSWQLIF | GSGTQLTVM |
| 144 | | | VTMNCSYK (SEQ ID | (SEQ ID | LIIL (SEQ ID | (SEQ ID | CEDTAVYF (SEQ ID NO: | (SEQ ID NO: | P (SEQ ID |
| | | | NO: 80) | NO: 3048) | NO: 6016) | NO: 8928) | 11952) | 25680) | NO: 16400) |
| Clonotype_ | 1 | TRB | EAAVTQSPRNKVAVTGGK | INNHNN | MYWYRQDTGHGLR | SYGAGS | TEKGDIPDGYKASRPSQENFSLILE | CASGDDWDQNTLYF | GAGTRLSVL |
| 145 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25681) | NO: |
| | | | | 2971) | | 8907) | | | 16365) |
| Clonotype_ | 1 | TRA | AQSVTQPDARVTVSEGAS | YSATPY | LFWYVQYPRQGLQ | YYSGDPV | GVNGFEAEFSKSNSSFHLRKASVH | CAVKGNAGAKLTF | GGGTRLTVR |
| 145 | | | LQLRCKYS (SEQ ID | (SEQ ID | LLLK (SEQ ID | V (SEQ | WSDSAVYF (SEQ ID NO: | (SEQ ID NO: | P (SEQ ID |
| | | | NO: 12) | NO: | NO: 5948) | ID NO: | 11892) | 25682) | NO: |
| | | | | 2980) | | 8916) | | | 16338) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDAEWDTQYF | GPGTRLLVL |
| 146 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25683) | NO: |
| | | | | 2971) | | 8907) | | | 16341) |
| Clonotype_ | 1 | TRA | GDSVIQMQGQVTLSENDF | TTGYPT | LFWYVQYSGEGPQ | VTTANNK | GSSRGFEATYDKGTTSFHLQKTSVQ | CAMSAASSGSWQLI | GSGTQLTVM |
| 146 | | | LFINCTYS (SEQ ID | (SEQ ID | LLLQ (SEQ ID | (SEQ ID | EIDSAVYY (SEQ ID NO: | F (SEQ ID NO: | P (SEQ ID |
| | | | NO: 456) | NO: | NO: 6392) | NO: 9054) | 12328) | 25684) | NO: 16400) |
| | | | | 3220) | | | | | |
| Clonotype_ | 1 | TRE | EAAVTQSPRNKVAVTGGK | NNHNN | MYWYRQDTGHGLR | SYGAGS | TEKGDIPDGYKASRPSQENFSLILE | CASGDAGNSDYTF | GSGTRLLVI |
| 147 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: 8907) | 11875) | 25685) | NO: 16337) |
| | | | | 2971) | | | | | |
| Clonotype_ | 1 | TRA | GDSVTQTEGQVTVSESKS | ATSIAYP | LFWYVRYPGEGLQ | VITAGQK | GSSRGFEATYNKETTSFHLQKASVQ | CALRPNMGYKLTF | GTGTSLLVD |
| 147 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 60) | ID NO: | NO: 5996) | NO: | 11932) | 25686) | NO: |
| | | | | 3098) | | 8964) | | | 16398) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDAGIQDTQYF | GPGTRLLVL |
| 148 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25687) | NO: |
| | | | | 2971) | | 8907) | | | 16341) |
| Clonotype_ | 1 | TRA | GDSVTQTEGPVTVSESES | ATSIAYP | LFWYVRYPGEGLQ | VITAGQK | ESDSAVYY (SEQ ID NO: | CASSANKVVF (SEQ | GTGTRLQVL |
| 148 | | | LIINCTYS (SEQ ID | N (SEQ | LLLK (SEQ ID | (SEQ ID | GSSRGFEATYNKETTSFHLQKASVQ | ID NO: 25688) | P (SEQ ID |
| | | | NO: 130) | ID NO: | NO: 5996) | NO: | 11932) | | NO: |
| | | | | 3098) | | 8964) | | | 16698) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDAGGGNERLF | GHGTKLSVL |
| 149 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25689) | NO: |
| | | | | 2971) | | 8907) | | | 16401) |
| Clonotype_ | 1 | TRA | QQQVRQSPQSLTVWEGET | NSAFDY | FPWYQQFPGEGPA | IRSVSDK | KEDGGFTIFFNKREKNLSLHIKDSQ | CAASESGGNYKPTF | GKGTSLVVH |
| 149 | | | AILNCSYE (SEQ ID | (SEQ ID | LLIA (SEQ ID | (SEQ ID | PGDSATYF (SEQ ID NO: | (SEQ ID NO: | P (SEQ ID |
| | | | NO: 68) | NO: 3052) | NO: 6020) | NO: 8988) | 24470) | 25690) | NO: 16394) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDATYNSPLYF | AAGTRLTVT |
| 150 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25691) | NO: |
| | | | | 2971) | | 8907) | | | 16381) |
| Clonotype_ | 1 | TRA | AQKVTQTQTSISVMEKTT | TQDSSYF | FLIR (SEQ ID | QDSYKKE | ATVGHYSLNFQKPKSSIGLIITATQ | CAMRDEGSAGNKLT | GIGTRVLVR |
| 150 | | | VTMDCVYE (SEQ ID | (SEQ ID | LFWYKQTASGEIV | N (SEQ | IEDSAVYF (SEQ ID NO: | F (SEQ ID NO: | P (SEQ ID |
| | | | NO: 44) | NO: | NO: 5942) | ID NO: | 11878) | 25692) | NO: |
| | | | | 3056) | | 8910) | | | 16580) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDAAENTLYF | GAGTRLSVL |
| 151 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25693) | NO: |
| | | | | 2971) | | 8907) | | | 16365) |
| Clonotype_ | 1 | TRA | GQQVQQSPASLVLQEGEN | TSLNS | MQWFYQRPGGSLV | NPSGT | KQSGRLTSTTVIKERRSSLHISSSQ | CAMDTGGADRLTF | GKGTQLIIQ |
| 151 | | | AELQCNFS (SEQ ID | (SEQ ID | SLFY (SEQ ID | (SEQ ID | TTDSGTYL (SEQ ID NO: | (SEQ ID NO: | P (SEQ ID |
| | | | NO: 10) | NO: | NO: 5946) | NO: | 11882) | 25694) | NO: |
| | | | | 2978) | | 8914) | | | 16360) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDAGSAETLYF | GSGTRLTVL |
| 152 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25695) | NO: 16325) |
| Clonotype_ | 1 | TRA | GDSVTQTEGLVTLTKGLP | TTYSPF | LFWYVQHLNEAPK | SSTDNKR | TEHQGFYATLHKSSSSFHLQKSSVQ | CALSSGSWQLIF | GSGTQLTVM |
| 152 | | | VMLNCTYQ (SEQ ID | (SEQ ID | LLLK (SEQ ID | (SEQ ID | LSDSALYF (SEQ ID NO: | (SEQ ID NO: | P (SEQ ID |
| | | | NO: 8) | NO: | NO: 5944) | NO: | 11880) | 15636) | NO: |
| | | | | 2976) | | 8912) | | | 16400) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDAGGSAETLY | GSGTRLTVL |
| 153 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25697) | NO: |
| | | | | 2971) | | 8907) | | | 16325) |
| Clonotype_ | 1 | TRA | QQQVRQSPQSLTVWEGET | DSTFNY | FPWYQQFPGEGPA | ISSVSDK | KEDGRFTIFFNKREKKLSLHITDSQ | CAASPNTGYQNFYF | GKGTSLTVI |
| 153 | | | AILNCSYE (SEQ ID | (SEQ ID | LLIS (SEQ ID | (SEQ ID | PGDSATYF (SEQ ID NO: | (SEQ ID NO: | P (SEQ ID |
| | | | NO: 68) | NO: | NO: 6004) | NO: 8972) | 11940) | 25698) | NO: 16366) |
| | | | | 3036) | | | | | |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDAGGRQNTLY | GAGTRLSVL |
| 154 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: 2971) | NO: 5939) | NO: 8907) | 11875) | 25699) | NO: 16365) |
| Clonotype_ | 1 | TRA | QQQVRQSPQSLTVWEGET | DSTFNY | FPWYQQFPGEGPA | IRSVSDK | KEDGRFTIFFNKREKKLSLHITDSQ | CAASADTGNYKYVF | GAGTRLKVI |
| 154 | | | AILNCSYE (SEQ ID | (SEQ ID | LLIS (SEQ ID | (SEQ ID | PGDSATYF (SEQ ID NO: 11940) | (SEQ ID NO: | A (SEQ ID |
| | | | NO: 68) | NO: | NO: 6004) | NO: | | 25700) | NO: |
| | | | | 3036) | | 8988) | | | 16380) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDAGGANSDYT | GSGTRLLVI |
| 155 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: 2971) | NO: 5939) | NO: 8907) | 11875) | 25701) | NO: |
| | | | | | | | | | 16337) |
| Clonotype_ | 1 | TRA | GQQVQQSPSSLVLQEGEN | STATQ | LQWFYQSPGGSLV | NPSGT | KHTGRLTSTTVTKERRSSLHISSSQ | CAPQQNTGYQNFYF | GKGTSLTVI |
| 155 | | | AELQCNFS (SEQ ID | (SEQ ID | SLLS (SEQ ID | (SEQ ID | TTDSGTYL (SEQ ID NO: | (SEQ ID NO: | P (SEQ ID |
| | | | NO: 322) | NO: | NO: 6258) | NO: | 12194) | 25702) | NO: |
| | | | | 3290) | | 8914) | | | 16366) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDGPWDTEVFF | GKGTRLTVV |
| 156 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25703) | NO: |
| | | | | 2971) | | 8907) | | | 16333) |
| Clonotype_ | 1 | TRA | DAKTTQPDSMESTEGETV | TISGNEY | IYWYRQVPLQGPE | GLQQN | TTNSMAFLAIASDRKSSTLILPHVS | CILRGTGSKLSF | GKGAKLTVS |
| 156 | | | HLPCSHA (SEQ ID NO: | (SEQ ID | YVTH (SEQ ID | (SEQ ID | LRDAAVYH (SEQ ID NO: | (SEQ ID NO: | P (SEQ ID |
| | | | 104) | NO: | NO: 6040) | NO: | 12266) | 25704) | NO: |
| | | | | 3072) | | 9008) | | | 16524) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDVTGGAREQY | GPGTRLTVL |
| 157 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25705) | NO: |
| | | | | 2971) | | 8907) | | | 16327) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGHTGYQNFY | GKGTSLTVI |
| 157 | | | VTMDCVYE (SEQ ID | (SEQ ID | FLIR (SEQ ID | N (SEQ | IEDSAVYF (SEQ ID NO: | F (SEQ ID NO: | P (SEQ ID |
| | | | NO: 44) | NO: | NO: 5942) | ID NO: | 11878) | 25706) | NO: |
| | | | | 3056) | | 8910) | | | 16366) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDVLGGIEQYF | GPGTRLTVL |
| 158 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25707) | NO: |
| | | | | | | | | | 16327) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREDDSGYNKLT | GKGTVLLVS |
| 158 | | | VTMDCVYE (SEQ ID | (SEQ ID | FLIR (SEQ ID | N (SEQ | IEDSAVYF (SEQ ID NO: | F (SEQ ID NO: | P (SEQ ID |
| | | | NO: 44) | NO: | NO: 5942) | ID NO: | 11878) | 25708) | NO: |
| | | | | 3056) | | 8910) | | | 16378) |
| Clonotype_ | | TRB | EAAVTQSPRNKVAVTGGK | NNHNN | MYWYRQDTGHGLR | SYGAGS | TEKGDIPDGYKASRPSQENFSLILE | CASGDVGQGFYAEQ | GPGTRLTVL |
| 159 | | | VTLSCNQT (SEQ ID | (SEQ ID | LIHY (SEQ ID | (SEQ ID | LATPSQTSVYF (SEQ ID NO: | FF (SEQ ID NO: | (SEQ ID |
| | | | NO: 3) | NO: | NO: 5939) | NO: | 11875) | 25709) | NO: |
| | | | | 2971) | | 8907) | | | 16327) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAVNYSNNRLTL | GKGTQVVVL |
| 159 | | | ASLNCTSS (SEQ ID | (SEQ ID | ALMS (SEQ ID | (SEQ ID | PSDSALYF (SEQ ID NO: | (SEQ ID NO: | P (SEQ ID |
| | | | NO: 64) | NO: | NO: 5938) | NO: | 11874) | 25710) | NO: |
| | | | | 2970) | | 8906) | | | 16644) |
| Clonotype_ | 1 | TRB | EAAVTQSPRNKVAVTGGK | INNHNN | MYWYRQDTGHGLR | SYGAGS | TEKGDIPDGYKASRPSQENFSLILE | CASGDVGGAHTGQL | GEGSKLTVL |
| 160 | | | VTLSCNQT (SEQ ID | (SEQ ID | LIHY (SEQ ID | (SEQ ID | LATPSQTSVYF (SEQ ID NO: | YF (SEQ ID NO: | (SEQ ID |
| | | | NO: 3) | NO: | NO: 5939) | NO: | 11875) | 25711) | NO: |
| | | | | 2971) | | 8907) | | | 16347) |
| Clonotype_ | 1 | TRA | QQQVRQSPQSLTVWEGET | DSTFDY | FPWYRQFPGKSPA | ISLVSNK | KEDGRFTIFFNKREKKLSLHITDSQ | CAASKAGNTRKLIF | GLGTTLQVQ |
| 160 | | | TILNCSYE (SEQ ID | (SEQ ID | LLIA (SEQ ID | (SEQ ID | PGDSATYF (SEQ ID NO: | (SEQ ID NO: | P (SEQ ID |
| | | | NO: 19528) | NO: | NO: 21968) | NO: | 11940) | 25712) | NO: 16326) |
| | | | | 3208) | | 23188) | | | |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDVGGHNTGQL | GEGSKLTVL |
| 161 | | | VTLSCNQT (SEQ ID | (SEQ ID | LIHY (SEQ ID | (SEQ ID | LATPSQTSVYF (SEQ ID NO: | YF (SEQ ID NO: | (SEQ ID |
| | | | NO: 3) | NO: 2971) | NO: 5939) | NO: 8907) | 11875) | 25713) | NO: |
| | | | | | | | | | 16347) |
| Clonotype_ | 1 | TRA | QQKVQQSPESLIVPEGGM | DRNVDY | FWWYRQHSGKSPK | IFSNGE | KEEGRFTVHLNKASLHTSLHIRDSQ | CAASTAGNTRKLIF | GLGTTLQVQ |
| 161 | | | ASLNCTSS (SEQ ID | (SEQ ID | MLMS (SEQ ID | (SEQ ID | PSDSALYL (SEQ ID NO: | (SEQ ID NO: | P (SEQ ID |
| | | | NO: 28) | NO: | NO: 5964) | NO: | 11900) | 25714) | NO: |
| | | | | 2996) | | 8932) | | | 16326) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDVETGGSQNT | GAGTRLSVL |
| 162 | | | VTLSCNQT (SEQ ID | (SEQ ID | LIHY (SEQ ID | (SEQ ID | LATPSQTSVYF (SEQ ID NO: | LYF (SEQ ID NO: | (SEQ ID |
| | | | NO: 3) | NO: | NO: 5939) | NO: | 11875) | 25715) | NO: |
| | | | | 2971) | | 8907) | | | 16365) |
| Clonotype_ | 1 | TRA | QQNVQQSPESLIVPEGAR | DSASQY | FWWYRQHSGKAPK | IFSNGE | KEEGRFTIHLNKASLHFSLHIRDSQ | CAVSPWTGGYKVVF | GSGTRLLVS |
| 162 | | | TSLNCTFS (SEQ ID | (SEQ ID | ALMS (SEQ ID | (SEQ ID | PSDSALYL (SEQ ID NO: | (SEQ ID NO: | P (SEQ ID |
| | | | NO: 76) | NO: | NO: 6012) | NO: | 11948) | 25716) | NO: |
| | | | | 3044) | | 8932) | | | 16354) |
| Clonotype_ | 1 | TRB | EAAVTQSPRNKVAVTGGK | INNHNN | MYWYRQDTGHGLR | SYGAGS | TEKGDIPDGYKASRPSQENFSLILE | CASGDSLSGNTLYF | GEGSRLIVV |
| 163 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25717) | NO: |
| | | | | 2971) | | 8907) | | | 16471) |
| Clonotype_ | 1 | TRA | GDSVTQTEGLVTLTEGLP | TIYSNPF | LLLK (SEQ ID | SSTDNKR | TEHQGFHATLHKSSSSFHLQKSSAQ | CALSGDRGSALGRL | GAGTQLIVI |
| 163 | | | VMLNCTYQ (SEQ ID | (SEQ ID | LFWYVHYLNESPR | (SEQ ID | LSDSALYY (SEQ ID NO: | HF (SEQ ID NO: | P (SEQ ID |
| | | | NO: 46) | NO: | NO: 6144) | NO: | 11918) | 25718) | NO: |
| | | | | 3014) | | 8912) | | | 16480) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDSGGVAETLY | GSGTRLTVL |
| 164 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: | NO: 5939) | NO: | 11875) | 25719) | NO: |
| | | | | 2971) | | 8907) | | | 16325) |
| Clonotype_ | 1 | TRA | QQQVRQSPQSLTVWEGET | NSAFDY | FPWYQQFPGEGPA | ILSVSDK | KEDGRFTIFFNKREKKLSLHIADSQ | CAAKGIASSSFSKL | GQGTSLSVV |
| 164 | | | AILNCSYE (SEQ ID | (SEQ ID | LLIS (SEQ ID | (SEQ ID | PGDSATYF (SEQ ID NO: | VF (SEQ ID NO: | P (SEQ ID |
| | | | NO: 68) | NO: | NO: 6004) | NO: | 11996) | 25720) | NO: |
| | | | | 3052) | | 9200) | | | 16422) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGDLSAETLYF | GSGTRLTVL |
| 165 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25721) | NO: |
| | | | | | | | | | 16325) |
| Clonotype_ | 1 | TRA | GDSVTQTEGLVTLTKGLP | TTYSPF | LFWYVQHLNEAPK | SSTDNKR | TEHQGFYATLHKSSSSFHLQKSSVQ | CALSSNSGTYQRF | GTGTKLQVV |
| 165 | | | VMLNCTYQ (SEQ ID | (SEQ ID | LLLK (SEQ ID | (SEQ ID | LSDSALYF (SEQ ID NO: | (SEQ ID NO: | P (SEQ ID |
| | | | NO: 8) | NO: | NO: 5944) | NO: | 11880) | 25722) | NO: |
| | | | | 2976) | | 8912) | | | 16432) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGAWGGEQYF | GPGTRLTVL |
| 166 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25723) | NO: |
| | | | | 2971) | | 8907) | | | 16327) |
| Clonotype_ | 1 | TRA | GQQVQQSPTSLVLQEGEN | TSLNS | MQWFYQRPGGSLI | NPSGT | KQSGRLTSTTVIKERRSSLHISSSQ | CATPADTNAYKVIF | GKGTHLHVL |
| 166 | | | AELQCNFS (SEQ ID | (SEQ ID | SVFY (SEQ ID | (SEQ ID | ITDSGTYL (SEQ ID NO: 12038) | (SEQ ID NO: | P (SEQ ID |
| | | | NO: 528) | NO: 2978) | NO: 6710) | NO: 8914) | | 25724) | NO: 16330) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGGQGAYEQYF | GPGTRLTVL |
| 167 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25725) | NO: |
| | | | | 2971) | | 8907) | | | 16327) |
| Clonotype_ | 1 | TRA | GEQVEQRPPHLSVREGDS | DPNSYY | FFWYKQEPGAGLQ | VFSSTEI | NEGQGFTVLLNKKDKQLSLNLTAAH | CAVRTGSGGKLTL | GAGTRLQVN |
| 167 | | | AVIICTYT (SEQ ID | (SEQ ID | LLMK (SEQ ID | (SEQ ID | PGDSAVYF (SEQ ID NO: | (SEQ ID NO: | L(SEQ ID |
| | | | NO: 108) | NO: | NO: 6044) | NO: | 11980) | 25726) | NO: |
| | | | | 3010) | | 8946) | | | 17020) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGGPGLGSETLY | GSGTRLTVL |
| 168 | | | VTLSCNQT (SEQ ID | (SEQ ID | LIHY (SEQ ID | (SEQ ID | LATPSQTSVYF (SEQ ID NO: | F (SEQ ID NO: | (SEQ ID |
| | | | NO: 3) | NO: 2971) | NO: 5939) | NO: 8907) | 11875) | 25727) | NO: 16325) |
| Clonotype_ | 1 | TRA | GDKVKQSPSALSLQEGTN | IAATT | VQWFLQNPRGSLI | LVPGT | KENGRLKSTFNSKESYSTLHIRDAQ | CAGYGGSGNKLIF | GTGTLLSVK |
| 168 | | | SALRCNFS (SEQ ID | (SEQ ID | NLFY (SEQ ID | (SEQ ID | LEDSGTYF (SEQ ID NO: | (SEQ ID NO: | P (SEQ ID |
| | | | NO: 404) | NO: | NO: 6486) | NO: | 12130) | 25728) | NO: |
| | | | | 3372) | | 9162) | | | 16350) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGGDREVFF | GKGTRLTVV |
| 169 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: 2971) | NO: 5939) | NO: 8907) | 11875) | 25729) | NO: |
| | | | | | | | | | 16333) |
| Clonotype_ | 1 | TRA | GNSVTQMQGQVTLSEEEF | TTGYPT | ILFWYVQYPGEGPQ | VTTANNK | ESDSAVYY (SEQ ID NO: | CVLGDHSNNRLTL | GKGTQVVVL |
| 169 | | | LFINCTYS (SEQ ID | (SEQ ID | LLLK (SEQ ID | (SEQ ID | GSSRGFEATYDKGTTSFHLQKASVQ | (SEQ ID NO: | P (SEQ ID |
| | | | NO: 252) | NO: | NO: 6086) | NO: | 12124) | 25730) | NO: |
| | | | | 3220) | | 9054) | | | 16644) |
| Clonotype_ | 1 | TRB | EAAVTQSPRNKVAVTGGK | NNHNN | MYWYRQDTGHGLR | SYGAGS | TEKGDIPDGYKASRPSQENFSLILE | CASGYWGNEQYF | GPGTRLTVL |
| 170 | | | VTLSCNQT (SEQ ID | (SEQ ID | LIHY (SEQ ID | (SEQ ID | LATPSQTSVYF (SEQ ID NO: | (SEQ ID NO: | (SEQ ID |
| | | | NO: 3) | NO: | NO: 5939) | NO: | 11875) | 25731) | NO: |
| | | | | 2971) | | 8907) | | | 16327) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMRGMATGGNNKL | GQGTVLSVI |
| 170 | | | VTMDCVYE (SEQ ID | (SEQ ID | FLIR (SEQ ID | N (SEQ | IEDSAVYF (SEQ ID NO: | TF (SEQ ID NO: | P (SEQ ID |
| | | | NO: 44) | NO: | NO: 5942) | ID NO: | 11878) | 25732) | NO: |
| | | | | 3056) | | 8910) | | | 16368) |
| Clonotype_ | 1 | TRB | EAAVTQSPRNKVTVTGGN | NSHNY | MYWYRQDTGHGLR | SYGAGN | ILQIGDVPDGYKATRTTQEDFFLLLE | CASTPGQSSYEQYF | GPGTRLTVL |
| 171 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25733) | NO: |
| | | | | 3005) | | 8941) | | | 16327) |
| Clonotype_ | 1 | TRA | QQKVQQSPESLIVPEGGM | DRNVDY | FWWYRQHSGKSPK | IFSNGE | KEEGRFTVHLNKASLHTSLHIRDSQ | CAAMLSGSFNKLTF | GAGTRLAVC |
| 171 | | | ASLNCTSS (SEQ ID | (SEQ ID | MLMS (SEQ ID | (SEQ ID | PSDSALYL (SEQ ID NO: 11900) | (SEQ ID NO: | P (SEQ ID |
| | | | NO: 28) | NO: 2996) | NO: 5964) | NO: 8932) | | 25734) | NO: 16692) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASRDSSGNTLYF | GEGSRLIVV |
| 172 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25735) | NO: |
| | | | | 3005) | | 8941) | | | 16471) |
| Clonotype_ | 1 | TRA | AQSVTQPDARVTVSEGAS | YSATPY | LFWYVQYPRQGLQ | | YYSGDPEFSKSNSSFHLQKASVHWS | CALSMLGGRALIF | GTGTTVSVS |
| 172 | | | LQLRCKYS (SEQ ID | (SEQ ID | LLLK (SEQ ID | | DSAVYF (SEQ ID NO: 24516) | (SEQ ID NO: | P (SEQ ID |
| | | | NO: 12) | NO: | NO: 5948) | | | 25736) | NO: |
| | | | | 2980) | | | | | 16348) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASRDNNQAPLF | GEGTRLSVL |
| 173 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25737) | NO: |
| | | | | 3005) | | 8941) | | | 16441) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAASKTSSSFSKLV | GQGTSLSVV |
| 173 | | | ASFNCTSS (SEQ ID | (SEQ ID | ALMS (SEQ ID | (SEQ ID | PSDSALYF (SEQ ID NO: 11874) | F (SEQ ID NO: | P (SEQ ID |
| | | | NO: 2) | NO: | NO: 5938) | NO: 8906) | | 25738) | NO: 16422) |
| | | | | 2970) | | | | | |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSTNNQAPLF | GEGTRLSVL |
| 174 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25739) | NO: |
| | | | | 3005) | | 8941) | | | 16441) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMRASNTDKVVF | GTGTRLQVS |
| 174 | | | VTMDCVYE (SEQ ID | (SEQ ID | FLIR (SEQ ID | N (SEQ | IEDSAVYF (SEQ ID NO: 11878) | (SEQ ID NO: | P (SEQ ID |
| | | | NO: 44) | NO: 3056) | NO: 5942) | ID NO: | | 25740) | NO: 16374) |
| | | | | | | 8910) | | | |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSEMGGRQDTQY | GPGTRLLVL |
| 175 | | | VTLSCRQT (SEQ ID | (SEQ ID | LIHY (SEQ ID | (SEQ ID | LASPSQTSLYF (SEQ ID NO: | F (SEQ ID NO: | (SEQ ID |
| | | | NO: 37) | NO: | NO: 5939) | NO: | 11909) | 25741) | NO: |
| | | | | 3005) | | 8941) | | | 16341) |
| Clonotype_ | 1 | TRA | GDSVTQKQGQVTLSEDDF | TTTYPT | LLWYVQYPGQGPQ | VTTANNK | GISRGFEATYDKGTTSFHLQKASVQ | CALVEGATGGNNKL | GQGTVLSVI |
| 175 | | | LFINCTYS (SEQ ID | (SEQ ID | LLLK (SEQ ID | (SEQ ID | ESDSAVYF (SEQ ID NO: | TF (SEQ ID NO: | P (SEQ ID |
| | | | NO: 510) | NO: 3478) | NO: 21948) | NO: 9054) | 12382) | 25742) | NO: |
| | | | | | | | | | 16368) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSEGTNQAPLF | GEGTRLSVL |
| 176 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25743) | NO: |
| | | | | 3005) | | 8941) | | | 16441) |
| Clonotype_ | 1 | TRA | GDSVTQTEGQVTVSESKS | ATSIAYP | LFWYVRYPGEGLQ | VITAGQK | GSSRGFEATYNKETTSFHLQKASVQ | CALSDQGGSAKLIF | GEGTKLTVS |
| 176 | | | LIINCTYS (SEQ ID | N (SEQ | LLLK (SEQ ID | (SEQ ID | ESDSAVYY (SEQ ID NO: | (SEQ ID NO: | S (SEQ ID |
| | | | NO: 60) | ID NO: | NO: 5996) | NO: | 11932) | 25672) | NO: |
| | | | | 3098) | | 8964) | | | 16384) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSEGTGRIDSGN | GEGSRLIVV |
| 177 | | | VTLSCRQT (SEQ ID | (SEQ ID | LIHY (SEQ ID | (SEQ ID | LASPSQTSLYF (SEQ ID NO: | TLYF (SEQ ID | (SEQ ID |
| | | | NO: 37) | NO: 3005) | NO: 5939) | NO: 8941) | 11909) | NO: 25745) | NO: 16471) |
| Clonotype_ | 1 | TRA | AQKVTQTQTSISVMEKTT | TRDSSYF | LFWYKQTASGEIV | QDSYKKE | ATEGHYSLNFQKPKSSIGLIITATQ | CAMREVTGANTGKL | GHGTILRVH |
| 177 | | | VTMDCVYE (SEQ ID | (SEQ ID | FLIR (SEQ ID | N (SEQ | IEDSAVYF (SEQ ID NO: | TF (SEQ ID NO: | P (SEQ ID |
| | | | NO: 44) | NO: | NO: 5942) | ID NO: | 11916) | 25746) | NO: |
| | | | | 2974) | | 8910) | | | 16356) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDSANTEVFF | GKGTRLTVV |
| 178 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: 3005) | NO: 5939) | NO: 8941) | 11909) | 25747) | NO: |
| | | | | | | | | | 16333) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGGGSAKLIF | GEGTKLTVS |
| 178 | | | VTMDCVYE (SEQ ID | (SEQ ID | FLIR (SEQ ID | N (SEQ | IEDSAVYF (SEQ ID NO: | (SEQ ID NO: | S (SEQ ID |
| | | | NO: 44) | NO: | NO: 5942) | ID NO: | 11878) | 25748) | NO: |
| | | | | 3056) | | 8910) | | | 16384) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDLGGSAETLY | GSGTRLTVL |
| 179 | | | VTLSCRQT (SEQ ID | (SEQ ID | LIHY (SEQ ID | (SEQ ID | LASPSQTSLYF (SEQ ID NO: | F (SEQ ID NO: | (SEQ ID |
| | | | NO: 37) | NO: | NO: 5939) | NO: | 11909) | 25749) | NO: |
| | | | | 3005) | | 8941) | | | 16325) |
| Clonotype_ | 1 | TRA | QQKVQQSPESLIVPEGAM | DSASQY | FAWYRQHSGKAPK | IFSNGE | KEEGRFTIHLNKASLHFSLHIRDSQ | CAVSGDSNYQLIW | GSGTKLIIK |
| 179 | | | TSLNCTFS (SEQ ID | (SEQ ID | ALMS (SEQ ID | (SEQ ID | PSDSALYL (SEQ ID NO: 11948) | (SEQ ID NO: | P (SEQ ID |
| | | | NO: 214) | NO: 3044) | NO: 6150) | NO: 8932) | | 25750) | NO: 16372) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDSSSYEQYF | GPGTRLTVL |
| 180 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25751) | NO: |
| | | | | 3005) | | 8941) | | | 16327) |
| Clonotype_ | 1 | TRA | GEQVEQLPSILRVQEGSS | DSASNY | FPWYKQEPGENPK | IRSNMER | KQIQELIVLLDKKAKRFSLHITDTQ | CAASEGGGSAKLIF | GEGTKLTVS |
| 180 | | | ASINCSYE (SEQ ID | (SEQ ID | LIID (SEQ ID | (SEQ ID | PGDSAMYF (SEQ ID NO: | (SEQ ID NO: | S (SEQ ID |
| | | | NO: 48) | NO: | NO: 5984) | NO: | 11920) | 25752) | NO: |
| | | | | 3016) | | 8952) | | | 16384) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDWQNTLYF | GAGTRLSVL |
| 181 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25753) | NO: |
| | | | | 3005) | | 8941) | | | 16365) |
| Clonotype_ | 1 | TRA | AQSVTQPDARVTVSEGAS | SSVTPY | LFWYVQYPRQGLQ | YYSGDPV | GVNGFEAEFSKSNSSFHLRKASVH | CASDQGGSAKLIF | GEGTKLTVS |
| 181 | | | LQLRCKYS (SEQ ID | (SEQ ID | LLLK (SEQ ID | V (SEQ | WSDSAVYF (SEQ ID NO: | (SEQ ID NO: | S (SEQ ID |
| | | | NO: 12) | NO: | NO: 5948) | ID NO: | 11892) | 25754) | NO: |
| | | | | 2988) | | 8916) | | | 16384) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDFRGGFF | GKGTRLTVV |
| 182 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25755) | NO: |
| | | | | 3005) | | 8941) | | | 16333) |
| Clonotype_ | 1 | TRA | GDSVTQTEGLVTVTEGLP | TTYLTIA | FFWYVQYLNEAPQ | SSTDNKR | TEHQGFHATLHKSSSSFHLQKSSAQ | CALSSGGSNAKLTF | GKGTKLSVK |
| 182 | | | VKLNCTYQ (SEQ ID | (SEQ ID | VLLK (SEQ ID | (SEQ ID | LSDSALYY (SEQ ID NO: | (SEQ ID NO: | S (SEQ ID |
| | | | NO: 116) | NO: | NO: 6052) | NO: | 11918) | 25756) | NO: |
| | | | | 3084) | | 8912) | | | 16342) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSETGGASAETL | GSGTRLTVL |
| 183 | | | VTLSCRQT (SEQ ID | (SEQ ID | LIHY (SEQ ID | (SEQ ID | LASPSQTSLYF (SEQ ID NO: | YF (SEQ ID NO: | (SEQ ID |
| | | | NO: 37) | NO: | NO: 5939) | NO: | 11909) | 25757) | NO: |
| | | | | 3005) | | 8941) | | | 16325) |
| Clonotype_ | 1 | TRA | GNSVTQMQGQVTLSEEEF | TTGYPT | LFWYVQYPGEGPQ | VTTANNK | GSSRGFEATYDKGTTSFHLQKASVQ | CVLGDRGQGGRALI | GTGTTVSVS |
| 183 | | | LFINCTYS (SEQ ID | (SEQ ID | LLLK (SEQ ID | (SEQ ID | ESDSAVYY (SEQ ID NO: 12124) | F (SEQ ID NO: | P (SEQ ID |
| | | | NO: 252) | NO: 3220) | NO: 6086) | NO: 9054) | | 25758) | NO: |
| | | | | | | | | | 16348) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSERLGPSNTGQ | GEGSKLTVL |
| 184 | | | VTLSCRQT (SEQ ID | (SEQ ID | LIHY (SEQ ID | (SEQ ID | LASPSQTSLYF (SEQ ID NO: | LYF (SEQ ID NO: | (SEQ ID |
| | | | NO: 37) | NO: | NO: 5939) | NO: | 11909) | 25759) | NO: |
| | | | | 3005) | | 8941) | | | 16347) |
| Clonotype_ | 1 | TRA | LAKTTQPPSMEAYEGQEV | NIATSEY | FIIQ (SEQ ID | GYKDYVV | NEVASLFISADRKLSTLSLPWVSLR | CIVTDRGFASALTF | GSGTKVIVL |
| 184 | | | NVSCSHT (SEQ ID NO: | (SEQ ID | IYWYRQVPHQGPQ | (SEQ ID | DAAVYY (SEQ ID NO: 11978) | (SEQ ID NO: | PY (SEQ ID |
| | | | 106) | NO: | NO: 6042) | NO: | | 25760) | NO: |
| | | | | 3074) | | 9010) | | | 17004) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSEGTGGYAEQF | GPGTRLTVL |
| 185 | | | VTLSCRQT (SEQ ID | (SEQ ID | LIHY (SEQ ID | (SEQ ID | LASPSQTSLYF (SEQ ID NO: | F (SEQ ID NO: | (SEQ ID |
| | | | NO: 37) | NO: | NO: 5939) | NO: | 11909) | 25761) | NO: |
| | | | | 3005) | | 8941) | | | 16327) |
| Clonotype_ | 1 | TRA | QQKVQQSPESLTVSEGAM | DGTSNN | FRWYRQHSAKGLE | IFSDGE | KEEGRFTAHLNRANLHVSLHIREPQ | CAVRGSWQLIF | GSGTQLTVM |
| 185 | | | ASLNCTFS (SEQ ID | (SEQ ID | VLVS (SEQ ID | (SEQ ID | PSDSAVYL (SEQ ID NO: | (SEQ ID NO: | P (SEQ ID |
| | | | NO: 92) | NO: | NO: 6028) | NO: | 11964) | 25762) | NO: |
| | | | | 3060) | | 8996) | | | 16400) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSEGGANERLFF | GHGTKLSVL |
| 186 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: 3005) | NO: 5939) | NO: 8941) | 11909) | 25763) | NO: |
| | | | | | | | | | 16401) |
| Clonotype_ | 1 | TRA | GDSVTQTEGQVTVSESKS | TTSIAYP | LFWYVRYPGEGLQ | VITAGQK | GSSRGFEATYNKETTSFHLQKASVQ | CALGDNVGDNSKLI | GLGTSLVVN |
| 186 | | | LIINCTYS (SEQ ID | N (SEQ | LLLK (SEQ ID | (SEQ ID | ESDSAVYY (SEQ ID NO: | W (SEQ ID NO: | P (SEQ ID |
| | | | NO: 60) | ID NO: | NO: 5996) | NO: | 11932) | 25764) | NO: |
| | | | | 3028) | | 8964) | | | 17292) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSEYRVANTEVF | GKGTRLTVV |
| 187 | | | VTLSCRQT (SEQ ID | (SEQ ID | LIHY (SEQ ID | (SEQ ID | LASPSQTSLYF (SEQ ID NO: | F (SEQ ID NO: | (SEQ ID |
| | | | NO: 37) | NO: | NO: 5939) | NO: | 11909) | 25765) | NO: |
| | | | | 3005) | | 8941) | | | 16333) |
| Clonotype_ | 1 | TRA | GDSVTQTEGQVALSEEDF | ASGYPA | LFWYVQYPGEGPQ | ASRDKEK | GSSRGFEATYDKGTTSFHLRKASVQ | CALGVMDYANKMIF | GLGTILRVR |
| 187 | | | LTIHCNYS (SEQ ID | (SEQ ID | FLFR (SEQ ID | (SEQ ID | ESDSAVYY (SEQ ID NO: | (SEQ ID NO: | P (SEQ ID |
| | | | NO: 14) | NO: | NO: 6050) | NO: | 11886) | 25766) | NO: |
| | | | | 3082) | | 8918) | | | 16416) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSEWDSNERLFF | GHGTKLSVL |
| 188 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25767) | NO: |
| | | | | 3005) | | 8941) | | | 16401) |
| Clonotype_ | 1 | TRA | AQKVTQTQTSISVMEKTT | TRDSSYF | LFWYKQTASGEIV | QDSYKKE | ATEGHYSLNFQKPKSSIGLIITATQ | CAMREGEGTGGNNK | GQGTVLSVI |
| 188 | | | VTMDCVYE (SEQ ID | (SEQ ID | FLIR (SEQ ID | N (SEQ | IEDSAVYF (SEQ ID NO: | LTF (SEQ ID NO: | P (SEQ ID |
| | | | NO: 44) | NO: | NO: 5942) | ID NO: | 11916) | 25768) | NO: |
| | | | | 2974) | | 8910) | | | 16368) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDKGLEVFF | GKGTRLTVV |
| 189 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25769) | NO: |
| | | | | 3005) | | 8941) | | | 16333) |
| Clonotype_ | 1 | TRA | QQKVQQSPESLIVPEGGM | DRNSQY | FWWYRQHSGEGPK | IFSNGD | KKEGRFTAHLNKASLYVSLHIKDSQ | CAVDGGSGNKLIF | GTGTLLSVK |
| 189 | | | ASLNCTFS (SEQ ID | (SEQ ID | ALMS (SEQ ID | (SEQ ID | PSDSALYF (SEQ ID NO: 12542) | (SEQ ID NO: | P (SEQ ID |
| | | | NO: 132) | NO: | NO: 5938) | NO: 9036) | | 25770) | NO: 16350) |
| | | | | 3100) | | | | | |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDPVSNERLFF | GHGTKLSVL |
| 190 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25771) | NO: |
| | | | | 3005) | | 8941) | | | 16401) |
| Clonotype_ | 1 | TRA | GEQVEQRPPHLSVPEGDS | DSATAY | FYWYKQEPGAGLQ | VFSNVDR | KEEQGLTVLLNKKDKQLSLNLTAAH | CAVIASSSFSKLVF | GQGTSLSVV |
| 190 | | | AVIICTYT (SEQ ID | (SEQ ID | LLMS (SEQ ID | (SEQ ID | PGDSAVYF (SEQ ID NO: | (SEQ ID NO: | P (SEQ ID |
| | | | NO: 100) | NO: | NO: 6036) | NO: | 11972) | 25772) | NO: |
| | | | | 2990) | | 9004) | | | 16422) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDDRGFAEQFF | GPGTRLTVL |
| 191 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25773) | NO: |
| | | | | 3005) | | 8941) | | | 16327) |
| Clonotype_ | 1 | TRA | GDSVTQTEGLVTLTKGLP | TTYSPF | LFWYVQHLNEAPK | SSTDNKR | TEHQGFYATLHKSSSSFHLQKSSVQ | CALSNYAQGLTF | GLGTRVSVF |
| 191 | | | VMLNCTYQ (SEQ ID | (SEQ ID | LLLK (SEQ ID | (SEQ ID | LSDSALYF (SEQ ID NO: | (SEQ ID NO: | P (SEQ ID |
| | | | NO: 8) | NO: | NO: 5944) | NO: 8912) | 11880) | 25774) | NO: 16344) |
| | | | | 2976) | | | | | |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDAGISNERLF | GHGTKLSVL |
| 192 | | | VTLSCRQT (SEQ ID | (SEQ ID | LIHY (SEQ ID | (SEQ ID | LASPSQTSLYF (SEQ ID NO: | F (SEQ ID NO: | (SEQ ID |
| | | | NO: 37) | NO: | NO: 5939) | NO: | 11909) | 25775) | NO: |
| | | | | 3005) | | 8941) | | | 16401) |
| Clonotype_ | 1 | TRA | QQQVRQSPQSLTVWEGET | NSAFDY | FPWYQQFPGEGPA | IRSVSDK | KEDGGFTIFFNKREKNLSLHIKDSQ | CAARYAQGLTF | GLGTRVSVF |
| 192 | | | AILNCSYE (SEQ ID | (SEQ ID | LLIA (SEQ ID | (SEQ ID | PGDSATYF (SEQ ID NO: | (SEQ ID NO: | P (SEQ ID |
| | | | NO: 68) | NO: | NO: 6020) | NO: | 24470) | 25776) | NO: |
| | | | | 3052) | | 8988) | | | 16344) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDALGGTETLY | GSGTRLTVL |
| 193 | | | VTLSCRQT (SEQ ID | (SEQ ID | LIHY | (SEQ ID | LASPSQTSLYF (SEQ ID NO: | F (SEQ ID NO: | (SEQ ID |
| | | | NO: 37) | NO: | (SEQ ID | NO: | 11909) | 25777) | NO: |
| | | | | 3005) | NO: 5939) | 8941) | | | 16325) |
| Clonotype_ | 1 | TRA | GDSVTQTEGLVTLTEGLP | TAYSTF | LFWYVQHLNEAPK | SSTDNKR | TEHQGFHATLHKSSSSFHLQKSSAQ | CALRGNNAGAKLTF | GGGTRLTVR |
| 193 | | | VMLNCTYQ (SEQ ID | (SEQ ID | LLLK (SEQ ID | (SEQ ID | LSDSALYY (SEQ ID NO: 11918) | (SEQ ID NO: | P (SEQ ID |
| | | | NO: 46) | NO: 3030) | NO: 5944) | NO: 8912) | | 25778) | NO: 16338) |
| Clonotype_ | 1 | TRB | EAAVTQSPRNKVTVTGGN | NSHNY (S) | MYWYRQDTGHGLR | SYGAGN | LQIGDVPDGYKATRTTQEDFFLLLE | CASSDAAGREQYF | GPGTRLTVL |
| 194 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: 8941) | 11909) | 25779) | NO: 16327) |
| | | | | 3005) | | | | | |
| Clonotype_ | 1 | TRA | GDSVTQTEGPVTVSESES | ATSIAYP | LFWYVRYPGEGLQ | VITAGQK | GSSRGFEATYNKETTSFHLQKASVQ | CALDYTNAYKVIF | GKGTHLHVL |
| 194 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 130) | ID NO: | NO: 5996) | NO: | 11932) | 25780) | NO: |
| | | | | 3098) | | 8964) | | | 16330) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDGDTLYF | GAGTRLSVL |
| 195 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25781) | NO: |
| | | | | 3005) | | 8941) | | | 16365) |
| Clonotype_ | 1 | TRA | GDSVTQTEGPVTVSESES | ATSIAYP | LFWYVRYPGEGLQ | VITAGQK | GSSRGFEATYNKETTSFHLQKASVQ | CALGDRTNAYKVIF | GKGTHLHVL |
| 195 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 130) | ID NO: | NO: 5996) | NO: 8964) | 11932) | 25782) | NO: 16330) |
| | | | | 3098) | | | | | |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDGGVAEQFF | GPGTRLTVL |
| 196 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: 3005) | NO: 5939) | NO: 8941) | 11909) | 25783) | NO: 16327) |
| Clonotype_ | 1 | TRA | GDSVTQTEGLVTLTEGLP | TAYSTF | ILFWYVQHLNEAPK | SSTDNKR | TEHQGFHATLHKSSSSFHLQKSSAQ | CALSDGNEKITF | GAGTKLTIK |
| 196 | | | VMLNCTYQ (SEQ ID | (SEQ ID | LLLK (SEQ ID | (SEQ ID | LSDSALYY (SEQ ID NO: | (SEQ ID NO: | P (SEQ ID |
| | | | NO: 46) | NO: | NO: 5944) | NO: | 11918) | 25784) | NO: |
| | | | | 3030) | | 8912) | | | 16512) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDGGNSDYTF | GSGTRLLVI |
| 197 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25785) | NO: |
| | | | | 3005) | | 8941) | | | 16337) |
| Clonotype_ | 1 | TRA | AQKVTQTQTSISVMEKTT | TRDSSYF | LFWYKQTASGEIV | QDSYKKE | ATEGHYSLNFQKPKSSIGLIITATQ | CAMREGDGNEKITF | GAGTKLTIK |
| 197 | | | VTMDCVYE (SEQ ID | (SEQ ID | FLIR (SEQ ID | N (SEQ | IEDSAVYF (SEQ ID NO: 11916) | (SEQ ID NO: | P (SEQ ID |
| | | | NO: 44) | NO: | NO: 5942) | ID NO: | | 25786) | NO: 16512) |
| | | | | 2974) | | 8910) | | | |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDVWDRGYEQY | GPGTRLTVL |
| 198 | | | VTLSCRQT (SEQ ID | (SEQ ID | LIHY (SEQ ID | (SEQ ID | LASPSQTSLYF (SEQ ID NO: | F (SEQ ID NO: | (SEQ ID |
| | | | NO: 37) | NO: | NO: 5939) | NO: | 11909) | 25787) | NO: |
| | | | | 3005) | | 8941) | | | 16327) |
| Clonotype_ | 1 | TRA | QQQVRQSPQSLTVWEGET | DSTFDY | FPWYRQFPGKSPA | ISLVSNK | KEDGRFTIFFNKREKKLSLHITDSQ | CAASSGANTGKLTF | GHGTILRVH |
| 198 | | | TILNCSYE (SEQ ID | (SEQ ID | LLIA (SEQ ID | (SEQ ID | PGDSATYF (SEQ ID NO: 11940) | (SEQ ID NO: | P (SEQ ID |
| | | | NO: 19528) | NO: | NO: 21968) | NO: | | 25788) | NO: |
| | | | | 3208) | | 23188) | | | 16356) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDVGANSDYTF | GSGTRLLVI |
| 199 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25789) | NO: |
| | | | | 3005) | | 8941) | | | 16337) |
| Clonotype_ | 1 | TRA | QQKVQQSPESLIVPEGGM | DRNVDY | FWWYRQHSGKSPK | IFSNGE | KEEGRFTVHLNKASLHTSLHIRDSQ | CAAHDSGYNKLTF | GKGTVLLVS |
| 199 | | | ASLNCTSS (SEQ ID | (SEQ ID | MLMS (SEQ ID | (SEQ ID | PSDSALYL (SEQ ID NO: | (SEQ ID NO: | P (SEQ ID |
| | | | NO: 28) | NO: | NO: 5964) | NO: | 11900) | 25790) | NO: |
| | | | | 2996) | | 8932) | | | 16378) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDVGGSAETLY | GSGTRLTVL |
| 200 | | | VTLSCRQT (SEQ ID | (SEQ ID | LIHY (SEQ ID | (SEQ ID | LASPSQTSLYF (SEQ ID NO: | F (SEQ ID NO: | (SEQ ID |
| | | | NO: 37) | NO: | NO: 5939) | NO: | 11909) | 25791) | NO: |
| | | | | 3005) | | 8941) | | | 16325) |
| Clonotype_ | 1 | TRA | QQQVRQSPQSLTVWEGET | DSTFDY | FPWYRLFPGESPA | IRPVSNK | PGDSATYF (SEQ ID NO: | CAASATGGNNKLTF | GQGTVLSVI |
| 200 | | | AILNCSYE (SEQ ID | (SEQ ID | LLIA (SEQ ID | (SEQ ID | KEDGRFTIFFNKREKKLSLHITDSQ | (SEQ ID NO: | P (SEQ ID |
| | | | NO: 68) | NO: | NO: 6176) | NO: | 11940) | 25792) | NO: |
| | | | | 3208) | | 9144) | | | 16368) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDLQGNTEVFF | GKGTRLTVV |
| 201 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: 3005) | NO: 5939) | NO: 8941) | 11909) | 25793) | NO: |
| | | | | | | | | | 16333) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMRYTGNYKYVF | GAGTRLKVI |
| 201 | | | VTMDCVYE (SEQ ID | (SEQ ID | FLIR (SEQ ID | N (SEQ | IEDSAVYF (SEQ ID NO: | (SEQ ID NO: | A (SEQ ID |
| | | | NO: 44) | NO: | NO: 5942) | ID NO: | 11878) | 25794) | NO: |
| | | | | 3056) | | 8910) | | | 16380) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSDLGGRDYAEQ | GPGTRLTVL |
| 202 | | | VTLSCRQT (SEQ ID | (SEQ ID | LIHY (SEQ ID | (SEQ ID | LASPSQTSLYF (SEQ ID NO: | FF (SEQ ID NO: | (SEQ ID |
| | | | NO: 37) | NO: | NO: 5939) | NO: | 11909) | 25795) | NO: |
| | | | | 3005) | | 8941) | | | 16327) |
| Clonotype_ | 1 | TRA | QQKVQQSPESLIVPEGGM | DRNVDY | FWWYRQHSGKSPK | IFSNGE | KEEGRFTVHLNKASLHTSLHIRDSQ | CAAKGNTGKLIF | GLGTTLQVQ |
| 202 | | | ASLNCTSS (SEQ ID | (SEQ ID | MLMS (SEQ ID | (SEQ ID | PSDSALYL (SEQ ID NO: | (SEQ ID NO: | P (SEQ ID |
| | | | NO: 28) | NO: | NO: 5964) | NO: | 11900) | 25796) | NO: |
| | | | | 2996) | | 8932) | | | 16326) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSGTGGANTLYF | GAGTRLSVL |
| 203 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25797) | NO: |
| | | | | 3005) | | 8941) | | | 16365) |
| Clonotype_ | 1 | TRA | GQGVEQPDNLMSVEGTFA | TSGFNG | LSWYQQREGHAPV | VVLDGL | KDSGHFSTFLSRSNGYSYLLLTELQ | CAVRDSTASLGKLQ | GTGTQVVVT |
| 203 | | | RVNCTYS (SEQ ID NO: | (SEQ ID | FLSY (SEQ ID | (SEQ ID | IKDSASYL (SEQ ID NO: | F (SEQ ID NO: | P (SEQ ID |
| | | | 4) | NO: | NO: 5940) | NO: | 11876) | 25798) | NO: |
| | | | | 2972) | | 8908) | | | 16488) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSGSNYAEQFF | GPGTRLTVL |
| 204 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25799) | NO: |
| | | | | 3005) | | 8941) | | | 16327) |
| Clonotype_ | 1 | TRA | QQNVQQSPESLIVPEGAR | DSASQY | FWWYRQHSGKAPK | IFSNGE | KEEGRFTIHLNKASLHFSLHIRDSQ | CAVSTGGADRLTF | GKGTQLIIQ |
| 204 | | | TSLNCTFS (SEQ ID | (SEQ ID | ALMS (SEQ ID | (SEQ ID | PSDSALYL (SEQ ID NO: | (SEQ ID NO: | P (SEQ ID |
| | | | NO: 76) | NO: | NO: 6012) | NO: | 11948) | 25800) | NO: |
| | | | | 3044) | | 8932) | | | 16360) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSGDWGDTQYF | GPGTRLLVL |
| 205 | | | VTLSCRQT (SEQ ID | (SEQ ID | LIHY (SEQ ID | (SEQ ID | LASPSQTSLYF (SEQ ID NO: | (SEQ ID NO: | (SEQ ID |
| | | | NO: 37) | NO: | NO: 5939) | NO: | 11909) | 25801) | NO: |
| | | | | 3005) | | 8941) | | | 16341) |
| Clonotype_ | 1 | TRA | GEQVEQRPPHLSVREGDS | DPNSYY | FFWYKQEPGAGLQ | VFSSTEI | NEGQGFTVLLNKKDKQLSLNLTAAH | CAVSDSGGNYKPTF | GKGTSLVVH |
| 205 | | | AVIICTYT (SEQ ID | (SEQ ID | LLMK (SEQ ID | (SEQ ID | PGDSAVYF (SEQ ID NO: | (SEQ ID NO: | P (SEQ ID |
| | | | NO: 108) | NO: 3010) | NO: 6044) | NO: 8946) | 11980) | 25802) | NO: |
| | | | | | | | | | 16394) |
| Clonotype_ | 1 | TRB | EAAVTQSPRNKVTVTGGN | NSHNY | MYWYRQDTGHGLR | SYGAGN | LQIGDVPDGYKATRTTQEDFFLLLE | CASSSGTGGTYEQY | GPGTRLTVL |
| 206 | | | VTLSCRQT (SEQ ID | (SEQ ID | LIHY (SEQ ID | (SEQ ID | LASPSQTSLYF (SEQ ID NO: | F (SEQ ID NO: | (SEQ ID |
| | | | NO: 37) | NO: 3005) | NO: 5939) | NO: 8941) | 11909) | 25803) | NO: |
| | | | | | | | | | 16327) |
| Clonotype_ | 1 | TRA | GDSVTQTEGQVALSEEDF | ASGYPA | LFWYVQYPGEGPQ | ASRDKEK | GSSRGFEATYDKGTTSFHLRKASVQ | CALGENYAQGLTF | GLGTRVSVF |
| 206 | | | LTIHCNYS (SEQ ID | (SEQ ID | FLFR (SEQ ID | (SEQ ID | ESDSAVYY (SEQ ID NO: | (SEQ ID NO: | P (SEQ ID |
| | | | NO: 14) | NO: | NO: 6050) | NO: | 11886) | 25804) | NO: |
| | | | | 3082) | | 8918) | | | 16344) |
| Clonotype_ | 7 | TRB | EAAVTQSPRNKVTVTGGN | NSHNY | MYWYRQDTGHGLR | SYGAGN | LQIGDVPDGYKATRTTQEDFFLLLE | CASGRTEVFF (SEQ | GKGTRLTVV |
| 207 | | | VTLSCRQT (SEQ ID | (SEQ ID | LIHY (SEQ ID | (SEQ ID | LASPSQTSLYF (SEQ ID NO: | ID NO: 25805) | (SEQ ID |
| | | | NO: 37) | NO: | NO: 5939) | NO: | 11909) | | NO: |
| | | | | 3005) | | 8941) | | | 16333) |
| Clonotype_ | 1 | TRA | QQQVRQSPQSLTVWEGET | NSAFDY | FPWYQQFPGEGPA | ILSVSDK | KEDGRFTIFFNKREKKLSLHIADSQ | CAASANSGTYQRF | GTGTKLQVV |
| 207 | | | AILNCSYE (SEQ ID | (SEQ ID | LLIS (SEQ ID | (SEQ ID | PGDSATYF (SEQ ID NO: | (SEQ ID NO: | P (SEQ ID |
| | | | NO: 68) | NO: 3052) | NO: 6004) | NO: 9200) | 11996) | 25806) | NO: |
| | | | | | | | | | 16432) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASTRTGGRDSDYT | GSGTRLLVI |
| 208 | | | VTLSCHQT (SEQ ID | (SEQ ID | LIHY (SEQ ID | (SEQ ID | LASLSQTAVYF (SEQ ID NO: | F (SEQ ID NO: | (SEQ ID |
| | | | NO: 7) | NO: 2975) | NO: 5939) | NO: 8911) | 11879) | 25807) | NO: |
| | | | | | | | | | 16337) |
| Clonotype_ | 1 | TRA | SQLAEENLWALSVHEGES | TSITA | LQWYRQKSGEGPA | IRSNERE | KRNGRLRATLDTSSQSSSLSITATR | CASNNYAQGLTF | GLGTRVSVF |
| 208 | | | VTVNCSYK (SEQ ID | (SEQ ID | QLIL (SEQ ID | (SEQ ID | CEDTAVYF (SEQ ID NO: | (SEQ ID | P (SEQ ID |
| | | | NO: 190) | NO: 2992) | NO: 5960) | NO: 8928) | 11896) | NO: 25808) | NO: 16344) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASTRGQKSYEQYF | GPGTRLTVL |
| 209 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: 2975) | NO: 5939) | NO: 8911) | 11879) | 25809) | NO: |
| | | | | | | | | | 16327) |
| Clonotype_ | 1 | TRA | QQQVRQSPQSLTVWEGET | DSTFNY | FPWYQQFPGEGPA | ISSVSDK | KEDGRFTIFFNKREKKLSLHITDSQ | CAASRGNNAGAKLT | GGGTRLTVR |
| 209 | | | AILNCSYE (SEQ ID | (SEQ ID | LLIS | (SEQ ID | PGDSATYF (SEQ ID NO: 11940) | F (SEQ ID NO: | P (SEQ ID |
| | | | NO: 68) | NO: | (SEQ ID | NO: | | 25810) | NO: |
| | | | | 3036) | NO: 6004) | 8972) | | | 16338) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSPGDGNTLYF | GEGSRLIVV |
| 210 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25811) | NO: |
| | | | | 2975) | | 8911) | | | 16471) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAARDSNYQLIW | GSGTKLIIK |
| 210 | | | ASLNCTSS (SEQ ID | (SEQ ID | ALMS (SEQ ID | (SEQ ID | PSDSALYF (SEQ ID NO: | (SEQ ID NO: | P (SEQ ID |
| | | | NO: 64) | NO: 2970) | NO: 5938) | NO: 8906) | 11874) | 15044) | NO: |
| | | | | | | | | | 16372) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSPGTAYEQYF | GPGTRLTVL |
| 211 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: 2975) | NO: 5939) | NO: 8911) | 11879) | 25813) | NO: |
| | | | | | | | | | 16327) |
| Clonotype_ | 1 | TRA | GQQVQQSPSSLVLQEGEN | STATQ | LQWFYQSPGGSLV | NPSGT | KHTGRLTSTTVTKERRSSLHISSSQ | CAIVPLTGNTRKLI | GLGTTLQVQ |
| 211 | | | AELQCNFS (SEQ ID | (SEQ ID | SLLS (SEQ ID | (SEQ ID | TTDSGTYL (SEQ ID NO: | F (SEQ ID NO: | P (SEQ ID |
| | | | NO: 322) | NO: | NO: 6258) | NO: | 12194) | 25814) | NO: |
| | | | | 3290) | | 8914) | | | 16326) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSLQGTEVFF | GKGTRLTVV |
| 212 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: 2975) | NO: 5939) | NO: 8911) | 11879) | 25815) | NO: |
| | | | | | | | | | 16333) |
| Clonotype_ | 1 | TRA | AQKVTQTQTSISVVEKTT | TRDSSYF | LFWYKQTASGEIV | QDSYKKE | ATVGHYSLNFQKPKSSIGLIITATQ | CAMREGGGSNAKLT | GKGTKLSVK |
| 212 | | | VTMDCVYE (SEQ ID | (SEQ ID | FLIR (SEQ ID | N (SEQ | IEDSAVYF (SEQ ID NO: | F (SEQ ID NO: | S (SEQ ID |
| | | | NO: 6) | NO: | NO: 5942) | ID NO: | 11878) | 25816) | NO: |
| | | | | 2974) | | 8910) | | | 16342) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSADRGGTEVFF | GKGTRLTVV |
| 213 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25817) | NO: |
| | | | | 2975) | | 8911) | | | 16333) |
| Clonotype_ | 1 | TRA | GEQVEQLPSILRVQERSS | NSASNY | FPWYKQEPGENPK | IRSNMER | KQIQGLIVLLDKKAKRFSLHITDTQ | CAASAYNNNAPRF | GAGTKLTVK |
| 213 | | | ASINCTYE (SEQ ID | (SEQ ID | LIID (SEQ ID | (SEQ ID | PADSAMYF (SEQ ID NO: | (SEQ ID NO: | P (SEQ ID |
| | | | NO: 182) | NO: | NO: 5984) | NO: | 12054) | 25818) | NO: |
| | | | | 3138) | | 8952) | | | 16424) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASRDNTGQLYF | GEGSKLTVL |
| 214 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: 2975) | NO: 5939) | NO: 8911) | 11879) | 25819) | NO: 16347) |
| Clonotype_ | 1 | TRA | LAKTTQPPSMEAYEGQEV | NIATSEY | FIIQ (SEQ ID | GYKDYVV | NEVASLFISADRKLSTLSLPWVSLR | CIVTDTGFASALTF | GSGTKVIVL |
| 214 | | | NVSCSHT (SEQ ID NO: | (SEQ ID | IYWYRQVPHQGPQ | (SEQ ID | DAAVYY (SEQ ID NO: 11978) | (SEQ ID NO: | PY (SEQ ID |
| | | | 106) | NO: | NO: 6042) | NO: | | 25820) | NO: |
| | | | | 3074) | | 9010) | | | 17004) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASRDRGREQYF | GPGTRLTVL |
| 215 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25821) | NO: |
| | | | | 2975) | | 8911) | | | 16327) |
| Clonotype_ | 1 | TRA | GEQVEQLPSILRVQEGSS | DSASNY | FPWYKQEPGENPK | IRSNMER | KQIQELIVLLDKKAKRFSLHITDTQ | CAASPNTNKVVF | GTGTRLQVL |
| 215 | | | ASINCSYE (SEQ ID | (SEQ ID | LIID (SEQ ID | (SEQ ID | PGDSAMYF (SEQ ID NO: | (SEQ ID NO: | P (SEQ ID |
| | | | NO: 48) | NO: | NO: 5984) | NO: | 11920) | 25822) | NO: |
| | | | | 3016) | | 8952) | | | 16698) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASRGLGAYEQYF | GPGTRLTVL |
| 216 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: 8911) | 11879) | 25823) | NO: 16327) |
| | | | | 2975) | | | | | |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGFSGSWQLI | GSGTQLTVM |
| 216 | | | VTMDCVYE (SEQ ID | (SEQ ID | FLIR (SEQ ID | N (SEQ | IEDSAVYF (SEQ ID NO: | F (SEQ ID NO: | P (SEQ ID |
| | | | NO: 44) | NO: | NO: 5942) | ID NO: | 11878) | 25824) | NO: |
| | | | | 3056) | | 8910) | | | 16400) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSTGGNYEQYF | GPGTRLTVL |
| 217 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25825) | NO: |
| | | | | 2975) | | 8911) | | | 16327) |
| Clonotype_ | 1 | TRA | AQSVTQPDARVTVSEGAS | SSVTPY | LFWYVQYPRQGLQ | YYSGDPV | GVNGFEAEFSKSNSSFHLRKASVH | CAVSSEDTGYQNFY | GKGTSLTVI |
| 217 | | | LQLRCKYS (SEQ ID | (SEQ ID | LLLK (SEQ ID | V (SEQ | WSDSAVYF (SEQ ID NO: | F (SEQ ID NO: | P (SEQ ID |
| | | | NO: 12) | NO: | NO: 5948) | ID NO: | 11892) | 25826) | NO: |
| | | | | 2988) | | 8916) | | | 16366) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSPTGGAGSDYT | GSGTRLLVI |
| 218 | | | VTLSCHQT (SEQ ID | (SEQ ID | LIHY (SEQ ID | (SEQ ID | LASLSQTAVYF (SEQ ID NO: | F (SEQ ID NO: | (SEQ ID |
| | | | NO: 7) | NO: | NO: 5939) | NO: | 11879) | 25827) | NO: |
| | | | | 2975) | | 8911) | | | 16337) |
| Clonotype_ | 1 | TRA | GDSVTQTEGLVTLTKGLP | TTYSPF | ILFWYVQHLNEAPK | SSTDNKR | TEHQGFYATLHKSSSSFHLQKSSVQ | CALSGAMSNYNVLY | GSGTKLTVE |
| 218 | | | VMLNCTYQ (SEQ ID | (SEQ ID | LLLK (SEQ ID | (SEQ ID | LSDSALYF (SEQ ID NO: | F (SEQ ID NO: | P (SEQ ID |
| | | | NO: 8) | NO: | NO: 5944) | NO: | 11880) | 25828) | NO: |
| | | | | 2976) | | 8912) | | | 16336) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSETYEQYF | GPGTRLTVL |
| 219 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25829) | NO: |
| | | | | 2975) | | 8911) | | | 16327) |
| Clonotype_ | 1 | TRA | TQTVSQSDAHVTVFEGDS | YGGSIY | LSWYIQHHGRGLQ | YYSGNPV | GVNGFEAEFSKSDSSFHLRKASVH | CAVSAAGSGGKLTL | GTGTRLQVN |
| 219 | | | VELRCNYS (SEQ ID | (SEQ ID | FLLK (SEQ ID | V (SEQ | WSDSAVYF (SEQ ID NO: | (SEQ ID NO: | L (SEQ ID |
| | | | NO: 110) | NO: | NO: 6046) | ID NO: | 11946) | 25830) | NO: |
| | | | | 3078) | | 9014) | | | 16448) |
| Clonotype_ | 1 | TRB | EAAVTQSPRSKVAVTGGK | INNHDY | MYWYRQDTGHGLR | SYVADS | TEKGDIPDGYKASRPSQENFSLILE | CASSDNSDYTF | GSGTRLLVI |
| 220 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25831) | NO: |
| | | | | 2975) | | 8911) | | | 16337) |
| Clonotype_ | 1 | TRA | GDQVEQSPSALSLHEGTD | TTMRS | VQWFRQNSRGSLI | LASGT | KENGRLKSAFDSKERRYSTLHIRDA | CAANTGANTGKLTF | GHGTILRVH |
| 220 | | | SALRCNFT (SEQ ID | (SEQ ID | SLFY (SEQ ID | (SEQ ID | QLEDSGTYF (SEQ ID NO: | (SEQ ID NO: | P (SEQ ID |
| | | | NO: 18) | NO: | NO: 5954) | NO: | 11890) | 25438) | NO: |
| | | | | 2986) | | 8922) | | | 16356) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSDSTEVFF | GKGTRLTVV |
| 221 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: 2975) | NO: 5939) | NO: 8911) | 11879) | 25833) | NO: 16333) |
| Clonotype_ | 1 | TRA | QQQVRQSPQSLTVWEGET | NSAFDY | FPWYQQFPGEGPA | ILSVSNK | KEDGRFTIFFNKREKKLSLHIADSQ | CAASEGNYKYVF | GAGTRLKVI |
| 221 | | | AILNCSYE (SEQ ID | (SEQ ID | LLIS (SEQ ID | (SEQ ID | PGDSATYF (SEQ ID NO: | (SEQ ID NO: | A (SEQ ID |
| | | | NO: 68) | NO: | NO: 6004) | NO: 9028) | 11996) | 25834) | NO: 16380) |
| | | | | 3052) | | | | | |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSERGANTEVFF | GKGTRLTVV |
| 222 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25835) | NO: |
| | | | | 2975) | | 8911) | | | 16333) |
| Clonotype_ | 1 | TRA | GEQVEQLPSSLIVQEGAS | DSASVY | FPWYKQEPGKRLQ | IRSNMER | KQNQRLTLLFDKKTKHLSLHITATQ | CSASPMDYANKMIF | GLGTILRVR |
| 222 | | | VLINCSYT (SEQ ID | (SEQ ID | FIID (SEQ ID | (SEQ ID | PGDSAIYF (SEQ ID NO: | (SEQ ID NO: | P (SEQ ID |
| | | | NO: 160) | NO: | NO: 6096) | NO: | 12032) | 25836) | NO: |
| | | | | 3128) | | 8952) | | | 16416) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSEGGWAETLYF | GSGTRLTVL |
| 223 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25837) | NO: |
| | | | | 2975) | | 8911) | | | 16325) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAASEEGGGSGNKL | GTGTLLSVK |
| 223 | | | ASFNCTSS (SEQ ID | (SEQ ID | ALMS (SEQ ID | (SEQ ID | PSDSALYF (SEQ ID NO: | IF (SEQ ID NO: | P (SEQ ID |
| | | | NO: 2) | NO: | NO: 5938) | NO: | 11874) | 25838) | NO: |
| | | | | 2970) | | 8906) | | | 16350) |
| Clonotype_ | 1 | TRB | EAAVTQSPRSKVAVTGGK | INNHDY | MYWYRQDTGHGLR | SYVADS | TEKGDIPDGYKASRPSQENFSLILE | CASSDPGRDTEVFF | GKGTRLTVV |
| 224 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 14927) | NO: |
| | | | | 2975) | | 8911) | | | 16333) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGGGGADRLT | GKGTQLIIQ |
| 224 | | | VTMDCVYE (SEQ ID | (SEQ ID | FLIR (SEQ ID | N (SEQ | IEDSAVYF (SEQ ID NO: | F (SEQ ID NO: | P (SEQ ID |
| | | | NO: 44) | NO: | NO: 5942) | ID NO: | 11878) | 14928) | NO: |
| | | | | 3056) | | 8910) | | | 16360) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSDEISNERLFF | GHGTKLSVL |
| 225 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: 2975) | NO: 5939) | NO: 8911) | 11879) | 25841) | NO: |
| | | | | | | | | | 16401) |
| Clonotype_ | 1 | TRA | GEQVEQLPSSLIVQEGAS | DSASVY | FPWYKQEPGKRLQ | IRSNMER | KQNQRLTLLFDKKTKHLSLHITATQ | CSASMGNAGAKLTF | GGGTRLTVR |
| 225 | | | VLINCSYT (SEQ ID | (SEQ ID | FIID (SEQ ID | (SEQ ID | PGDSAIYF (SEQ ID NO: | (SEQ ID NO: | P (SEQ ID |
| | | | NO: 160) | NO: | NO: 6096) | NO: | 12032) | 25842) | NO: |
| | | | | 3128) | | 8952) | | | 16338) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSDDANSDYTF | GSGTRLLVI |
| 226 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25843) | NO: |
| | | | | 2975) | | 8911) | | | 16337) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGPSNTDKVV | GTGTRLQVS |
| 226 | | | VTMDCVYE (SEQ ID | (SEQ ID | FLIR (SEQ ID | N (SEQ | IEDSAVYF (SEQ ID NO: 11878) | F (SEQ ID NO: | P (SEQ ID |
| | | | NO: 44) | NO: | NO: 5942) | ID NO: | | 25844) | NO: 16374) |
| | | | | 3056) | | 8910) | | | |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSDAGSHSGNTL | GEGSRLIVV |
| 227 | | | VTLSCHQT (SEQ ID | (SEQ ID | LIHY (SEQ ID | (SEQ ID | LASLSQTAVYF (SEQ ID NO: | YF (SEQ ID NO: | (SEQ ID |
| | | | NO: 7) | NO: | NO: 5939) | NO: | 11879) | 25845) | NO: |
| | | | | 2975) | | 8911) | | | 16471) |
| Clonotype_ | 1 | TRA | AQKVTQTQTSISVMEKTT | TRDSSYF | FLIR (SEQ ID | QDSYKKE | ATEGHYSLNFQKPKSSIGLIITATQ | CAMREVNTGANTGK | GHGTILRVH |
| 227 | | | VTMDCVYE (SEQ ID | (SEQ ID | LFWYKQTASGEIV | N (SEQ | IEDSAVYF (SEQ ID NO: | LTF (SEQ ID NO: | P (SEQ ID |
| | | | NO: 44) | NO: | NO: 5942) | ID NO: | 11916) | 25436) | NO: |
| | | | | 2974) | | 8910) | | | 16356) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSDGTGGSAETL | GSGTRLTVL |
| 228 | | | VTLSCHQT (SEQ ID | (SEQ ID | LIHY (SEQ ID | (SEQ ID | LASLSQTAVYF (SEQ ID NO: | YF (SEQ ID NO: | (SEQ ID |
| | | | NO: 7) | NO: 2975) | NO: 5939) | NO: 8911) | 11879) | 25847) | NO: |
| | | | | | | | | | 16325) |
| Clonotype_ | 1 | TRA | GQQVKQSSPSLTVQEGGI | NDMFDY | FAWYKKYPDNSPT | VRSNVDK | REDGRFTVFLNKSGKHFSLHITASQ | CAAGAGGYQNFYF | GKGTSLTVI |
| 228 | | | LILNCDYE (SEQ ID | (SEQ ID | LLIS (SEQ ID | (SEQ ID | PEDTAVYL (SEQ ID NO: | (SEQ ID NO: | P (SEQ ID |
| | | | NO: 78) | NO: 3046) | NO: 6014) | NO: 8982) | 11950) | 25848) | NO: 16366) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSDVGEQYF | GPGTRLTVL |
| 229 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25849) | NO: |
| | | | | 2975) | | 8911) | | | 16327) |
| Clonotype_ | 1 | TRA | GDQVKQSPSALSLQEGTN | IATTT | VQWFLQNPRGSLM | LVPGT | KENGRLKSTFNSKESYSTLHIRDAQ | CAANSGGSNAKLTF | GKGTKLSVK |
| 229 | | | SALRCNFS (SEQ ID | (SEQ ID | NLFY (SEQ ID | (SEQ ID | LEDSGTYF (SEQ ID NO: | (SEQ ID NO: | S (SEQ ID |
| | | | NO: 258) | NO: | NO: 6194) | NO: | 12130) | 25850) | NO: |
| | | | | 3226) | | 9162) | | | 16342) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSDVRVNSPLYF | AAGTRLTVT |
| 230 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25851) | NO: |
| | | | | 2975) | | 8911) | | | 16381) |
| Clonotype_ | 1 | TRA | SQWGEENLQALSIQEGED | TYTTV | VHWYRQDSGRGPA | IRSNERE | KRSGRLRATLDTSSQSSSLSITAAQ | CATDGGNYAQGLTF | GLGTRVSVF |
| 230 | | | VTMNCSYK (SEQ ID | (SEQ ID | LIIL (SEQ ID | (SEQ ID | CEDTAVYF (SEQ ID NO: | (SEQ ID NO: | P (SEQ ID |
| | | | NO: 80) | NO: | NO: 6016) | NO: | 11952) | 25852) | NO: |
| | | | | 3048) | | 8928) | | | 16344) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSGLGGRGAETL | GSGTRLTVL |
| 231 | | | VTLSCHQT (SEQ ID | (SEQ ID | LIHY (SEQ ID | (SEQ ID | LASLSQTAVYF (SEQ ID NO: | YF (SEQ ID NO: | (SEQ ID |
| | | | NO: 7) | NO: | NO: 5939) | NO: | 11879) | 25853) | NO: |
| | | | | 2975) | | 8911) | | | 16325) |
| Clonotype_ | 1 | TRA | AQKVTQTQTSISVMEKTT | TRDSSYF | LFWYKQTASGEIV | QDSYKKE | ATEGHYSLNFQKPKSSIGLIITATQ | CAMREVHTGANTGK | GHGTILRVH |
| 231 | | | VTMDCVYE (SEQ ID | (SEQ ID | FLIR (SEQ ID | N (SEQ | IEDSAVYF (SEQ ID NO: 11916) | LTF (SEQ ID NO: | P (SEQ ID |
| | | | NO: 44) | NO: | NO: 5942) | ID NO: | | 25854) | NO: |
| | | | | 2974) | | 8910) | | | 16356) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSGGGNEQYF | GPGTRLTVL |
| 232 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25855) | NO: |
| | | | | 2975) | | 8911) | | | 16327) |
| Clonotype_ | 1 | TRA | GEQVEQLPSSLIVQEGAS | DSASVY | FPWYKQEPGKRLQ | IRSNMER | KQTQRLTLLFDKKTKHLSLHITATQ | CSASSQGGRALIF | GTGTTVSVS |
| 232 | | | VLINCSYT (SEQ ID | (SEQ ID | FIID (SEQ ID | (SEQ ID | PGDSAIYF (SEQ ID NO: | (SEQ ID NO: | P (SEQ ID |
| | | | NO: 160) | NO: | NO: 6096) | NO: | 24636) | 25856) | NO: |
| | | | | 3128) | | 8952) | | | 16348) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSGTLNSGNTLY | GEGSRLIVV |
| 233 | | | VTLSCHQT (SEQ ID | (SEQ ID | LIHY (SEQ ID | (SEQ ID | LASLSQTAVYF (SEQ ID NO: | F (SEQ ID NO: | (SEQ ID |
| | | | NO: 7) | NO: 2975) | NO: 5939) | NO: 8911) | 11879) | 25857) | NO: |
| | | | | | | | | | 16471) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREVNTGANTGK | GHGTILRVH |
| 233 | | | VTMDCVYE (SEQ ID | (SEQ ID | FLIR (SEQ ID | N (SEQ | IEDSAVYF (SEQ ID NO: 11878) | LTF (SEQ ID NO: | P (SEQ ID |
| | | | NO: 44) | NO: 3056) | NO: 5942) | ID NO: | | 25436) | NO: |
| | | | | | | 8910) | | | 16356) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSGTANSDYTF | GSGTRLLVI |
| 234 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: 2975) | NO: 5939) | NO: 8911) | 11879) | 25859) | NO: |
| | | | | | | | | | 16337) |
| Clonotype_ | 1 | TRA | GQQVQQSPASLVLQEGEN | TSLNS | MQWFYQRPGGSLV | NPSGT | KHSGRLTSTTVIKERRSSLHISSSQ | CALEDNYAQGLTF | GLGTRVSVF |
| 234 | | | AELQCNFS (SEQ ID | (SEQ ID | SLFY (SEQ ID | (SEQ ID | TTDSGTYL (SEQ ID NO: 11928) | (SEQ ID NO: | P (SEQ ID |
| | | | NO: 10) | NO: 2978) | NO: 5946) | NO: 8914) | | 25860) | NO: 16344) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSGRDWDQDTQY | GPGTRLLVL |
| 235 | | | VTLSCHQT (SEQ ID | (SEQ ID | LIHY (SEQ ID | (SEQ ID | LASLSQTAVYF (SEQ ID NO: | F (SEQ ID NO: | (SEQ ID |
| | | | NO: 7) | NO: 2975) | NO: 5939) | NO: 8911) | 11879) | 25861) | NO: |
| | | | | | | | | | 16341) |
| Clonotype_ | 1 | TRA | GQQVKQSSPSLTVQEGGI | NDMEDY | FAWYKKYPDNSPT | VRSNVDK | REDGRLTVFLNKSGKHFSLHITASQ | CAAGEGTGSKLSF | GKGAKLTVS |
| 235 | | | SILNCDYE (SEQ ID | (SEQ ID | LLIS (SEQ ID | (SEQ ID | PEDTAVYL (SEQ ID NO: | (SEQ ID NO: | P (SEQ ID |
| | | | NO: 248) | NO: | NO: 6014) | NO: 8982) | 12120) | 25862) | NO: |
| | | | | 3046) | | | | | 16524) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSSLGNTGQLYF | GEGSKLTVL |
| 236 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25863) | NO: |
| | | | | 2975) | | 8911) | | | 16347) |
| Clonotype_ | 1 | TRA | AQKVTQTQTSISVMEKTT | TRDSSYF | LFWYKQTASGEIV | QDSYKKE | ATEGHYSLNFQKPKSSIGLIITATQ | CAMRESGGSNYKLT | GKGTLLTVT |
| 236 | | | VTMDCVYE (SEQ ID | (SEQ ID | FLIR (SEQ ID | N (SEQ | IEDSAVYF (SEQ ID NO: | F (SEQ ID NO: | P (SEQ ID |
| | | | NO: 44) | NO: | NO: 5942) | ID NO: | 11916) | 25864) | NO: |
| | | | | 2974) | | 8910) | | | 16642) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASSWGGAAEQFF | GPGTRLTVL |
| 237 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25865) | NO: |
| | | | | 2975) | | 8911) | | | 16327) |
| Clonotype_ | 1 | TRA | AQSVDQPDAHVTLSEGAS | YSAAPY | LFWYVQYPGQSLQ | YITGDTV | KGTKGFEAEFRKSNSSFNLKKSPAH | CALEGRSGGSNYKL | GKGTLLTVT |
| 237 | | | LELRCSYS (SEQ ID | (SEQ ID | FLLK (SEQ ID | V (SEQ | WSDSAKYF (SEQ ID NO: 12152) | TF (SEQ ID NO: | P (SEQ ID |
| | | | NO: 280) | NO: | NO: 6216) | ID NO: | | 25866) | NO: 16642) |
| | | | | 3248) | | 9184) | | | |
| Clonotype_ | 1 | TRB | EAAVTQSPRSKVAVTGGK | NNHDY | MYWYRQDTGHGLR | SYVADS | TEKGDIPDGYKASRPSQENFSLILE | CASRDRGDYTF | GSGTRLLVI |
| 238 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25867) | NO: |
| | | | | 2975) | | 8911) | | | 16337) |
| Clonotype_ | 1 | TRA | GDKVKQSPSALSLQEGTN | IAATT | VQWFLQNPRGSLI | LVPGT | KENGRLKSAFDSKESYSTLHIRDAQ | CAAANYGNEKITF | GAGTKLTIK |
| 238 | | | SALRCNFS (SEQ ID | (SEQ ID | NLFY (SEQ ID | (SEQ ID | LEDSGTYF (SEQ ID NO: | (SEQ ID NO: | P (SEQ ID |
| | | | NO: 404) | NO: | NO: 6486) | NO: | 12276) | 15494) | NO: |
| | | | | 3372) | | 9162) | | | 16512) |
| Clonotype_ | 1 | TRB | EAAVTQSPRSKVAVTGGK | NNHDY | MYWYRQDTGHGLR | SYVADS | TEKGDIPDGYKASRPSQENFSLILE | CASTGGYTLYF | GAGTRLSVL |
| 239 | | | VTLSCHQT (SEQ ID | (SEQ ID | LIHY (SEQ ID | (SEQ ID | LASLSQTAVYF (SEQ ID NO: | (SEQ ID NO: | (SEQ ID |
| | | | NO: 7) | NO: | NO: 5939) | NO: | 11879) | 25869) | NO: |
| | | | | 2975) | | 8911) | | | 16365) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGDQGGSAKL | GEGTKLTVS |
| 239 | | | VTMDCVYE (SEQ ID | (SEQ ID | FLIR (SEQ ID | N (SEQ | IEDSAVYF (SEQ ID NO: | IF (SEQ ID NO: | S (SEQ ID |
| | | | NO: 44) | NO: | NO: 5942) | ID NO: | 11878) | 25870) | NO: |
| | | | | 3056) | | 8910) | | | 16384) |
| Clonotype_ | 1 | TRB | DTTVKQNPRYKLARVGKP | MNHDT | MYWYQKKPNQAPK | YYDKIL | NREADTFEKFQSSRPNNSFCSLYIG | CASSTRGREQYF | GPGTRLTVL |
| 240 | | | VNLICSQT (SEQ ID | (SEQ ID | LLLF (SEQ ID | (SEQ ID | SAGLEYSAMYL (SEQ ID NO: | (SEQ ID NO: | (SEQ ID |
| | | | NO: 299) | NO: 3267) | NO: 6235) | NO: 9203) | 12171) | 25871) | NO: 16327) |
| Clonotype_ | 1 | TRA | DAKTTQPDSMESTEGETV | TISGNEY | IYWYRQVPLQGPE | GLQQN | TTNSMAFLAIASDRKSSTLILPHVS | CILRANSGTYQRF | GTGTKLQVV |
| 240 | | | HLPCSHA (SEQ ID NO: | (SEQ ID | YVTH (SEQ ID | (SEQ ID | LRDAAVYH (SEQ ID NO: | (SEQ ID NO: | P (SEQ ID |
| | | | 104) | NO: | NO: 6040) | NO: | 12266) | 25872) | NO: |
| | | | | 3072) | | 9008) | | | 16432) |
| Clonotype_ | 1 | TRB | DTTVKQNPRYKLARVGKP | MNHDT | MYWYQKKPNQAPK | YYDKIL | NREADTFEKFQSSRPNNSFCSLYIG | CASSRGTGVGNTLY | GEGSRLIVV |
| 241 | | | VNLICSQT (SEQ ID | (SEQ ID | LLLF (SEQ ID | ID NO: | SAGLEYSAMYL (SEQ ID NO: | F (SEQ ID NO: | (SEQ ID |
| | | | NO: 299) | NO: | NO: 6235) | 9203) | 12171) | 25873) | NO: |
| | | | | 3267) | | | | | 16471) |
| Clonotype_ | 1 | TRA | GDSVTQTEGQVTVSESKS | ATSIAYP | LFWYVRYPGEGLQ | VITAGQK | GSSRGFEATYNKETTSFHLQKASVQ | CALSALTSGGNYKP | GKGTSLVVH |
| 241 | | | LIINCTYS (SEQ ID | N (SEQ | LLLK (SEQ ID | (SEQ ID | ESDSAVYY (SEQ ID NO: | TF (SEQ ID NO: | P (SEQ ID |
| | | | NO: 60) | ID NO: | NO: 5996) | NO: | 11932) | 25874) | NO: |
| | | | | 3098) | | 8964) | | | 16394) |
| Clonotype_ | 1 | TRB | DTTVKQNPRYKLARVGKP | MNHDT | MYWYQKKPNQAPK | YYDKIL | NREADTFEKFQSSRPNNSFCSLYIG | CASSRGDWGLYEQY | GPGTRLTVL |
| 242 | | | VNLICSQT (SEQ ID | (SEQ ID | LLLF (SEQ ID | (SEQ ID | SAGLEYSAMYL (SEQ ID NO: | F (SEQ ID NO: | (SEQ ID |
| | | | NO: 299) | NO: | NO: 6235) | NO: | 12171) | 25875) | NO: |
| | | | | 3267) | | 9203) | | | 16327) |
| Clonotype_ | 1 | TRA | AQRVTQVQPTGSSQWGEE | TSEYFYR | IFWYRQLFSGEMV | PSFDTQ | RSGRYSVVFQKSFKSISLVISASQ | CALSEPRGSALGRL | GAGTQLIVI |
| 242 | | | VTLDCSYE (SEQ ID | (SEQ ID | FLIY (SEQ ID | N (SEQ | PEDSGTYF (SEQ ID NO: 12182) | HF (SEQ ID NO: | P (SEQ ID |
| | | | NO: 310) | NO: | NO: 6246) | ID NO: | | 25876) | NO: |
| | | | | 3278) | | 9214) | | | 16480) |
| Clonotype_ | 1 | TRB | DTTVKQNPRYKLARVGKP | MNHDT | MYWYQKKPNQAPK | YYDKIL | NREADTFEKFQSSRPNNSFCSLYIG | CASSSGQGNTEVFF | GKGTRLTVV |
| 243 | | | VNLICSQT (SEQ ID | (SEQ ID | LLLF (SEQ ID | (SEQ ID | SAGLEYSAMYL (SEQ ID NO: | (SEQ ID NO: | (SEQ ID |
| | | | NO: 299) | NO: 3267) | NO: 6235) | NO: 9203) | 12171) | 25877) | NO: |
| | | | | | | | | | 16333) |
| Clonotype_ | 1 | TRA | GDQVKQSPSALSLQEGTN | IATTT | VQWFLQNPRGSLM | LVPGT | KENGRLKSTFNSKESYSTLHIRDAQ | CAADGNNRIFF | GDGTQLVVK |
| 243 | | | SALRCNFS (SEQ ID | (SEQ ID | NLFY (SEQ ID | (SEQ ID | LEDSGTYF (SEQ ID NO: | (SEQ ID NO: | P (SEQ ID |
| | | | NO: 258) | NO: | NO: 6194) | NO: | 12130) | 25878) | NO: |
| | | | | 3226) | | 9162) | | | 16328) |
| Clonotype_ | 1 | TRB | DTTVKQNPRYKLARVGKP | MNHDT | MYWYQKKPNQAPK | YYDKIL | NREADTFEKFQSSRPNNSFCSLYIG | CASSRDRDEQYF | GPGTRLTVL |
| 244 | | | VNLICSQT (SEQ ID | (SEQ ID | LLLF (SEQ ID | (SEQ ID | SAGLEYSAMYL (SEQ ID NO: | (SEQ ID NO: | (SEQ ID |
| | | | NO: 299) | NO: | NO: 6235) | NO: | 12171) | 25879) | NO: |
| | | | | 3267) | | 9203) | | | 16327) |
| Clonotype_ | 1 | TRA | AQKVTQTQTSISVMEKTT | TRDSSYF | LFWYKQTASGE IV | QDSYKKE | ATEGHYSLNFQKPKSSIGLIITATQ | CAMREGAGTGSKLS | GKGAKLTVS |
| 244 | | | VTMDCVYE (SEQ ID | (SEQ ID | FLIR (SEQ ID | N (SEQ | IEDSAVYF (SEQ ID NO: | F (SEQ ID NO: | P (SEQ ID |
| | | | NO: 44) | NO: | NO: 5942) | ID NO: | 11916) | 25880) | NO: |
| | | | | 2974) | | 8910) | | | 16524) |
| Clonotype_ | 1 | TRB | DTTVKQNPRYKLARVGKP | MNHDT | MYWYQKKPNQAPK | YYDKIL | NREADTFEKFQSSRPNNSFCSLYIG | CASSALRTEVFF | GKGTRLTVV |
| 245 | | | VNLICSQT (SEQ ID | (SEQ ID | LLLF (SEQ ID | (SEQ ID | SAGLEYSAMYL (SEQ ID NO: | (SEQ ID NO: | (SEQ ID |
| | | | NO: 299) | NO: | NO: 6235) | NO: | 12171) | 25881) | NO: |
| | | | | 3267) | | 9203) | | | 16333) |
| Clonotype_ | 1 | TRA | GDSVTQTEGQVTVSESKS | ATSIGYP | LFWYVRYPGEGLQ | VITAGQK | GSSRGFEATYNKEATSFHLQKASVQ | CALSASSGSWQLIF | GSGTQLTVM |
| 245 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 60) | ID NO: | NO: 5996) | NO: | 12094) | 25882) | NO: |
| | | | | 3330) | | 8964) | | | 16400) |
| Clonotype_ | 1 | TRB | DTTVKQNPRYKLARVGKP | MNHDT | MYWYQKKPNQAPK | YYDKIL | NREADTFEKFQSSRPNNSFCSLYIG | CASSSGTNTGQLYF | GEGSKLTVL |
| 246 | | | VNLICSQT (SEQ ID | (SEQ ID | LLLF (SEQ ID | (SEQ ID | SAGLEYSAMYL (SEQ ID NO: | (SEQ ID NO: | (SEQ ID |
| | | | NO: 299) | NO: | NO: 6235) | NO: | 12171) | 25883) | NO: |
| | | | | 3267) | | 9203) | | | 16347) |
| Clonotype_ | 1 | TRA | GDSVTQTEGPVTVSESES | ATSIAYP | LFWYVRYPGEGLQ | VITAGQK | GSSRGFEATYNKETTSFHLQKASVQ | CALGDRASLGKLQF | GTGTQVVVT |
| 246 | | | LIINCTYS (SEQ ID | N (SEQ | LLLK (SEQ ID | (SEQ ID | ESDSAVYY (SEQ ID NO: | (SEQ ID NO: | P (SEQ ID |
| | | | NO: 130) | ID NO: | NO: 5996) | NO: 8964) | 11932) | 25884) | NO: 16488) |
| | | | | 3098) | | | | | |
| Clonotype_ | 1 | TRB | DTTVKQNPRYKLARVGKP | MNHDT | MYWYQKKPNQAPK | YYDKIL | NREADTFEKFQSSRPNNSFCSLYIG | CASSSGTSSYEQYF | GPGTRLTVL |
| 247 | | | VNLICSQT (SEQ ID | (SEQ ID | LLLF (SEQ ID | (SEQ ID | SAGLEYSAMYL (SEQ ID NO: | (SEQ ID NO: | (SEQ ID |
| | | | NO: 299) | NO: | NO: 6235) | NO: | 12171) | 25885) | NO: |
| | | | | 3267) | | 9203) | | | 16327) |
| Clonotype_ | 1 | TRA | GEKVEQHESTLSVREGDS | DTASSY | FPWYKQEAGKGLH | IRSNVDR | KQSQRLIVLLDKKAKRFSLHITATQ | CAAASSGSWQLIF | GSGTQLTVM |
| 247 | | | AVINCTYT (SEQ ID | (SEQ ID | FVID (SEQ ID | (SEQ ID | PEDSAIYF (SEQ ID NO: | (SEQ ID NO: | P (SEQ ID |
| | | | NO: 54) | NO: | NO: 5994) | NO: | 11926) | 25886) | NO: |
| | | | | 3022) | | 8958) | | | 16400) |
| Clonotype_ | 1 | TRB | DAGVTQTPRHEVAEKGQT | SGHND | LFWYRQTKIQGLE | FRSKSL | MEDGGAFKDRFKAEMLNSSFSTLKI | CASSLDNTGQLYF | GEGSKLTVL |
| 248 | | | IILKCEPV (SEQ ID | (SEQ ID | LLSY (SEQ ID | (SEQ ID | PTEPKDSAVYL (SEQ ID NO: | (SEQ ID NO: | (SEQ ID |
| | | | NO: 1239) | NO: | NO: 7175) | NO: | 13111) | 25887) | NO: |
| | | | | 4207) | | 10143) | | | 16347) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMRVTYGGSGNKL | GIGTLLSVK |
| 248 | | | VTMDCVYE (SEQ ID | (SEQ ID | FLIR (SEQ ID | N (SEQ | IEDSAVYF (SEQ ID NO: 11878) | IF (SEQ ID NO: | P (SEQ ID |
| | | | NO: 44) | NO: 3056) | NO: 5942) | ID NO: | | 25888) | NO: |
| | | | | | | 8910) | | | 16456) |
| Clonotype_ | 1 | TRB | DAGVTQTPRHEVAEKGQT | SGHND | LFWYRQTKIQGLE | FRSKSL | MEDGGAFKDRFKAEMLNSSFSTLKI | CASSLGTGGPFSNE | GHGTKLSVL |
| 249 | | | IILKCEPV (SEQ ID | (SEQ ID | LLSY (SEQ ID | (SEQ ID | PTEPKDSAVYL (SEQ ID NO: | RLFF (SEQ ID | (SEQ ID |
| | | | NO: 1239) | NO: | NO: 7175) | NO: | 13111) | NO: 25889) | NO: 16401) |
| | | | | 4207) | | 10143) | | | |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREGEGNNRIFF | GDGTQLVVK |
| 249 | | | VTMDCVYE (SEQ ID | (SEQ ID | FLIR (SEQ ID | N (SEQ | IEDSAVYF (SEQ ID NO: 11878) | (SEQ ID NO: | P (SEQ ID |
| | | | NO: 44) | NO: | NO: 5942) | ID NO: | | 25890) | NO: 16328) |
| | | | | 3056) | | 8910) | | | |
| Clonotype_ | 1 | TRB | AQTIHQWPVAEIKAVGSP | GKSSPN | LYWYWQATGGTLQ | SITVG | VESVVQLNLSASRPKDDQFILSTE | CAWTLTGGASQNTL | GAGTRLSVL |
| 250 | | | LSLGCTIK (SEQ ID | (SEQ ID | QLFY (SEQ ID | (SEQ ID | KLLLSHSGFYL (SEQ ID NO: | YF (SEQ ID NO: | (SEQ ID |
| | | | NO: 35) | NO: | NO: 5971) | NO: | 11907) | 25891) | NO: |
| | | | | 3003) | | 8939) | | | 16365) |
| Clonotype_ | 1 | TRA | QQKVQQSPESLIVPEGGM | DRNVDY | FWWYRQHSGKSPK | IFSNGE | KEEGRFTVHLNKASLHTSLHIRDSQ | CAASGYSNNRLTL | GKGTQVVVL |
| 250 | | | ASLNCTSS (SEQ ID | (SEQ ID | MLMS (SEQ ID | (SEQ ID | PSDSALYL (SEQ ID NO: | (SEQ ID NO: | P (SEQ ID |
| | | | NO: 28) | NO: | NO: 5964) | NO: | 11900) | 25892) | NO: |
| | | | | 2996) | | 8932) | | | 16644) |
| Clonotype_ | 1 | TRB | AQTIHQWPVAEIKAVGSP | GKSSPN | LYWYWQATGGTLQ | SITVG | VESVVQLNLSASRPKDDQFILSTE | CAWRSGTANTEVFF | GKGTRLTVV |
| 251 | | | LSLGCTIK (SEQ ID | (SEQ ID | QLFY (SEQ ID | (SEQ ID | KLLLSHSGFYL (SEQ ID NO: | (SEQ ID NO: | (SEQ ID |
| | | | NO: 35) | NO: | NO: 5971) | NO: | 11907) | 25893) | NO: |
| | | | | 3003) | | 8939) | | | 16333) |
| Clonotype_ | 1 | TRA | GQQVQQSPSSLVLQEGEN | STATQ | LQWFYQSPGGSLV | NPSGT | KHTGRLTSTTVTKERRSSLHISSSQ | CAIERNMGYKLTF | GTGTSLLVD |
| 251 | | | AELQCNFS (SEQ ID | (SEQ ID | SLLS (SEQ ID | (SEQ ID | TTDSGTYL (SEQ ID NO: | (SEQ ID NO: | P (SEQ ID |
| | | | NO: 322) | NO: | NO: 6258) | NO: | 12194) | 25894) | NO: |
| | | | | 3290) | | 8914) | | | 16398) |
| Clonotype_ | 1 | TRB | AQTIHQWPVAEIKAVGSP | GKSSPN | LYWYWQATGGTLQ | SITVG | VESVVQLNLSASRPKDDQFILSTE | CAWSPGLGSYEQYF | GPGTRLTVL |
| 252 | | | LSLGCTIK (SEQ ID | (SEQ ID | QLFY (SEQ ID | (SEQ ID | KLLLSHSGFYL (SEQ ID NO: | (SEQ ID NO: | (SEQ ID |
| | | | NO: 35) | NO: | NO: 5971) | NO: | 11907) | 25895) | NO: |
| | | | | 3003) | | 8939) | | | 16327) |
| Clonotype_ | 1 | TRA | GDSVTQTEGLVTLTKGLP | TTYSPF | LFWYVQHLNEAPK | SSTDNKR | TEHQGFYATLHKSSSSFHLQKSSVQ | CALSASSGSWQLIF | GSGTQLTVM |
| 252 | | | VMLNCTYQ (SEQ ID | (SEQ ID | LLLK (SEQ ID | (SEQ ID | LSDSALYF (SEQ ID NO: | (SEQ ID NO: | P (SEQ ID |
| | | | NO: 8) | NO: | NO: 5944) | NO: | 11880) | 25882) | NO: |
| | | | | 2976) | | 8912) | | | 16400) |
| Clonotype_ | 1 | TRB | AQTIHQWPVAEIKAVGSP | GKSSPN | LYWYWQATGGTLQ | SITVG | VESVVQLNLSASRPKDDQFILSTE | CAWSLGVANERLFF | GHGTKLSVL |
| 253 | | | LSLGCTIK (SEQ ID | (SEQ ID | QLFY (SEQ ID | (SEQ ID | KLLLSHSGFYL (SEQ ID NO: | (SEQ ID NO: | (SEQ ID |
| | | | NO: 35) | NO: | NO: 5971) | NO: | 11907) | 25897) | NO: |
| | | | | 3003) | | 8939) | | | 16401) |
| Clonotype_ | 1 | TRA | GDSVTQTEGLVTLTEGLP | STYSPF | LFWYVQHLNEAPK | SFTDNKR | PEHQGFHATLHKSSSSFHLQKSSAQ | CASNSGGSNAKLTF | GKGTKLSVK |
| 253 | | | VMLNCTYQ (SEQ ID | (SEQ ID | LLLK (SEQ ID | (SEQ ID | LSDSALYY (SEQ ID NO: 11944) | (SEQ ID NO: | S (SEQ ID |
| | | | NO: 46) | NO: | NO: 5944) | NO: | | 25898) | NO: |
| | | | | 3040) | | 8950) | | | 16342) |
| Clonotype_ | 1 | TRB | AQTIHQWPVAEIKAVGSP | GKSSPN | LYWYWQATGGTLQ | SITVG | VESVVQLNLSASRPKDDQFILSTE | CAWSNRGYEQYF | GPGTRLTVL |
| 254 | | | LSLGCTIK (SEQ ID | (SEQ ID | QLFY (SEQ ID | (SEQ ID | KLLLSHSGFYL (SEQ ID NO: | (SEQ ID NO: | (SEQ ID |
| | | | NO: 35) | NO: | NO: 5971) | NO: 8939) | 11907) | 25899) | NO: 16327) |
| | | | | 3003) | | | | | |
| Clonotype_ | 1 | TRA | GEQVEQRPPHLSVPEGDS | DSATAY | FYWYKQEPGAGLQ | VFSNVDR | KEEQGLTVLLNKKDKQLSLNLTAAH | CAVSDTGGADRLTF | GKGTQLIIQ |
| 254 | | | AVIICTYT (SEQ ID | (SEQ ID | LLMS (SEQ ID | (SEQ ID | PGDSAVYF (SEQ ID NO: | (SEQ ID NO: | P (SEQ ID |
| | | | NO: 100) | NO: | NO: 6036) | NO: | 11972) | 25900) | NO: |
| | | | | 2990) | | 9004) | | | 16360) |
| Clonotype_ | 1 | TRB | AQTIHQWPVAEIKAVGSP | GKSSPN | LYWYWQATGGTLQ | SITVG | VESVVQLNLSASRPKDDQFILSTE | CAWSQRDRGYEQYF | GPGTRLTVL |
| 255 | | | LSLGCTIK (SEQ ID | (SEQ ID | QLFY (SEQ ID | (SEQ ID | KLLLSHSGFYL (SEQ ID NO: | (SEQ ID NO: | (SEQ ID |
| | | | NO: 35) | NO: | NO: 5971) | NO: 8939) | 11907) | 25901) | NO: 16327) |
| | | | | 3003) | | | | | |
| Clonotype_ | 1 | TRA | QQKVQQSPESLIVPEGAM | DSASQY | FAWYRQHSGKAPK | IFSNGE | KEEGRFTIHLNKASLHFSLHIRDSQ | CAVRVSGSFNKLTF | GAGTRLAVC |
| _255 | | | TSLNCTFS (SEQ ID | (SEQ ID | ALMS (SEQ ID | (SEQ ID | PSDSALYL (SEQ ID NO: 11948) | (SEQ ID NO: | P (SEQ ID |
| | | NO: 214) | NO: 3044) | NO: 6150) | NO: 8932) | | 25902) | NO: | 16692) |
| Clonotype_ | 1 | TRB | AQTIHQWPVAEIKAVGSP | GKSSPN | LYWYWQATGGTLQ | SITVG | VESVVQLNLSASRPKDDQFILSTE | CAWSLDNSGNTLYF | GEGSRLIVV |
| 256 | | | LSLGCTIK (SEQ ID | (SEQ ID | QLFY (SEQ ID | (SEQ ID | KLLLSHSGFYL (SEQ ID NO: | (SEQ ID NO: | (SEQ ID |
| | | | NO: 35) | NO: | NO: 5971) | NO: | 11907) | 25903) | NO: |
| | | | | 3003) | | 8939) | | | 16471) |
| Clonotype_ | 1 | TRA | GEQVEQRPPHLSVREGDS | DSATAY | FSWYKQEAGAGLQ | VLSNVDR | KEEQGLTVLLNKKDKRLSLNLTAAH | CAVSASSGSWQLIF | GSGTQLTVM |
| 256 | | | AIIICTYT (SEQ ID | (SEQ ID | LLMS (SEQ ID | (SEQ ID | PGDSAVYF (SEQ ID NO: | (SEQ ID NO: | P (SEQ ID |
| | | | NO: 22) | NO: | NO: 5958) | NO: | 11894) | 16268) | NO: |
| | | | | 2990) | | 8926) | | | 16400) |
| Clonotype_ | 1 | TRB | AQTIHQWPVAEIKAVGSP | GKSSPN | LYWYWQATGGTLQ | SITVG | VESVVQLNLSASRPKDDQFILSTE | CAWSLEGEPYEQYF | GPGTRLTVL |
| 257 | | | LSLGCTIK (SEQ ID | (SEQ ID | QLFY (SEQ ID | (SEQ ID | KLLLSHSGFYL (SEQ ID NO: | (SEQ ID NO: | (SEQ ID |
| | | | NO: 35) | NO: | NO: 5971) | NO: | 11907) | 25905) | NO: |
| | | | | 3003) | | 8939) | | | 16327) |
| Clonotype_ | 1 | TRA | GEQVEQR PPHLSVREGDS | DPNSYY | FFWYKQEPGAGLQ | VFSSTEI | NEGQGFTVLLNKKDKQLSLNLTAAH | CAVSAGYSNNRLTL | GKGTQVVVL |
| 257 | | | AVIICTYT (SEQ ID | (SEQ ID | LLMK (SEQ ID | (SEQ ID | PGDSAVYF (SEQ ID NO: | (SEQ ID NO: | P (SEQ ID |
| | | | NO: 108) | NO: | NO: 6044) | NO: | 11980) | 25906) | NO: |
| | | | | 3010) | | 8946) | | | 16644) |
| Clonotype_ | 1 | TRB | AQTIHQWPVAEIKAVGSP | GKSSPN | LYWYWQATGGTLQ | SITVG | VESVVQLNLSASRPKDDQFILSTE | CAWSYMNSDYTF | GSGTRLLVI |
| 258 | | | LSLGCTIK (SEQ ID | (SEQ ID | QLFY (SEQ ID | (SEQ ID | KLLLSHSGFYL (SEQ ID NO: | (SEQ ID NO: | (SEQ ID |
| | | | NO: 35) | NO: | NO: 5971) | NO: | 11907) | 25907) | NO: |
| | | | | 3003) | | 8939) | | | 16337) |
| Clonotype_ | 1 | TRA | AQKVTQTQTSISVMEKTT | TRDSSYF | LFWYKQTASGEIV | QDSYKKE | ATEGHYSLNFQKPKSSIGLIITATQ | CAMRVASSGSWQLI | GSGTQLTVM |
| 258 | | | VTMDCVYE (SEQ ID | (SEQ ID | FLIR (SEQ ID | N (SEQ | IEDSAVYF (SEQ ID NO: 11916) | F (SEQ ID NO: | P (SEQ ID |
| | | | NO: 44) | NO: | NO: 5942) | ID NO: | | 25908) | NO: 16400) |
| | | | | 2974) | | 8910) | | | |
| Clonotype_ | 1 | TRB | GPKVLQIPSHQIIDMGQM | SNHLY | FYWYKQILGQQME | FYNGKV | MEKSKLFKDQFSVERPDGSYFTLKI | CASSPYWGAYEQYF | GPGTRLTVL |
| 259 | | | VTLNCDPV (SEQ ID | (SEQ ID | FLVN (SEQ ID | (SEQ ID | PTALEDSAVYF (SEQ ID NO: | (SEQ ID NO: | (SEQ ID |
| | | | NO: 91) | NO: | NO: 6027) | NO: | 11963) | 25909) | NO: |
| | | | | 3059) | | 8995) | | | 16327) |
| Clonotype_ | 1 | TRA | GDSVTQTEGPVTLSEGTS | TKQYPT | LFWYVQYPGEGPQ | VPKANEK | GSSRGFEATYNKEATSFHLQKASVQ | CALSETGGSNAKLT | GKGTKLSVK |
| 259 | | | LTVNCSYE (SEQ ID | (SEQ ID | LLFK (SEQ ID | (SEQ ID | ESDSAVYY (SEQ ID NO: | F (SEQ ID NO: | S (SEQ ID |
| | | | NO: 38) | NO: | NO: 5974) | NO: | 12094) | 25910) | NO: |
| | | | | 3006) | | 8942) | | | 16342) |
| Clonotype_ | 1 | TRB | GPKVLQIPSHQIIDMGQM | SNHLY | FYWYKQILGQQME | FYNGKV | MEKSKLFKDQFSVERPDGSYFTLKI | CASSPDWGDTQYF | GPGTRLLVL |
| 260 | | | VTLNCDPV (SEQ ID | (SEQ ID | FLVN (SEQ ID | (SEQ ID | PTALEDSAVYF (SEQ ID NO: | (SEQ ID NO: | (SEQ ID |
| | | | NO: 91) | NO: | NO: 6027) | NO: | 11963) | 25911) | NO: |
| | | | | 3059) | | 8995) | | | 16341) |
| Clonotype_ | 1 | TRA | AQSVTQPDARVTVSEGAS | YSGTPY | LFWYVQYPRQGLQ | YYSGDPV | GVNGFEAEFSKSNSSFHLRKASVH | CVLSDYGNEKITF | GAGTKLTIK |
| 260 | | | LQLRCKYS (SEQ ID | (SEQ ID | LLLK (SEQ ID | V (SEQ | WSDSAVYF (SEQ ID NO: | (SEQ ID NO: | P (SEQ ID |
| | | | NO: 12) | NO: | NO: 5948) | ID NO: | 11892) | 25912) | NO: |
| | | | | 3054) | | 8916) | | | 16512) |
| Clonotype_ | 1 | TRB | GPKVLQIPSHQIIDMGQM | SNHLY | FYWYKQILGQQME | FYNGKV | MEKSKLFKDQFSVERPDGSYFTLKI | CASRGDRANERLFF | GHGTKLSVL |
| 261 | | | VTLNCDPV (SEQ ID | (SEQ ID | FLVN (SEQ ID | (SEQ ID | PTALEDSAVYF (SEQ ID NO: | (SEQ ID NO: | (SEQ ID |
| | | | NO: 91) | NO: | NO: 6027) | NO: | 11963) | 25913) | NO: |
| | | | | 3059) | | 8995) | | | 16401) |
| Clonotype_ | 1 | TRA | SQWGEENLQALSIQEGED | TYTTV | VHWYRQDSGRGPA | IRSNERE | KRSGRLRATLDTSSQSSSLSITAAQ | CATDTEGADRLTF | GKGTQLIIQ |
| 261 | | | VTMNCSYK (SEQ ID | (SEQ ID | LIIL (SEQ ID | (SEQ ID | CEDTAVYF (SEQ ID NO: | (SEQ ID NO: | P (SEQ ID |
| | | | NO: 80) | NO: | NO: 6016) | NO: | 11952) | 25914) | NO: |
| | | | | 3048) | | 8928) | | | 16360) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSENERLFF | GHGTKLSVL |
| 262 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25915) | NO: |
| | | | | 2995) | | 8931) | | | 16401) |
| Clonotype_ | 1 | TRA | QQKVQQSPESLTVSEGAM | DGTSNN | FRWYRQHSAKGLE | IFSDGE | KEEGRFTAHLNRANLHVSLHIREPQ | CAVPNSAGNKLTF | GIGTRVLVR |
| 262 | | | ASLNCTFS (SEQ ID | (SEQ ID | VLVS (SEQ ID | (SEQ ID | PSDSAVYL (SEQ ID NO: | (SEQ ID NO: | P (SEQ ID |
| | | | NO: 92) | NO: | NO: 6028) | NO: | 11964) | 15686) | NO: |
| | | | | 3060) | | 8996) | | | 16580) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSGTFYAEQFF | GPGTRLTVL |
| 263 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25917) | NO: |
| | | | | 2995) | | 8931) | | | 16327) |
| Clonotype_ | 1 | TRA | GQQVQQSPASLVLQEGEN | TSLNS | MQWFYQRPEGSLV | NPSGT | KQSGRLTSTTVIKERRSSLHISSSQ | CATNYGNEKITF | GAGTKLTIK |
| 263 | | | AELQCNFS (SEQ ID | (SEQ ID | SLFY (SEQ ID | (SEQ ID | ITDSGTYL (SEQ ID NO: | (SEQ ID NO: | P (SEQ ID |
| | | | NO: 10) | NO: | NO: 6192) | NO: | 12038) | | NO: |
| | | | | 2978) | | 8914) | | 25918) | 16512) |
| Clonotype_ | 1 | TRB | GGIITQTPKFLIGQEGQK | FNHDT | MYWYRQDSGKGLR | SITEND | LQKGDLSEGYDASREKKSSFSLTVT | CASRDNYAEQFF | GPGTRLTVL |
| 264 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25919) | NO: |
| | | | | 2995) | | 8931) | | | 16327) |
| Clonotype_ | 1 | TRA | QQKVQQSPESLIVPEGGM | DRNVDY | FWWYRQHSGKSPK | IFSNGE | KEEGRFTVHLNKASLHTSLHIRDSQ | CAARTGGYKVVF | GSGTRLLVS |
| 264 | | | ASLNCTSS (SEQ ID | (SEQ ID | MLMS (SEQ ID | (SEQ ID | PSDSALYL (SEQ ID NO: | (SEQ ID NO: | P (SEQ ID |
| | | | NO: 28) | NO: | NO: 5964) | NO: | 11900) | 25644) | NO: |
| | | | | 2996) | | 8932) | | | 16354) |
| Clonotype_ | 1 | TRB | GGIITQTPKFLIGQEGQK | FNHDT | MYWYRQDSGKGLR | SITEND | LQKGDLSEGYDASREKKSSFSLTVT | CASRDNNNQAPLF | GEGTRLSVL |
| 265 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: 2995) | NO: 5963) | NO: 8931) | 11899) | 15213) | NO: 16441) |
| Clonotype_ | 1 | TRA | GDSVTQTEGQVTVSESKS | ATSIGYP | LFWYVRYPGEGLQ | VITAGQK | GSSRGFEATYNKEATSFHLQKASVQ | CALSDPLYQGGRAL | GTGTTVSVS |
| 265 | | | LIINCTYS (SEQ ID | N (SEQ | LLLK (SEQ ID | (SEQ ID | ESDSAVYY (SEQ ID NO: | IF (SEQ ID NO: | P (SEQ ID |
| | | | NO: 60) | ID NO: | NO: 5996) | NO: | 12094) | 25922) | NO: |
| | | | | 3330) | | 8964) | | | 16348) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSTGTGDNSPLY | AAGTRLTVT |
| 266 | | | LTLKCQQN (SEQ ID | (SEQ ID | LIYY (SEQ ID | (SEQ ID | SAQKNEMAVFL (SEQ ID NO: | F (SEQ ID NO: | (SEQ ID |
| | | | NO: 27) | NO: | NO: 5963) | NO: | 11899) | 25923) | NO: |
| | | | | 2995) | | 8931) | | | 16381) |
| Clonotype_ | 1 | TRA | GEQVEQLPSSLIVQEGAS | DSASVY | FPWYKQEPGKRLQ | IRSNMER | KQNQRLTLLFDKKTKHLSLHITATQ | CSASNNYAQGLTF | GLGTRVSVF |
| 266 | | | VLINCSYT (SEQ ID | (SEQ ID | FIID (SEQ ID | (SEQ ID | PGDSAIYF (SEQ ID NO: | (SEQ ID NO: | P (SEQ ID |
| | | | NO: 160) | NO: | NO: 6096) | NO: | 12032) | 25394) | NO: |
| | | | | 3128) | | 8952) | | | 16344) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSTGVWDTLYF | GAGTRLSVL |
| 267 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25925) | NO: |
| | | | | 2995) | | 8931) | | | 16365) |
| Clonotype_ | 1 | TRA | TQTVSQSDAHVTVFEGDS | YGGSIY | ILSWYIQHHGRGLQ | YYSGNPV | GVNGFEAEFSKSDSSFHLRKASVH | CAVSAGNNAPRF | GAGTKLTVK |
| 267 | | | VELRCNYS (SEQ ID | (SEQ ID | FLLK (SEQ ID | V (SEQ | WSDSAVYF (SEQ ID NO: | (SEQ ID NO: | P (SEQ ID |
| | | | NO: 110) | NO: | NO: 6046) | ID NO: | 11946) | 25926) | NO: |
| | | | | 3078) | | 9014) | | | 16424) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSRTTGPNSDYT | GSGTRLLVI |
| 268 | | | LTLKCQQN (SEQ ID | (SEQ ID | LIYY (SEQ ID | (SEQ ID | SAQKNEMAVFL (SEQ ID NO: | F (SEQ ID NO: | (SEQ ID |
| | | | NO: 27) | NO: | NO: 5963) | NO: | 11899) | 25927) | NO: |
| | | | | 2995) | | 8931) | | | 16337) |
| Clonotype_ | 1 | TRA | GDSVTQKQGQVTLSEDDF | TTTYPT | LLWYVQYLGQGPQ | VTTANNK | GISRGFEATYDKGTTSFHLQKASVQ | CALDTNAYKVIF | GKGTHLHVL |
| 268 | | | LFINCTYS (SEQ ID | (SEQ ID | LLLK (SEQ ID | (SEQ ID | ESDSAVYF (SEQ ID NO: | (SEQ ID NO: | P (SEQ ID |
| | | | NO: 510) | NO: | NO: 22268) | NO: | 12382) | 25928) | NO: |
| | | | | 3478) | | 9054) | | | 16330) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSITSANTEVFF | GKGTRLTVV |
| 269 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25929) | NO: |
| | | | | 2995) | | 8931) | | | 16333) |
| Clonotype_ | 1 | TRA | GDSVTQTEGLVTLTEGLP | STYSPF | LFWYVQHLNEAPK | SFTDNKR | PEHQGFHATLHKSSSSFHLQKSSAQ | CADTGANTGKLTF | GHGTILRVH |
| 269 | | | VMLNCTYQ (SEQ ID | (SEQ ID | LLLK (SEQ ID | (SEQ ID | LSDSALYY (SEQ ID NO: | (SEQ ID NO: | P (SEQ ID |
| | | | NO: 46) | NO: 3040) | NO: 5944) | NO: 8950) | 11944) | 25930) | NO: |
| | | | | | | | | | 16356) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSIRGWGTEVFF | GKGTRLTVV |
| 270 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25931) | NO: |
| | | | | 2995) | | 8931) | | | 16333) |
| Clonotype_ | 1 | TRA | QQQVRQSPQSLTVWEGET | NSAFDY | FPWYQQFPGEGPA | IRSVSDK | KEDGRFTIFFNKREKKLSLHITDSQ | CAASGGGYQNFYF | GKGTSLTVI |
| 270 | | | AILNCSYE (SEQ ID | (SEQ ID | LLIA (SEQ ID | (SEQ ID | PGDSATYF (SEQ ID NO: | (SEQ ID NO: | P (SEQ ID |
| | | | NO: 68) | NO: 3052) | NO: 6020) | NO: 8988) | 11940) | 25932) | NO: 16366) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSIEGRYTEVFF | GKGTRLTVV |
| 271 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: 2995) | NO: 5963) | NO: 8931) | 11899) | 25933) | NO: 16333) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMREAGYQNFYF | GKGTSLTVI |
| 271 | | | VTMDCVYE (SEQ ID | (SEQ ID | FLIR (SEQ ID | N (SEQ | IEDSAVYF (SEQ ID NO: 11878) | (SEQ ID NO: | P (SEQ ID |
| | | | NO: 44) | NO: 3056) | NO: 5942) | ID NO: | | 25934) | NO: 16366) |
| | | | | | | 8910) | | | |
| Clonotype_ | 1 | TRB | GGIITQTPKFLIGQEGQK | FNHDT | MYWYRQDSGKGLR | SITEND | LQKGDLSEGYDASREKKSSFSLTVT | CASSIGGRDTQYF | GPGTRLLVL |
| 272 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25935) | NO: |
| | | | | 2995) | | 8931) | | | 16341) |
| Clonotype_ | 1 | TRA | GDQVKQSPSALSLQEGTN | IATTT | VQWFLQNPRGSLM | LVPGT | KENGRLKSTFNSKESYSTLHIRDAQ | CAAGTDYANKMIF | GLGTILRVR |
| 272 | | | SALRCNFS (SEQ ID | (SEQ ID | NLFY (SEQ ID | (SEQ ID | LEDSGTYF (SEQ ID NO: | (SEQ ID NO: | P (SEQ ID |
| | | | NO: 258) | NO: | NO: 6194) | NO: | 12130) | 25936) | NO: |
| | | | | 3226) | | 9162) | | | 16416) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSIGIYEQYF | GPGTRLTVL |
| 273 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25937) | NO: |
| | | | | 2995) | | 8931) | | | 16327) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMRNSAGNKLTF | GIGTRVLVR |
| 273 | | | VTMDCVYE (SEQ ID | (SEQ ID | FLIR (SEQ ID | N (SEQ | IEDSAVYF (SEQ ID NO: 11878) | (SEQ ID NO: | P (SEQ ID |
| | | | NO: 44) | NO: 3056) | NO: 5942) | ID NO: | | 25938) | NO: |
| | | | | | | 8910) | | | 16580) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSMRGGAEEQYF | GPGTRLTVL |
| 274 | | | LTLKCQQN (SEQ ID | (SEQ ID | LIYY (SEQ ID | (SEQ ID | SAQKNEMAVEL (SEQ ID NO: | (SEQ ID NO: | (SEQ ID |
| | | | NO: 27) | NO: 2995) | NO: 5963) | NO: 8931) | 11899) | 25939) | NO: |
| | | | | | | | | | 16327) |
| Clonotype_ | 1 | TRA | SQLAEENSWALSVHEGES | TSITA | LQWYRQKSGKGPA | IRSNERE | KRNGRLRATLDTSSQSSSLSITATR | CATENNMGYKLTF | GTGTSLLVD |
| 274 | | | VTVNCSYK (SEQ ID | (SEQ ID | QLIL (SEQ ID | (SEQ ID | CEDTAVYF (SEQ ID NO: | (SEQ ID NO: | P (SEQ ID |
| | | | NO: 188) | NO: | NO: 6124) | NO: | 11896) | 25940) | NO: |
| | | | | 2992) | | 8928) | | | 16398) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSMGTKEVFF | GKGTRLTVV |
| 275 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25941) | NO: |
| | | | | 2995) | | 8931) | | | 16333) |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMKEDTNTGKLTF | GDGTVLTVK |
| 275 | | | VTMDCVYE (SEQ ID | (SEQ ID | FLIR (SEQ ID | N (SEQ | IEDSAVYF (SEQ ID NO: | (SEQ ID NO: | P (SEQ ID |
| | | | NO: 44) | NO: | NO: 5942) | ID NO: | 11878) | 25942) | NO: |
| | | | | 3056) | | 8910) | | | 16516) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSMGTGVAEQFF | GPGTRLTVL |
| 276 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25943) | NO: |
| | | | | 2995) | | 8931) | | | 16327) |
| Clonotype_ | 1 | TRA | AQSVDQPDAHVTLYEGAS | YSAAPY | LFWYVQYPGQSLQ | YITGDAV | KGTKGFEAEFRKSNSSFNLKKSPAH | CALEGRNSGGSNYK | GKGTLLTVT |
| 276 | | | LELRCSYS (SEQ ID | (SEQ ID | FLLK (SEQ ID | V (SEQ | WSDSAKYF (SEQ ID NO: | LTF (SEQ ID NO: | P (SEQ ID |
| | | | NO: 398) | NO: | NO: 6216) | ID NO: | 12152) | 15238) | NO: |
| | | | | 3248) | | 9302) | | | 16642) |
| Clonotype_ | 1 | TRB | GGIITQTPKFLIGQEGQK | FNHDT (S) | MYWYRQDSGKGLR | SITEND | LQKGDLSEGYDASREKKSSFSLTVT | CASSMGANSDYTF | GSGTRLLVI |
| 277 | | | LTLKCQQN (SEQ ID | (SEQ ID | LIYY (SEQ ID | (SEQ ID | SAQKNEMAVEL (SEQ ID NO: | (SEQ ID NO: | (SEQ ID |
| | | | NO: 27) | NO: | NO: 5963) | NO: | 11899) | 25945) | NO: |
| | | | | 2995) | | 8931) | | | 16337) |
| Clonotype_ | 1 | TRA | GEKVEQHESTLSVREGDS | DTASSY | FPWYKQEAGKGLH | IRSNVDR | KQSQRLIVLLDKKAKRFSLHITATQ | CAASTRGGSNYKLT | GKGTLLTVT |
| 277 | | | AVINCTYT (SEQ ID | (SEQ ID | FVID (SEQ ID | (SEQ ID | PEDSAIYF (SEQ ID NO: | F (SEQ ID NO: | P (SEQ ID |
| | | | NO: 54) | NO: 3022) | NO: 5994) | NO: 8958) | 11926) | 25946) | NO: 16642) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSMGGATEVFF | GKGTRLTVV |
| 278 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25947) | NO: |
| | | | | 2995) | | 8931) | | | 16333) |
| Clonotype_ | 1 | TRA | GEKVEQHESTLSVREGDS | DTASSY | FPWYKQEAGKGLH | IRSNVDR | KQSQRLIVLLDKKAKRFSLHITATQ | CAASRSGGSNYKLT | GKGTLLTVT |
| 278 | | | AVINCTYT (SEQ ID | (SEQ ID | FVID (SEQ ID | (SEQ ID | PEDSAIYF (SEQ ID NO: | F (SEQ ID NO: | P (SEQ ID |
| | | | NO: 54) | NO: 3022) | NO: 5994) | NO: 8958) | 11926) | 25948) | NO: |
| | | | | | | | | | 16642) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSMVGNTGQLYF | GEGSKLTVL |
| 279 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: 2995) | NO: 5963) | NO: 8931) | 11899) | 25949) | NO: |
| | | | | | | | | | 16347) |
| Clonotype_ | 1 | TRA | QQQVRQSPQSLTVWEGET | DSTFDY | FPWYRLFPGESPA | IRPVSNK | KEDGRFTIFFNKREKKLSLHITDSQ | CAAPTGANTGKLTF | GHGTILRVH |
| 279 | | | AILNCSYE (SEQ ID | (SEQ ID | LLIA (SEQ ID | (SEQ ID | PGDSATYF (SEQ ID NO: | (SEQ ID NO: | P (SEQ ID |
| | | | NO: 68) | NO: | NO: 6176) | NO: | 11940) | 25950) | NO: |
| | | | | 3208) | | 9144) | | | 16356) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSMSGTEVFF | GKGTRLTVV |
| 280 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: 2995) | NO: 5963) | NO: 8931) | 11899) | 25951) | NO: |
| | | | | | | | | | 16333) |
| Clonotype_ | 1 | TRA | GDSVTQTEGQVTVSESKS | ATSIAYP | LFWYVRYPGEGLQ | VITAGQK | GSSRGFEATYNKETTSFHLQKASVQ | CALRANSGGSNAKL | GKGTKLSVK |
| 280 | | | LIINCTYS (SEQ ID | N (SEQ | LLLK (SEQ ID | (SEQ ID | ESDSAVYY (SEQ ID NO: | TF (SEQ ID NO: | S (SEQ ID |
| | | | NO: 60) | ID NO: | NO: 5996) | NO: | 11932) | 25952) | NO: |
| | | | | 3098) | | 8964) | | | 16342) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSMWDTSEVFF | GKGTRLTVV |
| 281 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25953) | NO: |
| | | | | 2995) | | 8931) | | | 16333) |
| Clonotype_ | 1 | TRA | GDSVTQTEGQVTVSESKS | TTSIAYP | LFWYVRYPGEGLQ | VITAGQK | GSSRGFEATYNKETTSFHLQKASVQ | CALGNSGGSNYKLT | GKGTLLTVT |
| 281 | | | LIINCTYS (SEQ ID | N (SEQ | LLLK (SEQ ID | (SEQ ID | ESDSAVYY (SEQ ID NO: | F (SEQ ID NO: | P (SEQ ID |
| | | | NO: 60) | ID NO: | NO: 5996) | NO: | 11932) | 25954) | NO: |
| | | | | 3028) | | 8964) | | | 16642) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSITGTNTGQLY | GEGSKLTVL |
| 282 | | | LTLKCQQN (SEQ ID | (SEQ ID | LIYY (SEQ ID | (SEQ ID | SAQKNEMAVFL (SEQ ID NO: | F (SEQ ID NO: | (SEQ ID |
| | | | NO: 27) | NO: | NO: 5963) | NO: | 11899) | 25955) | NO: |
| | | | | 2995) | | 8931) | | | 16347) |
| Clonotype_ | 1 | TRA | GEQVEQRPPHLSVREGDS | DPNSYY | FFWYKQEPGASLQ | VFSSTEI | NEGQGFTVLLNKKDKRLSLNLTAAH | CAVSAYGNEKITF | GAGTKLTIK |
| 282 | | | AVITCTYT (SEQ ID | (SEQ ID | LLMK (SEQ ID | (SEQ ID | PGDSAAYF (SEQ ID NO: | (SEQ ID NO: | P (SEQ ID |
| | | | NO: 42) | NO: | NO: 5978) | NO: | 11914) | 25956) | NO: |
| | | | | 3010) | | 8946) | | | 16512) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSISERLFF | GHGTKLSVL |
| 283 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25957) | NO: |
| | | | | 2995) | | 8931) | | | 16401) |
| Clonotype_ | 1 | TRA | QQKVQQSPESLSVPEGGM | DRNFQY | FWWYRQHSGEGPK | IFSDGD | KKEGRFTAHLNKASLHVSLHIRDSQ | CAAPANYGNEKITF | GAGTKLTIK |
| 283 | | | ASFNCTSS (SEQ ID | (SEQ ID | ALMS (SEQ ID | (SEQ ID | PSDSALYF (SEQ ID NO: | (SEQ ID NO: | P (SEQ ID |
| | | | NO: 2) | NO: | NO: 5938) | NO: | 11874) | 25958) | NO: |
| | | | | 2970) | | 8906) | | | 16512) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSIGNNQDTQYF | GPGTRLLVL |
| 284 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25959) | NO: |
| | | | | 2995) | | 8931) | | | 16341) |
| Clonotype_ | 1 | TRA | AQSVTQPDARVTVSEGAS | YSGTPY | LFWYVQYPRQGLQ | YYSGDPV | GVNGFEAEFSKSNSSFHLRKASVH | CVLSASNNRIFF | GDGTQLVVK |
| 284 | | | LQLRCKYS (SEQ ID | (SEQ ID | LLLK (SEQ ID | V (SEQ | WSDSAVYF (SEQ ID NO: | (SEQ ID NO: | P (SEQ ID |
| | | | NO: 12) | NO: | NO: 5948) | ID NO: | 11892) | 16320) | NO: |
| | | | | 3054) | | 8916) | | | 16328) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSIGTAYEQYF | GPGTRLTVL |
| 285 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25961) | NO: |
| | | | | 2995) | | 8931) | | | 16327) |
| Clonotype_ | 1 | TRA | GDSVTQKQGQVTLSEDDF | TTTYPT | LFWYVQYPGQGPQ | VTTANNK | GISRGFEATYDKGTTSFHLQKASVQ | CALNYGNEKITF | GAGTKLTIK |
| 285 | | | LFINCTYS (SEQ ID | (SEQ ID | LLLK (SEQ ID | (SEQ ID | ESDSAVYF (SEQ ID NO: | (SEQ ID NO: | P (SEQ ID |
| | | | NO: 510) | NO: 3478) | NO: 6446) | NO: 9054) | 12382) | 25962) | NO: |
| | | | | | | | | | 16512) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSILGDQDTQYF | GPGTRLLVL |
| 286 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25963) | NO: |
| | | | | 2995) | | 8931) | | | 16341) |
| Clonotype_ | 1 | TRA | TQTVSQSDAHVTVFEGDS | YGGSTY | ILSWYIQHHGRGLQ | YYSGNPV | GVNGFEAEFSKSDSSFHLRKASVH | CAVSASGSWQLIF | GSGTQLTVM |
| 286 | | | VELRCNYS (SEQ ID | (SEQ ID | FLLK (SEQ ID | V (SEQ | WSDSAVYF (SEQ ID NO: | (SEQ ID NO: | P (SEQ ID |
| | | | NO: 110) | NO: | NO: 6046) | ID NO: | 11946) | 25964) | NO: |
| | | | | 3078) | | 9014) | | | 16400) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSHWGGDTGQLY | GEGSKLTVL |
| 287 | | | LTLKCQQN (SEQ ID | (SEQ ID | LIYY (SEQ ID | (SEQ ID | SAQKNEMAVFL (SEQ ID NO: | F (SEQ ID NO: | (SEQ ID |
| | | | NO: 27) | NO: 2995) | NO: 5963) | NO: 8931) | 11899) | 25965) | NO: |
| | | | | | | | | | 16347) |
| Clonotype_ | 1 | TRA | TQTVSQSDAHVTVFEGDS | YGGSIY | LSWYIQHHGHGLQ | YYSGNPV | GVNGFEAEFSKSDSSFHLRKASVH | CAVSAEESNYQLIW | GSGTKLIIK |
| 287 | | | VELRCNYS (SEQ ID | (SEQ ID | FLLK (SEQ ID | V (SEQ | WSDSAVYF (SEQ ID NO: | (SEQ ID NO: | P (SEQ ID |
| | | | NO: 110) | NO: | NO: 6518) | ID NO: | 11946) | 25966) | NO: |
| | | | | 3078) | | 9014) | | | 16372) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSPTGRDEQYF | GPGTRLTVL |
| 288 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25967) | NO: |
| | | | | 2995) | | 8931) | | | 16327) |
| Clonotype_ | 1 | TRA | GEQVEQLPSSLIVQEGAS | DSASVY | FPWYKQEPGKRLQ | IRSNMER | KQNQRLTLLFDKKTKHLSLHITATQ | CSATFNTDKVVF | GTGTRLQVS |
| 288 | | | VLINCSYT (SEQ ID | (SEQ ID | FIID (SEQ ID | (SEQ ID | PGDSAIYF (SEQ ID NO: | (SEQ ID NO: | P (SEQ ID |
| | | | NO: 160) | NO: | NO: 6096) | NO: | 12032) | 25968) | NO: |
| | | | | 3128) | | 8952) | | | 16374) |
| Clonotype_ | 1 | TRB | GGIITQTPKFLIGQEGQK | FNHDT | LIYY | SITEND | LQKGDLSEGYDASREKKSSFSLTVT | CASSRGTGGPEQFF | GPGTRLTVL |
| 289 | | | LTLKCQQN (SEQ ID | (SEQ ID | (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25969) | NO: 16327) |
| | | | | 2995) | MYWYRQDSGKGLR | 8931) | | | |
| Clonotype_ | 1 | TRA | LAKTTQPPSMEAYEGQEV | NIATSEY | IYWYRQVPHQGPQ | GYKDYVV | NEVASLFISADRKLSTLSLPWVSLR | CIVTDIGNYAQGLT | GLGTRVSVF |
| 289 | | | NVSCSHT (SEQ ID NO: | (SEQ ID | FIIQ (SEQ ID | (SEQ ID | DAAVYY (SEQ ID NO: 11978) | F (SEQ ID NO: | P (SEQ ID |
| | | | 106) | NO: 3074) | NO: 6042) | NO: 9010) | | 25970) | NO: |
| | | | | | | | | | 16344) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSLSTYNSPLYF | AAGTRLTVT |
| 290 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25971) | NO: |
| | | | | 2995) | | 8931) | | | 16381) |
| Clonotype_ | 1 | TRA | QQKVQQSPESLIVPEGGM | DRNVDY | FWWYRQHSGKSPK | IFSNGE | KEEGRFTVHLNKASLHTSLHIRDSQ | CAASGKSNNRIFF | GDGTQLVVK |
| 290 | | | ASLNCTSS (SEQ ID | (SEQ ID | MLMS (SEQ ID | (SEQ ID | PSDSALYL (SEQ ID NO: | (SEQ ID NO: | P (SEQ ID |
| | | | NO: 28) | NO: | NO: 5964) | NO: | 11900) | 25972) | NO: |
| | | | | 2996) | | 8932) | | | 16328) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSEDSYAEQFF | GPGTRLTVL |
| 291 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25973) | NO: |
| | | | | 2995) | | 8931) | | | 16327) |
| Clonotype_ | 1 | TRA | GDSVIQMQGQVTLSENDF | TTGYPT | LFWYVQYSGEGPQ | VTTANNK | GSSRGFEATYDKGTTSFHLQKTSVQ | CAMRVGGSNAKLTF | GKGTKLSVK |
| 291 | | | LFINCTYS (SEQ ID | (SEQ ID | LLLQ (SEQ ID | (SEQ ID | EIDSAVYY (SEQ ID NO: | (SEQ ID NO: | S (SEQ ID |
| | | | NO: 456) | NO: | NO: 6392) | NO: | 12328) | 25974) | NO: |
| | | | | 3220) | | 9054) | | | 16342) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSGVGADTQYF | GPGTRLLVL |
| 292 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25975) | NO: |
| | | | | 2995) | | 8931) | | | 16341) |
| Clonotype_ | 1 | TRA | QQQVRQSPQSLTVWEGET | DSTFNY | FPWYQQFPGEGPA | IRSVSDK | KEDGRFTIFFNKREKKLSLHITDSQ | CAASESSGSWQLIF | GSGTQLTVM |
| 292 | | | AILNCSYE (SEQ ID | (SEQ ID | LLIS (SEQ ID | (SEQ ID | PGDSATYF (SEQ ID NO: | (SEQ ID NO: | P (SEQ ID |
| | | | NO: 68) | NO: | NO: 6004) | NO: | 11940) | 25976) | NO: |
| | | | | 3036) | | 8988) | | | 16400) |
| Clonotype_ | 1 | TRB | GGIITQTPKFLIGQEGQK | FNHDT | MYWYRQDSGKGLR | SITEND | LQKGDLSEGYDASREKKSSFSLTVT | CASSWEGTGQLYF | GEGSKLTVL |
| 293 | | | LTLKCQQN (SEQ ID | (SEQ ID | LIYY (SEQ ID | (SEQ ID | SAQKNEMAVFL (SEQ ID NO: | (SEQ ID NO: | (SEQ ID |
| | | | NO: 27) | NO: | NO: 5963) | NO: | 11899) | 25977) | NO: |
| | | | | 2995) | | 8931) | | | 16347) |
| Clonotype_ | 1 | TRA | GDSVTQTEGLVTLTKGLP | TTYSPF | LFWYVQHLNEAPK | SSTDNKR | TEHQGFYATLHKSSSSFHLQKSSVQ | CALSDQTGFASALT | GSGTKVIVL |
| 293 | | | VMLNCTYQ (SEQ ID | (SEQ ID | LLLK (SEQ ID | (SEQ ID | LSDSALYF (SEQ ID NO: | F (SEQ ID NO: | PY (SEQ ID |
| | | | NO: 8) | NO: | NO: 5944) | NO: | 11880) | 25978) | NO: |
| | | | | 2976) | | 8912) | | | 17004) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCRRDRDYAEQFF | GPGTRLTVL |
| 294 | | | VNLRCILK (SEQ ID | (SEQ ID | WLFT (SEQ ID | (SEQ ID | ANMSQGRTLY (SEQ ID NO: | (SEQ ID NO: | (SEQ ID |
| | | | NO: 23) | NO: | NO: 5959) | NO: | 11895) | 25979) | NO: |
| | | | | 2991) | | 8927) | | | 16327) |
| Clonotype_ | 1 | TRA | LAKTTQPPSMEAYEGQEV | NIATSEY | FIIQ (SEQ ID | GYKDYVV | NEVASLFISADRKLSTLSLPWVSLR | CIVTGDDSGYNKLT | GKGTVLLVS |
| 294 | | | NVSCSHT (SEQ ID NO: | (SEQ ID | IYWYRQVPHQGPQ | (SEQ ID | DAAVYY (SEQ ID NO: 11978) | F (SEQ ID NO: | P (SEQ ID |
| | | | 106) | NO: | NO: 6042) | NO: | | 25980) | NO: |
| | | | | 3074) | | 9010) | | | 16378) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSADRDWGGYNY | GPGTRLTVL |
| 295 | | | VNLRCILK (SEQ ID | (SEQ ID | WLFT (SEQ ID | (SEQ ID | ANMSQGRTLY (SEQ ID NO: | AEQFF (SEQ ID | (SEQ ID |
| | | | NO: 23) | NO: 2991) | NO: 5959) | NO: 8927) | 11895) | NO: 25981) | NO: 16327) |
| Clonotype_ | 1 | TRA | GEQVEQRPPHLSVREGDS | DPNSYY | FFWYKQEPGAGLQ | VFSSTEI | NEGQGFTVLLNKKDKQLSLNLTAAH | CAVSDHSNNRIFF | GDGTQLVVK |
| 295 | | | AVIICTYT (SEQ ID | (SEQ ID | LLMK (SEQ ID | (SEQ ID | PGDSAVYF (SEQ ID NO: | (SEQ ID NO: | P (SEQ ID |
| | | | NO: 108) | NO: | NO: 6044) | NO: | 11980) | 25982) | NO: |
| | | | | 3010) | | 8946) | | | 16328) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSADRDRYAEQF | GPGTRLTVL |
| 296 | | | VNLRCILK (SEQ ID | (SEQ ID | WLFT (SEQ ID | (SEQ ID | ANMSQGRTLY (SEQ ID NO: | F (SEQ ID NO: | (SEQ ID |
| | | | NO: 23) | NO: | NO: 5959) | NO: 8927) | 11895) | 25983) | NO: 16327) |
| | | | | 2991) | | | | | |
| Clonotype_ | 1 | TRA | AQKVTQTQTSISVMEKTT | TQDSSYF | LFWYKQTASGEIV | QDSYKKE | ATVGHYSLNFQKPKSSIGLIITATQ | CAMRDTGYQNFYF | GKGTSLTVI |
| 296 | | | VTMDCVYE (SEQ ID | (SEQ ID | FLIR (SEQ ID | N (SEQ | IEDSAVYF (SEQ ID NO: | (SEQ ID NO: | P (SEQ ID |
| | | | NO: 44) | NO: | NO: 5942) | ID NO: | 11878) | 25984) | NO: |
| | | | | 3056) | | 8910) | | | 16366) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSADFERLFF | GHGTKLSVL |
| 297 | | | VNLRCILK (SEQ ID | (SEQ ID | WLFT (SEQ ID | (SEQ ID | ANMSQGRTLY (SEQ ID NO: | (SEQ ID NO: | (SEQ ID |
| | | | NO: 23) | NO: | NO: 5959) | NO: 8927) | 11895) | 25985) | NO: 16401) |
| | | | | 2991) | | | | | |
| Clonotype_ | 1 | TRA | AQSVTQPDARVTVSEGAS | YSATPY | LFWYVQYPRQGLQ | YYSGDPV | GVNGFEAEFSKSNSSFHLRKASVH | CAGDTNAYKVIF | GKGTHLHVL |
| 297 | | | LQLRCKYS (SEQ ID | (SEQ ID | LLLK (SEQ ID | V (SEQ | WSDSAVYF (SEQ ID NO: | (SEQ ID NO: | P (SEQ ID |
| | | | NO: 12) | NO: | NO: 5948) | ID NO: | 11892) | 25986) | NO: |
| | | | | 2980) | | 8916) | | | 16330) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSAGDNSYNSPL | AAGTRLTVT |
| 298 | | | VNLRCILK (SEQ ID | (SEQ ID | WLFT (SEQ ID | (SEQ ID | ANMSQGRTLY (SEQ ID NO: | YF (SEQ ID NO: | (SEQ ID |
| | | | NO: 23) | NO: | NO: 5959) | NO: 8927) | 11895) | 25987) | NO: 16381) |
| | | | | 2991) | | | | | |
| Clonotype_ | 1 | TRA | GDSVTQTEGLVTLTEGLP | STYSPF | LFWYVQHLNEAPK | SFTDNKR | PEHQGFHATLHKSSSSFHLQKSSAQ | CAANYNQGKLIF | GQGTKLSIK |
| 298 | | | VMLNCTYQ (SEQ ID | (SEQ ID | LLLK (SEQ ID | (SEQ ID | LSDSALYY (SEQ ID NO: | (SEQ ID NO: | P (SEQ ID |
| | | | NO: 46) | NO: | NO: 5944) | NO: | 11944) | 25988) | NO: |
| | | | | 3040) | | 8950) | | | 16332) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSAGGADAEQFF | GPGTRLTVL |
| 299 | | | VNLRCILK (SEQ ID | (SEQ ID | WLFT (SEQ ID | (SEQ ID | ANMSQGRTLY (SEQ ID NO: | (SEQ ID NO: | (SEQ ID |
| | | | NO: 23) | NO: | NO: 5959) | NO: 8927) | 11895) | 25989) | NO: 16327) |
| | | | | 2991) | | | | | |
| Clonotype_ | 1 | TRA | LAKTTQPPSMEAYEGQEV | NIATSEY | TYWYRQVPHQGPQ | GYKDYVV | NEVASLFISADRKLSTLSLPWVSLR | CIVTANNRIFF | GDGTQLVVK |
| 299 | | | NVSCSHT (SEQ ID NO: | (SEQ ID | FIIQ (SEQ ID | (SEQ ID | DAAVYY (SEQ ID NO: 11978) | (SEQ ID NO: | P (SEQ ID |
| | | | 106) | NO: 3074) | NO: 6042) | NO: 9010) | | 25990) | NO: |
| | | | | | | | | | 16328) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSAGQNQAPLF | GEGTRLSVL |
| 300 | | | VNLRCILK (SEQ ID | (SEQ ID | WLFT (SEQ ID | (SEQ ID | ANMSQGRTLY (SEQ ID NO: | (SEQ ID NO: | (SEQ ID |
| | | | NO: 23) | NO: | NO: 5959) | NO: 8927) | 11895) | 25991) | NO: 16441) |
| | | | | 2991) | | | | | |
| Clonotype_ | 1 | TRA | GEQVEQRPPHLSVPEGDS | DSATAY | FYWYKQEPGAGLQ | VFSNVDR | KEEQGLTVLLNKKDKQLSLNLTAAH | CAVSYTGGADRLTF | GKGTQLIIQ |
| 300 | | | AVIICTYT (SEQ ID | (SEQ ID | LLMS (SEQ ID | (SEQ ID | PGDSAVYF (SEQ ID NO: | (SEQ ID NO: | P (SEQ ID |
| | | | NO: 100) | NO: | NO: 6036) | NO: | 11972) | 25992) | NO: |
| | | | | 2990) | | 9004) | | | 16360) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSASSGGKDTQY | GPGTRLLVL |
| 301 | | | VNLRCILK (SEQ ID | (SEQ ID | WLFT (SEQ ID | (SEQ ID | ANMSQGRTLY (SEQ ID NO: | F (SEQ ID NO: | (SEQ ID |
| | | | NO: 23) | NO: | NO: 5959) | NO: | 11895) | 25993) | NO: |
| | | | | 2991) | | 8927) | | | 16341) |
| Clonotype_ | 1 | TRA | SQLAEENSWALSVHEGES | TSITA | LQWYRQKSGKGPA | IRSNERE | KRNGRLRATLDTSSQSSSLSITATR | CDTNAYKVIF (SEQ | GKGTHLHVL |
| 301 | | | VTVNCSYK (SEQ ID | (SEQ ID | QLIL (SEQ ID | (SEQ ID | CEDTAVYF (SEQ ID NO: | ID NO: 25994) | P (SEQ ID |
| | | | NO: 188) | NO: | NO: 6124) | NO: | 11896) | | NO: |
| | | | | 2992) | | 8928) | | | 16330) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSAGQANSDYTF | GSGTRLLVI |
| 302 | | | VNLRCILK (SEQ ID | (SEQ ID | WLFT (SEQ ID | (SEQ ID | ANMSQGRTLY (SEQ ID NO: | (SEQ ID NO: | (SEQ ID |
| | | | NO: 23) | NO: | NO: 5959) | NO: | 11895) | 25995) | NO: |
| | | | | 2991) | | 8927) | | | 16337) |
| Clonotype_ | 1 | TRA | QQQVRQSPQSLTVWEGET | DSTFNY | FPWYQQFPGEGPA | IRSVSDK | KEDGRFTIFFNKREKKLSLHITDSQ | CAARLTGNTRKLIF | GLGTTLQVQ |
| 302 | | | AILNCSYE (SEQ ID | (SEQ ID | LLIS (SEQ ID | (SEQ ID | PGDSATYF (SEQ ID NO: | (SEQ ID NO: | P (SEQ ID |
| | | | NO: 68) | NO: | NO: 6004) | NO: | 11940) | 25996) | NO: 16326) |
| | | | | 3036) | | 8988) | | | |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSAGLGDAEQFF | GPGTRLTVL |
| 303 | | | VNLRCILK (SEQ ID | (SEQ ID | WLFT (SEQ ID | (SEQ ID | ANMSQGRTLY (SEQ ID NO: | (SEQ ID NO: | (SEQ ID |
| | | | NO: 23) | NO: | NO: 5959) | NO: | 11895) | 25997) | NO: |
| | | | | 2991) | | 8927) | | | 16327) |
| Clonotype_ | | TRA | SQLAEENLWALSVHEGES | TSITA | LQWYRQKSGEGPA | IRSNERE | KRNGRLRATLDTSSQSSSLSITATR | CATSSGQKLVF | GQGTILKVY |
| 303 | | | VTVNCSYK (SEQ ID | (SEQ ID | QLIL (SEQ ID | (SEQ ID | CEDTAVYF (SEQ ID NO: | (SEQ ID NO: | L (SEQ ID |
| | | | NO: 190) | NO: | NO: 5960) | NO: | 11896) | 25998) | NO: |
| | | | | 2992) | | 8928) | | | 16414) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSGLGQDTQYF | GPGTRLLVL |
| 304 | | | VNLRCILK (SEQ ID | (SEQ ID | WLFT (SEQ ID | (SEQ ID | ANMSQGRTLY (SEQ ID NO: | (SEQ ID NO: | (SEQ ID |
| | | | NO: 23) | NO: | NO: 5959) | NO: 8927) | 11895) | 25999) | NO: 16341) |
| | | | | 2991) | | | | | |
| Clonotype_ | 1 | TRA | AQSVTQPDARVTVSEGAS | SSVTPY | LFWYVQYPRQGLQ | YYSGDPV | GVNGFEAEFSKSNSSFHLRKASVH | CAVSMNTGGADRLT | GKGTQLIIQ |
| 304 | | | LQLRCKYS (SEQ ID | (SEQ ID | LLLK (SEQ ID | V (SEQ | WSDSAVYF (SEQ ID NO: | F (SEQ ID NO: | P (SEQ ID |
| | | | NO: 12) | NO: | NO: 5948) | ID NO: | 11892) | 26000) | NO: |
| | | | | 2988) | | 8916) | | | 16360) |
| Clonotype_ | 1 | TRB | VTLLEQNPRWRLVPRGQA | NSQYPW | MSWYQQDLQKQLQ | LRSPGD | KEVKSLPGADYLATRVTDTELRLQV | CTCSGGLGGHSAET | GSGTRLTVL |
| 305 | | | VNLRCILK (SEQ ID | (SEQ ID | WLFT (SEQ ID | (SEQ ID | ANMSQGRTLY (SEQ ID NO: | LYF (SEQ ID NO: | (SEQ ID |
| | | | NO: 23) | NO: | NO: 5959) | NO: | 11895) | 26001) | NO: |
| | | | | 2991) | | 8927) | | | 16325) |
| Clonotype_ | 1 | TRA | QQQVRQSPQSLTVWEGET | NSAFDY | FPWYQQFPGEGPA | IRSVSDK | KEDGRFTIFFNKREKKLSLHITDSQ | CAASHSGGSNYKLT | GKGTLLTVT |
| 305 | | | AILNCSYE (SEQ ID | (SEQ ID | LLIA (SEQ ID | (SEQ ID | PGDSATYF (SEQ ID NO: | F (SEQ ID NO: | P (SEQ ID |
| | | | NO: 68) | NO: | NO: 6020) | NO: | 11940) | 26002) | NO: |
| | | | | 3052) | | 8988) | | | 16642) |

TABLE 3
TCR sequences from one mouse, cohort 1, RPRPSQPSSL (SEQ ID NO: 32957) vaccination

| Clono- | | | | | | | | | |
|---|---|---|---|---|---|---|---|---|---|
| type | Freq | Chain | fwr1 | cdr1 | fwr2 | cdr2 | fwr3 | cdr3 | fwr4 |
| Clono- | 352 | TRB | ETAVFQTPNYHVTQVGNEV | LGHDT | MYWYKQDSKKLLK | YNNKQL | IVNETVPRRFSPQSSDKAHLNLRIKS | CASSSTGANERLF | GHGTKLSV |
| type_1 | | | SFNCKQT (SEQ ID NO: | (SEQ ID | IMFS (SEQ ID | (SEQ ID | VEPEDSAVYL (SEQ ID NO: | F (SEQ ID NO: 31089) | L (SEQ |
| | | | 99) | NO: | NO: 6035) | NO: 9003) | 11971) | | ID NO: |
| | | | | 3067) | | | | | 16401) |
| Clono- | 325 | TRA | QQKVQQSPESLIVPEGAMT | DSASQY | FAWYRQHSGKAPK | IFSNGE | KEEGRFTIHLNKASLHFSLHIRDSQP | CAVSSYSNYQLIW | GSGTKLII |
| type_1 | | | SLNCTFS (SEQ ID NO: | (SEQ ID | ALMS (SEQ ID | (SEQ ID | SDSALYL (SEQ ID NO: 11948) | (SEQ ID NO: 31090) | KP (SEQ |
| | | | 214) | NO: | NO: 6150) | NO: 8932) | | | ID NO: |
| | | | | 3044) | | | | | 16372) |
| Clono- | 6 | TRB | GGIITQTPKFLIGQEGQKL | FNHDT | MYWYRQDSGKGLR | SITEND | LQKGDLSEGYDASREKKSSFSLTVTS | CASSIGTGDNQAP | GEGTRLSV |
| type_2 | | | TLKCQQN (SEQ ID NO: | (SEQ ID | LLIY (SEQ | (SEQ ID | AQKNEMAVFL (SEQ ID NO: | LF (SEQ ID NO: | L (SEQ ID |
| | | | 27) | NO: | ID NO: 5963) | NO: 8931) | 11899) | 25393) | NO: |
| | | | | 2995) | | | | | 16441) |
| Clono- | 6 | TRA | GEQVEQLPSSLIVQEGASV | DSASVY | FPWYKQEPGKRLQ | IRSNMER | KQTQRLTLLFDKKTKHLSLHITATQP | CSASNNYAQGLTF | GLGTRVSV |
| type_2 | | | LINCSYT (SEQ ID NO: | (SEQ ID | FIID (SEQ ID | (SEQ ID | GDSAIYF (SEQ ID NO: 24636) | (SEQ ID NO: | FP (SEQ |
| | | | 160) | NO: | NO: 6096) | NO: | | 25394) | ID NO: |
| | | | | 3128) | | 8952) | | | 16344) |
| Clono- | 5 | TRB | ETAVFQTPNYHVTQVGNEV | LGHDT | MYWYKQDSKKLLK | YNNKQL | IVNETVPRRFSPQSSDKAHLNLRIKS | CASSGDWGARAET | GSGTRLTV |
| type_3 | | | SFNCKQT (SEQ ID NO: | (SEQ ID | IMFS (SEQ ID | (SEQ ID | VEPEDSAVYL (SEQ ID NO: | LYF (SEQ ID | L |
| | | | 99) | NO: | NO: 6035) | NO: | 11971) | NO: 31093) | (SEQ ID |
| | | | | 3067) | | 9003) | | | NO: |
| | | | | | | | | | 16325) |
| Clono- | 5 | TRA | GEKVEQHQSTLSVREGDSA | DTASSY | FPWYKQEAGKSLH | IRSNVDR | KQSQRLTVLLDKKAKRFSLHITATQP | CAASIGNYAQGLT | GLGTRVSV |
| type_3 | | | VINCTYT (SEQ ID NO: | (SEQ ID | FVID (SEQ ID | (SEQ ID | EDSAIYF (SEQ ID NO: 12238) | F (SEQ ID NO: | FP (SEQ |
| | | | 366) | NO: | NO: 5990) | NO: 8958) | | 31094) | ID NO: |
| | | | | 3022) | | | | | 16344) |
| Clono- | 2 | TRB | EAAVTQSPRNKVAVTGGKV | NNHNN | MYWYRQDTGHGLR | SYGAGS | TEKGDIPDGYKASRPSQENFSLILEL | CASGDGGNSDYTF | GSGTRLLV |
| type_4 | | | TLSCNQT (SEQ ID NO: | (SEQ ID | LIHY (SEQ ID | (SEQ ID | ATPSQTSVYF (SEQ ID NO: | (SEQ ID NO: 31095) | I (SEQ ID |
| | | | 3) | NO: | NO: 5939) | NO: 8907) | 11875) | | NO: |
| | | | | 2971) | | | | | 16337) |
| Clono- | 2 | TRA | GEQVEQLPSILRVQEGSSA | NSASNY | FPWYKQEPGENPK | IRSNMER | KQTQGLIVLLDKKAKRFSLHITDTQP | CAAGNSGTYQRF | GTGTKLQV |
| type_4 | | | SINCTYE (SEQ ID NO: | (SEQ ID | LIID (SEQ ID | (SEQ ID | GDSAMYF (SEQ ID NO: 12042) | (SEQ ID NO: 31096) | VP (SEQ |
| | | | 170) | NO: | NO: 5984) | NO: 8952) | | | ID NO: |
| | | | | 3138) | | | | | 16432) |
| Clono- | 2 | TRB | EAAVTQSPRNKVAVTGGKV | NNHNN | MYWYRQDTGHGLR | SYGAGS | TEKGDIPDGYKASRPSQENFSLILEL | CASGDWGRTGQLY | GEGSKLTV |
| type_5 | | | TLSCNQT (SEQ ID NO: | (SEQ ID | LIHY (SEQ ID | (SEQ ID | ATPSQTSVYF (SEQ ID NO: | F (SEQ ID NO: 31097) | L (SEQ |
| | | | 3) | NO: | NO: 5939) | NO: 8907) | 11875) | | ID NO: |
| | | | | 2971) | | | | | 16347) |
| Clono- | 2 | TRA | SQLAEENPWALSVHEGESV | TSITA | LQWYRQKSGEGPA | IRSNERE | KRNGRLRATLDTSSQSSSLSITATRC | CATIGDNNNRIFF | GDGTQLVV |
| type_5 | | | TVNCSYK (SEQ ID NO: | (SEQ ID | QLIL (SEQ ID | (SEQ ID | EDTAVYF (SEQ ID NO: 11896) | (SEQ ID NO: | KP (SEQ |
| | | | 24) | NO: | NO: 5960) | NO: | | 31098) | ID NO: |
| | | | | 2992) | | 8928) | | | 16328) |
| Clono- | 2 | TRB | EAAVTQSPRNKVAVTGGKV | NNHNN | MYWYRQDTGHGLR | SYGAGS | TEKGDIPDGYKASRPSQENFSLILEL | CASGGPGQEQYF | GPGTRLTV |
| type_6 | | | TLSCNQT (SEQ ID NO: | (SEQ ID | LIHY (SEQ ID | (SEQ ID | ATPSQTSVYF (SEQ ID NO: | (SEQ ID NO: 31099) | L (SEQ |
| | | | 3) | NO: | NO: 5939) | NO: | 11875) | | ID NO: |
| | | | | 2971) | | 8907) | | | 16327) |
| Clono- | 2 | TRA | GDSVTQTEGQVALSEEDFL | ASGYPA | LFWYVQYPGEGPQ | ASRDKEK | GSSRGFEATYDKGTTSFHLRKASVQE | CALGRSSNTNKVV | GTGTRLQV |
| type_6 | | | TIHCNYS (SEQ ID NO: | (SEQ ID | FLFR (SEQ ID | (SEQ ID | SDSAVYY (SEQ ID NO: 11886) | F (SEQ ID NO: | LP (SEQ |
| | | | 14) | NO: | NO: 6050) | NO: 8918) | | 31100) | ID NO: |
| | | | | 3082) | | | | | 16698) |
| Clono- | 2 | TRB | GGIITQTPKFLIGQEGQKL | FNHDT | MYWYRQDSGKGLR | SITEND | LQKGDLSEGYDASREKKSSFSLTVTS | CASSPGTIFSGNT | GEGSRLIV |
| type_7 | | | TLKCQQN (SEQ ID NO: | (SEQ ID | LIYY (SEQ ID | SEQ ID | AQKNEMAVFL (SEQ ID NO: | LYF (SEQ ID | V (SEQ |
| | | | 27) | NO: | NO: 5963) | NO: 8931) | 11899) | NO: 31101) | ID NO: |
| | | | | 2995) | | | | | 16471) |
| Clono- | 2 | TRA | QQKVQQSPESLIVPEGAMT | DSASQY | FAWYRQHSGKAPK | IFSNGE | KEEGRFTIHLNKASLHFSLHIRDSQP | CAVSSYSNYQLIW | GSGTKLII |
| type_7 | | | SLNCTFS (SEQ ID NO: | (SEQ ID | ALMS (SEQ ID | (SEQ ID | SDSALYL (SEQ ID NO: 11948) | (SEQ ID NO: 31090) | KP (SEQ |
| | | | 214) | NO: | NO: 6150) | NO: 8932) | | | ID NO: |
| | | | | 3044) | | | | | 16372) |
| Clono- | 1 | TRB | NTKITQSPRYLILGRANKS | LGHNA | MYWYKQSAEKPPE | YNLKQL | IRNETVPSRFIPECPDSSKLLLHISA | CASSQEGGREQYF | GPGTRLTV |
| type_8 | | | LECEQH (SEQ ID NO: | (SEQ ID | LMFL (SEQ ID | (SEQ ID | VDPEDSAVYF (SEQ ID NO: | (SEQ ID NO: | L (SEQ ID |
| | | | 95) | NO: | NO: 6031) | NO: 8999) | 11967) | 31103) | NO: |
| | | | | 2983) | | | | | 16327) |
| Clono- | 1 | TRA | GEQVEQLPSSLIVQEGASV | DSASVY | FPWYKQEPGKRLQ | IRSNMER | KQNQRLTLLFDKKTKHLSLHITATQP | CSATYRGSALGRL | GAGTQLIV |
| type_8 | | | LINCSYT (SEQ ID NO: | (SEQ ID | FIID (SEQ ID | (SEQ ID | GDSAIYF (SEQ ID NO: 12032) | HF (SEQ ID NO: | IP (SEQ |
| | | | 160) | NO: | NO: 6096) | NO: | | 31104) | ID NO: |
| | | | | 3128) | | 8952) | | | 16480) |
| Clono- | 1 | TRB | NTKITQSPRYLILGRANKS | LGHNA | MYWYKQSAEKPPE | YNLKQL | IRNETVPSRFIPECPDSSKLLLHISA | CASSQDDRDEQYF | GPGTRLTV |
| type_9 | | | LECEQH (SEQ ID NO: | (SEQ ID | LMFL (SEQ ID | (SEQ ID | VDPEDSAVYF (SEQ ID NO: | (SEQ ID NO: | L (SEQ ID |
| | | | 95) | NO: | NO: 6031) | NO: 8999) | 11967) | 31105) | NO: |
| | | | | 2983) | | | | | 16327) |
| Clono- | 1 | TRA | QQKVQQSPESLIVPEGAMT | DSASQY | FAWYRQHSGKAPK | IFSNGE | KEEGRFTIHLNKASLHFSLHIRDSQP | CAVSSYSNYQLIW | GSGTKLII |
| type_9 | | | SLNCTFS (SEQ ID NO: | (SEQ ID | ALMS (SEQ ID | (SEQ ID | SDSALYL (SEQ ID NO: 11948) | (SEQ ID NO: | KP (SEQ |
| | | | 214) | NO: | NO: 6150) | NO: | | 31090) | ID NO: |
| | | | | 3044) | | 8932) | | | 16372) |
| Clono- | 1 | TRB | NTKITQSPRYLILGRANKS | LGHNA | MYWYKQSAEKPPE | YNLKQL | IRNETVPSRFIPECPDSSKLLLHISA | CASSQDGGAGEQY | GPGTRLTV |
| type_10 | | | LECEQH (SEQ ID NO: | (SEQ ID | LMFL (SEQ ID | (SEQ ID | VDPEDSAVYF (SEQ ID NO: | F (SEQ ID NO: | L (SEQ ID |
| | | | 95) | NO: | NO: 6031) | NO: 8999) | 11967) | 31107) | NO: |
| | | | | 2983) | | | | | 16327) |
| Clono- | 1 | TRA | GDQVEQSPSALSLHEGTDS | TTMRS | VQWFRQNSRGSLI | LASGT | KENGRLKSAFDSKERRYSTLHIRDAQ | CAAEENYAQGLTF | GLGTRVSV |
| type_10 | | | ALRCNFT (SEQ ID NO: | (SEQ ID | SLFY (SEQ ID | (SEQ ID | LEDSGTYF (SEQ ID NO: 11890) | (SEQ ID NO: | FP (SEQ |
| | | | 18) | NO: | NO: 5954) | NO: 8922) | | 31108) | ID NO: |
| | | | | 2986) | | | | | 16344) |
| Clono- | 1 | TRB | NTKITQSPRYLILGRANKS | LGHNA | MYWYKQSAEKPPE | YNLKQL | IRNETVPSRFIPECPDSSKLLLHISA | CASSQGLGGYEQY | GPGTRLTV |
| type_11 | | | LECEQH (SEQ ID NO: | (SEQ ID | LMFL (SEQ ID | (SEQ ID | VDPEDSAVYF (SEQ ID NO: | F (SEQ ID NO: | L (SEQ ID |
| | | | 95) | NO: | NO: 6031) | NO: 8999) | 11967) | 31109) | NO: |
| | | | | 2983) | | | | | 16327) |
| Clono- | 1 | TRA | QQKVQQSPESLIVPEGAMT | DSASQY | FAWYRQHSGKAPK | IFSNGE | KEEGRFTIHLNKASLHFSLHIRDSQP | CAVDTGNYKYVF ( | GAGTRLKV |
| type_11 | | | SLNCTFS (SEQ ID NO: | (SEQ ID | ALMS (SEQ ID | (SEQ ID | SDSALYL (SEQ ID NO: 11948) | SEQ ID NO: | IA (SEQ |
| | | | 214) | NO: | NO: 6150) | NO: | | 31110) | ID NO: |
| | | | | 3044) | | 8932) | | | 16380) |
| Clono- | 1 | TRB | NTKITQSPRYLILGRANKS | LGHNA | MYWYKQSAEKPPE | YNLKQL | IRNETVPSRFIPECPDSSKLLLHISA | CASRDWDAYEQYF | GPGTRLTV |
| type_12 | | | LECEQH (SEQ ID NO: | (SEQ ID | LMFL (SEQ ID | (SEQ ID | VDPEDSAVYF (SEQ ID NO: | (SEQ ID NO: | L (SEQ ID |
| | | | 95) | NO: | NO: 6031) | NO: | 11967) | 31111) | NO: |
| | | | | 2983) | | 8999) | | | 16327) |
| Clono- | 1 | TRA | AQKVTQTQTSISVMEKTTV | TRDSSYF | LFWYKQTASGEIV | QDSYKKE | ATEGHYSLNFQKPKSSIGLIITATQI | CAMREGSNQGGSA | GEGTKLTV |
| type_12 | | | TMDCVYE (SEQ ID NO: | (SEQ ID | FLIR (SEQ ID | N (SEQ | EDSAVYF (SEQ ID NO: 11916) | KLIF ( SEQ ID | SS (SEQ |
| | | | 44) | NO: | NO: 5942) | ID NO: | | NO: 31112) | ID NO: |
| | | | | 2974) | | 8910) | | | 16384) |
| Clono- | 1 | TRB | NTKITQSPRYLILGRANKS | LGHNA | MYWYKQSAEKPPE | YNLKQL | IRNETVPSRFIPECPDSSKLLLHISA | CASSLGEDAEQFF | GPGTRLTV |
| type_13 | | | LECEQH (SEQ ID NO: | (SEQ ID | LMFL (SEQ ID | (SEQ ID | VDPEDSAVYF (SEQ ID NO: | (SEQ ID NO: | L (SEQ ID |
| | | | 95) | NO: | NO: 6031) | NO: | 11967) | 31113) | NO: |
| | | | | 2983) | | 8999) | | | 16327) |
| Clono | 1 | TRA | GDSVTQTEGQVTVSESKSL | ATSIGYP | LFWYVRYPGEGLQ | VITAGQK | GSSRGFEATYNKEATSFHLQKASVQE | CALSMGGYKLTF | GTGTSLLV |
| type_13 | | | IINCTYS (SEQ ID NO: | N (SEQ | LLLK (SEQ ID | (SEQ ID | SDSAVYY (SEQ ID NO: 12094) | SEQ ID NO: | DP (SEQ |
| | | | 60) | ID NO: | NO: 5996) | NO: | | (31114) | ID NO: |
| | | | | 3330) | | 8964) | | | 16398) |
| Clono- | 1 | TRB | EAGVTQSPRYAVLQEGQAV | SGHDT | LYWYQQPRDQGPQ | FRDEAV | IDNSQLPSDRFSAVRPKGTNSTLKIQ | CASSPTLYEQYF | GPGTRLTV |
| type_14 | | | SFWCDPI (SEQ ID NO: | (SEQ ID | LLVY (SEQ ID | (SEQ ID | SAKQGDTATYL (SEQ ID NO: | (SEQ ID NO: | L (SEQ ID |
| | | | 1) | NO: | NO: 5937) | NO: | 11873) | 31115) | NO: |
| | | | | 2969) | | 8905) | | | 16327) |
| Clono- | 1 | TRA | AQSVTQPDARVTVSEGASL | YSATPY | ILFWYVQYPRQGLQ | YYSGDPV | QGVNGFEAEFSKSNSSFHLRKASVHW | CAVSTGANTGKLT | GHGTILRV |
| type_14 | | | QLRCKYS (SEQ ID NO: | (SEQ ID | LLLK (SEQ ID | V (SEQ | SDSAVYF (SEQ ID NO: 11892) | F (SEQ ID NO: 31116) | HP (SEQ |
| | | | 12) | NO: | NO: 5948) | ID NO: | | | ID NO: |
| | | | | 2980) | | 8916) | | | 16356) |
| Clono- | 1 | TRB | EAGVTQSPRYAVLQEGQAV | SGHDT | LYWYQQPRDQGPQ | FRDEAV | IDNSQLPSDRFSAVRPKGTNSTLKIQ | CASSPGLGYAEQF | GPGTRLTV |
| type_15 | | | SFWCDPI (SEQ ID NO: | (SEQ ID | LLVY (SEQ ID | (SEQ ID | SAKQGDTATYL (SEQ ID NO: | F (SEQ ID NO: | L (SEQ ID |
| | | | 1) | NO: | NO: 5937) | NO: 8905) | 11873) | 31117) | NO: |
| | | | | 2969) | | | | | 16327) |
| Clono- | 1 | TRA | GDQVEQSPSALSLHEGTGS | TTMRA | VQWFQQNSRGSLI | LASGT | KENGRLKSTFNSKESYSTLHIRDAQL | CAASNTNKVVF | GTGTRLQV |
| type_15 | | | ALRCNFT (SEQ ID NO: | (SEQ ID | NLFY (SEQ ID | (SEQ ID | EDSGTYF (SEQ ID NO: 12130) | (SEQ ID NO: | LP (SEQ |
| | | | 112) | NO: | NO: 6222) | NO: | | 31118) | ID NO: |
| | | | | 3080) | | 8922) | | | 16698) |
| Clono- | 1 | TRB | EAGVTQSPRYAVLQEGQAV | SGHDT | LYWYQQPRDQGPQ | FRDEAV | IDNSQLPSDRFSAVRPKGTNSTLKIQ | CASSFTLGEYEQY | GPGTRLTV |
| type_16 | | | SFWCDPI (SEQ ID NO: | (SEQ ID | LLVY (SEQ ID | (SEQ ID | SAKQGDTATYL (SEQ ID NO: | F (SEQ ID NO: | L (SEQ ID |
| | | | 1) | NO: | NO: 5937) | NO: | 11873) | 31119) | NO: |
| | | | | 2969) | | 8905) | | | 16327) |
| Clono- | 1 | TRA | LAKTTQPPSMEAYEGQEVN | NIATSEY | IYWYRQVPHQGPQ | GYKDYVV | NEVASLFISADRKLSTLSLPWVSLRD | CIVTDMGTGGYKV | GSGTRLLV |
| type_16 | | | VSCSHT (SEQ ID NO: | (SEQ ID | FIIQ (SEQ ID | (SEQ ID | AAVYY (SEQ ID NO: 11978) | VF (SEQ ID NO: | SP (SEQ |
| | | | 106) | NO: | NO: 6042) | NO: 9010) | | 31120) | ID NO: |
| | | | | 3074) | | | | | 16354) |
| Clono- | 1 | TRB | DPKIIQKPKYLVAVTGSEK | LGHNA | MYWYRQSAKKPLE | YSYQKL | MDNQTASSRFQPQSSKKNHLDLQITA | CASSQEGPGNTLY | GEGSRLIV |
| type_17 | | | ILICEQY (SEQ ID NO: | (SEQ ID | FMFS (SEQ ID | (SEQ ID | LKPDDSATYF (SEQ ID NO: | F (SEQ ID NO: 31121) | V (SEQ ID |
| | | | 15) | NO: | NO: 5951) | NO: | 11887) | | NO: |
| | | | | 2983) | | 8919) | | | 16471) |
| Clono- | 1 | TRA | GDSVTQTEGPVTVSESESL | ATSIAYP | LFWYVRYPGEGLQ | VITAGQK | GSSRGFEATYNKETTSFHLQKASVQE | CALGNNNNAPRF | GAGTKLSV |
| type_17 | | | IINCTYS (SEQ ID NO: | N (SEQ | LLLK (SEQ ID | (SEQ ID | SDSAVYY (SEQ ID NO: 11932) | S(EQ ID NO: 31122) | KP (SEQ |
| | | | 130) | ID NO: | NO: 5996) | NO: 8964) | | | ID NO: |
| | | | | 3098) | | | | | 16426) |
| Clono- | 1 | TRB | DPKIIQKPKYLVAVTGSEK | LGHNA | MYWYRQSAKKPLE | YSYQKL | MDNQTASSRFQPQSSKKNHLDLQITA | CASSQYEQYF | GPGTRLTV |
| type_18 | | | ILICEQY (SEQ ID NO: | (SEQ ID | FMFS (SEQ ID | (SEQ ID | LKPDDSATYF (SEQ ID NO: | (SEQ ID NO: | L (SEQ ID |
| | | | 15) | NO: | NO: 5951) | NO: | 11887) | 31123) | NO: |
| | | | | 2983) | | 8919) | | | 16327) |
| Clono- | 1 | TRA | GDQVEQSPSALSLHEGTGS | TTMRA | VQWFRKNSRGSLI | LASGT | KENGRLKSAFDSKERYSTLHIRDAQL | CAAFNMGYKLTF | GTGTSLLV |
| type_18 | | | ALRCNFT (SEQ ID NO: | (SEQ ID | NLFY (SEQ ID | (SEQ ID | EDSGTYF (SEQ ID NO: 11984) | (SEQ ID NO: | DP (SEQ |
| | | | 112) | NO: | NO: 6048) | NO: | | 31124) | ID NO: |
| | | | | 3080) | | 8922) | | | 16398) |
| Clono- | 1 | TRB | ETAVFQTPNYHVTQVGNEV | LGHDT | MYWYKQDSKKLLK | YNNKQL | IVNETVPRRFSPQSSDKAHLNLRIKS | CASSPRQGTNTLY | GAGTRLSV |
| type_19 | | | SENCKQT (SEQ ID NO: | (SEQ ID | IMFS (SEQ ID | (SEQ ID | VEPEDSAVYL (SEQ ID NO: | F (SEQ ID NO: | L (SEQ ID |
| | | | 99) | NO: | NO: 6035) | NO: | 11971) | 31125) | NO: |
| | | | | 3067) | | 9003) | | | 16365) |
| Clono- | 1 | TRA | GEKVEQHESTLSVREGDSA | DTASSY | FPWYKQEAGKSLH | IRSNVDR | KQSQRLIVLLDKKAKRFSLHITATQP | CAASIVGDNSKLI | GLGTSLVV |
| type_19 | | | VINCTYT (SEQ ID NO: | (SEQ ID | FVID (SEQ ID | (SEQ ID | EDSAIYF (SEQ ID NO: 11926) | W (SEQ ID NO: | NP (SEQ |
| | | | 54) | NO: | NO: 5990) | NO: | | 31126) | ID NO: |
| | | | | 3022) | | 8958) | | | 17292) |
| Clono- | 1 | TRB | ETAVFQTPNYHVTQVGNEV | LGHDT | MYWYKQDSKKLLK | YNNKQL | IVNETVPRRFSPQSSDKAHLNLRIKS | CASSSRQNTEVFF | GKGTRLTV |
| type 20 | | | SFNCKQT (SEQ ID NO: | (SEQ ID | IMFS (SEQ ID | (SEQ ID | VEPEDSAVYL (SEQ ID NO: | (SEQ ID NO: | V (SEQ ID |
| | | | 99) | NO: | NO: 6035) | NO: 9003) | 11971) | 31127) | NO: |
| | | | | 3067) | | | | | 16333) |
| Clono- | 1 | TRA | GQQVQQSPASLVLQEGENA | TSLNS | MQWFYQRPGGSLV | NPSGT | KHSGRLTSTTVIKERRSSLHISSSQT | CALERSYGNEKIT | GAGTKLTI |
| type_20 | | | ELQCNFS (SEQ ID NO: | (SEQ ID | SLFY (SEQ ID | (SEQ ID | TDSGTYL (SEQ ID NO: 11928) | F (SEQ ID NO: | KP (SEQ |
| | | | 10) | NO: | NO: 5946) | NO: 8914) | | 31128) | ID NO: |
| | | | | 2978) | | | | | 16512) |
| Clono- | 1 | TRB | ETAVFQTPNYHVTQVGNEV | LGHDT | MYWYKQDSKKLLK | YNNKQL | IVNETVPRRFSPQSSDKAHLNLRIKS | CASISRDSSYEQY | GPGTRLTV |
| type_21 | | | SFNCKQT (SEQ ID NO: | (SEQ ID | IMFS (SEQ ID | (SEQ ID | VEPEDSAVYL (SEQ ID NO: | F (SEQ ID NO: | L (SEQ ID |
| | | | 99) | NO: | NO: 6035) | NO: | 11971) | 31129) | NO: |
| | | | | 3067) | | 9003) | | | 16327) |
| Clono- | 1 | TRA | GQQVQQSPASLVLQEGENA | TSLNS | MQWFYQRPEGSLV | NPSGT | KQSGRLTSTTVIKERRSSLHISSSQI | CALNSAGNKLTF | GIGTRVLV |
| type_21 | | | ELQCNFS (SEQ ID NO: | (SEQ ID | SLFY (SEQ ID | (SEQ ID | TDSGTYL (SEQ ID NO: 12038) | (SEQ ID NO: 16304) | RP (SEQ |
| | | | 10) | NO: | NO: 6192) | NO: 8914) | | | ID NO: |
| | | | | 2978) | | | | | 16580) |
| Clono- | 1 | TRB | EAAVTQSPRNKVAVTGGKV | NNHNN | MYWYRQDTGHGLR | SYGAGS | TEKGDIPDGYKASRPSQENFSLILEL | CASGDARQDQDTQ | GPGTRLLV |
| type_22 | | | TLSCNQT (SEQ ID NO: | (SEQ ID | LIHY (SEQ ID | (SEQ ID | ATPSQTSVYF (SEQ ID NO: | YF (SEQ ID NO: | L (SEQ ID |
| | | | 3) | NO: | NO: 5939) | NO: 8907) | 11875) | 31131) | NO: |
| | | | | 2971) | | | | | 16341) |
| Clono- | 1 | TRA | GEQVEQLPSILRVQEGSSA | DSASNY | FPWYKQEPGENPK | IRSNMER | KQIQELIVLLDKKAKRFSLHITDTQP | CAAPDASSGSWQL | GSGTQLTV |
| type_22 | | | SINCSYE (SEQ ID NO: | (SEQ ID | LIID (SEQ ID | (SEQ ID | GDSAMYF (SEQ ID NO: 11920) | IF (SEQ ID NO: | MP (SEQ |
| | | | 48) | NO: | NO: 5984) | NO: | | 31132) | ID NO: |
| | | | | 3016) | | 8952) | | | 16400) |
| Clono- | 1 | TRB | EAAVTQSPRNKVAVTGGKV | NNHNN | MYWYRQDTGHGLR | SYGAGS | TEKGDIPDGYKASRPSQENFSLILEL | CASGGTGANTGQL | GEGSKLTV |
| type_23 | | | TLSCNQT (SEQ ID NO: | (SEQ ID | LIHY (SEQ ID | S(EQ ID | ATPSQTSVYF (SEQ ID NO: | YF (SEQ ID NO: | L (SEQ |
| | | | 3) | NO: | NO: 5939) | NO: | 11875) | 31133) | ID NO: |
| | | | | 2971) | | 8907) | | | 16347) |
| Clono- | 1 | TRA | AQKVTQTQTSISVMEKTTV | TQDSSYF | LFWYKQTASGEIV | QDSYKKE | ATVGHYSLNFQKPKSSIGLIITATQI | CAMREGVPGANTG | GHGTILRV |
| type_23 | | | TMDCVYE (SEQ ID NO: | (SEQ ID | FLIR (SEQ ID | N (SEQ | EDSAVYF (SEQ ID NO: 11878) | KLTF (SEQ ID | HP (SEQ |
| | | | 44) | NO: | NO: 5942) | ID NO: | | NO: 31134) | ID NO: |
| | | | | 3056) | | 8910) | | | 16356) |
| Clono- | 1 | TRB | EAAVTQSPRNKVTVTGGNV | NSHNY | MYWYRQDTGHGLR | SYGAGN | LQIGDVPDGYKATRTTQEDFFLLLEL | CASSEEENTEVFF | GKGTRLTV |
| type_24 | | | TLSCRQT (SEQ ID NO: | (SEQ ID | LIHY (SEQ ID | (SEQ ID | ASPSQTSLYF (SEQ ID NO: | (SEQ ID NO: 31135) | V (SEQ ID |
| | | | 37) | NO: | NO: 5939) | NO: 8941) | 11909) | | NO: |
| | | | | 3005) | | | | | 16333) |
| Clono- | 1 | TRA | GDSVIQMQGQVTLSENDFL | TTGYPT | LFWYVQYSGEGPQ | VTTANNK | GSSRGFEATYDKGTTSFHLQKTSVQE | CAMRDNTGANTGK | GHGTILRV |
| type_24 | | | FINCTYS (SEQ ID NO: | (SEQ ID | LLLQ (SEQ ID | (SEQ ID | IDSAVYY (SEQ ID NO: 12328) | LTF (SEQ ID | HP (SEQ |
| | | | 456) | NO: | NO: 6392) | NO: 9054) | | NO: 31136) | ID NO: |
| | | | | 3220) | | | | | 16356) |
| Clono- | 1 | TRB | EAAVTQSPRNKVTVTGGNV | NSHNY | MYWYRQDTGHGLR | SYGAGN | LQIGDVPDGYKATRTTQEDFFLLLEL | CASSDLGPQDTQY | GPGTRLLV |
| type_25 | | | TLSCRQT (SEQ ID NO: | (SEQ ID | LIHY (SEQ ID | (SEQ ID | ASPSQTSLYF (SEQ ID NO: | F (SEQ ID NO: | L (SEQ ID |
| | | | 37) | NO: | NO: 5939) | NO: | 11909) | 31137) | NO: |
| | | | | 3005) | | 8941) | | | 16341) |
| Clono- | 1 | TRA | GDSVTQTEGQVTVSESKSL | TTSIAYP | LFWYVRYPGEGLQ | VITAGQK | GSSRGFEATYNKETTSFHLQKASVQE | CALGPNYGGSGNK | GTGTLLSV |
| type_25 | | | IINCTYS (SEQ ID NO: | N (SEQ | LLLK (SEQ ID | (SEQ ID | SDSAVYY (SEQ ID NO: 11932) | LIF (SEQ ID | KP (SEQ |
| | | | 60) | ID NO: | NO: 5996) | NO: 8964) | | NO: 31138) | ID NO: |
| | | | | 3028) | | | | | 16350) |
| Clono- | 1 | TRB | EAAVTQSPRNKVTVTGGNV | NSHNY | MYWYRQDTGHGLR | SYGAGN | LQIGDVPDGYKATRTTQEDFFLLLEL | CASSDAGTAPQDT | GPGTRLLV |
| type_26 | | | TLSCRQT (SEQ ID NO: | (SEQ ID | LIHY (SEQ ID | (SEQ ID | ASPSQTSLYF (SEQ ID NO: | QYF (SEQ ID | L (SEQ ID |
| | | | 37) | NO: | NO: 5939) | NO: 8941) | 11909) | NO: 31139) | NO: |
| | | | | 3005) | | | | | 16341) |
| Clono- | 1 | TRA | LAKTTQPPSMEAYEGQEVN | NIATSEY | IYWYRQVPHQGPQ | GYKDYVV | NEVASLFISADRKLSTLSLPWVSLRD | CIVTDMGGSALGR | GAGTQLIV |
| type_26 | | | VSCSHT (SEQ ID NO: | (SEQ ID | FIIQ (SEQ ID | (SEQ ID | AAVYY (SEQ ID NO: 11978) | LHF (SEQ ID | IP (SEQ |
| | | | 106) | NO: | NO: 6042) | NO: 9010) | | NO: 31140) | ID NO: |
| | | | | 3074) | | | | | 16480) |
| Clono- | 1 | TRB | EAAVTQSPRNKVTVTGGNV | NSHNY | MYWYRQDTGHGLR | SYGAGN | LQIGDVPDGYKATRTTQEDFFLLLEL | CASSDAGGGLYAE | GPGTRLTV |
| type_27 | | | TLSCRQT (SEQ ID NO: | (SEQ ID | LIHY (SEQ ID | (SEQ ID | ASPSQTSLYF (SEQ ID NO: | QFF ( SEQ ID | L (SEQ |
| | | | 37) | NO: | NO: 5939) | NO: 8941) | 11909) | NO: 31141) | ID NO: |
| | | | | 3005) | | | | | 16327) |
| Clono- | 1 | TRA | AQSVTQPDARVTVSEGASL | SSVTPY | LFWYVQYPRQGLQ | YYSGDPV | QGVNGFEAEFSKSNSSFHLRKASVHW | CVVSGNYAQGLTF | GLGTRVSV |
| type_27 | | | QLRCKYS (SEQ ID NO: | (SEQ ID | LLLK (SEQ ID | V (SEQ | SDSAVYF (SEQ ID NO: 11892) | (SEQ ID NO: 31142) | FP (SEQ |
| | | | 12) | NO: | NO: 5948) | ID NO: | | | ID NO: |
| | | | | 2988) | | 8916) | | | 16344) |
| Clono- | 1 | TRB | EAAVTQSPRNKVTVTGGNV | NSHNY | MYWYRQDTGHGLR | SYGAGN | LQIGDVPDGYKATRTTQEDFFLLLEL | CASSDGWQNTLYF | GAGTRLSV |
| type_28 | | | TLSCRQT (SEQ ID NO: | (SEQ ID | LIHY (SEQ ID | (SEQ ID | ASPSQTSLYF (SEQ ID NO: | (SEQ ID NO: | L (SEQ |
| | | | 37) | NO: | NO: 5939) | NO: | 11909) | 31143) | ID NO: |
| | | | | 3005) | | 8941) | | | 16365) |
| Clono- | 1 | TRA | QEKVQQSPESLIVPEGAMS | NSASQS | IWWYQQHPGKGPE | IFSNGN | KKEGRLTVYLNRASLHVSLHIRDSQP | CAGSSNTNKVVF | GTGTRLQV |
| type 28 | | | SLNCTFS (SEQ ID NO: | (SEQ ID | ALIS (SEQ ID | (SEQ ID | SDSAVYL (SEQ ID NO: 11968) | (SEQ ID NO: | LP (SEQ |
| | | | 96) | NO: | NO: 6032) | NO: | | 31144) | ID NO: |
| | | | | 3064) | | 9000) | | | 16698) |
| Clono- | 1 | TRB | EAAVTQSPRNKVTVTGGNV | NSHNY | MYWYRQDTGHGLR | SYGAGN | LQIGDVPDGYKATRTTQEDFFLLLEL | CASSGWDTSGNTL | GEGSRLIV |
| type_29 | | | TLSCRQT (SEQ ID NO: | (SEQ ID | LIHY (SEQ ID | (SEQ ID | ASPSQTSLYF (SEQ ID NO: | YF (SEQ ID NO: | V (SEQ |
| | | | 37) | NO: | NO: 5939) | NO: 8941) | 11909) | 31145) | ID NO: |
| | | | | 3005) | | | | | 16471) |
| Clono- | 1 | TRA | QQQVRQSPQSLTVWEGETA | NSAFDY | FPWYQQFPGEGPA | IRSVSDK | KEDGGFTIFFNKREKNLSLHIKDSQP | CAASEGNNNRIFF | GDGTQLVV |
| type_29 | | | ILNCSYE (SEQ ID NO: | (SEQ ID | LLIA (SEQ ID | (SEQ ID | GDSATYF (SEQ ID NO: 24470) | (SEQ ID NO: | KP (SEQ |
| | | | 68) | NO: | NO: 6020) | NO: | | 31146) | ID NO: |
| | | | | 3052) | | 8988) | | | 16328) |
| Clono- | 1 | TRB | EAAVTQSPRNKVTVTGGNV | NSHNY | MYWYRQDTGHGLR | SYGAGN | LQIGDVPDGYKATRTTQEDFFLLLEL | CASSVRVEEQYF | GPGTRLTV |
| type_30 | | | TLSCRQT (SEQ ID NO: | (SEQ ID | LIHY (SEQ ID | (SEQ ID | ASPSQTSLYF (SEQ ID NO: | (SEQ ID NO: | L (SEQ ID |
| | | | 37) | NO: | NO: 5939) | NO: | 11909) | 31147) | NO: |
| | | | | 3005) | | 8941) | | | 16327) |
| Clono- | 1 | TRA | GEQVEQLPSILRVQEGSSA | DSASNY | FPWYKQEPGENPK | IRSNMER | KQIQELIVLLDKKAKRFSLHITDTQP | CAASGNNAGAKLT | GGGTRLTV |
| type_30 | | | SINCSYE (SEQ ID NO: | (SEQ ID | LIID (SEQ ID | (SEQ ID | GDSAMYF (SEQ ID NO: 11920) | F (SEQ ID NO: | RP (SEQ |
| | | | 48) | NO: | NO: 5984) | NO: 8952) | | 31148) | ID NO: |
| | | | | 3016) | | | | | 16338) |
| Clono- | 1 | TRB | EAAVTQSPRNKVTVTGGNV | NSHNY | MYWYRQDTGHGLR | SYGAGN | LQIGDVPDGYKATRTTQEDFFLLLEL | CASGRKDSPLYF | AAGTRLTV |
| type_31 | | | TLSCRQT (SEQ ID NO: | (SEQ ID | LIHY (SEQ ID | (SEQ ID | ASPSQTSLYF (SEQ ID NO: | (SEQ ID NO: | T (SEQ ID |
| | | | 37) | NO: | NO: 5939) | NO: 8941) | 11909) | 31149) | NO: |
| | | | | 3005) | | | | | 16381) |
| Clono- | 1 | TRA | AQKVTQTQTSISVMEKTTV | TQDSSYF | LFWYKQTASGEIV | QDSYKKE | ATVGHYSLNFQKPKSSIGLIITATQI | CAMREGGDSNNRI | GDGTQLVV |
| type_31 | | | TMDCVYE (SEQ ID NO: | (SEQ ID | FLIR (SEQ ID | N (SEQ | EDSAVYF (SEQ ID NO: 11878) | FF | KP (SEQ |
| | | | 44) | NO: | NO: 5942) | ID NO: | | (SEQ ID NO: 31150) | ID NO: |
| | | | | 3056) | | 8910) | | | 16328) |
| Clono- | 1 | TRB | EAAVTQSPRSKVAVTGGKV | NNHDY | MYWYRQDTGHGLR | SYVADS | TEKGDIPDGYKASRPSQENFSLILEL | CASTPTGGAQETL | GSGTRLTV |
| type_32 | | | TLSCHQT (SEQ ID NO: | (SEQ ID | LIHY (SEQ ID | (SEQ ID | ASLSQTAVYF (SEQ ID NO: | YF (SEQ ID NO: | L (SEQ |
| | | | 7) | NO: | NO: 5939) | NO: 8911) | 11879) | 31151) | ID NO: |
| | | | | 2975) | | | | | 16325) |
| Clono- | 1 | TRA | QQQVRQSPQSLTVWEGETA | DSTFNY | FPWYQQFPGEGPA | IRSVSDK | KEDGRFTIFFNKREKKLSLHITDSQP | CAASHRGSALGRL | GAGTQLIV |
| type_32 | | | ILNCSYE (SEQ ID NO: | (SEQ ID | LLIS (SEQ ID | (SEQ ID | GDSATYF (SEQ ID NO: 11940) | HF (SEQ ID NO: | IP (SEQ |
| | | | 68) | NO: | NO: 6004) | NO: | | 31152) | ID NO: |
| | | | | 3036) | | 8988) | | | 16480) |
| Clono- | 1 | TRB | EAAVTQSPRSKVAVTGGKV | NNHDY | MYWYRQDTGHGLR | SYVADS | TEKGDIPDGYKASRPSQENFSLILEL | CASSPGSGSSGNT | GEGSRLIV |
| type_33 | | | TLSCHQT (SEQ ID NO: | (SEQ ID | LIHY (SEQ ID | (SEQ ID | ASLSQTAVYF (SEQ ID NO: | LYF (SEQ ID | V (SEQ |
| | | | 7) | NO: | NO: 5939) | NO: 8911) | 11879) | NO: 31153) | ID NO: |
| | | | | 2975) | | | | | 16471) |
| Clono- | 1 | TRA | AQKVTQTQTSISVMEKTTV | TRDSSYF | LFWYKQTASGEIV | QDSYKKE | ATEGHYSLNFQKPKSSIGLIITATQI | CAMREVESGANTG | GHGTILRV |
| type_33 | | | TMDCVYE (SEQ ID NO: | (SEQ ID | FLIR (SEQ ID | N (SEQ | EDSAVYF (SEQ ID NO: 11916) | KLTF (SEQ ID | HP (SEQ |
| | | | 44) | NO: | NO: 5942) | ID NO: | | NO: 31154) | ID NO: |
| | | | | 2974) | | 8910) | | | 16356) |
| Clono- | 1 | TRB | EAAVTQSPRSKVAVTGGKV | NNHDY | MYWYRQDTGHGLR | SYVADS | TEKGDIPDGYKASRPSQENFSLILEL | CASSDGGEDTQYF | GPGTRLLV |
| type_34 | | | TLSCHQT (SEQ ID NO: | (SEQ ID | LIHY (SEQ ID | (SEQ ID | ASLSQTAVYF (SEQ ID NO: | (SEQ ID NO: | L (SEQ |
| | | | 7) | NO: | NO: 5939) | NO: | 11879) | 31155) | ID NO: |
| | | | | 2975) | | 8911) | | | 16341) |
| Clono- | 1 | TRA | GDSVTQTEGLVTLTKGLPV | TTYSPF | LFWYVQHLNEAPK | SSTDNKR | TEHQGFYATLHKSSSSFHLQKSSVQL | CALTAGAKLTF | GGGTRLTV |
| type_34 | | | MLNCTYQ (SEQ ID NO: | SEQ ID | LLLK (SEQ ID | (SEQ ID | SDSALYF (SEQ ID NO: 11880) | (SEQ ID NO: 31156) | RP (SEQ |
| | | | 8) | NO: | NO: 5944) | NO: 8912) | | | ID NO: |
| | | | | 2976) | | | | | 16338) |
| Clono- | 1 | TRB | DTTVKQNPRYKLARVGKPV | MNHDT | MYWYQKKPNQAPK | YYDKIL | NREADTFEKFQSSRPNNSFCSLYIGS | CASSSPQNTLYF | GAGTRLSV |
| type_35 | | | NLICSQT (SEQ ID NO: | (SEQ ID | LLLF (SEQ ID | (SEQ ID | AGLEYSAMYL (SEQ ID NO: | (SEQ ID NO: | L (SEQ |
| | | | 299) | NO: | NO: 6235) | NO: | 12171) | 31157) | ID NO: |
| | | | | 3267) | | 9203) | | | 16365) |
| Clono | 1 | TRA | QQKVQQSPESLSVPEGGMA | DRNFQY | FWWYRQHSGEGPK | IFSDGD | KKEGRFTAHLNKASLHVSLHIRDSQP | CAASGSGGSNAKL | GKGTKLSV |
| type_35 | | | SLNCTSS (SEQ ID NO: | S(EQ ID | ALMS (SEQ ID | (SEQ ID | SDSALYF (SEQ ID NO: 11874) | TF (SEQ ID NO: | KS (SEQ |
| | | | 64) | NO: | NO: 5938) | NO: | | 31158) | ID NO: |
| | | | | 2970) | | 8906) | | | 16342) |
| Clono- | 1 | TRB | AQTIHQWPVAEIKAVGSPL | GKSSPN | LYWYWQATGGTLQ | SITVG | QVESVVQLNLSASRPKDDQFILSTEK | CAWRPGQGVSDYT | GSGTRLLV |
| type_36 | | | SLGCTIK (SEQ ID NO: | (SEQ ID | QLFY (SEQ ID | (SEQ ID | LLLSHSGFYL (SEQ ID NO: | F (SEQ ID NO: | I (SEQ |
| | | | 35) | NO: | NO: 5971) | NO: | 11907) | 31159) | ID NO: |
| | | | | 3003) | | 8939) | | | 16337) |
| Clono- | 1 | TRA | AQKVTQTQTSISVMEKTTV | TQDSSYF | LFWYKQTASGEIV | QDSYKKE | ATVGHYSLNFQKPKSSIGLIITATQI | CAMRESNYGNEKI | GAGTKLTI |
| type_36 | | | TMDCVYE (SEQ ID NO: | (SEQ ID | FLIR (SEQ ID | N (SEQ | EDSAVYF (SEQ ID NO: 11878) | TF (SEQ ID NO: | KP (SEQ |
| | | | 44) | NO: | NO: 5942) | ID NO: | | 31160) | ID NO: |
| | | | | 3056) | | 8910) | | | 16512) |
| Clono- | 1 | TRB | AQTIHQWPVAEIKAVGSPL | GKSSPN | LYWYWQATGGTLQ | SITVG | QVESVVQLNLSASRPKDDQFILSTEK | CAWSPSFSYNSPL | AAGTRLTV |
| type_37 | | | SLGCTIK (SEQ ID NO: | (SEQ ID | QLFY (SEQ ID | (SEQ ID | LLLSHSGFYL (SEQ ID NO: | YF (SEQ ID NO: | T (SEQ |
| | | | 35) | NO: | NO: 5971) | NO: 8939) | 11907) | 31161) | ID NO: |
| | | | | 3003) | | | | | 16381) |
| Clono- | 1 | TRA | AQSVTQPDARVTVSEGASL | YFGTPY | LFWYVQYPRQGLQ | YYPGDPV | QGVNGFEAEFSKSNSSFHLRKASVHW | CAVSITGTGSKLS | GKGAKLTV |
| type_37 | | | QLRCKYS (SEQ ID NO: | (SEQ ID | LLLK (SEQ ID | V (SEQ | SDWAVYF (SEQ ID NO: 11924) | F (SEQ ID NO: 31162) | SP (SEQ |
| | | | 12) | NO: | NO: 5948) | ID NO: | | | ID NO: |
| | | | | 3020) | | 8956) | | | 16524) |
| Clono- | 1 | TRB | GGIITQTPKFLIGQEGQKL | FNHDT | MYWYRQDSGKGLR | SITEND | LQKGDLSEGYDASREKKSSFSLTVTS | CASSPGTIFSGNT | GEGSRLIV |
| type_38 | | | TLKCQQN (SEQ ID NO: | (SEQ ID | LIYY (SEQ ID | (SEQ ID | AQKNEMAVFL (SEQ ID NO: | LYF (SEQ ID | V (SEQ |
| | | | 27) | NO: | NO: 5963) | NO: 8931) | 11899) | NO: 31101) | ID NO: |
| | | | | 2995) | | | | | 16471) |
| Clono- | 1 | TRA | GDSVTQTEGQVTVSESKSL | TTSIAYP | LFWYVRYPGEGLQ | VITAGQK | GSSRGFEATYNKETTSFHLQKASVQE | CALGESGQKLVF | GQGTILKV |
| type_38 | | | IINCTYS (SEQ ID NO: | N (SEQ | LLLK (SEQ ID | (SEQ ID | SDSAVYY (SEQ ID NO: 11932) | (SEQ ID NO: 31164) | YL (SEQ |
| | | | 60) | ID NO: | NO: 5996) | NO: 8964) | | | ID NO: |
| | | | | 3028) | | | | | 16414) |
| Clono- | 1 | TRB | GGIITQTPKFLIGQEGQKL | FNHDT | MYWYRQDSGKGLR | SITEND | LQKGDLSEGYDASREKKSSFSLTVTS | CASSIWPEQYF | GPGTRLTV |
| type_39 | | | TLKCQQN (SEQ ID NO: | (SEQ ID | LIYY (SEQ ID | (SEQ ID | AQKNEMAVFL (SEQ ID NO: | (SEQ ID NO: 31165) | L (SEQ |
| | | | 27) | NO: | NO: 5963) | NO: | 11899) | | ID NO: |
| | | | | 2995) | | 8931) | | | 16327) |
| Clono- | 1 | TRA | AQSVDQPDAHVTLSEGASL | YSAAPY | LFWYVQYPGQSLQ | YITGDTV | KGTKGFEAEFRKSNSSFNLKKSPAHW | CALEGRNTGANTG | GHGTILRV |
| type_39 | | | ELRCSYS (SEQ ID NO: | (SEQ ID | FLLK (SEQ ID | V (SEQ | SDSAKYF (SEQ ID NO: 12152) | KLTF (SEQ ID | HP (SEQ |
| | | | 280) | NO: | NO: 6216) | ID NO: | | NO: 31166) | ID NO: |
| | | | | 3248) | | 9184) | | | 16356) |
| Clono- | 1 | TRB | GGIITQTPKFLIGQEGQKL | FNHDT | MYWYRQDSGKGLR | SITEND | LQKGDLSEGYDASREKKSSFSLTVTS | CASSIWDRNTEVF | GKGTRLTV |
| type_40 | | | TLKCQQN (SEQ ID NO: | (SEQ ID | LIYY (SEQ ID | (SEQ ID | AQKNEMAVFL (SEQ ID NO: | F (SEQ ID NO: | V (SEQ |
| | | | 27) | NO: | NO: 5963) | NO: | 11899) | 31167) | ID NO: |
| | | | | 2995) | | 8931) | | | 16333) |
| Clono- | 1 | TRA | AQKVTQTQTSISVMEKTTV | TRDSSYF | LFWYKQTASGEIV | QDSYKKE | ATEGHYSLNFQKPKSSIGLIITATQI | CAMREGPATGGNN | GQGTVLSV |
| type_40 | | | TMDCVYE (SEQ ID NO: | (SEQ ID | FLIR (SEQ ID | N (SEQ | EDSAVYF (SEQ ID NO: 11916) | KLTF (SEQ ID | IP (SEQ |
| | | | 44) | NO: | NO: 5942) | ID NO: | | NO: 31168) | ID NO: |
| | | | | 2974) | | 8910) | | | 16368) |
| Clono- | 1 | TRB | GGIITQTPKFLIGQEGQKL | FNHDT | MYWYRQDSGKGLR | SITEND | LQKGDLSEGYDASREKKSSFSLTVTS | CASSIGTEVFF | GKGTRLTV |
| type_41 | | | TLKCQQN (SEQ ID NO: | (SEQ ID | LIYY (SEQ ID | (SEQ ID | AQKNEMAVFL (SEQ ID NO: | (SEQ ID NO: | V (SEQ |
| | | | 27) | NO: | NO: 5963) | NO: | 11899) | 31169) | ID NO: |
| | | | | 2995) | | 8931) | | | 16333) |
| Clono- | 1 | TRA | GDSVTQTEGQVALSEEDFL | ASGYPT | LFWYVQYPGEGPQ | ASRDKEK | GSSRGFEATYDKGTTSFHLRKASVQE | CALSEFNGNEKIT | GAGTKLTI |
| type_41 | | | TIHCNYS (SEQ ID NO: | (SEQ ID | LLFR (SEQ ID | (SEQ ID | SDSAVYY (SEQ ID NO: 11886) | F (SEQ ID NO: 31170) | KP (SEQ |
| | | | 14) | NO: | NO: 5950) | NO: 8918) | | | ID NO: |
| | | | | 2982) | | | | | 16512) |
| Clono- | 1 | TRB | GGIITQTPKFLIGQEGQKL | FNHDT | MYWYRQDSGKGLR | SITEND | LQKGDLSEGYDASREKKSSFSLTVTS | CASSMGQGSDYTF | GSGTRLLV |
| type_42 | | | TLKCQQN (SEQ ID NO: | (SEQ ID | LIYY (SEQ ID | (SEQ ID | AQKNEMAVFL (SEQ ID NO: | (SEQ ID NO: 16225) | I (SEQ |
| | | | 27) | NO: | NO: 5963) | NO: 8931) | 11899) | | ID NO: |
| | | | | 2995) | | | | | 16337) |
| Clono- | 1 | TRA | GDSVTQTEGQVTVSESKSL | ATSIAYP | LFWYVRYPGEGLQ | VITAGQK | GSSRGFEATYNKETTSFHLQKASVQE | CALSENYAQGLTF | GLGTRVSV |
| type_42 | | | IINCTYS (SEQ ID NO: | N (SEQ | LLLK (SEQ ID | (SEQ ID | SDSAVYY (SEQ ID NO: 11932) | (SEQ ID NO: | FP (SEQ |
| | | | 60) | ID NO: | NO: 5996) | NO: 8964) | | 31172) | ID NO: |
| | | | | 3098) | | | | | 16344) |
| Clono- | 1 | TRB | GGIITQTPKFLIGQEGQKL | FNHDT | MYWYRQDSGKGLR | SITEND | LQKGDLSEGYDASREKKSSFSLTVTS | CASANRGADTQYF | GPGTRLLV |
| type_43 | | | TLKCQQN (SEQ ID NO: | (SEQ ID | LIYY (SEQ ID | (SEQ ID | AQKNEMAVFL (SEQ ID NO: | (SEQ ID NO: | L (SEQ |
| | | | 27) | NO: | NO: 5963) | NO: 8931) | 11899) | 31173) | ID NO: |
| | | | | 2995) | | | | | 16341) |
| Clono- | 1 | TRA | QQKVQQSPESLIVPEGAMT | DSASQY | FAWYRQHSGKAPK | IFSNGE | KEEGRFTIHLNKASLHFSLHIRDSQP | CAVSMGGYQNFYF | GKGTSLTV |
| type_43 | | | SLNCTFS (SEQ ID NO: | (SEQ ID | ALMS (SEQ ID | (SEQ ID | SDSALYL (SEQ ID NO: 11948) | (SEQ ID NO: 31174) | IP (SEQ |
| | | | 214) | NO: | NO: 6150) | NO: 8932) | | | ID NO: |
| | | | | 3044) | | | | | 16366) |
| Clono | 1 | TRB | VTLLEQNPRWRLVPRGQAV | NSQYPW | MSWYQQDLQKQLQ | LRSPGD | KEVKSLPGADYLATRVTDTELRLQVA | CTCRTGGGGSNER | GHGTKLSV |
| type_44 | | | NLRCILK (SEQ ID NO: | (SEQ ID | WLFT (SEQ ID | (SEQ ID | NMSQGRTLY (SEQ ID NO: | LFF (SEQ ID | L (SEQ |
| | | | 23) | NO: | NO: 5959) | NO: | 11895) | NO: 31175) | ID NO: |
| | | | | 2991) | | 8927) | | | 16401) |
| Clono- | 1 | TRA | SQLAEENLWALSVHEGESV | TSITA | LQWYRQKSGEGPA | IRSNERE | KRNGRLRATLDTSSQSSSLSITATRC | CGTGSGGKLTL | GTGTRLQV |
| type_44 | | | TVNCSYK (SEQ ID NO: | (SEQ ID | QLIL (SEQ ID | (SEQ ID | EDTAVYF (SEQ ID NO: 11896) | (SEQ ID NO: | NL (SEQ |
| | | | 190) | NO: | NO: 5960) | NO: | | 31176) | ID NO: |
| | | | | 2992) | | 8928) | | | 16448) |
| Clono- | 1 | TRB | VTLLEQNPRWRLVPRGQAV | NSQYPW | MSWYQQDLQKQLQ | LRSPGD | KEVKSLPGADYLATRVTDTELRLQVA | CTCSAGGQDTQYF | GPGTRLLV |
| type_45 | | | NLRCILK (SEQ ID NO: | (SEQ ID | WLFT (SEQ ID | (SEQ ID | NMSQGRTLY (SEQ ID NO: | (SEQ ID NO: | L (SEQ |
| | | | 23) | NO: | NO: 5959) | NO: 8927) | 11895) | 31177) | ID NO: |
| | | | | 2991) | | | | | 16341) |
| Clono- | 1 | TRA | GEQVEQLPSSLIVQEGASV | DSASVY | FPWYKQEPGKRLQ | IRSNMER | KQNQRLTLLFDKKTKHLSLHITATQP | CSASDGGSGNKLI | GTGTLLSV |
| type_45 | | | LINCSYT (SEQ ID NO: | (SEQ ID | FIID (SEQ ID | (SEQ ID | GDSAIYF (SEQ ID NO: 12032) | F (SEQ ID NO: | KP (SEQ |
| | | | 160) | NO: | NO: 6096) | NO: 8952) | | 31178) | ID NO: |
| | | | | 3128) | | | | | 16350) |
| Clono- | 1 | TRB | VTLLEQNPRWRLVPRGQAV | NSQYPW | MSWYQQDLQKQLQ | LRSPGD | KEVKSLPGADYLATRVTDTELRLQVA | CTCSAPGTGGYQN | GAGTRLSV |
| type_46 | | | NLRCILK (SEQ ID NO: | (SEQ ID | WLFT (SEQ ID | (SEQ ID | NMSQGRTLY (SEQ ID NO: | TLYF (SEQ ID | L (SEQ |
| | | | 23) | NO: | NO: 5959) | NO: 8927) | 11895) | NO: 31179) | ID NO: |
| | | | | 2991) | | | | | 16365) |
| Clono- | 1 | TRA | GEKVEQHESTLSVREGDSA | DTASSY | FPWYKQEAGKSLH | IRSNVDR | KQSQRLIVLLDKKAKRFSLHITATQP | CAASMWTGGYKVV | GSGTRLLV |
| type_46 | | | VINCTYT (SEQ ID NO: | (SEQ ID | FVID (SEQ ID | (SEQ ID | EDSAIYF (SEQ ID NO: 11926) | F (SEQ ID NO: 31180) | SP (SEQ |
| | | | 54) | NO: | NO: 5990) | NO: 8958) | | | ID NO: |
| | | | | 3022) | | | | | 16354) |
| Clono- | 1 | TRB | VTLLEQNPRWRLVPRGQAV | NSQYPW | MSWYQQDLQKQLQ | LRSPGD | KEVKSLPGADYLATRVTDTELRLQVA | CTCSAPGFQNTLY | GAGTRLSV |
| type_47 | | | NLRCILK (SEQ ID NO: | (SEQ ID | WLFT (SEQ ID | (SEQ ID | NMSQGRTLY (SEQ ID NO: | F (SEQ ID NO: | L (SEQ |
| | | | 23) | NO: | NO: 5959) | NO: | 11895) | 31181) | ID NO: |
| | | | | 2991) | | 8927) | | | 16365) |
| Clono- | 1 | TRA | GDSVTQKEGLVTLTEGLPV | TIYSNAF | LFWYVHYLNESPR | SSTDNKR | TEHQGFHATLHKSSSSFHLQKSSAQL | CALRDSGYNKLTF | GKGTVLLV |
| type_47 | | | MLNCTYQ (SEQ ID NO: | (SEQ ID | LLLK (SEQ ID | (SEQ ID | SDSALYY (SEQ ID NO: 11918) | (SEQ ID NO: 31182) | SP (SEQ |
| | | | 890) | NO: | NO: 6144) | NO: 8912) | | | ID NO: |
| | | | | 3858) | | | | | 16378) |
| Clono- | 1 | TRB | VTLLEQNPRWRLVPRGQAV | NSQYPW | MSWYQQDLQKQLQ | LRSPGD | KEVKSLPGADYLATRVTDTELRLQVA | CTCSGDWGAYEQY | GPGTRLTV |
| type_48 | | | NLRCILK (SEQ ID NO: | (SEQ ID | WLFT (SEQ ID | (SEQ ID | NMSQGRTLY (SEQ ID NO: | F (SEQ ID NO: | L (SEQ ID |
| | | | 23) | NO: | NO: 5959) | NO: | 11895) | 31183) | NO: |
| | | | | 2991) | | 8927) | | | 16327) |
| Clono- | 1 | TRA | GDSVTQTEGQVALSEEDFL | ASGYPT | LFWYVQYPGEGPQ | ASRDKEK | GSSRGFEATYDKGTTSFHLRKASVQE | CALRDNNNAPRF | GAGTKLSV |
| type_48 | | | TIHCNYS (SEQ ID NO: | (SEQ ID | LLFR (SEQ ID | (SEQ ID | SDSAVYY (SEQ ID NO: 11886) | (SEQ ID NO: | KP (SEQ |
| | | | 14) | NO: | NO: 5950) | NO: | | 31184) | ID NO: |
| | | | | 2982) | | 8918) | | | 16426) |

### Example 2: Isolation of ALK-Specific TCRs from Peripheral Blood of HLA-A*02:01 and HLA-B*07:02 Patients with NSCLC and Healthy Donors

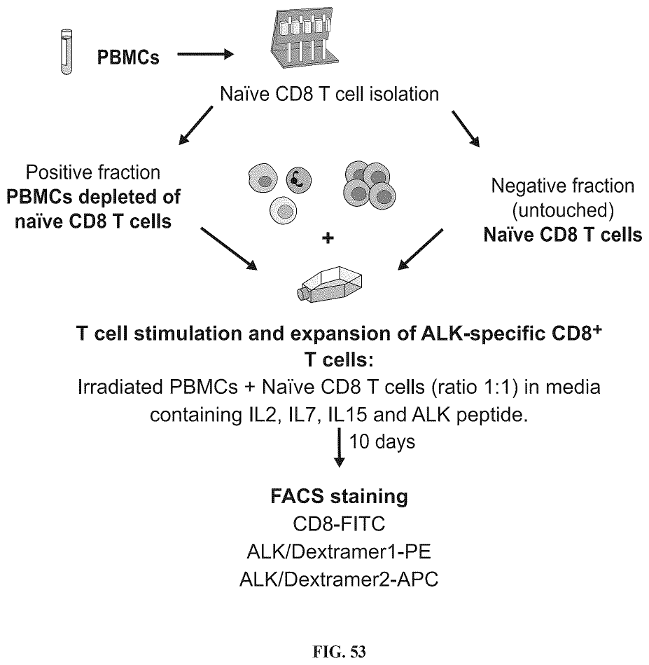

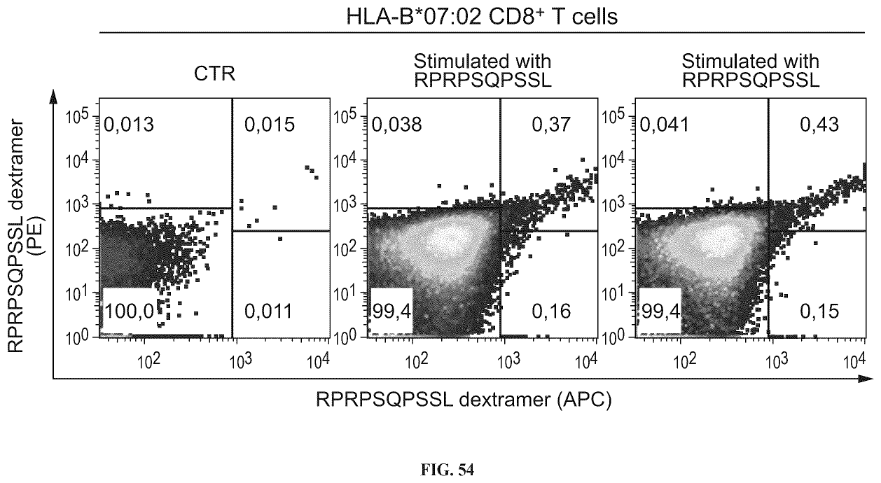

Data is generated supporting the feasibility of isolating CD8+ T cells reactive against the ALK peptide RPRPSQPSSL (SEQ ID NO: 32957) from peripheral blood of HLA-B*07:02 healthy donors. Untouched naïve CD8+ T cells are purified from PBMCs and stimulated in vitro with autologous irradiated antigen presenting cells at a ratio 1:1 and cultured in presence of 50 IU/ml IL2, 25 ng/ml IL7, 25 ng/ml IL15 and 1 uM RPRPSQPSSL (SEQ ID NO: 32957) peptide for 10 days. RPRPSQPSSL (SEQ ID NO: 32957) peptide and all the cytokines are replaced every 2-3 days (FIG. 53). The presence of CD8+ T cells reactive against RPRPSQPSSL (SEQ ID NO: 32957) are identified by staining the CD8+ T cells with two Dextramers® that bind the RPRPSQPSSL (SEQ ID NO: 32957) peptide with its MHC-I (HLA-B*0) 7:02) and are conjugated with different fluorochromes (FIG. 54).

To isolate TCRs recognizing human ALK peptides PBMCs are collected from HLA-A*02:01 and HLA-B*07:02 healthy donors and HLA-A*02:01 and HLA-B*07:02 patients with NSCLC, who consented to correlative IRB approved protocols (DF/HCC 02-180, 11-104 and 17-000). Autologous Dendritic Cells (DCs) are isolated from PBMCs by stimulation with GM-CSF and IL4 and DCs maturation is induced by using a synthetic analog of dsRNA known as polyinosinic-polycytidylic acid (poly I:C). In parallel, purified CD8+ T cells from PBMCs are cultured with autologous mature DCs (mDCs) pulsed with each human ALK peptide at a ratio 20:1 in the presence of human IL7, IL15 and IL12. Fourteen days later, a second round of CD8+T stimulation is performed with peptide-pulsed mDCs in the presence of human IL2. Fourteen days after the second stimulation, the presence of ALK-specific T cell responses is evaluated by IFNγ ELISPOT assay and CD8+ T cells are sorted by using human Dextramers that combined each human ALK peptide with the corresponding human MCH-I (HLA-B*07:02 or HLA-A*02:01). Sorted cells are subjected to single cell sequencing (10× Genomics®).

### Example 3: Isolation of ALK-Specific TCRs from Tumor Infiltrating Lymphocytes (TILs) of Patients with ALK Positive Tumors and HLA-A*02:01 or HLA-B*07:02 Genotype

To isolate TCRs recognizing human ALK peptides from TILs, tumor biopsies and/or tumor specimens from HLA-A*02:01 and HLA-B*07:02 patients with ALK+NSCLC who consented to correlative IRB-approved protocols (DF/HCC 02-180, 11-104 and 17-000) are collected. Tumor specimens are cut into small fragments that are plated in a 24 well plate to allow the TILs to migrate out of the fragments and expand individually in vitro. Tumor fragments are cultured in RPMI complete media supplemented with 10% human AB serum and 6000 IU/mL IL-2 and until T cell growth is observed. IL-2 and media is replaced every 2-3 days.

Next, TILs are expanded by using irradiated autologous PBMCs at a ratio of 1 to 100 in media supplemented with 5% human AB serum, 3000 IU/ml of IL-2 and 30 ng/mL OKT3 antibody and CD8+ T cells are enriched by negative selection with StemCell Technology™ reagents.

Autologous Dendritic Cells (DCs) are isolated from patient's PBMCs by stimulation with GM-CSF and IL4 and DCs maturation is induced by using a synthetic analog of dsRNA known as polyinosinic-polycytidylic acid (poly I:C).

Next, the enriched population of CD8+ T cells obtained by TILs is cultured with autologous mature DCs (mDCs) pulsed with each human ALK peptide at a ratio 20:1 in the presence of human IL7, IL15 and IL12. Fourteen days later, a second round of CD8+T stimulation is performed with peptide-pulsed mDCs in the presence of human IL-2. Fourteen days after the second stimulation, the presence of ALK-specific T cell responses is evaluated by IFNγ ELISPOT assay and CD8+ T cells are enriched by sorting with human Dextramers® that combine each RPRPSQPSSL (SEQ ID NO: 32957), IVRCIGVSL (SEQ ID NO: 32958) or AMLDLLHVA (SEQ ID NO: 32961) peptide with the human HLA-B*07:02 or HLA-A*02:01. Sorted cells are subjected to single cell sequencing (10× Genomics®).

### Example 4: Production of ALK-TCR T Cells

The paired α and β sequence of all the expanded ALK-specific clonotypes (VDJ count≥4) isolated from HLA-B*0) 7:02 mice vaccinated with RPRPSQPSSL (SEQ ID NO: 32957) was cloned into pMSCV-IRES-GFP II retroviral vector. The transcription of each ALK-TCR construct was driven by the endogenous 5′LTR of the pMSCV-IRES-GFP II retroviral vector.

Next, the CD3⁺ T cells were purified by negative selection from Peripheral Blood Mononuclear Cells of healthy donors (PMBCs) obtained from the Boston Children's Hospital Blood Donor Center. CD3⁺ T cells were activated in vitro with anti-CD3, and anti-CD28 beads (ratio cells:beads cells 1:1) in media supplemented with 10 ng/ml rhIL-7, and 5 ng/ml rhIL15 for two days. On day two 2.5×10\hat{ }⁵ CD3⁺ T cells were transduced with retroviral supernatants using Retronectin® coated plates prepared following manufacturer's instructions. On day five transduced CD3⁺ T cells were collected and expanded in vitro for eleven days in media supplemented with 10 ng/ml rhIL-7 and 5 ng/ml rhIL15 that were replaced every 2 days. On day eleven, CD8⁺ T cells were further purified from CD3⁺ T cells by depleting the CD4⁺ T cell population with positive selection and kept in culture for 2 more days in the absence of rhIL7 and rhIL15.

A set of ALK-TCR T cells in which endogenous TCRs were knocked out were also produced. Endogenous TCRs were knocked out in the T cells using the following crRNA guides to knock out the endogenous TRAC gene: AGAGTCTCTCAGCTGGTACA (SEQ ID NO: 32988) (Ren J. et al., Clin Cancer Res (2017) 23 (9); 2255-2266.) and the TRBC1 and TRBC2 genes: AGAGATCTCCCACACCCAAA (SEQ ID NO: 32986) (Morton L. T. et al., Molecular Therapy (2020) 28 (1): 64-74). Alt-R® CRISPR-Cas9 crRNA 200 μM was mixed with Alt-R® CRISPR-Cas9 tracrRNA 200 μM in a ratio of 1:1. RNAs were annealed by incubation at 95° for 5 minutes. 200 pmol of gRNA was then mixed with 120 pmol SpCas9 nuclease and cells were then treated with 4 μl of RNP. When the double knock-out of TRAC and TRBC1/2 was performed, TRAC and TRBC1/2 RNPs were prepared separately and then cells were treated with 4 μl of each RNP.

Next, CD3⁺ T cells were purified by negative selection from Peripheral Blood Mononuclear Cells of healthy donors (PMBCs) obtained from the Boston Children's Hospital Blood Donor Center. CD3⁺ T cells were activated in vitro with anti-CD3, and anti-CD28 beads (ratio cells:beads cells 1:1) in media supplemented with 10 ng/ml rhIL-7, and 5 ng/ml rhIL15 for two days. On day two anti-CD3 and anti-CD28 beads were magnetically removed, CD3⁺ T cells were washed with PBS and 1×10\hat{ }⁶ cells were resuspended in Lonza electroporation buffer P3 supplemented with 100 uM (100 pmol) of electroporation enhancer. Next, 4 μl of each RNP was added to the reaction mix. 23 μl of reaction mix was then transferred into the cuvette strips and CD3⁺ T cells were electroporated with 4D nucleofector (Lonza), program EO115. Immediately after electroporation, CD3⁺ T cells were incubated with an additional 100 μl of warm T cell media and were transferred into 48 well plates containing 300 μl of T-cell media supplemented with 10 ng/ml IL7 and 5 ng/ml IL15. On day three 2.5×10\hat{ }⁵ CD3⁺ T cells were transduced with retroviral supernatants using Retronectin® coated plates prepared following manufacturer's instructions. On day six transduced CD3⁺ T cells were collected and and expanded in vitro for nine days in media supplemented with 10 ng/ml rhIL-7 and 5 ng/ml rhIL15 that were replaced every 2 days. The knock-out efficiency was assessed on day nine by analyzing the expression of human TCRαβ with flow cytometry. On day twelve, CD8 T cells were further purified from CD3⁺ T cells by depleting the CD4⁺ T cell population with positive selection and kept in culture for 2 more days in the absence of rhIL7 and rhIL15.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of some embodiments herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The invention may be related to any of International Patent Application No. PCT/US20/51237, filed Sep. 17, 2020, International Patent Application No. PCT/US21/15519, filed Jan. 28, 2021, or U.S. Provisional Application No. 63/339,018, filed May 6, 2022, the entirety of each of which is incorporated herein by reference for all purposes.

What is claimed is:

1. A transgenic effector cell comprising a heterologous polynucleotide encoding a T cell receptor (TCR) polypeptide that specifically binds an anaplastic lymphoma kinase (ALK) peptide in the context of specific HLA molecules.

2. The transgenic effector cell of claim 1, wherein the ALK peptide has at least about 85% amino acid sequence identity to a peptide selected from the group consisting of: RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); AMLDLLHVA (SEQ ID NO: 32961); GGDLKSFLRETRPRPSQPSSLAMLDLLHVA (SEQ ID NO: 32962); FNHQNIVRCIGVSL (SEQ ID NO: 32963); and GGDLKSFLRETRPRPSQPSSLAM (SEQ ID NO: 32964).

3. The transgenic effector cell of claim 1, wherein the T cell receptor polypeptide comprises:
(i) a complementarity determining region (CDR) 1, wherein the sequence of CDR 1 is any CDR I sequence disclosed in SEQ ID NOs 2969-4452, 20513-21122, 28105-28240, 29793-29820, or 30321-30416, or any sequence that has at least 85% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least 85% identity thereto; and/or
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least 85% identity thereto.

4. A polypeptide that specifically binds an ALK peptide or an antigen binding fragment thereof, the polypeptide comprising:
(i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least 85% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least 85% identity thereto; and/or
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least 85% identity thereto.

5. The polypeptide of claim 4, wherein the ALK peptide has at least about 90% amino acid sequence identity to a peptide selected from the group consisting of RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); AMLDLLHVA (SEQ ID NO: 32961); GGDLKSFLRETRPRPSQPSSLAMLDLLHVA (SEQ ID NO: 32962); FNHQNIVRCIGVSL (SEQ ID NO: 32963); and GGDLKSFLRETRPRPSQPSSLAM (SEQ ID NO: 32964).

6. A polynucleotide encoding the polypeptide of claim 5.

7. A vector comprising the polynucleotide of claim 6.

8. The vector of claim 7, wherein the vector is a viral vector.

9. A pharmaceutical composition comprising an effector cell of claim 1, including a cell comprising a heterologous polynucleotide encoding a T-cell receptor (TCR) polypeptide that specifically binds an anaplastic lymphoma kinase (ALK) peptide.

10. The pharmaceutical composition of claim 9, wherein the ALK peptide has at least about 85% amino acid sequence identity to a peptide selected from the group consisting of RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); AMLDLLHVA (SEQ ID NO: 32961); GGDLKSFLRETRPRPSQPSSLAMLDLLHVA (SEQ ID NO: 32962); FNHQNIVRCIGVSL (SEQ ID NO: 32963); and GGDLKSFLRETRPRPSQPSSLAM (SEQ ID NO: 32964).

11. A pharmaceutical composition comprising an antibody that specifically binds an ALK peptide, or an antigen binding fragment thereof, the antibody comprising:
(i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least 85% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least 85% identity thereto; and/or
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least 85% identity thereto.

12. The pharmaceutical composition of claim 11, wherein the ALK peptide has at least about 85% amino acid sequence identity to a peptide selected from the group consisting of RPRPSQPSSL (SEQ ID NO: 32957); IVRCIGVSL (SEQ ID NO: 32958); VPRKNITLI (SEQ ID NO: 32959); TAAEVSVRV (SEQ ID NO: 32960); AMLDLLHVA (SEQ ID NO: 32961); GGDLKSFLRETRPRPSQPSSLAMLDLLHVA (SEQ ID NO: 32962); FNHQNIVRCIGVSL (SEQ ID NO: 32963); and GGDLKSFLRETRPRPSQPSSLAM (SEQ ID NO: 32964).

13. A method for treating a subject having an ALK-positive neoplasia or an ALK-positive neoplasia that is resistant to ALK tyrosine kinase inhibitor therapy, the method comprising administering to the subject an effective amount of a transgenic effector cell of claim 1, thereby treating the subject.

14. A method for killing an ALK-positive neoplastic cell, reducing or inhibiting the growth of an ALK positive neoplasia, or reducing metastasis or inhibiting the development of metastasis in a subject having an ALK-positive neoplasia, the method comprising administering to the subject a transgenic effector cell comprising a heterologous polynucleotide encoding a T-cell receptor (TCR) polypeptide that specifically binds an anaplastic lymphoma kinase (ALK) peptide, thereby reducing or inhibiting the development of metastasis in the subject.

15. A method for treating an human leukocyte antigen (HLA) serotype A*02:01 subject, HLA-B*07:02 subject, having an anaplastic lymphoma kinase (ALK)-rearranged Non-Small Cell Lung Cancer (NSCLC) or anaplastic large cell lymphoma (ALCL) or one of said neoplasias that is resistant to ALK tyrosine kinase inhibitor therapy, the method comprising administering to the subject a transgenic effector cell of claim 1, including a cell comprising a heterologous polynucleotide encoding a T-cell receptor (TCR) polypeptide that specifically binds an anaplastic lymphoma kinase (ALK) peptide, thereby treating the subject.

16. The method of claim 15, wherein the method reduces or otherwise treats metastasis in the subject.

17. The method of claim 15, wherein the ALK rearrangement is an echinoderm microtubule-associate protein-like 4-ALK rearrangement (EML4-ALK).

18. A method of killing or reducing the growth of an ALK-positive neoplastic cell, the method comprising contacting the ALK-positive neoplastic cell with a transgenic effector cell comprising a heterologous polynucleotide encoding a TCR polypeptide that specifically binds an ALK peptide,
wherein the TCR polypeptide comprises each of:
(i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least 85% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least 85% identity thereto; and
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least 85% identity thereto.

19. A method of treating or reducing the growth of an ALK-positive neoplasia in a selected subject, the method comprising contacting the ALK-positive neoplasia with a transgenic effector cell comprising a heterologous polynucleotide encoding a TCR polypeptide that specifically binds an ALK peptide,
wherein the TCR polypeptide comprises each of:
(i) a complementarity determining region (CDR) 1, wherein the sequence of CDR 1 is any CDR 1 sequence disclosed in SEQ ID NOs 2969-4452, 20513-21122, 28105-28240, 29793-29820, or 30321-30416, or any sequence that has at least 85% identity thereto;
(ii) a CDR 2, wherein the sequence of CDR 2 is any CDR 2 sequence disclosed in SEQ ID NOs 8905-10388, 22953-23562, 28649-28784, 29905-29932, or 30705-30800, or any sequence that has at least 85% identity thereto; and
(iii) a CDR 3, wherein the sequence of CDR 3 is any CDR 3 sequence disclosed in SEQ ID NOs 14841-16324, 25393-26002, 29193-29328, 30017-30044, or 31089-31184, or any sequence that has at least 85% identity thereto,
and wherein the subject is selected for expression of an HLA-A*02:01 serotype or is selected for expression of an HLA-B*07:02 serotype.

20. A kit comprising an agent for administration to a subject with one or more ALK-positive cancers, wherein the agent comprises the pharmaceutical composition of claim 9.
